SEQUENCE LISTING (114 sequences)

SEQ ID NO: 1 - DNA, 2313 bp, Chicken anemia virus
cgagtggttactattccatcaccattctagcctgtacacagaaagtcaagatggacgaatcgctcgacttcgctcgcgattcgtcgaaggcggggggccggaggccccccggtggcccccctccaacgagtggagcacgtacaggggggtacgtcatccgtacaggggggtacgtcatccgtacaggggggtacgtcacaaagaggcgttcccgtacaggggggtacgtcacgcgtacaggggggtacgtcacagccaatcaaaagctgccacgttgcgaaagtgacgtttcgaaaatgggcggcgcaagcctctctatatattgagcgcacataccggtcggcagtaggtatacgcaaggcggtccgggtggatgcacgggaacggcggacaaccggccgctgggggcagtgaatcggcgcttagccgagaggggcaacctgggcccagcggagccgcgcaggggcaagtaatttcaaatgaacgctctccaagaagatactccacccggaccatcaacggtgttcaggccaccaacaagttcacggccgttggaaacccctcactgcagagagatccggattggtatcgctggaattacaatcactctatcgctgtgtggctgcgcgaatgctcgcgctcccacgctaagatctgcaactgcggacaattcagaaagcactggtttcaagaatgtgccggacttgaggaccgatcaacccaagcctccctcgaagaagcgatcctgcgacccctccgagtacagggtaagcgagctaaaagaaagcttgattaccactactcccagccgaccccgaaccgcaaaaaggcgtataagactgtaagatggcaagacgagctcgcagaccgagaggccgattttactccttcagaagaggacggtggcaccacctcaagcgacttcgacgaagatataaatttcgacatcggaggagacagcggtatcgtagacgagcttttaggaaggcctttcacaacccccgccccggtacgtatagtgtgaggctgccgaacccccaatctactatgactatccgcttccaaggggtcatctttctcacggaaggactcattctgcctaaaaacagcacagcggggggctatgcagaccacatgtacggggcgagagtcgccaagatctctgtgaacctgaaagagttcctgctagcctcaatgaacctgacatacgtgagcaaaatcggaggccccatcgccggtgagttgattgcggacgggtctaaatcacaagccgcggacaattggcctaattgctggctgccgctagataataacgtgccctccgctacaccatcggcatggtggagatgggccttaatgatgatgcagcccacggactcttgccggttctttaatcacccaaagcagatgaccctgcaagacatgggtcgcatgtttgggggctggcacctgttccgacacattgaaacccgctttcagctccttgccactaagaatgagggatccttcagccccgtggcgagtcttctctcccagggagagtacctcacgcgtcgggacgatgttaagtacagcagcgatcaccagaaccggtggcaaaaaggcggacaaccgatgacggggggcattgcttatgcgaccgggaaaatgagacccgacgagcaacagtaccctgctatgcccccagaccccccgatcatcaccgctactacagcgcaaggcacgcaagtccgctgcatgaatagcacgcaagcttggtggtcatgggacacatatatgagctttgcaacactcacagcactcggtgcacaatggtcttttcctccagggcaacgttcagtttctagacggtccttcaaccaccacaaggcgagaggagccggggaccccaagggccagagatggcacacgctggtgccgctcggcacggagaccatcaccgacagctacatgtcagcacccgcatcagagctggacactaatttctttacgctttacgtagcgcaaggcacaaataagtcgcaacagtacaagttcggcacagctacatacgcgctaaaggagccggtaatgaagagcgatgcatgggcagtggtacgcgtccagtcggtctggcagctgggtaacaggcagaggccatacccatgggacgtcaactgggcgaacagcaccatgtactgggggacgcagccctgaaaaggggggggggctaaagccccccccccttaaacccccccctgggggggattcccccccagaccccccctttatatagcactcaataaacgcagaaaatagatttatcgcactatc

SEQ ID NO: 2 - DNA, 2319 bp, Artificial Sequence
  Description of Artificial Sequence: Synthetic polynucleotide
gaattcctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctgccaccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaataagatacattgatgagtttggacaaaccacaactagaatgcagtgaaaaaaatgctttatttgtgaaatttgtgatgctattgctttatttgtaaccattataagctgcaataaacaagttcctttcacaacccccgccccggtacgtatagtgtgaggctgccgaacccccaatctactatgactatccgcttccaaggggtcatctttctcacggaaggactcattctgcctaaaaacagcacagcggggggctatgcagaccacatgtacggggcgagagtcgccaagatctctgtgaacctgaaagagttcctgctagcctcaatgaacctgacatacgtgagcaaaatcggaggccccatcgccggtgagttgattgcggacgggtctaaatcacaagccgcggacaattggcctaattgctggctgccgctagataataacgtgccctccgctacaccatcggcatggtggagatgggccttaatgatgatgcagcccacggactcttgccggttctttaatcacccaaagcagatgaccctgcaagacatgggtcgcatgtttgggggctggcacctgttccgacacattgaaacccgctttcagctccttgccactaagaatgagggatccttcagccccgtggcgagtcttctctcccagggagagtacctcacgcgtcgggacgatgttaagtacagcagcgatcaccagaaccggtggcaaaaaggcggacaaccgatgacggggggcattgcttatgcgaccgggaaaatgagacccgacgagcaacagtaccctgctatgcccccagaccccccgatcatcaccgctactacagcgcaaggcacgcaagtccgctgcatgaatagcacgcaagcttggtggtcatgggacacatatatgagctttgcaacactcacagcactcggtgcacaatggtcttttcctccagggcaacgttcagtttctagacggtccttcaaccaccacaaggcgagaggagccggggaccccaagggccagagatggcacacgctggtgccgctcggcacggagaccatcaccgacagctacatgtcagcacccgcatcagagctggacactaatttctttacgctttacgtagcgcaaggcacaaataagtcgcaacagtacaagttcggcacagctacatacgcgctaaaggagccggtaatgaagagcgatgcatgggcagtggtacgcgtccagtcggtctggcagctgggtaacaggcagaggccatacccatgggacgtcaactgggcgaacagcaccatgtactgggggacgcagccctgaaaaggggggggggctaaagccccccccccttaaacccccccctgggggggattcccccccagaccccccctttatatagcactcaataaacgcagaaaatagatttatcgcactatc

SEQ ID NO: 3 - DNA, 2319 bp, Artificial Sequence
  Description of Artificial Sequence: Synthetic polynucleotide
gaattccgagtggttactattccatcaccattctagcctgtacacagaaagtcaagatggacgaatcgctcgacttcgctcgcgattcgtcgaaggcggggggccggaggccccccggtggcccccctccaacgagtggagcacgtacaggggggtacgtcatccgtacaggggggtacgtcatccgtacaggggggtacgtcacactgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctgccaccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaataagatacattgatgagtttggacaaaccacaactagaatgcagtgaaaaaaatgctttatttgtgaaatttgtgatgctattgctttatttgtaaccattataagctgcaataaacaagttgcctcaatgaacctgacatacgtgagcaaaatcggaggccccatcgccggtgagttgattgcggacgggtctaaatcacaagccgcggacaattggcctaattgctggctgccgctagataataacgtgccctccgctacaccatcggcatggtggagatgggccttaatgatgatgcagcccacggactcttgccggttctttaatcacccaaagcagatgaccctgcaagacatgggtcgcatgtttgggggctggcacctgttccgacacattgaaacccgctttcagctccttgccactaagaatgagggatccttcagccccgtggcgagtcttctctcccagggagagtacctcacgcgtcgggacgatgttaagtacagcagcgatcaccagaaccggtggcaaaaaggcggacaaccgatgacggggggcattgcttatgcgaccgggaaaatgagacccgacgagcaacagtaccctgctatgcccccagaccccccgatcatcaccgctactacagcgcaaggcacgcaagtccgctgcatgaatagcacgcaagcttggtggtcatgggacacatatatgagctttgcaacactcacagcactcggtgcacaatggtcttttcctccagggcaacgttcagtttctagacggtccttcaaccaccacaaggcgagaggagccggggaccccaagggccagagatggcacacgctggtgccgctcggcacggagaccatcaccgacagctacatgtcagcacccgcatcagagctggacactaatttctttacgctttacgtagcgcaaggcacaaataagtcgcaacagtacaagttcggcacagctacatacgcgctaaaggagccggtaatgaagagcgatgcatgggcagtggtacgcgtccagtcggtctggcagctgggtaacaggcagaggccatacccatgggacgtcaactgggcgaacagcaccatgtactgggggacgcagccctgaaaaggggggggggctaaagccccccccccttaaacccccccctgggggggattcccccccagaccccccctttatatagcactcaataaacgcagaaaatagatttatcgcactatc

SEQ ID NO: 4 - DNA, 2319 bp, Artificial Sequence
  Description of Artificial Sequence: Synthetic polynucleotide
gaattccgagtggttactattccatcaccattctagcctgtacacagaaagtcaagatggacgaatcgctcgacttcgctcgcgattcgtcgaaggcggggggccggaggccccccggtggcccccctccaacgagtggagcacgtacaggggggtacgtcatccgtacaggggggtacgtcatccgtacaggggggtacgtcacaaagaggcgttcccgtacaggggggtacgtcacgcgtacaggggggtacgtcacagccaatcaaaagctgccacgttgcgaaagtgacgtttcgaaaatgggcggcgcaagcctctctatatattgagcgcacataccggtcggcagtaggtatacgcaaggcggtccgggtggttgcacgggaacggcggacaaccggccctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctgccaccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaataagatacattgatgagtttggacaaaccacaactagaatgcagtgaaaaaaatgctttatttgtgaaatttgtgatgctattgctttatttgtaaccattataagctgcaataaacaagttctttaatcacccaaagcagatgaccctgcaagacatgggtcgcatgtttgggggctggcacctgttccgacacattgaaacccgctttcagctccttgccactaagaatgagggatccttcagccccgtggcgagtcttctctcccagggagagtacctcacgcgtcgggacgatgttaagtacagcagcgatcaccagaaccggtggcaaaaaggcggacaaccgatgacggggggcattgcttatgcgaccgggaaaatgagacccgacgagcaacagtaccctgctatgcccccagaccccccgatcatcaccgctactacagcgcaaggcacgcaagtccgctgcatgaatagcacgcaagcttggtggtcatgggacacatatatgagctttgcaacactcacagcactcggtgcacaatggtcttttcctccagggcaacgttcagtttctagacggtccttcaaccaccacaaggcgagaggagccggggaccccaagggccagagatggcacacgctggtgccgctcggcacggagaccatcaccgacagctacatgtcagcacccgcatcagagctggacactaatttctttacgctttacgtagcgcaaggcacaaataagtcgcaacagtacaagttcggcacagctacatacgcgctaaaggagccggtaatgaagagcgatgcatgggcagtggtacgcgtccagtcggtctggcagctgggtaacaggcagaggccatacccatgggacgtcaactgggcgaacagcaccatgtactgggggacgcagccctgaaaaggggggggggctaaagccccccccccttaaacccccccctgggggggattcccccccagaccccccctttatatagcactcaataaacgcagaaaatagatttatcgcactatc

SEQ ID NO: 5 - DNA, 2319 bp, Artificial Sequence
  Description of Artificial Sequence: Synthetic polynucleotide
gaattccgagtggttactattccatcaccattctagcctgtacacagaaagtcaagatggacgaatcgctcgacttcgctcgcgattcgtcgaaggcggggggccggaggccccccggtggcccccctccaacgagtggagcacgtacaggggggtacgtcatccgtacaggggggtacgtcatccgtacaggggggtacgtcacaaagaggcgttcccgtacaggggggtacgtcacgcgtacaggggggtacgtcacagccaatcaaaagctgccacgttgcgaaagtgacgtttcgaaaatgggcggcgcaagcctctctatatattgagcgcacataccggtcggcagtaggtatacgcaaggcggtccgggtggttgcacgggaacggcggacaaccggccgctgggggcagtgaatcggcgcttagccgagaggggcaacctgggcccagcggagccgcgcaggggcaagtaatttcaattgaacgctctccaagaagatactccacccggaccatcaacggtgttcaggccaccaacaagttcacggccgttggaaacccctcactgcagagagatccggattggtatcgctggaattactgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctgccaccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaataagatacattgatgagtttggacaaaccacaactagaatgcagtgaaaaaaatgctttatttgtgaaatttgtgatgctattgctttatttgtaaccattataagctgcaataaacaagttaccggtggcaaaaaggcggacaaccgatgacggggggcattgcttatgcgaccgggaaaatgagacccgacgagcaacagtaccctgctatgcccccagaccccccgatcatcaccgctactacagcgcaaggcacgcaagtccgctgcatgaatagcacgcaagcttggtggtcatgggacacatatatgagctttgcaacactcacagcactcggtgcacaatggtcttttcctccagggcaacgttcagtttctagacggtccttcaaccaccacaaggcgagaggagccggggaccccaagggccagagatggcacacgctggtgccgctcggcacggagaccatcaccgacagctacatgtcagcacccgcatcagagctggacactaatttctttacgctttacgtagcgcaaggcacaaataagtcgcaacagtacaagttcggcacagctacatacgcgctaaaggagccggtaatgaagagcgatgcatgggcagtggtacgcgtccagtcggtctggcagctgggtaacaggcagaggccatacccatgggacgtcaactgggcgaacagcaccatgtactgggggacgcagccctgaaaaggggggggggctaaagccccccccccttaaacccccccctgggggggattcccccccagaccccccctttatatagcactcaataaacgcagaaaatagatttatcgcactatc

SEQ ID NO: 6 - DNA, 2319 bp, Artificial Sequence
  Description of Artificial Sequence: Synthetic polynucleotide
gaattccgagtggttactattccatcaccattctagcctgtacacagaaagtcaagatggacgaatcgctcgacttcgctcgcgattcgtcgaaggcggggggccggaggccccccggtggcccccctccaacgagtggagcacgtacaggggggtacgtcatccgtacaggggggtacgtcatccgtacaggggggtacgtcacaaagaggcgttcccgtacaggggggtacgtcacgcgtacaggggggtacgtcacagccaatcaaaagctgccacgttgcgaaagtgacgtttcgaaaatgggcggcgcaagcctctctatatattgagcgcacataccggtcggcagtaggtatacgcaaggcggtccgggtggttgcacgggaacggcggacaaccggccgctgggggcagtgaatcggcgcttagccgagaggggcaacctgggcccagcggagccgcgcaggggcaagtaatttcaattgaacgctctccaagaagatactccacccggaccatcaacggtgttcaggccaccaacaagttcacggccgttggaaacccctcactgcagagagatccggattggtatcgctggaattacaatcactctatcgctgtgtggctgcgcgaatgctcgcgctcccacgctaagatctgcaactgcggacaattcagaaagcactggtttcaagaatgtgccggacttgaggaccgatcaacccaagcctccctcgaagaagcgatcctgcgacccctccgagtacagggtaagcgagctaaaagaaagcttgattaccactctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctgccaccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaataagatacattgatgagtttggacaaaccacaactagaatgcagtgaaaaaaatgctttatttgtgaaatttgtgatgctattgctttatttgtaaccattataagctgcaataaacaagttacactcacagcactcggtgcacaatggtcttttcctccagggcaacgttcagtttctagacggtccttcaaccaccacaaggcgagaggagccggggaccccaagggccagagatggcacacgctggtgccgctcggcacggagaccatcaccgacagctacatgtcagcacccgcatcagagctggacactaatttctttacgctttacgtagcgcaaggcacaaataagtcgcaacagtacaagttcggcacagctacatacgcgctaaaggagccggtaatgaagagcgatgcatgggcagtggtacgcgtccagtcggtctggcagctgggtaacaggcagaggccatacccatgggacgtcaactgggcgaacagcaccatgtactgggggacgcagccctgaaaaggggggggggctaaagccccccccccttaaacccccccctgggggggattcccccccagaccccccctttatatagcactcaataaacgcagaaaatagatttatcgcactatc

SEQ ID NO: 7 - DNA, 2319 bp, Artificial Sequence
  Description of Artificial Sequence: Synthetic polynucleotide
gaattccgagtggttactattccatcaccattctagcctgtacacagaaagtcaagatggacgaatcgctcgacttcgctcgcgattcgtcgaaggcggggggccggaggccccccggtggcccccctccaacgagtggagcacgtacaggggggtacgtcatccgtacaggggggtacgtcatccgtacaggggggtacgtcacaaagaggcgttcccgtacaggggggtacgtcacgcgtacaggggggtacgtcacagccaatcaaaagctgccacgttgcgaaagtgacgtttcgaaaatgggcggcgcaagcctctctatatattgagcgcacataccggtcggcagtaggtatacgcaaggcggtccgggtggttgcacgggaacggcggacaaccggccgctgggggcagtgaatcggcgcttagccgagaggggcaacctgggcccagcggagccgcgcaggggcaagtaatttcaattgaacgctctccaagaagatactccacccggaccatcaacggtgttcaggccaccaacaagttcacggccgttggaaacccctcactgcagagagatccggattggtatcgctggaattacaatcactctatcgctgtgtggctgcgcgaatgctcgcgctcccacgctaagatctgcaactgcggacaattcagaaagcactggtttcaagaatgtgccggacttgaggaccgatcaacccaagcctccctcgaagaagcgatcctgcgacccctccgagtacagggtaagcgagctaaaagaaagcttgattaccactactcccagccgaccccgaaccgcaaaaaggcgtataagactgtaagttggcaagacgagctcgcagaccgagaggccgattttactccttcagaagaggacggtggcaccacctcaagcgacttcgacgaagatataaatttcgacatcggaggagacagcggtatcgtagacgagcttttaggaaggcctttcacaaccctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctgccaccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaataagatacattgatgagtttggacaaaccacaactagaatgcagtgaaaaaaatgctttatttgtgaaatttgtgatgctattgctttatttgtaaccattataagctgcaataaacaagtttacgctttacgtagcgcaaggcacaaataagtcgcaacagtacaagttcggcacagctacatacgcgctaaaggagccggtaatgaagagcgatgcatgggcagtggtacgcgtccagtcggtctggcagctgggtaacaggcagaggccatacccatgggacgtcaactgggcgaacagcaccatgtactgggggacgcagccctgaaaaggggggggggctaaagccccccccccttaaacccccccctgggggggattcccccccagaccccccctttatatagcactcaataaacgcagaaaatagatttatcgcactatc

SEQ ID NO: 8 - DNA, 2319 bp, Artificial Sequence
  Description of Artificial Sequence: Synthetic polynucleotide
gaattccgagtggttactattccatcaccattctagcctgtacacagaaagtcaagatggacgaatcgctcgacttcgctcgcgattcgtcgaaggcggggggccggaggccccccggtggcccccctccaacgagtggagcacgtacaggggggtacgtcatccgtacaggggggtacgtcatccgtacaggggggtacgtcacaaagaggcgttcccgtacaggggggtacgtcacgcgtacaggggggtacgtcacagccaatcaaaagctgccacgttgcgaaagtgacgtttcgaaaatgggcggcgcaagcctctctatatattgagcgcacataccggtcggcagtaggtatacgcaaggcggtccgggtggttgcacgggaacggcggacaaccggccgctgggggcagtgaatcggcgcttagccgagaggggcaacctgggcccagcggagccgcgcaggggcaagtaatttcaattgaacgctctccaagaagatactccacccggaccatcaacggtgttcaggccaccaacaagttcacggccgttggaaacccctcactgcagagagatccggattggtatcgctggaattacaatcactctatcgctgtgtggctgcgcgaatgctcgcgctcccacgctaagatctgcaactgcggacaattcagaaagcactggtttcaagaatgtgccggacttgaggaccgatcaacccaagcctccctcgaagaagcgatcctgcgacccctccgagtacagggtaagcgagctaaaagaaagcttgattaccactactcccagccgaccccgaaccgcaaaaaggcgtataagactgtaagttggcaagacgagctcgcagaccgagaggccgattttactccttcagaagaggacggtggcaccacctcaagcgacttcgacgaagatataaatttcgacatcggaggagacagcggtatcgtagacgagcttttaggaaggcctttcacaacccccgccccggtacgtatagtgtgaggctgccgaacccccaatctactatgactatccgcttccaaggggtcatctttctcacggaaggactcattctgcctaaaaacagcacagcggggggctatgcagaccacatgtacggggcgagagtcgccaagatctctgtgaacctgaaagagttcctgctagcctcaatgaacctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctgccaccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaataagatacattgatgagtttggacaaaccacaactagaatgcagtgaaaaaaatgctttatttgtgaaatttgtgatgctattgctttatttgtaaccattataagctgcaataaacaagttagccctgaaaaggggggggggctaaagccccccccccttaaacccccccctgggggggattcccccccagaccccccctttatatagcactcaataaacgcagaaaatagatttatcgcactatc

SEQ ID NO: 9 - DNA, 2259 bp, Artificial Sequence
  Description of Artificial Sequence: Synthetic polynucleotide
gaattccgagtggttactattccatcaccattctagcctgtacacagaaagtcaagatggacgaatcgctcgacttcgctcgcgattcgtcgaaggcggggggccggaggccccccggtggcccccctccaacgagtggagcacgtacaggggggtacgtcatccgtacaggggggtacgtcatccgtacaggggggtacgtcacaaagaggcgttcccgtacaggggggtacgtcacgcgtacaggggggtacgtcacagccaatcaaaagctgccacgttgcgaaagtgacgtttcgaaaatgggcggcgcaagcctctctatatattgagcgcacataccggtcggcagtaggtatacgcaaggcggtccgggtggatgcacgggaacggcggacaaccggccgctgggggcagtgaatcggcgcttagccgagaggggcaacctgggcccagcggagccgcgcaggggcaagtaatttcaaatgaacgctctccaagaagatactccacccggaccatcaacggtgttcaggccaccaacaagttcacggccgttggaaacccctcactgcagagagatccggattggtatcgctggaattacaatcactctatcgctgtgtggctgcgcgaatgctcgcgctcccacgctaagatctgcaactgcggacaattcagaaagcactggtttcaagaatgtgccggacttgaggaccgatcaacccaagcctccctcgaagaagcgatcctgcgacccctccgagtacagggtaagcgagctaaaagaaagcttgattaccactactcccagccgaccccgaaccgcaaaaaggcgtataagactgtaagatggcaagacgagctcgcagaccgagaggccgattttactccttcagaagaggacggtggcaccacctcaagcgacttcgacgaagatataaatttcgacatcggaggagacagcggtatcgtagacgagcttttaggaaggcctttcacaacccccgccccggtacgtatagtgtgaggctgccgaacccccaatctactatgactatccgcttccaaggggtcatctttctcacggaaggactcattctgcctaaaaacagcacagcggggggctatgcagaccacatgtacggggcgagagtcgccaagatctctgtgaacctgaaagagttcctgctagcctcaatgaacctgacatacgtgagcaaaatcggaggccccatcgccggtgagttgattgcggacgggtctaaatcctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctgccaccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaataagatacattgatgagtttggacaaaccacaactagaatgcagtgaaaaaaatgctttatttgtgaaatttgtgatgctattgctttatttgtaaccattataagctgcaataaacaagtt

SEQ ID NO: 10 - DNA, 2259 bp, Artificial Sequence
  Description of Artificial Sequence: Synthetic polynucleotide
gaattccgagtggttactattccatcaccattctagcctgtacacagaaagtcaagatggacgaatcgctcgacttcgctcgcgattcgtcgaaggcggggggccggaggccccccggtggcccccctccaacgagtggagcacgtacaggggggtacgtcatccgtacaggggggtacgtcatccgtacaggggggtacgtcacaaagaggcgttcccgtacaggggggtacgtcacgcgtacaggggggtacgtcacagccaatcaaaagctgccacgttgcgaaagtgacgtttcgaaaatgggcggcgcaagcctctctatatattgagcgcacataccggtcggcagtaggtatacgcaaggcggtccgggtggttgcacgggaacggcggacaaccggccgctgggggcagtgaatcggcgcttagccgagaggggcaacctgggcccagcggagccgcgcaggggcaagtaatttcaattgaacgctctccaagaagatactccacccggaccatcaacggtgttcaggccaccaacaactgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctgccaccatggtgcccaagaagaagaggaaagtctccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaaaaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgcgccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacatagtgatgaactacatcagaaacctggactctgagactggggccatggtgaggctgctcgaggatggggactgataagatacattgatgagtttggacaaaccacaactagaatgcagtgaaaaaaatgctttatttgtgaaatttgtgatgctattgctttatttgtaaccattataagctgcaataaacaagttggcagaggccatacccatgggacgtcaactgggcgaacagcaccatgtactgggggacgcagccctgaaaaggggggggggctaaagccccccccccttaaacccccccctgggggggattcccccccagaccccccctttatatagcactcaataaacgcagaaaatagatttatcgcactatc

SEQ ID NO: 11 - DNA, 2319 bp, Artificial Sequence
  Description of Artificial Sequence: Synthetic polynucleotide
gaattccgagtggttactattccatcaccattctagcctgtacacagaaagtcaagatggacgaatcgctcgacttcgctcgcgattcgtcgaaggcggggggccggaggccccccggtggcccccctccaacgagtggagcacgtacaggggggtacgtcatccgtacaggggggtacgtcatccgtacaggggggtacgtcacaaagaggcgttcccgtacaggggggtacgtcacgcgtacaggggggtacgtcacagccaatcaaaagctgccacgttgcgaaagtgacgtttcgaaaatgggcggcgcaagcctctctatatattgagcgcacataccggtcggcagtaggtatacgcaaggcggtccgggtggttgcacgggaacggcggacaaccggccgctgggggcagtgaatcggcgcttagccgagaggggcaacctgggcccagcggagccgcgcaggggcaagtaatttcaattgaacgctctccaagaagatactccacccggaccatcaacggtgttcaggccaccaacaagttcacggccgttggaaacccctcactgcagagagatccggattggtatcgctggaattactgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagatacattgatgagtttggacaaaccacaactagaatgcagtgaaaaaaatgctttatttgtgaaatttgtgatgctattgctttatttgtaaccattataagctgcaataaacaagtttcacagcactcggtgcacaatggtcttttcctccagggcaacgttcagtttctagacggtccttcaaccaccacaaggcgagaggagccggggaccccaagggccagagatggcacacgctggtgccgctcggcacggagaccatcaccgacagctacatgtcagcacccgcatcagagctggacactaatttctttacgctttacgtagcgcaaggcacaaataagtcgcaacagtacaagttcggcacagctacatacgcgctaaaggagccggtaatgaagagcgatgcatgggcagtggtacgcgtccagtcggtctggcagctgggtaacaggcagaggccatacccatgggacgtcaactgggcgaacagcaccatgtactgggggacgcagccctgaaaaggggggggggctaaagccccccccccttaaacccccccctgggggggattcccccccagaccccccctttatatagcactcaataaacgcagaaaatagatttatcgcactatc

SEQ ID NO: 12 - DNA, 2319 bp, Artificial Sequence
  Description of Artificial Sequence: Synthetic polynucleotide
gaattccgagtggttactattccatcaccattctagcctgtacacagaaagtcaagatggacgaatcgctcgacttcgctcgcgattcgtcgaaggcggggggccggaggccccccggtggcccccctccaacgagtggagcacgtacaggggggtacgtcatccgtacaggggggtacgtcatccgtacaggggggtacgtcacaaagaggcgttcccgtacaggggggtacgtcacgcgtacaggggggtacgtcacagccaatcaaaagctgccacgttgcgaaagtgacgtttcgaaaatgggcggcgcaagcctctctatatattgagcgcacataccggtcggcagtaggtatacgcaaggcggtccgggtggttgcacgggaacggcggacaaccggccgctgggggcagtgaatcggcgcttagccgagaggggcaacctgggcccagcggagccgcgcaggggcaagtaatttcaattgaacgctctccaagaagatactccacccggaccatcaacggtgttcaggccaccaacaagttcacggccgttggaaacccctcactgcagagagatccggattggtatcgctggaattactgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctgccaccatggcgtctaaagtgtatgacccagagcagagaaaacgaatgatcaccggcccccaatggtgggctcggtgcaagcagatgaatgtgcttgatagctttataaactactatgatagtgagaaacacgcagaaaatgccgtgatattcttgcatgggaacgcagcatcttcttacctttggaggcatgttgttccacatatcgaaccagtagccaggtgcatcatcccagatttgattggtatggggaaatctggcaagagtggcaacggtagttatcgactgcttgaccattataagtatctgaccgcctggttcgagctccttaaccttccaaaaaaaataatttttgtgggccatgactggggcgcatgtcttgcattccactacagttatgaacatcaagataagattaaggccatagtacacgcagagtccgtggttgacgttatcgagtcttgggatgaatggcctgatattgaagaggacatagcacttattaagagcgaggagggagagaagatggttcttgaaaacaacttttttgtcgaaaccatgttgccaagcaagataatgcgaaagctggaaccagaggaattcgcagcctatcttgagccgttcaaggagaaaggggaggtaagacgcccgacattgtcatggcctcgagaaatccccctcgtcaaaggtggtaaaccagatgtggttcaaattgtcaggaactataacgcctacctcagggcgtcagatgacctccccaaaatgtttatagagtctgatccgggatttttctctaatgccatagtagagggtgctaagaagttccctaacactgaatttgtaaaagttaaagggctccatttctcacaggaagatgcgccagatgaaatgggcaaatacattaaaagtttcgtagagcgggtactgaaaaatgagcagtaataagatacattgatgagtttggacaaaccacaactagaatgcagtgaaaaaaatgctttatttgtgaaatttgtgatgctattgctttatttgtaaccattataagctgcaataaacaagttgcacaaataagtcgcaacagtacaagttcggcacagctacatacgcgctaaaggagccggtaatgaagagcgatgcatgggcagtggtacgcgtccagtcggtctggcagctgggtaacaggcagaggccatacccatgggacgtcaactgggcgaacagcaccatgtactgggggacgcagccctgaaaaggggggggggctaaagccccccccccttaaacccccccctgggggggattcccccccagaccccccctttatatagcactcaataaacgcagaaaatagatttatcgcactatc

SEQ ID NO: 13 - DNA, 2319 bp, Artificial Sequence
  Description of Artificial Sequence: Synthetic polynucleotide
gaattccgagtggttactattccatcaccattctagcctgtacacagaaagtcaagatggacgaatcgctcgacttcgctcgcgattcgtcgaaggcggggggccggaggccccccggtggcccccctccaacgagtggagcacgtacaggggggtacgtcatccgtacaggggggtacgtcatccgtacaggggggtacgtcacaaagaggcgttcccgtacaggggggtacgtcacgcgtacaggggggtacgtcacagccaatcaaaagctgccacgttgcgaaagtgacgtttcgaaaatgggcggcgcaagcctctctatatattgagcgcacataccggtcggcagtaggtatacgcaaggcggtccgggtggttgcacgggaacggcggacaaccggccgctgggggcagtgaatcggcgcttagccgagaggggcaacctgggcccagcggagccgcgcaggggcaagtaatttcaattgaacgctctccaagaagatactccacccggaccatcaacggtgttcaggccaccaacaagttcacggccgttggaaacccctcactgcagagagatccggattggtatcgctggaattactgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagtaagatacattgatgagtttggacaaaccacaactagaatgcagtgaaaaaaatgctttatttgtgaaatttgtgatgctattgctttatttgtaaccattataagctgcaataaacaagttttgcaacactcacagcactcggtgcacaatggtcttttcctccagggcaacgttcagtttctagacggtccttcaaccaccacaaggcgagaggagccggggaccccaagggccagagatggcacacgctggtgccgctcggcacggagaccatcaccgacagctacatgtcagcacccgcatcagagctggacactaatttctttacgctttacgtagcgcaaggcacaaataagtcgcaacagtacaagttcggcacagctacatacgcgctaaaggagccggtaatgaagagcgatgcatgggcagtggtacgcgtccagtcggtctggcagctgggtaacaggcagaggccatacccatgggacgtcaactgggcgaacagcaccatgtactgggggacgcagccctgaaaaggggggggggctaaagccccccccccttaaacccccccctgggggggattcccccccagaccccccctttatatagcactcaataaacgcagaaaatagatttatcgcactatc

SEQ ID NO: 14 - DNA, 6794 bp, Artificial Sequence
  Description of Artificial Sequence: Synthetic polynucleotide
gaattccgagtggttactattccatcaccattctagcctgtacacagaaagtcaagatggacgaatcgctcgacttcgctcgcgattcgtcgaaggcggggggccggaggccccccggtggcccccctccaacgagtggagcacgtacaggggggtacgtcatccgtacaggggggtacgtcatccgtacaggggggtacgtcacaaagaggcgttcccgtacaggggggtacgtcacgcgtacaggggggtacgtcacagccaatcaaaagctgccacgttgcgaaagtgacgtttcgaaaatgggcggcgcaagcctctctatatattgagcgcacataccggtcggcagtaggtatacgcaaggcggtccgggtggttgcacgggaacggcggacaaccggccgctgggggcagtgaatcggcgcttagccgagaggggcaacctgggcccagcggagccgcgcaggggcaagtaatttcaattgaacgctctccaagaagatactccacccggaccatcaacggtgttcaggccaccaacaagttcacggccgttggaaacccctcactgcagagagatccggattggtatcgctggaattacaatcactctatcgctgtgtggctgcgcgaatgctcgcgctcccacgctaagatctgcaactgcggacaattcagaaagcactggtttcaagaatgtgccggacttgaggaccgatcaacccaagcctccctcgaagaagcgatcctgcgacccctccgagtacagggtaagcgagctaaaagaaagcttgattaccactactcccagccgaccccgaaccgcaaaaaggcgtataagactgtaagttggcaagacgagctcgcagaccgagaggccgattttactccttcagaagaggacggtggcaccacctcaagcgacttcgacgaagatataaatttcgacatcggaggagacagcggtatcgtagacgagcttttaggaaggcctttcacaaccctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctgccaccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaataagatacattgatgagtttggacaaaccacaactagaatgcagtgaaaaaaatgctttatttgtgaaatttgtgatgctattgctttatttgtaaccattataagctgcaataaacaagtttacgctttacgtagcgcaaggcacaaataagtcgcaacagtacaagttcggcacagctacatacgcgctaaaggagccggtaatgaagagcgatgcatgggcagtggtacgcgtccagtcggtctggcagctgggtaacaggcagaggccatacccatgggacgtcaactgggcgaacagcaccatgtactgggggacgcagccctgaaaaggggggggggctaaagccccccccccttaaacccccccctgggggggattcccccccagaccccccctttatatagcactcaataaacgcagaaaatagatttatcgcactatcgaattccgagtggttactattccatcaccattctagcctgtacacagaaagtcaagatggacgaatcgctcgacttcgctcgcgattcgtcgaaggcggggggccggaggccccccggtggcccccctccaacgagtggagcacgtacaggggggtacgtcatccgtacaggggggtacgtcatccgtacaggggggtacgtcacaaagaggcgttcccgtacaggggggtacgtcacgcgtacaggggggtacgtcacagccaatcaaaagctgccacgttgcgaaagtgacgtttcgaaaatgggcggcgcaagcctctctatatattgagcgcacataccggtcggcagtaggtatacgcaaggcggtccgggtggatgcacgggaacggcggacaaccggccgctgggggcagtgaatcggcgcttagccgagaggggcaacctgggcccagcggagccgcgcaggggcaagtaatttcaaatgaacgctctccaagaagatactccacccggaccatcaacggtgttcaggccaccaacaagttcacggccgttggaaacccctcactgcagagagatccggattggtatcgctggaattacaatcactctatcgctgtgtggctgcgcgaatgctcgcgctcccacgctaagatctgcaactgcggacaattcagaaagcactggtttcaagaatgtgccggacttgaggaccgatcaacccaagcctccctcgaagaagcgatcctgcgacccctccgagtacagggtaagcgagctaaaagaaagcttgattaccactactcccagccgaccccgaaccgcaaaaaggcgtataagactgtaagatggcaagacgagctcgcagaccgagaggccgattttactccttcagaagaggacggtggcaccacctcaagcgacttcgacgaagatataaatttcgacatcggaggagacagcggtatcgtagacgagcttttaggaaggcctttcacaacccccgccccggtacgtatagtgtgaggctgccgaacccccaatctactatgactatccgcttccaaggggtcatctttctcacggaaggactcattctgcctaaaaacagcacagcggggggctatgcagaccacatgtacggggcgagagtcgccaagatctctgtgaacctgaaagagttcctgctagcctcaatgaacctgacatacgtgagcaaaatcggaggccccatcgccggtgagttgattgcggacgggtctaaatcacaagccgcggacaattggcctaattgctggctgccgctagataataacgtgccctccgctacaccatcggcatggtggagatgggccttaatgatgatgcagcccacggactcttgccggttctttaatcacccaaagcagatgaccctgcaagacatgggtcgcatgtttgggggctggcacctgttccgacacattgaaacccgctttcagctccttgccactaagaatgagggatccttcagccccgtggcgagtcttctctcccagggagagtacctcacgcgtcgggacgatgttaagtacagcagcgatcaccagaaccggtggcaaaaaggcggacaaccgatgacggggggcattgcttatgcgaccgggaaaatgagacccgacgagcaacagtaccctgctatgcccccagaccccccgatcatcaccgctactacagcgcaaggcacgcaagtccgctgcatgaatagcacgcaagcttggtggtcatgggacacatatatgagctttgcaacactcacagcactcggtgcacaatggtcttttcctccagggcaacgttcagtttctagacggtccttcaaccaccacaaggcgagaggagccggggaccccaagggccagagatggcacacgctggtgccgctcggcacggagaccatcaccgacagctacatgtcagcacccgcatcagagctggacactaatttctttacgctttacgtagcgcaaggcacaaataagtcgcaacagtacaagttcggcacagctacatacgcgctaaaggagccggtaatgaagagcgatgcatgggcagtggtacgcgtccagtcggtctggcagctgggtaacaggcagaggccatacccatgggacgtcaactgggcgaacagcaccatgtactgggggacgcagccctgaaaaggggggggggctaaagccccccccccttaaacccccccctgggggggattcccccccagaccccccctttatatagcactcaataaacgcagaaaatagatttatcgcactatctgaatgagacgcacgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattctctggctaactagagaacccactgcttactaggcgtctcagaat

SEQ ID NO: 15 - DNA, 6673 bp, Artificial Sequence
  Description of Artificial Sequence: Synthetic polynucleotide
gaattccgagtggttactattccatcaccattctagcctgtacacagaaagtcaagatggacgaatcgctcgacttcgctcgcgattcgtcgaaggcggggggccggaggccccccggtggcccccctccaacgagtggagcacgtacaggggggtacgtcatccgtacaggggggtacgtcatccgtacaggggggtacgtcacaaagaggcgttcccgtacaggggggtacgtcacgcgtacaggggggtacgtcacagccaatcaaaagctgccacgttgcgaaagtgacgtttcgaaaatgggcggcgcaagcctctctatatattgagcgcacataccggtcggcagtaggtatacgcaaggcggtccgggtggttgcacgggaacggcggacaaccggccgctgggggcagtgaatcggcgcttagccgagaggggcaacctgggcccagcggagccgcgcaggggcaagtaatttcaattgaacgctctccaagaagatactccacccggaccatcaacggtgttcaggccaccaacaagttcacggccgttggaaacccctcactgcagagagatccggattggtatcgctggaattacaatcactctatcgctgtgtggctgcgcgaatgctcgcgctcccacgctaagatctgcaactgcggacaattcagaaagcactggtttcaagaatgtgccggacttgaggaccgatcaacccaagcctccctcgaagaagcgatcctgcgacccctccgagtacagggtaagcgagctaaaagaaagcttgattaccactactcccagccgaccccgaaccgcaaaaaggcgtataagactgtaagttggcaagacgagctcgcagaccgagaggccgattttactccttcagaagaggacggtggcaccacctcaagcgacttcgacgaagatataaatttcgacatcggaggagacagcggtatcgtagacgagcttttaggaaggcctttcacaaccctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctgccaccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaataagatacattgatgagtttggacaaaccacaactagaatgcagtgaaaaaaatgctttatttgtgaaatttgtgatgctattgctttatttgtaaccattataagctgcaataaacaagtttacgctttacgtagcgcaaggcacaaataagtcgcaacagtacaagttcggcacagctacatacgcgctaaaggagccggtaatgaagagcgatgcatgggcagtggtacgcgtccagtcggtctggcagctgggtaacaggcagaggccatacccatgggacgtcaactgggcgaacagcaccatgtactgggggacgcagccctgaaaaggggggggggctaaagccccccccccttaaacccccccctgggggggattcccccccagaccccccctttatatagcactcaataaacgcagaaaatagatttatcgcactatcgaattccgagtggttactattccatcaccattctagcctgtacacagaaagtcaagatggacgaatcgctcgacttcgctcgcgattcgtcgaaggcggggggccggaggccccccggtggcccccctccaacgagtggagcacgtacaggggggtacgtcatccgtacaggggggtacgtcatccgtacaggggggtacgtcacaaagaggcgttcccgtacaggggggtacgtcacgcgtacaggggggtacgtcacagccaatcaaaagctgccacgttgcgaaagtgacgtttcgaaaatgggcggcgcaagcctctctatatattgagcgcacataccggtcggcagtaggtatacgcaaggcggtccgggtggatgcacgggaacggcggacaaccggccgctgggggcagtgaatcggcgcttagccgagaggggcaacctgggcccagcggagccgcgcaggggcaagtaatttcaaatgaacgctctccaagaagatactccacccggaccatcaacggtgttcaggccaccaacaagttcacggccgttggaaacccctcactgcagagagatccggattggtatcgctggaattacaatcactctatcgctgtgtggctgcgcgaatgctcgcgctcccacgctaagatctgcaactgcggacaattcagaaagcactggtttcaagaatgtgccggacttgaggaccgatcaacccaagcctccctcgaagaagcgatcctgcgacccctccgagtacagggtaagcgagctaaaagaaagcttgattaccactactcccagccgaccccgaaccgcaaaaaggcgtataagactgtaagatggcaagacgagctcgcagaccgagaggccgattttactccttcagaagaggacggtggcaccacctcaagcgacttcgacgaagatataaatttcgacatcggaggagacagcggtatcgtagacgagcttttaggaaggcctttcacaacccccgccccggtacgtatagtgtgaggctgccgaacccccaatctactatgactatccgcttccaaggggtcatctttctcacggaaggactcattctgcctaaaaacagcacagcggggggctatgcagaccacatgtacggggcgagagtcgccaagatctctgtgaacctgaaagagttcctgctagcctcaatgaacctgacatacgtgagcaaaatcggaggccccatcgccggtgagttgattgcggacgggtctaaatcacaagccgcggacaattggcctaattgctggctgccgctagataataacgtgccctccgctacaccatcggcatggtggagatgggccttaatgatgatgcagcccacggactcttgccggttctttaatcacccaaagcagatgaccctgcaagacatgggtcgcatgtttgggggctggcacctgttccgacacattgaaacccgctttcagctccttgccactaagaatgagggatccttcagccccgtggcgagtcttctctcccagggagagtacctcacgcgtcgggacgatgttaagtacagcagcgatcaccagaaccggtggcaaaaaggcggacaaccgatgacggggggcattgcttatgcgaccgggaaaatgagacccgacgagcaacagtaccctgctatgcccccagaccccccgatcatcaccgctactacagcgcaaggcacgcaagtccgctgcatgaatagcacgcaagcttggtggtcatgggacacatatatgagctttgcaacactcacagcactcggtgcacaatggtcttttcctccagggcaacgttcagtttctagacggtccttcaaccaccacaaggcgagaggagccggggaccccaagggccagagatggcacacgctggtgccgctcggcacggagaccatcaccgacagctacatgtcagcacccgcatcagagctggacactaatttctttacgctttacgtagcgcaaggcacaaataagtcgcaacagtacaagttcggcacagctacatacgcgctaaaggagccggtaatgaagagcgatgcatgggcagtggtacgcgtccagtcggtctggcagctgggtaacaggcagaggccatacccatgggacgtcaactgggcgaacagcaccatgtactgggggacgcagccctgatgaatgagacgcacgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattctctggctaactagagaacccactgcttactaggcgtctca

SEQ ID NO: 16 - DNA, 4793 bp, Artificial Sequence
  Description of Artificial Sequence: Synthetic polynucleotide
gaattccgagtggttactattccatcaccattctagcctgtacacagaaagtcaagatggacgaatcgctcgacttcgctcgcgattcgtcgaaggcggggggccggaggccccccggtggcccccctccaacgagtggagcacgtacaggggggtacgtcatccgtacaggggggtacgtcatccgtacaggggggtacgtcacaaagaggcgttcccgtacaggggggtacgtcacgcgtacaggggggtacgtcacagccaatcaaaagctgccacgttgcgaaagtgacgtttcgaaaatgggcggcgcaagcctctctatatattgagcgcacataccggtcggcagtaggtatacgcaaggcggtccgggtggttgcacgggaacggcggacaaccggccgctgggggcagtgaatcggcgcttagccgagaggggcaacctgggcccagcggagccgcgcaggggcaagtaatttcaattgaacgctctccaagaagatactccacccggaccatcaacggtgttcaggccaccaacaagttcacggccgttggaaacccctcactgcagagagatccggattggtatcgctggaattacaatcactctatcgctgtgtggctgcgcgaatgctcgcgctcccacgctaagatctgcaactgcggacaattcagaaagcactggtttcaagaatgtgccggacttgaggaccgatcaacccaagcctccctcgaagaagcgatcctgcgacccctccgagtacagggtaagcgagctaaaagaaagcttgattaccactactcccagccgaccccgaaccgcaaaaaggcgtataagactgtaagttggcaagacgagctcgcagaccgagaggccgattttactccttcagaagaggacggtggcaccacctcaagcgacttcgacgaagatataaatttcgacatcggaggagacagcggtatcgtagacgagcttttaggaaggcctttcacaaccctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctgccaccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaataagatacattgatgagtttggacaaaccacaactagaatgcagtgaaaaaaatgctttatttgtgaaatttgtgatgctattgctttatttgtaaccattataagctgcaataaacaagtttacgctttacgtagcgcaaggcacaaataagtcgcaacagtacaagttcggcacagctacatacgcgctaaaggagccggtaatgaagagcgatgcatgggcagtggtacgcgtccagtcggtctggcagctgggtaacaggcagaggccatacccatgggacgtcaactgggcgaacagcaccatgtactgggggacgcagccctgaaaaggggggggggctaaagccccccccccttaaacccccccctgggggggattcccccccagaccccccctttatatagcactcaataaacgcagaaaatagatttatcgcactatcgaattccgagtggttactattccatcaccattctagcctgtacacagaaagtcaagatggacgaatcgctcgacttcgctcgcgattcgtcgaaggcggggggccggaggccccccggtggcccccctccaacgagtggagcacgtacaggggggtacgtcatccgtacaggggggtacgtcatccgtacaggggggtacgtcacaaagaggcgttcccgtacaggggggtacgtcacgcgtacaggggggtacgtcacagccaatcaaaagctgccacgttgcgaaagtgacgtttcgaaaatgggcggcgcaagcctctctgaatgagacgcacgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattctctggctaactagagaacccactgcttactaggcgtctca

SEQ ID NO: 17 - DNA, 125 bp, Chicken anemia virus
agccctgaaaaggggggggggctaaagccccccccccttaaacccccccctgggggggattcccccccagaccccccctttatatagcactcaataaacgcagaaaatagatttatcgcactatc

SEQ ID NO: 18-99 - skipped (82 entries)

SEQ ID NO: 100 - DNA, 2319 bp, Chicken anemia virus
gaattccgagtggttactattccatcaccattctagcctgtacacagaaagtcaagatggacgaatcgctcgacttcgctcgcgattcgtcgaaggcggggggccggaggccccccggtggcccccctccaacgagtggagcacgtacaggggggtacgtcatccgtacaggggggtacgtcatccgtacaggggggtacgtcacaaagaggcgttcccgtacaggggggtacgtcacgcgtacaggggggtacgtcacagccaatcaaaagctgccacgttgcgaaagtgacgtttcgaaaatgggcggcgcaagcctctctatatattgagcgcacataccggtcggcagtaggtatacgcaaggcggtccgggtggatgcacgggaacggcggacaaccggccgctgggggcagtgaatcggcgcttagccgagaggggcaacctgggcccagcggagccgcgcaggggcaagtaatttcaaatgaacgctctccaagaagatactccacccggaccatcaacggtgttcaggccaccaacaagttcacggccgttggaaacccctcactgcagagagatccggattggtatcgctggaattacaatcactctatcgctgtgtggctgcgcgaatgctcgcgctcccacgctaagatctgcaactgcggacaattcagaaagcactggtttcaagaatgtgccggacttgaggaccgatcaacccaagcctccctcgaagaagcgatcctgcgacccctccgagtacagggtaagcgagctaaaagaaagcttgattaccactactcccagccgaccccgaaccgcaaaaaggcgtataagactgtaagatggcaagacgagctcgcagaccgagaggccgattttactccttcagaagaggacggtggcaccacctcaagcgacttcgacgaagatataaatttcgacatcggaggagacagcggtatcgtagacgagcttttaggaaggcctttcacaacccccgccccggtacgtatagtgtgaggctgccgaacccccaatctactatgactatccgcttccaaggggtcatctttctcacggaaggactcattctgcctaaaaacagcacagcggggggctatgcagaccacatgtacggggcgagagtcgccaagatctctgtgaacctgaaagagttcctgctagcctcaatgaacctgacatacgtgagcaaaatcggaggccccatcgccggtgagttgattgcggacgggtctaaatcacaagccgcggacaattggcctaattgctggctgccgctagataataacgtgccctccgctacaccatcggcatggtggagatgggccttaatgatgatgcagcccacggactcttgccggttctttaatcacccaaagcagatgaccctgcaagacatgggtcgcatgtttgggggctggcacctgttccgacacattgaaacccgctttcagctccttgccactaagaatgagggatccttcagccccgtggcgagtcttctctcccagggagagtacctcacgcgtcgggacgatgttaagtacagcagcgatcaccagaaccggtggcaaaaaggcggacaaccgatgacggggggcattgcttatgcgaccgggaaaatgagacccgacgagcaacagtaccctgctatgcccccagaccccccgatcatcaccgctactacagcgcaaggcacgcaagtccgctgcatgaatagcacgcaagcttggtggtcatgggacacatatatgagctttgcaacactcacagcactcggtgcacaatggtcttttcctccagggcaacgttcagtttctagacggtccttcaaccaccacaaggcgagaggagccggggaccccaagggccagagatggcacacgctggtgccgctcggcacggagaccatcaccgacagctacatgtcagcacccgcatcagagctggacactaatttctttacgctttacgtagcgcaaggcacaaataagtcgcaacagtacaagttcggcacagctacatacgcgctaaaggagccggtaatgaagagcgatgcatgggcagtggtacgcgtccagtcggtctggcagctgggtaacaggcagaggccatacccatgggacgtcaactgggcgaacagcaccatgtactgggggacgcagccctgaaaaggggggggggctaaagccccccccccttaaacccccccctgggggggattcccccccagaccccccctttatatagcactcaataaacgcagaaaatagatttatcgcactatc

SEQ ID NO: 101 - protein, 17 aa, Unknown
  Description of Unknown: nuclear localization signal sequence
RRARRPRGRFYAFRRGR

SEQ ID NO: 102 - protein, 24 aa, Unknown
  Description of Unknown: nuclear localization signal sequence
KRLRRRYKFRHRRRQRYRRRAFRK

SEQ ID NO: 103 - protein, 9 aa, Unknown
  Description of Unknown: nuclear export signal sequence
IFLTEGLIL

SEQ ID NO: 104 - protein, 11 aa, Unknown
  Description of Unknown: nuclear export signal sequence
LKEFLLASMNL

SEQ ID NO: 105 - protein, 13 aa, Unknown
  Description of Unknown: nuclear export signal sequence
ELDTNFFTLYVAQ

SEQ ID NO: 106 - protein, 10 aa, Unknown
  Description of Unknown: CAV VP2 sequence
ICNCGQFRKH

SEQ ID NO: 107 - protein, 21 aa, Unknown
  Description of Unknown: CAV VP2 sequence
WLRECSRSHAKICNCGQFRKH

SEQ ID NO: 108 - protein, 21 aa, Unknown
  Description of Unknown: CAV VP1 sequence
RRRYKFRHRRQRYRRRAFRKH

SEQ ID NO: 109 - protein, 17 aa, Unknown
  Description of Unknown: CAV VP1 sequence
SRRSFNHHKARGAGDPK

SEQ ID NO: 110 - protein, 20 aa, Unknown
  Description of Unknown: nuclear localization signal sequence
RRARRPRGRFYAFRRGRWHH

SEQ ID NO: 111 - protein, 21 aa, Unknown
  Description of Unknown: CAV VP2 sequence
  MOD_RES (2)..(8): Any amino acid
  MOD_RES (10)..(12): Any amino acid
  MOD_RES (14)..(14): Any amino acid
  MOD_RES (16)..(20): Any amino acid
WXXXXXXXHXXXCXCXXXXXH

SEQ ID NO: 112 - DNA, 22 bp, Artificial Sequence
  Description of Artificial Sequence: Synthetic primer
ttggaaacccctcactgcagag

SEQ ID NO: 113 - DNA, 21 bp, Artificial Sequence
  Description of Artificial Sequence: Synthetic primer
ctgaattgtccgcagttgcag

SEQ ID NO: 114 - DNA, 21 bp, Artificial Sequence
  Description of Artificial Sequence: Synthetic probe
ctggaattacaatcactctat

(19) United States
(12) Patent Application Publication
(10) Pub. No.: US 2024/0000914 A1
(43) Pub. Date: Jan. 4, 2024

(54) CHICKEN ANEMIA VIRUS (CAV)-BASED VECTORS

(71) Applicant: FLAGSHIP PIONEERING INNOVATIONS V, INC., Cambridge, MA (US)

(72) Inventors: Simon DELAGRAVE, Sudbury, MA (US); Fernando Martin DIAZ, New York, NY (US); Jared David PITTS, Jamaica Plain, MA (US); Kevin James LEBO, Weymouth, MA (US); Joseph Louis TIMPONA, Boston, MA (US); Nicole Marie BOISVERT, Lowell, MA (US)

(21) Appl. No.: 18/250,621

(22) PCT Filed: Oct. 29, 2021
(86) PCT No.: PCT/US2021/057292

§ 371 Date: Apr. 26, 2023

Related U.S. Application Data
(60) Provisional application No. 63/147,087, filed on Feb. 8, 2021.
(60) Provisional application No. 63/107,149, filed on Oct. 29, 2020.

(51) Int. Cl. A61K 39/12; C12N 7/00; C12N 15/86; C12N 1/04

(52) U.S. Cl. CPC A61K 39/12; C12N 7/00; C12N 15/86; C12N 1/04; C12N 2750/00021; C12N 2750/00023; C12N 2750/00034; C12N 2750/00043; A61K 2039/5256

(57) ABSTRACT

This invention relates generally to compositions for making C A Vectors and uses thereof.

### BACKGROUND

Chicken anemia virus (CAV) is a non-enveloped, single stranded DNA virus of the Gyrovirus genus, which infects chickens. The CAV genome encodes three open reading frames encoding VP1, VP2, and VP3 (also called Apoptin). VP1 is the major component responsible for capsid assembly.

### SUMMARY

This disclosure provides vectors, other compositions, and related methods, for infecting or modulating mammalian or avian cells. Generally, vectors disclosed herein include a genetic element that comprises a CAV sequence, or a sequence with homology thereto, and a proteinaceous exterior. In some embodiments, the genetic element is enclosed by a proteinaceous exterior comprising a CAV capsid protein or by a CAV capsid.

The present disclosure also provides constructs for producing a CAVector (e.g., a synthetic CAVector) that can be used as a delivery vehicle, e.g., for delivering genetic material, for delivering an effector, e.g., a payload, or for delivering a therapeutic agent or a therapeutic effector to a eukaryotic cell (e.g., a mammalian cell or tissue, e.g., a human cell or a human tissue). Generally, a CAVector comprises a genetic element comprising one or more nucleic acid sequences having substantial (e.g., at least about 75%, 80%, 85%, 90%, 95%, 96%, 97%, 98%, 99%, or 100%) sequence identity to a nucleic acid sequence of a CAV genome (e.g., one or more of a CAV 5′ UTR, repeat region, CAAT signal, TATA box, VP2 gene, Apoptin gene, VP1, 3′ UTR, GC-rich region, polyA signal sequence, e.g., as described herein, or a functional fragment thereof). In some embodiments, the CAVector also comprises a proteinaceous exterior comprising a polypeptide having substantial (e.g., at least about 75%, 80%, 85%, 90%, 95%, 96%, 97%, 98%, 99%, or 100%) sequence identity to a chicken anemia virus (CAV) VP1 molecule.

In some embodiments, a CAVector comprises a genetic element (e.g., a genetic element comprising or encoding an effector, e.g., an exogenous or endogenous effector, e.g., a therapeutic effector) encapsulated in a proteinaceous exterior (e.g., a proteinaceous exterior comprising a CAV capsid protein, e.g., a CAV VP1 protein or a polypeptide encoded by a CAV VP1 nucleic acid, e.g., as described herein). In some embodiments, the proteinaceous exterior is capable of introducing the genetic element into a cell (e.g., a mammalian cell, e.g., a human cell). In some embodiments, the CAVector is an infectious vehicle or particle comprising a proteinaceous exterior comprising a polypeptide encoded by a CAV VP1 nucleic acid (e.g., as described herein). The genetic element of a CAVector of the present disclosure is typically a circular and/or single-stranded DNA molecule (e.g., circular and single stranded). In some embodiments, the genetic element includes a protein binding sequence that binds to the proteinaceous exterior enclosing it, or a polypeptide attached thereto, which may facilitate enclosure of the genetic element within the proteinaceous exterior and/or enrichment of the genetic element, relative to other nucleic acids, within the proteinaceous exterior.

In some instances, the genetic element is provided using a genetic element construct, e.g., as described herein. Generally, the genetic element construct comprises a nucleic acid sequence corresponding to the sequence of the genetic element to be enclosed in a proteinaceous exterior to form a CAVector. In some instances, the genetic element construct is circular or linear. In some instances, the genetic element is circular. In some instances, the genetic element is single-stranded. In some instances, the genetic element is double-stranded. In some instances, the genetic element is DNA. In some embodiments, a genetic element suitable for enclosure in a proteinaceous exterior can be produced via rolling circle replication of the genetic element sequence in the genetic element construct.

In some instances, the genetic element comprises or encodes an effector (e.g., a nucleic acid effector, such as a non-coding RNA, or a polypeptide effector, e.g., a protein), e.g., which can be expressed in a cell (e.g., a target cell). In some embodiments, the effector is a therapeutic agent or a therapeutic effector, e.g., as described herein. In some embodiments, the effector is endogenous or exogenous, e.g., to a wild-type CAV and/or to the target cell. In some embodiments, the effector is exogenous to a wild-type CAV and/or to the target cell. In some embodiments, the CAVector can deliver an effector into a cell by contacting the cell and introducing a genetic element encoding the effector into the cell, such that the effector is made or expressed by the cell. In certain instances, the effector is endogenous to the target cell (e.g., provided in increased amounts by the CAVector). In other instances, the effector is exogenous to the target cell. The effector can, in some instances, modulate a function of the cell or modulate (e.g., increase or decrease) an activity or level of a target molecule in the cell. For example, the effector can decrease levels of a target protein in the cell. In another example, the effector can increase levels of a target protein in the cell. In some embodiments, the CAVector can deliver and express an effector, e.g., an exogenous protein, in vivo. CAVectors can be used, for example, to deliver genetic material to a target cell, tissue, or subject; to deliver an effector to a target cell, tissue, or subject; or for treatment of diseases and disorders, e.g., by delivering an effector that can operate as a therapeutic agent in a desired cell, tissue, or subject.

In some embodiments, the methods and compositions (e.g., genetic element constructs) described herein can be used to produce a synthetic CAVector, e.g., in a host cell. A synthetic CAVector has at least one structural difference compared to a wild-type virus (e.g., a wild-type CAV, e.g., a described herein), e.g., a difference in the genetic element relative to the genome of the wild-type virus and/or a difference in a structural protein (e.g., a protein in the proteinaceous exterior, e.g., a capsid protein, e.g., a VP1 molecule) relative to the wild-type virus. In some embodiments, the difference comprises one or more of a deletion, insertion, substitution, or other modification (e.g., enzymatic modification), relative to the wild-type virus. Generally, synthetic CAVectors include an exogenous genetic element enclosed within a proteinaceous exterior (e.g., comprising a CAV VP1 molecule), which can be used for delivering the genetic element, or an effector (e.g., an exogenous effector or an endogenous effector) encoded therein (e.g., a polypeptide or nucleic acid effector), into eukaryotic (e.g., human) cells.

In an aspect, the instant disclosure provides a CAVector comprising: (i) a genetic element comprising a promoter element and a sequence encoding an effector (e.g., an endogenous or exogenous effector), and a protein binding sequence (e.g., an exterior protein binding sequence, e.g., a packaging signal); and (ii) a proteinaceous exterior; wherein the genetic element is enclosed within the proteinaceous exterior (e.g., a capsid). In some embodiments, the CAVector is capable of delivering the genetic element into a eukaryotic (e.g., mammalian, e.g., human) cell. In some embodiments, the genetic element is a single-stranded and/or circular DNA. Alternatively or in combination, the genetic element has one, two, three, or all of the following properties: is circular, is single-stranded, it integrates into the genome of a cell at a frequency of less than about 0.0001%, 0.001%, 0.005%, 0.01%, 0.05%, 0.1%, 0.5%, 1%, 1.5%, or 2% of the genetic element that enters the cell, and/or it integrates into the genome of a target cell at less than 1, 2, 3, 4, 5, 6, 7, 8, 9, 10, 15, 20, 25, or 30 copies per genome. In some embodiments, integration frequency is determined by quantitative gel purification assay of genomic DNA separated from free vector, e.g., as described in Wang et al. (2004, Gene Therapy 11: 711-721, incorporated herein by reference in its entirety). In some embodiments, the genetic element is enclosed within the proteinaceous exterior. In some embodiments, the CAVector is capable of delivering the genetic element into a eukaryotic cell. In some embodiments, the genetic element comprises a nucleic acid sequence (e.g., a nucleic acid sequence of between 300-4000 nucleotides, e.g., between 300-3500 nucleotides, between 300-3000 nucleotides, between 300-2500 nucleotides, between 300-2000 nucleotides, between 300-1500 nucleotides, between 1000-4000 nucleotides, between 2000-4000 nucleotides, between 1000-3000 nucleotides, between 2000-2500 nucleotides, or between 2000-3000 nucleotides) having at least 75% (e.g., at least 75, 76, 77, 78, 79, 80, 90, 91, 92, 93, 94, 95, 96, 97, 98, 99, or 100%) sequence identity to a sequence of a wild-type CAV, e.g., a wild-type CAV sequence as described herein). In some embodiments, the genetic element comprises a nucleic acid sequence (e.g., a nucleic acid sequence of at least 300 nucleotides, 500 nucleotides, 1000 nucleotides, 1500 nucleotides, 2000 nucleotides, 2500 nucleotides, 3000 nucleotides or more) having at least 75% (e.g., at least 75, 76, 77, 78, 79, 80, 90, 91, 92, 93, 94, 95, 96, 97, 98, 99, or 100%) sequence identity to a sequence of a wild-type CAV sequence (e.g., a wild-type CAV sequence as described herein). In some embodiments, the nucleic acid sequence is codon-optimized, e.g., for expression in a mammalian (e.g., human) cell. In some embodiments, at least 50%, 60%, 70%, 80%, 90%, 95%, 96%, 97%, 98%, 99%, or 100% of the codons in the nucleic acid sequence are codon-optimized, e.g., for expression in a mammalian (e.g., human) cell.

In some embodiments, the genetic element constructs described herein comprise a first copy of a genetic element sequence (e.g., a mutant chicken anemia virus (CAV) genome) and at least a portion of a second copy of a genetic element sequence (e.g., a CAV genome or a fragment thereof), arranged in tandem. Genetic element constructs having such a structure are generally referred to herein as tandem constructs. Such tandem constructs are used for producing a CAVector genetic element. The first copy of the genetic element sequence and the second copy of the genetic element sequence may, in some instances, be immediately adjacent to each other on the genetic acid construct. In other instances, the first copy of the genetic element sequence and the second copy of the genetic element sequence may be separated, e.g., by a spacer sequence. In some embodiments, the second copy of the genetic element sequence, or the portion thereof, comprises an upstream replication-facilitating sequence (uRFS), e.g., as described herein. In some embodiments, the second copy of the genetic element sequence, or the portion thereof, comprises a downstream replication-facilitating sequence (dRFS), e.g., as described herein. In some embodiments, the uRFS and/or dRFS comprises an origin of replication (ORI) (e.g., a mammalian ORI or an insect ORI) or portion thereof. In some embodiments, the uRFS and/or dRFS does not comprise an origin of replication. In some embodiments, the uRFS and/or dRFS comprises a hairpin loop (e.g., in the 5′ UTR). In some embodiments, a tandem construct produces higher levels of a genetic element than an otherwise similar construct lacking the second copy of the genetic element or portion thereof. Without being bound by theory, a tandem construct described herein may, in some embodiments, replicate by rolling circle replication. In some embodiments, a tandem construct is a plasmid.

A tandem construct may, in some instances, include a first copy of the sequence of the genetic element and a second copy of the sequence of the genetic element, or a portion thereof (e.g., an uRFS or a dRFS). It is understood that the second copy can be an identical copy of the first copy or a portion thereof, or can comprise one or more sequence differences, e.g., substitutions. In some instances, the second copy of the genetic element sequence or portion thereof (e.g., an uRFS) is positioned 5′ relative to the first copy of the genetic element sequence. In some instances, the second copy of the genetic element sequence or portion thereof (e.g., a dRFS) is positioned 3′ relative to the first copy of the genetic element sequence. In some instances, the second copy of the genetic element sequence or portion thereof and the first copy of the genetic element sequence are adjacent to each other in the tandem construct. In some instances, the second copy of the genetic element sequence or portion thereof and the first copy of the genetic element sequence are separated, e.g., by a spacer sequence.

In some embodiments, the tandem constructs described herein can be used to produce the genetic element of an infectious (e.g., to a human cell) CAVector, vehicle, or particle comprising a capsid (e.g., a capsid comprising a CAV ORF, e.g., VP1, polypeptide) encapsulating a genetic element comprising a protein binding sequence that binds to the capsid and a heterologous (to the CAV) sequence encoding a therapeutic effector. In embodiments, the CAVector is capable of delivering the genetic element into a mammalian, e.g., human, cell. In some embodiments, the genetic element has less than about 6% (e.g., less than 10%, 9%, 8%, 7%, 6%, 5.5%, 5%, 4.5%, 4%, 3.5%, 3%, 2.5%, 2%, 1.5%, or less) identity to a wild type CAV genome sequence. In some embodiments, the genetic element has no more than 1.5%, 2%, 2.5%, 3%, 3.5%, 4%, 4.5%, 5%, 5.5% or 6% identity to a wild type CAV genome sequence. In some embodiments, the genetic element has at least about 2% to at least about 5.5% (e.g., 2 to 5%, 3% to 5%, 4% to 5%) identity to a wild type CAV. In some embodiments, the genetic element has greater than about 2000, 3000, 4000, 4500, or 5000 nucleotides of non-viral sequence (e.g., non CAV genome sequence). In some embodiments, the genetic element has greater than about 2000 to 5000, 2500 to 4500, 3000 to 4500, 2500 to 4500, 3500, or 4000, 4500 (e.g., between about 3000 to 4500) nucleotides of non-viral sequence (e.g., non CAV genome sequence). In some embodiments, the genetic element is a single-stranded, circular DNA. Alternatively or in combination, the genetic element has one, two or 3 of the following properties: is circular, is single stranded, it integrates into the genome of a cell at a frequency of less than about 0.001%, 0.005%, 0.01%, 0.05%, 0.1%, 0.5%, 1%, 1.5%, or 2% of the genetic element that enters the cell, it integrates into the genome of a target cell at less than 1, 2, 3, 4, 5, 6, 7, 8, 9, 10, 15, 20, 25, or 30 copies per genome or integrates at a frequency of less than about 0.0001%, 0.001%, 0.005%, 0.01%, 0.05%, 0.1%, 0.5%, 1%, 1.5%, or 2% of the genetic element that enters the cell (e.g., by comparing integration frequency into genomic DNA relative to genetic element sequences from cell lysates). In some embodiments, integration frequency is determined by quantitative gel purification assay of genomic DNA separated from free vector, e.g., as described in Wang et al. (2004, Gene Therapy 11: 711-721, incorporated herein by reference in its entirety).

In some embodiments of the systems and methods herein, a CAVector is made by introducing into a cell a first nucleic acid molecule that is a genetic element or genetic element construct, e.g., a tandem construct, and a second nucleic acid molecule encoding a proteinaceous exterior, e.g., a capsid protein. In some embodiments, the first nucleic acid molecule and the second nucleic acid molecule are attached to each other (e.g., in a genetic element construct described herein, e.g., in cis). In some embodiments, the first nucleic acid molecule and the second nucleic acid molecule are separate (e.g, in trans). In some embodiments, the first nucleic acid molecule is a plasmid, cosmid, bacmid, minicircle, or artificial chromosome. In some embodiments, the second nucleic acid molecule is a plasmid, cosmid, bacmid, minicircle, or artificial chromosome. In some embodiments, the second nucleic acid molecule is integrated into the genome of the host cell.

In some embodiments, the method further includes introducing the first nucleic acid molecule and/or the second nucleic acid molecule into the host cell. In some embodiments, the second nucleic acid molecule is introduced into the host cell prior to, concurrently with, or after the first nucleic acid molecule. In other embodiments, the second nucleic acid molecule is integrated into the genome of the host cell. In some embodiments, the second nucleic acid molecule is or comprises or is part of a helper construct, helper virus or other helper vector.

In some embodiments, CAVs or CAVectors, as described herein, can be used as effective delivery vehicles for introducing an agent, such as an effector described herein, to a target cell, e.g., a target cell in a subject to be treated.

In some embodiments, a CAVector described herein comprises a proteinaceous exterior comprising a polypeptide (e.g., a synthetic polypeptide, e.g., an VP1 molecule) comprising (e.g., in series):
(i) a first region comprising an arginine-rich region, e.g., a sequence of at least about 40 amino acids comprising at least 60%, 70%, or 80% basic residues (e.g., arginine, lysine, or a combination thereof),
(ii) a second region comprising a jelly-roll domain, e.g., a sequence comprising at least 6 beta strands, e.g., 6, 7 or 8 beta strands arranged in two antiparallel beta sheets which pack together across a hydrophobic interface, and
(iii) optionally wherein the polypeptide has an amino acid sequence having less than 100%, 99%, 98%, 95%, 90%, 85%, 80% sequence identity to a wild type CAV VP1 protein, e.g., as described herein.

In some embodiments, a CAVector described herein comprises a proteinaceous exterior comprising a polypeptide comprising any one or more of (e.g., 1, 2, 3, 4, or all of):
(i) a nuclear localization signal (NLS) comprising the amino acid sequence RRARRPRGRFYAFRRGR, or an amino acid sequence having at least 75%, 80%, 85%, 90%, 95%, 96%, 97%, 98%, or 99% sequence identity thereto;
(ii) a nuclear localization signal (NLS) comprising the amino acid sequence KRLRRRYKFRHRRRQRYRRRAFRK, or an amino acid sequence having at least 75%, 80%, 85%, 90%, 95%, 96%, 97%, 98%, or 99% sequence identity thereto;
(iii) a nuclear export signal (NES) comprising the amino acid sequence IFLTEGLIL, or an amino acid sequence having at least 75%, 80%, 85%, 90%, 95%, 96%, 97%, 98%, or 99% sequence identity thereto;
(iv) a nuclear export signal (NES) comprising the amino acid sequence LKEFLLASMNL, or an amino acid sequence having at least 75%, 80%, 85%, 90%, 95%, 96%, 97%, 98%, or 99% sequence identity thereto;
(v) a nuclear export signal (NES) comprising the amino acid sequence ELDTNFFTLYVAQ, or an amino acid sequence having at least 75%, 80%, 85%, 90%, 95%, 96%, 97%, 98%, or 99% sequence identity thereto;
e.g., as described in Cheng et al. (2019, Virol. J. 16:45; incorporated herein by reference in its entirety).

In an aspect, the invention features an isolated nucleic acid molecule (e.g., a genetic element construct) comprising the sequence of a genetic element comprising a promoter element operably linked to a sequence encoding an effector, e.g., a payload, and an exterior protein binding sequence. In some embodiments, the exterior protein binding sequence includes a sequence at least 75% (at least 80%, 85%, 90%, 95%, 97%, 100%) identical to a 5′UTR sequence of an CAV, e.g., as disclosed herein. In embodiments, the genetic element is a single-stranded DNA, is circular, integrates at a frequency of less than about 0.001%, 0.005%, 0.01%, 0.05%, 0.1%, 0.5%, 1%, 1.5%, or 2% of the genetic element that enters the cell, and/or integrates into the genome of a target cell at less than 1, 2, 3, 4, 5, 6, 7, 8, 9, 10, 15, 20, 25, or 30 copies per genome or integrates at a frequency of less than about 0.001%, 0.005%, 0.01%, 0.05%, 0.1%, 0.5%, 1%, 1.5%, or 2% of the genetic element that enters the cell. In some embodiments, integration frequency is determined by quantitative gel purification assay of genomic DNA separated from free vector, e.g., as described in Wang et al. (2004, Gene Therapy 11: 711-721, incorporated herein by reference in its entirety). In embodiments, the effector does not originate from TTV and is not an SV40-miR-S1. In embodiments, the nucleic acid molecule does not comprise the polynucleotide sequence of TTMV-LY2. In embodiments, the promoter element directs expression of the effector in a eukaryotic (e.g., mammalian, e.g., human) cell. In embodiments, the effector is a mammalian nucleic acid or polypeptide (e.g., a mammalian, e.g., a human, polypeptide or nucleic acid).

In some embodiments, the nucleic acid molecule is circular. In some embodiments, the nucleic acid molecule is linear. In some embodiments, a nucleic acid molecule described herein comprises one or more modified nucleotides (e.g., a base modification, sugar modification, or backbone modification).

In some embodiments, the nucleic acid molecule comprises a sequence encoding an VP1 molecule (e.g., an CAV VP1 protein, e.g., as described herein). In some embodiments, the nucleic acid molecule comprises a sequence encoding an VP2 molecule (e.g., an CAV VP2 protein, e.g., as described herein). In some embodiments, the nucleic acid molecule comprises a sequence encoding an Apoptin molecule (e.g., an CAV Apoptin protein, e.g., as described herein). In an aspect, the invention features a genetic element comprising one, two, or three of: (i) a promoter element and a sequence encoding an effector, e.g., an exogenous or endogenous effector; (ii) at least 72, 73, 74, 75, 76, 77, 78, 79, 80, 90, 100, or 150 nucleotides having at least 75% (e.g., at least 75, 76, 77, 78, 79, 80, 90, 91, 92, 93, 94, 95, 96, 97, 98, 99, or 100%) sequence identity to a wild-type CAV sequence; or at least 100 (e.g., at least 300, 500, 1000, 1500) contiguous nucleotides having at least 72% (e.g., at least 72, 73, 74, 75, 76, 77, 78, 79, 80, 90, 91, 92, 93, 94, 95, 96, 97, 98, 99, or 100%) sequence identity to a wild-type CAV sequence; and (iii) a protein binding sequence, e.g., an exterior protein binding sequence, and wherein the genetic element construct is a single-stranded DNA; and wherein the genetic element construct is circular, integrates at a frequency of less than about 0.001%, 0.005%, 0.01%, 0.05%, 0.1%, 0.5%, 1%, 1.5%, or 2% of the genetic element that enters the cell, and/or integrates into the genome of a target cell at less than 1, 2, 3, 4, 5, 6, 7, 8, 9, 10, 15, 20, 25, or 30 copies per genome In some embodiments, a genetic element encoding an effector (e.g., an exogenous or endogenous effector, e.g., as described herein) is codon optimized. In some embodiments, the genetic element is circular. In some embodiments, the genetic element is linear. In some embodiments, a genetic element described herein comprises one or more modified nucleotides (e.g., a base modification, sugar modification, or backbone modification). In some embodiments, the genetic element comprises a sequence encoding an VP1 molecule (e.g., a CAV VP1 protein, e.g., as described herein). In some embodiments, the genetic element comprises a sequence encoding a VP2 molecule (e.g., a CAV VP2 protein, e.g., as described herein). In some embodiments, the genetic element comprises a sequence encoding an Apoptin molecule (e.g., a CAV Apoptin protein, e.g., as described herein).

In an aspect, the invention features a host cell comprising a tandem construct as described herein. In some embodiments, the host cell comprises: (a) a nucleic acid molecule comprising a sequence encoding one or more of a VP1 molecule, a VP2 molecule, or a Apoptin molecule (e.g, a sequence encoding a CAV VP1 polypeptide described herein), e.g., wherein the nucleic acid molecule is a plasmid, is a viral nucleic acid, or is integrated into a chromosome; and (b) a genetic element, wherein the genetic element comprises (i) a promoter element operably linked to a nucleic acid sequence (e.g., a DNA sequence) encoding an effector (e.g., an exogenous effector or an endogenous effector) and (ii) a protein binding sequence that binds the polypeptide of (a), wherein optionally the genetic element does not encode a VP1 polypeptide (e.g., a VP1 protein). For example, the host cell comprises (a) and (b) either in cis (both part of the same nucleic acid molecule) or in trans (each part of a different nucleic acid molecule). In embodiments, the genetic element of (b) is a circular, single-stranded DNA. In some embodiments, the host cell is a manufacturing cell line, e.g., as described herein. In some embodiments, the host cell is adherent or in suspension, or both. In some embodiments, the host cell or helper cell is grown in a microcarrier. In some embodiments, the host cell or helper cell is compatible with cGMP manufacturing practices. In some embodiments, the host cell or helper cell is grown in a medium suitable for promoting cell growth. In certain embodiments, once the host cell or helper cell has grown sufficiently (e.g., to an appropriate cell density), the medium may be exchanged with a medium suitable for production of CAVectors by the host cell or helper cell.

In an aspect, the invention features a pharmaceutical composition comprising a CAVector (e.g., a synthetic CAVector) as described herein. In embodiments, the pharmaceutical composition further comprises a pharmaceutically acceptable carrier or excipient. In embodiments, the pharmaceutical composition comprises a unit dose comprising about 10⁵-10¹⁴ (e.g., about 10⁶-10¹³, 10⁷-10¹², 10⁸-10¹¹, or 10⁹-10¹⁰) genome equivalents of the CAVector per kilogram of a target subject. In some embodiments, the pharmaceutical composition comprising the preparation is stable over an acceptable period of time and temperature, and/or is compatible with the desired route of administration and/or any devices this route of administration will require, e.g., needles or syringes. In some embodiments, the pharmaceutical composition is formulated for administration as a single dose or multiple doses. In some embodiments, the pharmaceutical composition is formulated at the site of administration, e.g., by a healthcare professional. In some embodiments, the pharmaceutical composition comprises a desired concentration of CAVector genomes or genomic equivalents (e.g., as defined by number of genomes per volume).

In an aspect, the invention features a pharmaceutical composition comprising a CAVector, wherein the composition meets the requirements of 21 C.F.R. §§ 610.12 and 610.13. For example, the pharmaceutical composition may have one, two, 3, 4, 5, 6, 7 or all 8 of the following characteristics:
(i) substantially lacks adventitious agents,
(ii) substantially lacks pyrogenic substances,
(iii) contains equal to or less endotoxin than a control reference or specification, e.g., a U.S. Pharmacopeia (USP) or FDA reference standard for endotoxin contamination,
(iv) contains equal to or less mycoplasma than a control reference or specification, e.g., a U.S. Pharmacopeia (USP) or FDA reference standard for mycoplasma contamination,
(v) contains less host cell DNA than a control reference standard, e.g., less than 10 ng of host cell DNA per dose, less than 5 ng of host cell DNA per dose,
(vi) contains less host cell protein (HCP) than a control reference standard, e.g., less than 100 ng/mL, less than 50 ng/mL, and/or less than 10 ng/dose, less than 5 ng/dose,
(vii) contains less than a threshold amount of non-infectious particles, e.g., meet a predetermined release specification for non-infectious particles relative to infectious particles, e.g., particles to infectious units <2000:1, <1000:1, <500:1, <300:1, <200:1, <100:1, or <50:1, and/or
(viii) contains less than a threshold amount of empty capsids (i.e., lacking a genome), e.g., meets a predetermined release specification for empty capsids.

In an aspect, the invention features a method of treating a disease or disorder in a subject, the method comprising administering to the subject a CAVector, e.g., a synthetic CAVector, e.g., as described herein.

In an aspect, the invention features a method of delivering an effector or payload (e.g., an endogenous or exogenous effector) to a cell, tissue or subject, the method comprising administering to the subject a CAVector, e.g., a synthetic CAVector, e.g., as described herein, wherein the CAVector comprises a nucleic acid sequence encoding the effector. In embodiments, the payload is a nucleic acid. In embodiments, the payload is a polypeptide.

In an aspect, the invention features a method of delivering an CAVector to a cell, comprising contacting the CAVector, e.g., a synthetic CAVector, e.g., as described herein, with a cell, e.g., a eukaryotic cell, e.g., a mammalian cell, e.g., in vivo or ex vivo.

In an aspect, the invention features a method of making a CAVector, e.g., a synthetic CAVector. The method includes:
(a) providing a host cell comprising:
   (i) a first nucleic acid molecule comprising a first copy of the nucleic acid sequence of a genetic element of a CAVector, e.g., as described herein, and a second copy of the nucleic acid sequence of a genetic element of a CAVector, or a portion thereof (e.g., an uRFS or a dRFS); and
   (ii) a second nucleic acid molecule encoding a CAV VP1 polypeptide, or one or more of an amino acid sequence chosen from a VP1, VP2, or Apoptin molecule, e.g., as described herein, or an amino acid sequence having at least 70% (e.g., at least 70%, 75%, 80%, 85%, 90%, 95%, 96%, 97%, 98%, 99%, or 100%) sequence identity thereto; and
(b) culturing the host cell under conditions suitable for replication (e.g., rolling circle replication) of the first copy of the nucleic acid sequence of the genetic element, thereby producing a genetic element;
optionally (c) culturing the host cell under conditions suitable for enclosure of the genetic element in a proteinaceous exterior (e.g., comprising a polypeptide encoded by the second nucleic acid molecule), and
optionally (d) harvesting CAVectors from the cell or cell culture.

In another aspect, the invention features a method of manufacturing an CAVector composition, comprising one or more of (e.g., all of) (a), (b), (c), and (d):
a) providing a host cell comprising, e.g., expressing one or more components (e.g., all of the components) of an CAVector, e.g., a synthetic CAVector, e.g., as described herein;
b) culturing the host cell under conditions suitable for producing a preparation of CAVectors from the host cell, wherein the CAVectors of the preparation comprise a proteinaceous exterior (e.g, comprising a CAVector VP1 polypeptide) encapsulating the genetic element (e.g., as described herein), thereby making a preparation of CAVectors;
optionally, c) harvesting the CAVectors from the host cell,
and
optionally, d) formulating the preparation of CAVectors, e.g., as a pharmaceutical composition suitable for administration to a subject.

For example, the host cell provided in this method of manufacturing comprises (a) a nucleic acid comprising a sequence encoding a CAV VP1 polypeptide described herein, wherein the nucleic acid is a plasmid, is a viral nucleic acid or genome, or is integrated into a helper cell chromosome; and (b) a tandem construct capable of producing a genetic element, wherein the genetic element comprises (i) a promoter element operably linked to a nucleic acid sequence (e.g., a DNA sequence) encoding an effector (e.g., an exogenous effector or an endogenous effector) and (i) a protein binding sequence (e.g, packaging signal) that binds the polypeptide of (a), wherein the host cell comprises (a) and (b) either in cis or in trans. In embodiments, the genetic element of (b) is circular, single-stranded DNA. In some embodiments, the host cell is a manufacturing cell line.

In some embodiments, the components of the CAVector are introduced into the host cell at the time of production (e.g., by transient transfection). In some embodiments, the host cell stably expresses the components of the CAVector (e.g., wherein one or more nucleic acids encoding the components of the CAVector are introduced into the host cell, or a progenitor thereof, e.g., by stable transfection).

In an aspect, the invention features a method of manufacturing a CAVector composition, comprising: a) providing a plurality of CAVectors described herein, or a preparation of CAVectors described herein; and b) formulating the CAVectors or preparation thereof, e.g., as a pharmaceutical composition suitable for administration to a subject.

In an aspect, the invention features a method of making a host cell, e.g., a first host cell or a producer cell, e.g., a population of first host cells, comprising a CAVector, the method comprising introducing a tandem construct capable of producing a genetic element, e.g., as described herein, to a host cell and culturing the host cell under conditions suitable for production of the CAVector. In embodiments, the method further comprises introducing a helper, e.g., a helper virus, to the host cell. In embodiments, the introducing comprises transfection (e.g., chemical transfection) or electroporation of the host cell with the CAVector.

In an aspect, the invention features a method of making a CAVector, comprising providing a host cell, e.g., a first host cell or producer cell, comprising a CAVector, e.g., as described herein, and purifying the CAVector from the host cell. In some embodiments, the method further comprises, prior to the providing step, contacting the host cell with a tandem construct or a CAVector, e.g., as described herein, and incubating the host cell under conditions suitable for production of the CAVector. In embodiments, the host cell is the first host cell or producer cell described in the above method of making a host cell. In embodiments, purifying the CAVector from the host cell comprises lysing the host cell.

In some embodiments, the method further comprises a second step of contacting the CAVector produced by the first host cell or producer cell with a second host cell, e.g., a permissive cell, e.g., a population of second host cells. In some embodiments, the method further comprises incubating the second host cell under conditions suitable for production of the CAVector. In some embodiments, the method further comprises purifying a CAVector from the second host cell, e.g., thereby producing a CAVector seed population. In embodiments, at least about 2-100-fold more of the CAVector is produced from the population of second host cells than from the population of first host cells. In embodiments, purifying the CAVector from the second host cell comprises lysing the second host cell. In some embodiments, the method further comprises a second step of contacting the CAVector produced by the second host cell with a third host cell, e.g., permissive cells, e.g., a population of third host cells. In some embodiments, the method further comprises incubating the third host cell under conditions suitable for production of the CAVector. In some embodiments, the method further comprises purifying a CAVector from the third host cell, e.g., thereby producing an CAVector stock population. In embodiments, purifying the CAVector from the third host cell comprises lysing the third host cell. In embodiments, at least about 2-100-fold more of the CAVector is produced from the population of third host cells than from the population of second host cells.

In some embodiments, the host cell is grown in a medium suitable for promoting cell growth. In certain embodiments, once the host cell has grown sufficiently (e.g., to an appropriate cell density), the medium may be exchanged with a medium suitable for production of CAVectors by the host cell. In some embodiments, CAVectors produced by a host cell separated from the host cell (e.g., by lysing the host cell) prior to contact with a second host cell. In some embodiments, CAVectors produced by a host cell are contacted with a second host cell without an intervening purification step.

In an aspect, the invention features a method of making a pharmaceutical CAVector preparation. The method comprises (a) making an CAVector preparation as described herein, (b) evaluating the preparation (e.g., a pharmaceutical CAVector preparation, CAVector seed population or the CAVector stock population) for one or more pharmaceutical quality control parameters, e.g., identity, purity, titer, potency (e.g., in genomic equivalents per CAVector particle), and/or the nucleic acid sequence, e.g., from the genetic element comprised by the CAVector, and (c) formulating the preparation for pharmaceutical use of the evaluation meets a predetermined criterion, e.g, meets a pharmaceutical specification. In some embodiments, evaluating identity comprises evaluating (e.g., confirming) the sequence of the genetic element of the CAVector, e.g., the sequence encoding the effector. In some embodiments, evaluating purity comprises evaluating the amount of an impurity, e.g., mycoplasma, endotoxin, host cell nucleic acids (e.g., host cell DNA and/or host cell RNA), animal-derived process impurities (e.g., serum albumin or trypsin), replication-competent agents (RCA), e.g., replication-competent virus or unwanted CAVectors (e.g., an CAVector other than the desired CAVector, e.g., a synthetic CAVector as described herein), free viral capsid protein, adventitious agents, and aggregates. In some embodiments, evaluating titer comprises evaluating the ratio of functional versus non-functional (e.g., infectious vs non-infectious) CAVectors in the preparation (e.g., as evaluated by HPLC). In some embodiments, evaluating potency comprises evaluating the level of CAVector function (e.g., expression and/or function of an effector encoded therein or genomic equivalents) detectable in the preparation.

In embodiments, the formulated preparation is substantially free of pathogens, host cell contaminants or impurities; has a predetermined level of non-infectious particles or a predetermined ratio of particles:infectious units (e.g., <300:1, <200:1, <100:1, or <50:1).

In some embodiments, multiple CAVectors can be produced in a single batch. In embodiments, the levels of the CAVectors produced in the batch can be evaluated (e.g., individually or together).

In an aspect, the invention features a host cell comprising:
(i) a first nucleic acid molecule comprising a tandem construct as described herein, and
(ii) optionally, a second nucleic acid molecule encoding one or more of an amino acid sequence chosen from a VP1, VP2 or Apoptin molecule, e.g., as described herein, or an amino acid sequence having at least about 70% (e.g., at least about 70, 80, 90, 95, 96, 97, 98, 99, or 100%) sequence identity thereto.

In an aspect, the invention features a reaction mixture comprising an CAVector described herein and a polynucleotide encoding an exterior protein, (e.g., an exterior protein capable of binding to the exterior protein binding sequence and, optionally, a lipid envelope), a polynucleotide encoding a replication protein (e.g., a polymerase), or any combination thereof.

In some embodiments, a CAVector (e.g., a synthetic CAVector) is isolated, e.g., isolated from a host cell and/or isolated from other constituents in a solution (e.g., a supernatant). In some embodiments, an CAVector (e.g., a synthetic CAVector) is purified, e.g., from a solution (e.g., a supernatant). In some embodiments, an CAVector is enriched in a solution relative to other constituents in the solution.

In some embodiments of any of the aforesaid CAVectors, compositions or methods, providing an CAVector comprises separating (e.g., harvesting) an CAVector from a composition comprising an CAVector-producing cell, e.g., as described herein. In other embodiments, providing an CAVector comprises obtaining an CAVector or a preparation thereof, e.g., from a third party.

In some embodiments of any of the aforesaid CAVectors, compositions or methods, the genetic element comprises an CAVector genome, e.g., as identified according to the methods described herein. In embodiments, the genetic element is capable of self-replication and/or self-amplification. In embodiments, the genetic element is not capable of self-replication and/or self-amplification. In embodiments, the genetic element is capable of replicating and/or being amplified in trans.

Additional features of any of the aforesaid CAVectors, compositions or methods include one or more of the following enumerated embodiments.

Those skilled in the art will recognize, or be able to ascertain using no more than routine experimentation, many equivalents to the specific embodiments of the invention described herein. Such equivalents are intended to be encompassed by the following enumerated embodiments.

### Enumerated Embodiments

1. A genetic element comprising:
a promoter element;
a nucleic acid sequence encoding an exogenous effector (e.g., a therapeutic exogenous effector), and
a protein binding sequence that specifically binds a CAV capsid polypeptide (e.g., a CAV VP1 molecule), e.g., with an affinity/specificity of less than about 10 μM (e.g., less than about 10, 9, 8, 7, 6, 5, 4, 3, 2, or 1 μM, e.g., less than about 900, 800, 700, 600, 500, 400, 300, 200, 100, 90, 80, 70, 60, 50, 40, 30, 20, or 10 nM).
2. A genetic element comprising:
a promoter element;
a nucleic acid sequence encoding an exogenous effector (e.g., a therapeutic exogenous effector), and
a protein binding sequence;
wherein the genetic element is capable of being packaged (e.g., specifically packaged) by a CAV VP1 molecule.
3. A genetic element comprising:
a promoter element;
a nucleic acid sequence encoding an exogenous effector (e.g., a therapeutic exogenous effector), and
a protein binding sequence that specifically binds to a CAV capsid polypeptide;
wherein the exogenous effector is:
   (a) codon optimized for expression in a human cell,
   (b) a human polypeptide or nucleic acid,
   (c) binds a human polypeptide or nucleic acid, or
   (d) has an activity in a human cell, e.g., modulates (e.g., increases or decreases) the activity and/or level of a human gene in the human cell).
4. The genetic element of any of the preceding embodiments, wherein the protein binding sequence comprises the nucleic acid sequence AGCCCTGAAAAGGGGGGGGGGCTAAAGCCCCCCCCCCTTAAACCCCCCCCTGGGGGGGATT CCCCCCCAGACCCCCCCTTTATATAGCACTCAATAAACGCAGAAAATAGATTTATCGCACTA TC (SEQ ID NO: 17), or a nucleic acid sequence having at least 75%, 80%, 85%, 90%, 95%, 96%, 97%, 98%, or 99% sequence identity thereto, or a reverse complement thereof.
5. A genetic element comprising:
a promoter element;
a nucleic acid sequence encoding an exogenous effector (e.g., a therapeutic exogenous effector), and
a nucleic acid sequence having at least 75%, 80%, 85%, 90%, 95%, 96%, 97%, 98%, 99%, or 100% sequence identity to nucleotides 1-374 of SEQ ID NO: 1, and/or a nucleic acid sequence having at least 75%, 80%, 85%, 90%, 95%, 96%, 97%, 98%, 99%, or 100% sequence identity to nucleotides 2195-2319 of SEQ ID NO: 100.
6. A genetic element comprising:
a promoter element;
a nucleic acid sequence encoding an exogenous effector (e.g., a therapeutic exogenous effector), and
at least 100, 200, 300, 400, 500, 600, 700, 800, 900, 1,000, 1,500, 2,000, 2,500, or 3,000 nucleotides having at least 70%, 75%, 80%, 85%, 90%, 95%, 96%, 97%, 98%, 99%, or 100% sequence identity to a contiguous portion of a CAV genome sequence (e.g., as described herein).
7. The genetic element of embodiment 6, comprising at least 1,000 nucleotides having at least 70%, 75%, 80%, 85%, 90%, 95%, 96%, 97%, 98%, 99%, or 100% sequence identity to a contiguous portion of a CAV genome sequence (e.g., as described herein).
8. A genetic element comprising:
a protein binding sequence that specifically binds a CAV capsid polypeptide e.g., with an affinity/specificity of less than about 10 μM (e.g., less than about 10, 9, 8, 7, 6, 5, 4, 3, 2, or 1 μM, e.g., less than about 900, 800, 700, 600, 500, 400, 300, 200, 100, 90, 80, 70, 60, 50, 40, 30, 20, or 10 nM),
wherein the genetic element does not comprise one or more of:
(i) a full length CAV VP1 gene (e.g., wherein the genetic element comprises one or more fragments of the CAV VP1 gene, e.g., less than about 500, 400, 300, 200, or 100 nucleotides of CAV VP1 gene sequence);
(ii) a full length CAV VP2 gene (e.g., wherein the genetic element comprises one or more fragments of the CAV VP2 gene, e.g., less than about 500, 400, 300, 200, or 100 nucleotides of CAV VP2 gene sequence); or
(ii) a full length CAV Apoptin gene (e.g., wherein the genetic element comprises one or more fragments of the CAV Apoptin gene, e.g., less than about 500, 400, 300, 200, or 100 nucleotides of CAV Apoptin gene sequence).
9. A genetic element comprising:
a protein binding sequence that specifically binds a CAV capsid polypeptide e.g., with an affinity/specificity of less than about 10 μM (e.g., less than about 10, 9, 8, 7, 6, 5, 4, 3, 2, or 1 μM, e.g., less than about 900, 800, 700, 600, 500, 400, 300, 200, 100, 90, 80, 70, 60, 50, 40, 30, 20, or 10 nM),
wherein the genetic element comprises one or more of:
(i) a nonfunctional CAV VP1 gene or a fragment thereof (e.g., a contiguous fragment of at least 25, 50, 100, 200, 300, 400, 500 or more bp), e.g., comprising a stop codon within the sequence of the CAV VP1 coding sequence, e.g., at the 5′ end of the CAV VP1 coding sequence;
(ii) a nonfunctional CAV VP2 gene or a fragment thereof (e.g., a contiguous fragment of at least 25, 50, 100, 200, 300, 400, 500 or more bp), e.g., comprising a stop codon within the sequence of the CAV VP2 coding sequence, e.g., at the 5′ end of the CAV VP2 coding sequence; or
(ii) a nonfunctional CAV Apoptin gene or a fragment thereof (e.g., a contiguous fragment of at least 25, 50, 100, 200, 300, 400, 500 or more bp), e.g., comprising a stop codon within the sequence of the CAV Apoptin coding sequence, e.g., at the 5′ end of the CAV Apoptin coding sequence.
10. The genetic element of embodiment 8 or 9, wherein the protein binding sequence comprises the nucleic acid sequence AGCCCTGAAAAGGGGGGGGGGCTAAAGCCCCCCCCCCTTAAACCCCCCCCTGGGGGGGATT CCCCCCCAGACCCCCCCTTTATATAGCACTCAATAAACGCAGAAAATAGATTTATCGCACTA TC (SEQ ID NO: 17), or a nucleic acid sequence having at least 75%, 80%, 85%, 90%, 95%, 96%, 97%, 98%, or 99% sequence identity thereto, or a reverse complement thereof.
11. The genetic element of any of the preceding embodiments, wherein the genetic element does not comprise a functional CAV Apoptin gene.
12. The genetic element of any of the preceding embodiments, wherein the genetic element does not comprise a functional CAV VP1 gene, a functional CAV VP2 gene, or a functional CAV Apoptin gene.
13. The genetic element of any of the preceding embodiments, wherein the genetic element does not comprise a truncated CAV VP1 gene (e.g., a contiguous sequence of less than 1200, 1100, 1000, 900, 800, 700, 600, 500, 400, 300, 200, 100, 50, 40, 30, or 20 nucleotides comprised in a CAV VP1 coding sequence).
14. The genetic element of any of the preceding embodiments, wherein the genetic element does not comprise a truncated CAV VP2 gene (e.g., a contiguous sequence of less than 600, 500, 400, 300, 200, 100, 50, 40, 30, or 20 nucleotides comprised in a CAV VP2 coding sequence).
15. The genetic element of any of the preceding embodiments, wherein the genetic element does not comprise a truncated CAV Apoptin gene (e.g., a contiguous sequence of less than 350, 300, 200, 100, 50, 40, 30, or 20 nucleotides comprised in a CAV Apoptin coding sequence).
16. The genetic element of any of the preceding embodiments, further comprising a promoter element and a nucleic acid sequence encoding an effector (e.g., an exogenous effector, e.g., a therapeutic effector).
17. The genetic element of any of the preceding embodiments, which comprises one or both of:
at least 10, 20, 30, 40, 50, 60, 70, 80, 90, 100, 150, 200, 250, 300, 400, 500, 600, 601, 602, 603, 604, 605, or 606 nucleotides having at least 70%, 75%, 80%, 85%, 90%, 95%, 96%, 97%, 98%, 99%, or 100% sequence identity to the sequence of nucleotides 1-606 of SEQ ID NO: 10, and/or
at least 10, 20, 30, 40, 50, 60, 70, 80, 90, 100, 105, 110, 115, 120, 121, 122, 123, or 124 nucleotides having at least 70%, 75%, 80%, 85%, 90%, 95%, 96%, 97%, 98%, 99%, or 100% sequence identity to the sequence of nucleotides 2195-2319 of SEQ ID NO: 10.
18. The genetic element of any of the preceding embodiments, which comprises a CAV UTR, e.g., a CAV 5′ UTR (e.g., as listed in any of Tables 1A, 1B, or 2-13), or a nucleic acid sequence having at least 75%, 80%, 85%, 90%, 95%, 96%, 97%, 98%, or 99% sequence identity thereto.
19. The genetic element of any of the preceding embodiments, which comprises a full length CAV VP1 gene.
20. The genetic element of any of the preceding embodiments, which comprises a full length CAV VP2 gene.
21. The genetic element of any of the preceding embodiments, which comprises a full length CAV Apoptin gene.
22. The genetic element of any of embodiments 1-18, 20, or 21, which does not comprise a full length CAV VP1 gene.
23. The genetic element of embodiment 22, which comprises 1-10, 10-50, 50-100, 100-200, 200-300, 300-400, 400-500, 500-600, 600-700, 700-800, 800-900, or 900-1000 nucleotides from the 5′ end of the CAV VP1 gene, or a sequence having at least 70%, 75%, 80%, 85%, 90%, 95%, 96%, 97%, 98%, 99%, or 100% identity thereto.
24. The genetic element of embodiment 22 or 23, which comprises 1-10, 10-50, 50-100, 100-200, 200-300, 300-400, 400-500, 500-600, 600-700, 700-800, 800-900, or 900-1000 nucleotides from the 3′ end of the CAV VP1 gene, or a sequence having at least 70%, 75%, 80%, 85%, 90%, 95%, 96%, 97%, 98%, 99%, or 100% identity thereto.
25. The genetic element of embodiment 22 or 23, which comprises less than 1349, 1340, 1330, 1320, 1310, 1300, 1250, 1200, 1100, 1000, 900, 800, 700, 600, 500, 400, 300, 200, 100, 50, 40, 30, 20, 10, 5, 4, 3, 2, or 1 nucleotides of the CAV VP1 gene sequence, or a sequence having at least 70%, 75%, 80%, 85%, 90%, 95%, 96%, 97%, 98%, 99%, or 100% identity thereto.
26. The genetic element of any of the preceding embodiments, which does not comprise a full length CAV VP2 gene.
27. The genetic element of embodiment 26, which comprises less than 226, 220, 210, 200, 190, 180, 170, 160, 150, 100, 50, 40, 30, 20, 10, or 5 nucleotides from the 5′ end of the CAV VP2 gene, or a sequence having at least 70%, 75%, 80%, 85%, 90%, 95%, 96%, 97%, 98%, 99%, or 100% identity thereto.
28. The genetic element of embodiment 26 or 27, which comprises less than 226, 220, 210, 200, 190, 180, 170, 160, 150, 100, 50, 40, 30, 20, 10, or 5 nucleotides from the 3′ end of the CAV VP2 gene, or a sequence having at least 70%, 75%, 80%, 85%, 90%, 95%, 96%, 97%, 98%, 99%, or 100% identity thereto.
29. The genetic element of embodiment 26 or 27, which comprises less than 650, 640, 630, 620, 610, 600, 550, 500, 450, 400, 350, 300, 250, 200, 190, 180, 170, 160, 150, 100, 50, 40, 30, 29, 28, 27, or 26 nucleotides of CAV VP2 gene sequence.
30. The genetic element of any of the preceding embodiments, which does not comprise a full length CAV Apoptin gene.
31. The genetic element of embodiment 30, which comprises 1-10, 10-50, 50-100, 100-200, 200-300, or 300-350 nucleotides from the 5′ end of CAV Apoptin gene, or a sequence having at least 70%, 75%, 80%, 85%, 90%, 95%, 96%, 97%, 98%, 99%, or 100% identity thereto.
32. The genetic element of embodiment 30 or 31, which comprises 1-10, 10-50, 50-100, 100-200, 200-300, or 300-350 nucleotides from the 3′ end of CAV Apoptin gene, or a sequence having at least 70%, 75%, 80%, 85%, 90%, 95%, 96%, 97%, 98%, 99%, or 100% identity thereto.
33. The genetic element of embodiment 30 or 31, which comprises less than 365, 360, 350, 340, 330, 320, 310, 300, 250, 200, 150, 100, 50, 40, 30, 20, 10, 5, 4, 3, 2, or 1 nucleotides of CAV Apoptin gene sequence.
34. The genetic element of any of the preceding embodiments, which is 1,000-1,500, 1,500-2,000, or 2,000-2,500, or less than 2,500, 2,400, 2,300, 2,200, 2,100, or 2,000 nucleotides in length.
35. The genetic element of any of the preceding embodiments, which is DNA, e.g., single stranded DNA.
36. The genetic element of any of the preceding embodiments, which is circular or linear.
37. The genetic element of any of the preceding embodiments, which produced using a circularized double-stranded DNA, e.g., wherein the circularized DNA was produced by in vitro circularization.
38. The genetic element of any of the preceding embodiments, which was produced using a tandem nucleic acid construct.
39. The genetic element of any of the preceding embodiments, wherein the promoter element is endogenous to a CAV.
40. The genetic element of any of embodiments 1-38, wherein the promoter element is exogenous to a CAV.
41. The genetic element of any of the preceding embodiments, comprising the nucleic acid sequence AGCCCTGAAAAGGGGGGGGGGCTAAAGCCCCCCCCCCTTAAACCCCCCCCTGGGGGGGATT CCCCCCCAGACCCCCCCTTTATATAGCACTCAATAAACGCAGAAAATAGATTTATCGCACTA TC (SEQ ID NO: 17), or a nucleic acid sequence having at least 75%, 80%, 85%, 90%, 95%, 96%, 97%, 98%, or 99% sequence identity thereto, or a reverse complement thereof.
42. The genetic element of any of the preceding embodiments, comprising nucleotides 1-374 of SEQ ID NO: 1, or a nucleic acid sequence having at least 75%, 80%, 85%, 90%, 95%, 96%, 97%, 98%, or 99% sequence identity thereto.
43. The genetic element of any of the preceding embodiments, comprising nucleotides 138-254 of SEQ ID NO: 1, or a nucleic acid sequence having at least 75%, 80%, 85%, 90%, 95%, 96%, 97%, 98%, or 99% sequence identity thereto.
44. The genetic element of any of the preceding embodiments, comprising nucleotides 255-260 of SEQ ID NO: 1, or a nucleic acid sequence having at least 75%, 80%, 85%, 90%, 95%, 96%, 97%, 98%, or 99% sequence identity thereto.
45. The genetic element of any of the preceding embodiments, comprising nucleotides 317-322 of SEQ ID NO: 1, or a nucleic acid sequence having at least 75%, 80%, 85%, 90%, 95%, 96%, 97%, 98%, or 99% sequence identity thereto.
46. The genetic element of any of the preceding embodiments, comprising nucleotides 374-1024 of SEQ ID NO: 1, or a nucleic acid sequence having at least 75%, 80%, 85%, 90%, 95%, 96%, 97%, 98%, or 99% sequence identity thereto.
47. The genetic element of any of the preceding embodiments, comprising nucleotides 480-845 of SEQ ID NO: 1, or a nucleic acid sequence having at least 75%, 80%, 85%, 90%, 95%, 96%, 97%, 98%, or 99% sequence identity thereto.
48. The genetic element of any of the preceding embodiments, comprising nucleotides 847-2196 of SEQ ID NO: 1, or a nucleic acid sequence having at least 75%, 80%, 85%, 90%, 95%, 96%, 97%, 98%, or 99% sequence identity thereto.
49. The genetic element of any of the preceding embodiments, comprising nucleotides 2197-2313 of SEQ ID NO: 1, or a nucleic acid sequence having at least 75%, 80%, 85%, 90%, 95%, 96%, 97%, 98%, or 99% sequence identity thereto.
50. The genetic element of any of the preceding embodiments, comprising nucleotides 2200-2266 of SEQ ID NO: 1, or a nucleic acid sequence having at least 75%, 80%, 85%, 90%, 95%, 96%, 97%, 98%, or 99% sequence identity thereto.
51. The genetic element of any of the preceding embodiments, comprising nucleotides 2281-2286 of SEQ ID NO: 1, or a nucleic acid sequence having at least 75%, 80%, 85%, 90%, 95%, 96%, 97%, 98%, or 99% sequence identity thereto.
52. The genetic element of any of the preceding embodiments, which does not comprise nucleotides 1-374 of SEQ ID NO: 1, or a nucleic acid sequence having at least 75%, 80%, 85%, 90%, 95%, 96%, 97%, 98%, or 99% sequence identity thereto.
53. The genetic element of any of the preceding embodiments, which does not comprise nucleotides 138-254 of SEQ ID NO: 1, or a nucleic acid sequence having at least 75%, 80%, 85%, 90%, 95%, 96%, 97%, 98%, or 99% sequence identity thereto.
54. The genetic element of any of the preceding embodiments, which does not comprise nucleotides 255-260 of SEQ ID NO: 1, or a nucleic acid sequence having at least 75%, 80%, 85%, 90%, 95%, 96%, 97%, 98%, or 99% sequence identity thereto.
55. The genetic element of any of the preceding embodiments, which does not comprise nucleotides 317-322 of SEQ ID NO: 1, or a nucleic acid sequence having at least 75%, 80%, 85%, 90%, 95%, 96%, 97%, 98%, or 99% sequence identity thereto.
56. The genetic element of any of the preceding embodiments, which does not comprise nucleotides 374-1024 of SEQ ID NO: 1, or a nucleic acid sequence having at least 75%, 80%, 85%, 90%, 95%, 96%, 97%, 98%, or 99% sequence identity thereto.
57. The genetic element of any of the preceding embodiments, which does not comprise nucleotides 480-845 of SEQ ID NO: 1, or a nucleic acid sequence having at least 75%, 80%, 85%, 90%, 95%, 96%, 97%, 98%, or 99% sequence identity thereto.
58. The genetic element of any of the preceding embodiments, which does not comprise nucleotides 847-2196 of SEQ ID NO: 1, or a nucleic acid sequence having at least 75%, 80%, 85%, 90%, 95%, 96%, 97%, 98%, or 99% sequence identity thereto.
59. The genetic element of any of the preceding embodiments, which does not comprise nucleotides 2197-2313 of SEQ ID NO: 1, or a nucleic acid sequence having at least 75%, 80%, 85%, 90%, 95%, 96%, 97%, 98%, or 99% sequence identity thereto.
60. The genetic element of any of the preceding embodiments, which does not comprise nucleotides 2200-2266 of SEQ ID NO: 1, or a nucleic acid sequence having at least 75%, 80%, 85%, 90%, 95%, 96%, 97%, 98%, or 99% sequence identity thereto.
61. The genetic element of any of the preceding embodiments, which does not comprise nucleotides 2281-2286 of SEQ ID NO: 1, or a nucleic acid sequence having at least 75%, 80%, 85%, 90%, 95%, 96%, 97%, 98%, or 99% sequence identity thereto.
62. The genetic element of any of the preceding embodiments, which comprises nucleotides 1-606 and/or nucleotides 1595-2319 of SEQ ID NO: 10, or a nucleic acid sequence having at least 75%, 80%, 85%, 90%, 95%, 96%, 97%, 98%, or 99% sequence identity thereto.
63. The genetic element of any of the preceding embodiments, which comprises nucleotides 1-806 and/or nucleotides 1795-2319 of SEQ ID NO: 10, or a nucleic acid sequence having at least 75%, 80%, 85%, 90%, 95%, 96%, 97%, 98%, or 99% sequence identity thereto.
64. The genetic element of any of the preceding embodiments, which comprises nucleotides 1-1006 and/or nucleotides 1995-2319 of SEQ ID NO: 10, or a nucleic acid sequence having at least 75%, 80%, 85%, 90%, 95%, 96%, 97%, 98%, or 99% sequence identity thereto.
65. The genetic element of any of the preceding embodiments, which comprises nucleotides 1-1206 and/or nucleotides 2195-2319 of SEQ ID NO: 10, or a nucleic acid sequence having at least 75%, 80%, 85%, 90%, 95%, 96%, 97%, 98%, or 99% sequence identity thereto.
66. The genetic element of any of the preceding embodiments, comprising nucleotides 1-379 of SEQ ID NO: 10, or a nucleic acid sequence having at least 75%, 80%, 85%, 90%, 95%, 96%, 97%, 98%, or 99% sequence identity thereto.
67. The genetic element of any of the preceding embodiments, comprising nucleotides 380-1030 of SEQ ID NO: 10, or a nucleic acid sequence having at least 75%, 80%, 85%, 90%, 95%, 96%, 97%, 98%, or 99% sequence identity thereto.
68. The genetic element of any of the preceding embodiments, comprising nucleotides 485-851 of SEQ ID NO: 10, or a nucleic acid sequence having at least 75%, 80%, 85%, 90%, 95%, 96%, 97%, 98%, or 99% sequence identity thereto.
69. The genetic element of any of the preceding embodiments, comprising nucleotides 853-2202 of SEQ ID NO: 10, or a nucleic acid sequence having at least 75%, 80%, 85%, 90%, 95%, 96%, 97%, 98%, or 99% sequence identity thereto.
70. The genetic element of any of the preceding embodiments, comprising nucleotides 2203-2319 of SEQ ID NO: 10, or a nucleic acid sequence having at least 75%, 80%, 85%, 90%, 95%, 96%, 97%, 98%, or 99% sequence identity thereto.
71. The genetic element of any of the preceding embodiments, which does not comprise nucleotides 138-254 of SEQ ID NO: 10, or a nucleic acid sequence having at least 75%, 80%, 85%, 90%, 95%, 96%, 97%, 98%, or 99% sequence identity thereto.
72. The genetic element of any of the preceding embodiments, which does not comprise nucleotides 255-260 of SEQ ID NO: 10, or a nucleic acid sequence having at least 75%, 80%, 85%, 90%, 95%, 96%, 97%, 98%, or 99% sequence identity thereto.
73. The genetic element of any of the preceding embodiments, which does not comprise nucleotides 317-322 of SEQ ID NO: 10, or a nucleic acid sequence having at least 75%, 80%, 85%, 90%, 95%, 96%, 97%, 98%, or 99% sequence identity thereto.
74. The genetic element of any of the preceding embodiments, which does not comprise nucleotides 374-1024 of SEQ ID NO: 10, or a nucleic acid sequence having at least 75%, 80%, 85%, 90%, 95%, 96%, 97%, 98%, or 99% sequence identity thereto.
75. The genetic element of any of the preceding embodiments, which does not comprise nucleotides 480-845 of SEQ ID NO: 10, or a nucleic acid sequence having at least 75%, 80%, 85%, 90%, 95%, 96%, 97%, 98%, or 99% sequence identity thereto.
76. The genetic element of any of the preceding embodiments, which does not comprise nucleotides 847-2196 of SEQ ID NO: 10, or a nucleic acid sequence having at least 75%, 80%, 85%, 90%, 95%, 96%, 97%, 98%, or 99% sequence identity thereto.
77. The genetic element of any of the preceding embodiments, which does not comprise nucleotides 2197-2313 of SEQ ID NO: 10, or a nucleic acid sequence having at least 75%, 80%, 85%, 90%, 95%, 96%, 97%, 98%, or 99% sequence identity thereto.
78. The genetic element of any of the preceding embodiments, which does not comprise nucleotides 2197-2313 of SEQ ID NO: 10, or a nucleic acid sequence having at least 75%, 80%, 85%, 90%, 95%, 96%, 97%, 98%, or 99% sequence identity thereto.
79. The genetic element of any of the preceding embodiments, which comprises one or more (e.g., 1, 2, 3, 4, 5, 6, 7, or all 8) of a repeat region, CAAT signal, TATA box, VP2, Apoptin, VP1, 3′ UTR, and/or GC-rich region from a wild-type CAV genome (e.g., a Cuxhaven 1 isolate genome, e.g., as shown in Table 1), or a nucleic acid sequence having at least 75%, 80%, 85%, 90%, 95%, 96%, 97%, 98%, or 99% sequence identity thereto.
80. The genetic element of any of the preceding embodiments, which comprises one or more (e.g., 1, 2, 3, 4, 5, 6, 7, or all 8) of a repeat region, CAAT signal, TATA box, VP2, Apoptin, VP1, 3′ UTR, and/or GC-rich region from a CAV isolate 1535TW genome, or a nucleic acid sequence having at least 75%, 80%, 85%, 90%, 95%, 96%, 97%, 98%, or 99% sequence identity thereto.
81. The genetic element of any of the preceding embodiments, which comprises one or more (e.g., 1, 2, 3, 4, 5, 6, 7, or all 8) of a repeat region, CAAT signal, TATA box, VP2, Apoptin, VP1, 3′ UTR, and/or GC-rich region from a CAV isolate N5 genome, or a nucleic acid sequence having at least 75%, 80%, 85%, 90%, 95%, 96%, 97%, 98%, or 99% sequence identity thereto.
82. The genetic element of any of the preceding embodiments, which comprises one or more (e.g., 1, 2, 3, 4, 5, 6, 7, or all 8) of a repeat region, CAAT signal, TATA box, VP2, Apoptin, VP1, 3′ UTR, and/or GC-rich region from a CAV isolate 1623TW genome, or a nucleic acid sequence having at least 75%, 80%, 85%, 90%, 95%, 96%, 97%, 98%, or 99% sequence identity thereto.
83. The genetic element of any of the preceding embodiments, which comprises one or more (e.g., 1, 2, 3, 4, 5, 6, 7, or all 8) of a repeat region, CAAT signal, TATA box, VP2, Apoptin, VP1, 3′ UTR, and/or GC-rich region from a CAV isolate CAV-EG-7 genome, or a nucleic acid sequence having at least 75%, 80%, 85%, 90%, 95%, 96%, 97%, 98%, or 99% sequence identity thereto.
84. The genetic element of any of the preceding embodiments, which comprises one or more (e.g., 1, 2, 3, 4, 5, 6, 7, or all 8) of a repeat region, CAAT signal, TATA box, VP2, Apoptin, VP1, 3′ UTR, and/or GC-rich region from a CAV isolate HLJ15108 genome, or a nucleic acid sequence having at least 75%, 80%, 85%, 90%, 95%, 96%, 97%, 98%, or 99% sequence identity thereto.
85. The genetic element of any of the preceding embodiments, which comprises one or more (e.g., 1, 2, 3, 4, 5, 6, 7, or all 8) of a repeat region, CAAT signal, TATA box, VP2, Apoptin, VP1, 3′ UTR, and/or GC-rich region from a CAV isolate LN1402 genome, or a nucleic acid sequence having at least 75%, 80%, 85%, 90%, 95%, 96%, 97%, 98%, or 99% sequence identity thereto.
86. The genetic element of any of the preceding embodiments, which comprises one or more (e.g., 1, 2, 3, 4, 5, 6, 7, or all 8) of a repeat region, CAAT signal, TATA box, VP2, Apoptin, VP1, 3′ UTR, and/or GC-rich region from a CAV isolate GD-F-12 genome, or a nucleic acid sequence having at least 75%, 80%, 85%, 90%, 95%, 96%, 97%, 98%, or 99% sequence identity thereto.
87. The genetic element of any of the preceding embodiments, which comprises one or more (e.g., 1, 2, 3, 4, 5, 6, 7, or all 8) of a repeat region, CAAT signal, TATA box, VP2, Apoptin, VP1, 3′ UTR, and/or GC-rich region from a CAV isolate GX1805 genome, or a nucleic acid sequence having at least 75%, 80%, 85%, 90%, 95%, 96%, 97%, 98%, or 99% sequence identity thereto.
88. The genetic element of any of the preceding embodiments, which comprises one or more (e.g., 1, 2, 3, 4, 5, 6, 7, or all 8) of a repeat region, CAAT signal, TATA box, VP2, Apoptin, VP1, 3′ UTR, and/or GC-rich region from a CAV isolate JL14026 genome, or a nucleic acid sequence having at least 75%, 80%, 85%, 90%, 95%, 96%, 97%, 98%, or 99% sequence identity thereto.
89. The genetic element of any of the preceding embodiments, which comprises one or more (e.g., 1, 2, 3, 4, 5, 6, 7, or all 8) of a repeat region, CAAT signal, TATA box, VP2, Apoptin, VP1, 3′ UTR, and/or GC-rich region from a CAV isolate HB1517 genome, or a nucleic acid sequence having at least 75%, 80%, 85%, 90%, 95%, 96%, 97%, 98%, or 99% sequence identity thereto.
90. The genetic element of any of the preceding embodiments, which comprises one or more (e.g., 1, 2, 3, 4, 5, 6, 7, or all 8) of a repeat region, CAAT signal, TATA box, VP2, Apoptin, VP1, 3′ UTR, and/or GC-rich region from a CAV isolate N1 genome, or a nucleic acid sequence having at least 75%, 80%, 85%, 90%, 95%, 96%, 97%, 98%, or 99% sequence identity thereto.
91. The genetic element of any of the preceding embodiments, which comprises one or more (e.g., 1, 2, 3, 4, 5, 6, 7, or all 8) of a repeat region, CAAT signal, TATA box, VP2, Apoptin, VP1, 3′ UTR, and/or GC-rich region from a CAV isolate N2 genome, or a nucleic acid sequence having at least 75%, 80%, 85%, 90%, 95%, 96%, 97%, 98%, or 99% sequence identity thereto.
92. The genetic element of any of the preceding embodiments, which comprises one or more (e.g., 1, 2, 3, 4, 5, 6, 7, or all 8) of a repeat region, CAAT signal, TATA box, VP2, Apoptin, VP1, 3′ UTR, and/or GC-rich region from a CAV isolate HN1504 genome, or a nucleic acid sequence having at least 75%, 80%, 85%, 90%, 95%, 96%, 97%, 98%, or 99% sequence identity thereto.
93. The genetic element of any of the preceding embodiments, which comprises one or more (e.g., 1, 2, 3, 4, 5, 6, 7, or all 8) of a repeat region, CAAT signal, TATA box, VP2, Apoptin, VP1, 3′ UTR, and/or GC-rich region from a CAV isolate N3 genome, or a nucleic acid sequence having at least 75%, 80%, 85%, 90%, 95%, 96%, 97%, 98%, or 99% sequence identity thereto.
94. The genetic element of any of the preceding embodiments, which comprises one or more (e.g., 1, 2, 3, 4, 5, 6, 7, or all 8) of a repeat region, CAAT signal, TATA box, VP2, Apoptin, VP1, 3′ UTR, and/or GC-rich region from an avian gyrovirus (e.g., a CAV-related avian gyrovirus, e.g., CAV-related avian gyrovirus 2, e.g., having the sequence of NCBI Accession No. NC_015396), or a nucleic acid sequence having at least 75%, 80%, 85%, 90%, 95%, 96%, 97%, 98%, or 99% sequence identity thereto.
95. The genetic element of any of the preceding embodiments, which comprises one or more (e.g., 1, 2, 3, 4, 5, 6, 7, or all 8) of a repeat region, CAAT signal, TATA box, VP2, Apoptin, VP1, 3′ UTR, and/or GC-rich region from a CAV genome listed in Table 17, or a nucleic acid sequence having at least 75%, 80%, 85%, 90%, 95%, 96%, 97%, 98%, or 99% sequence identity thereto.
96. The genetic element of any of the preceding embodiments, which does not comprise a nucleic acid sequence encoding a CAV VP1 protein, or a functional fragment or variant thereof.
97. The genetic element of any of the preceding embodiments, which does not comprise a nucleic acid sequence encoding the arginine-rich region of a CAV VP1 protein.
98. The genetic element of any of the preceding embodiments, which does not comprise a nucleic acid sequence encoding the jelly-roll domain of a CAV VP1 protein.
99. The genetic element of any of the preceding embodiments, which does not comprise a nucleic acid sequence encoding a CAV VP2 protein, or a functional fragment or variant thereof.
100. The genetic element of embodiment 99, which does not comprise a nucleic acid sequence encoding the amino acid sequence I⁹⁴CNCGQFRKH¹⁰³.
101. The genetic element of embodiment 99 or 100, which does not comprise a nucleic acid sequence encoding the amino acid sequence WLRECSRSHAKICNCGQFRKH.
102. The genetic element of embodiment 100 or 101, which does not comprise a nucleic acid sequence encoding an amino acid sequence forming a metal ion coordination site (e.g., a Zn2+ coordination site).
103. The genetic element of any of the preceding embodiments, which does not comprise a nucleic acid sequence encoding a CAV Apoptin protein, or a functional fragment or variant thereof.
104. The genetic element of any of the preceding embodiments, comprising a nucleic acid sequence encoding a CAV VP1 protein, or a functional fragment or variant thereof.
105. The genetic element of embodiment 104, wherein the CAV VP1 protein comprises an arginine-rich region.
106. The genetic element of embodiment 104 or 105, wherein the CAV VP1 protein comprises a jelly-roll domain.
107. The genetic element of any of embodiments 104-106, wherein the CAV VP1 protein comprises one or more DNA-binding motifs.
108. The genetic element of any of embodiments 104-107, wherein the CAV VP1 protein comprises a DNA-binding motif comprising the amino acid sequence RRARRPRGRFYAFRRGR, or an amino acid sequence having no more than 1, 2, 3, 4, 5, 6, 7, 8, 9, or 10 amino acid differences therefrom.
109. The genetic element of any of embodiments 104-108, wherein the CAV VP1 protein comprises a DNA-binding motif comprising the amino acid sequence RRRYKFRHRRQRYRRRAFRKH, or an amino acid sequence having no more than 1, 2, 3, 4, 5, 6, 7, 8, 9, or 10 amino acid differences therefrom.
110. The genetic element of any of embodiments 104-109, wherein the CAV VP1 protein comprises a DNA-binding motif comprising the amino acid sequence SRRSFNHHKARGAGDPK, or an amino acid sequence having no more than 1, 2, 3, 4, 5, 6, 7, 8, 9, or 10 amino acid differences therefrom.
111. The genetic element of any of embodiments 104-110, wherein the CAV VP1 protein comprises one or more (e.g., two) nuclear localization signals (NLS).
112. The genetic element of embodiment 111, wherein the NLS comprises the amino acid sequence RRARRPRGRFYAFRRGRWHH, or an amino acid sequence having no more than 1, 2, 3, 4, 5, 6, 7, 8, 9, or 10 amino acid differences therefrom,
113. The genetic element of embodiment 111, wherein the NLS comprises the amino acid sequence KRLRRRYKFRHRRRQRYRRRAFRK, or an amino acid sequence having no more than 1, 2, 3, 4, 5, 6, 7, 8, 9, or 10 amino acid differences therefrom,
114. The genetic element of any of embodiments 104-113, wherein the CAV VP1 protein comprises one or more (e.g., two or three) nuclear export signals (NES).
115. The genetic element of embodiment 114, wherein the NES comprises the amino acid sequence IFLTEGLIL, or an amino acid sequence having no more than 1, 2, 3, 4, or 5 amino acid differences therefrom,
116. The genetic element of embodiment 114, wherein the NES comprises the amino acid sequence LKEFLLASMNL, or an amino acid sequence having no more than 1, 2, 3, 4, or 5 amino acid differences therefrom,
117. The genetic element of embodiment 114, wherein the NES comprises the amino acid sequence ELDTNFFTLYVAQ, or an amino acid sequence having no more than 1, 2, 3, 4, 5, 6, 7, 8, 9, or 10 amino acid differences therefrom,
118. The genetic element of any of the preceding embodiments, comprising a nucleic acid sequence encoding a CAV VP2 protein, or a functional fragment or variant thereof.
119. The genetic element of embodiment 118, wherein the CAV VP2 protein, or the functional fragment thereof, comprises the amino acid sequence I⁹⁴CNCGQFRKH¹⁰³.
120. The genetic element of embodiment 118, wherein the CAV VP2 protein, or the functional fragment thereof, comprises the amino acid sequence WX₇HX₃CXCX₅H.
121. The genetic element of embodiment 1220, wherein each X can be any amino acid.
122. The genetic element of embodiment 120 or 121, wherein each Xₙ comprises n amino acids.
123. The genetic element of any of embodiments 118-122, wherein the CAV VP2 protein, or the functional fragment thereof, comprises the amino acid sequence WLRECSRSHAKICNCGQFRKH.
124. The genetic element of any of embodiments 119-123, wherein the cysteine and histidine residues of the amino acid sequence form a metal ion coordination site (e.g., a Zn2+ coordination site).
125. The genetic element of any of the preceding embodiments, comprising a nucleic acid sequence encoding a CAV Apoptin protein, or a functional fragment or variant thereof
126. A nucleic acid construct comprising the nucleic acid sequence of a genetic element of any of the preceding embodiments.
127. The nucleic acid construct of embodiment 126, which is DNA, e.g., single stranded or double stranded DNA.
128. The nucleic acid construct of embodiment 126 or 127, which comprises a backbone region suitable for replication of the nucleic acid construct, e.g., for replication in a bacterial cell.
129. The nucleic acid construct of embodiment 128, wherein the backbone region comprises one or both of an origin of replication and a selectable marker.
130. The nucleic acid construct of any of embodiments 126-129, which further comprises a CAV tandem region placed in tandem with the genetic element, wherein the CAV tandem region comprises a CAV genome or fragment thereof, or a sequence having at least 70%, 75%, 80%, 85%, 90%, 95%, 96%, 97%, 98%, 99%, or 100% sequence identity thereto.
131. The nucleic acid construct of embodiment 130, wherein the genetic element is positioned 3′ relative to the CAV tandem region.
132. The nucleic acid construct of embodiment 131, wherein the CAV tandem region comprises the repeats (or a fragment thereof, e.g., a fragment comprising 1, 2, 3, 4, or 5 of the repeats), promoter, open reading frames, and hairpin of a wild-type CAV genome sequence, e.g., as described herein, e.g., in any of Tables 1A, 1B, or 17.
133. The nucleic acid construct of embodiment 131, wherein the CAV tandem region comprises the promoter, open reading frames, and hairpin of a wild-type CAV genome sequence, e.g., as described herein, e.g., in any of Tables 1A, 1B, or 17.
134. The nucleic acid construct of embodiment 131, wherein the CAV tandem region comprises the open reading frames, and hairpin of a wild-type CAV genome sequence, e.g., as described herein, e.g., in any of Tables 1A, 1B, or 17.
135. The nucleic acid construct of embodiment 131, wherein the CAV tandem region comprises a fragment of the open reading frames (e.g., a 3′ fragment of the open reading frames, e.g., comprising the 5, 10, 20, 30, 40, 50, 60, 70, 80, 90, 100, 110, 120, 130, 140, 150, 200, 300, 400, or 500 3′-most nucleotides of the open reading frames) and hairpin of a wild-type CAV genome sequence, e.g., as described herein, e.g., in any of Tables 1A, 1B, or 17.
136. The nucleic acid construct of embodiment 131, wherein the CAV tandem region does not comprise the repeats (or a fragment thereof, e.g., a fragment comprising 1, 2, 3, 4, or 5 of the repeats) of a wild-type CAV genome sequence, e.g., as described herein, e.g., in any of Tables 1A, 1B, or 17.
137. The nucleic acid construct of embodiment 131, wherein the CAV tandem region does not comprise the repeats and/or promoter of a wild-type CAV genome sequence, e.g., as described herein, e.g., in any of Tables 1A, 1B, or 17.
138. The nucleic acid construct of embodiment 131, wherein the CAV tandem region does not comprise the repeats, promoter, and/or open reading frames of a wild-type CAV genome sequence, e.g., as described herein, e.g., in any of Tables 1A, 1B, or 17.
139. The nucleic acid construct of embodiment 131, wherein the CAV tandem region does not comprise the repeats, promoter, and/or at least a fragment of the open reading frames (e.g., a 3′ fragment of the open reading frames, e.g., comprising the 5, 10, 20, 30, 40, 50, 60, 70, 80, 90, 100, 110, 120, 130, 140, 150, 200, 300, 400, or 500 3′-most nucleotides of the open reading frames) and hairpin of a wild-type CAV genome sequence, e.g., as described herein, e.g., in any of Tables 1A, 1B, or 17.
140. The nucleic acid construct of embodiment 130, wherein the genetic element is positioned 5′ relative to the CAV tandem region.
141. The nucleic acid construct of embodiment 140, wherein the CAV tandem region comprises the repeats, promoter, open reading frames, and hairpin of a wild-type CAV genome sequence, e.g., as described herein, e.g., in any of Tables 1A, 1B, or 17.
142. The nucleic acid construct of embodiment 140, wherein the CAV tandem region comprises the repeats, promoter, and open reading frames of a wild-type CAV genome sequence, e.g., as described herein, e.g., in any of Tables 1A, 1B, or 17.
143. The nucleic acid construct of embodiment 140, wherein the CAV tandem region comprises the repeats, promoter, and a fragment of the open reading frames (e.g., a 5′ fragment of the open reading frames, e.g., comprising the 5, 10, 20, 30, 40, 50, 60, 70, 80, 90, 100, 110, 120, 130, 140, 150, 200, 300, 400, or 500 5′-most nucleotides of the open reading frames) of a wild-type CAV genome sequence, e.g., as described herein, e.g., in any of Tables 1A, 1B, or 17.
144. The nucleic acid construct of embodiment 140, wherein the CAV tandem region comprises the repeats of a wild-type CAV genome sequence, e.g., as described herein, e.g., in any of Tables 1A, 1B, or 17.
145. The nucleic acid construct of embodiment 140, wherein the CAV tandem region comprises a fragment of the repeats of a wild-type CAV genome sequence, e.g., as described herein, e.g., in any of Tables 1A, 1B, or 17.
146. The nucleic acid construct of embodiment 140, wherein the CAV tandem region does not comprise the hairpin of a wild-type CAV genome sequence, e.g., as described herein, e.g., in any of Tables 1A, 1B, or 17.
147. The nucleic acid construct of embodiment 140, wherein the CAV tandem region does not comprise the open reading frames and/or the hairpin of a wild-type CAV genome sequence, e.g., as described herein, e.g., in any of Tables 1A, 1B, or 17.
148. The nucleic acid construct of embodiment 140, wherein the CAV tandem region does not comprise a fragment of the open reading frames (e.g., a 5′ fragment of the open reading frames, e.g., comprising the 5, 10, 20, 30, 40, 50, 60, 70, 80, 90, 100, 110, 120, 130, 140, 150, 200, 300, 400, or 500 5′-most nucleotides of the open reading frames) and/or the hairpin of a wild-type CAV genome sequence, e.g., as described herein, e.g., in any of Tables 1A, 1B, or 17.
149. The nucleic acid construct of embodiment 140, wherein the CAV tandem region does not comprise the promoter, open reading frames, and/or hairpin of a wild-type CAV genome sequence, e.g., as described herein, e.g., in any of Tables 1A, 1B, or 17.
150. The nucleic acid construct of embodiment 140, wherein the CAV tandem region does not comprise the repeats, promoter, open reading frames, and/or hairpin of a wild-type CAV genome sequence, e.g., as described herein, e.g., in any of Tables 1A, 1B, or 17.
151. The nucleic acid construct of embodiment 140, wherein the CAV tandem region does not comprise a fragment of the repeats, the promoter, the open reading frames, and/or the hairpin of a wild-type CAV genome sequence, e.g., as described herein, e.g., in any of Tables 1A, 1B, or 17.
152. The nucleic acid construct of any of the preceding embodiments, wherein the CAV tandem region comprises at least 10, 20, 30, 40, 50, 60, 70, 80, 90, 100, 200, 300, 400, 500, 600, 700, 800, 900, 1000, 1500, 2000, 2100, 2200, 2300, 2310, 2311, or 2312 contiguous nucleotides of a wild-type CAV genome sequence (e.g., as described herein, e.g., in any of Tables 1A, 1B, or 17), or a nucleic acid sequence having at least 75%, 80%, 85%, 90%, 95%, 96%, 97%, 98%, or 99% sequence identity thereto.
153. The nucleic acid construct of any of the preceding embodiments, wherein the CAV tandem region comprises no more than 10, 20, 30, 40, 50, 60, 70, 80, 90, 100, 200, 300, 400, 500, 600, 700, 800, 900, 1000, 1500, 2000, 2100, 2200, 2300, 2310, 2311, or 2312 contiguous nucleotides of a wild-type CAV genome sequence (e.g., as described herein, e.g., in any of Tables 1A, 1B, or 17), or a nucleic acid sequence having at least 75%, 80%, 85%, 90%, 95%, 96%, 97%, 98%, or 99% sequence identity thereto.
154. The nucleic acid construct of any of the preceding embodiments, wherein the CAV tandem region comprises between 1-10, 10-20, 20-30, 30-40, 40-50, 50-60, 60-70, 70-80, 80-90, 90-100, 100-200, 200-300, 300-400, 400-500, 500-600, 600-700, 700-800, 800-900, 900-1000, 1000-1500, 1500-2000, 2000-2100, 2100-2200, 2200-2300, 2300-2310, or 2310-2313 contiguous nucleotides of a wild-type CAV genome sequence (e.g., as described herein, e.g., in any of Tables 1A, 1B, or 17), or a nucleic acid sequence having at least 75%, 80%, 85%, 90%, 95%, 96%, 97%, 98%, or 99% sequence identity thereto.
155. A nucleic acid construct (e.g., a plasmid) comprising one, two, or all three of:
(a) a CAV VP1 gene, or a nucleic acid sequence having at least 75%, 80%, 85%, 90%, 95%, 96%, 97%, 98%, or 99% sequence identity thereto;
(b) a CAV VP2 gene, or a nucleic acid sequence having at least 75%, 80%, 85%, 90%, 95%, 96%, 97%, 98%, or 99% sequence identity thereto; and/or
(c) a CAV Apoptin gene, or a nucleic acid sequence having at least 75%, 80%, 85%, 90%, 95%, 96%, 97%, 98%, or 99% sequence identity thereto;
wherein the nucleic acid construct does not comprise a CAV packaging signal, and/or wherein the nucleic acid construct is incapable of being packaged by a CAV VP1 molecule.
156. The nucleic acid construct of embodiment 155, wherein the CAV packaging signal comprises the nucleic acid sequence AGCCCTGAAAAGGGGGGGGGGCTAAAGCCCCCCCCCCTTAAACCCCCCCCTGGGGGGGATT CCCCCCCAGACCCCCCCTTTATATAGCACTCAATAAACGCAGAAAATAGATTTATCGCACTA TC (SEQ ID NO: 17), or a nucleic acid sequence having at least 75%, 80%, 85%, 90%, 95%, 96%, 97%, 98%, or 99% sequence identity thereto, or a reverse complement thereof.
157. The nucleic acid construct of embodiment 155, comprising a nucleic acid sequence encoding a CAV VP1 protein, or a functional fragment or variant thereof.
158. The nucleic acid construct of embodiment 157, wherein the CAV VP1 protein comprises an arginine-rich region.
159. The nucleic acid construct of embodiment 157 or 158, wherein the CAV VP1 protein comprises a jelly-roll domain.
160. The nucleic acid construct of any of embodiments 157-159, wherein the CAV VP1 protein comprises one or more DNA-binding motifs.
161. The nucleic acid construct of any of embodiments 157-160, wherein the CAV VP1 protein comprises a DNA-binding motif comprising the amino acid sequence RRARRPRGRFYAFRRGR, or an amino acid sequence having no more than 1, 2, 3, 4, 5, 6, 7, 8, 9, or 10 amino acid differences therefrom.
162. The nucleic acid construct of any of embodiments 157-161, wherein the CAV VP1 protein comprises a DNA-binding motif comprising the amino acid sequence RRRYKFRHRRQRYRRRAFRKH, or an amino acid sequence having no more than 1, 2, 3, 4, 5, 6, 7, 8, 9, or 10 amino acid differences therefrom.
163. The nucleic acid construct of any of embodiments 157-162, wherein the CAV VP1 protein comprises a DNA-binding motif comprising the amino acid sequence SRRSFNHHKARGAGDPK, or an amino acid sequence having no more than 1, 2, 3, 4, 5, 6, 7, 8, 9, or 10 amino acid differences therefrom.
164. The nucleic acid construct of any of embodiments 157-163, wherein the CAV VP1 protein comprises one or more (e.g., two) nuclear localization signals (NLS).
165. The nucleic acid construct of embodiment 164, wherein the NLS comprises the amino acid sequence RRARRPRGRFYAFRRGRWHH, or an amino acid sequence having no more than 1, 2, 3, 4, 5, 6, 7, 8, 9, or 10 amino acid differences therefrom,
166. The nucleic acid construct of embodiment 164, wherein the NLS comprises the amino acid sequence KRLRRRYKFRHRRRQRYRRRAFRK, or an amino acid sequence having no more than 1, 2, 3, 4, 5, 6, 7, 8, 9, or 10 amino acid differences therefrom,
167. The nucleic acid construct of any of embodiments 157-166, wherein the CAV VP1 protein comprises one or more (e.g., two or three) nuclear export signals (NES).
168. The nucleic acid construct of embodiment 167, wherein the NES comprises the amino acid sequence IFLTEGLIL, or an amino acid sequence having no more than 1, 2, 3, 4, or 5 amino acid differences therefrom,
169. The nucleic acid construct of embodiment 167, wherein the NES comprises the amino acid sequence LKEFLLASMNL, or an amino acid sequence having no more than 1, 2, 3, 4, or 5 amino acid differences therefrom,
170. The nucleic acid construct of embodiment 167, wherein the NES comprises the amino acid sequence ELDTNFFTLYVAQ, or an amino acid sequence having no more than 1, 2, 3, 4, 5, 6, 7, 8, 9, or 10 amino acid differences therefrom,
171. The nucleic acid construct of any of the preceding embodiments, comprising a nucleic acid sequence encoding a CAV VP2 protein, or a functional fragment or variant thereof.
172. The nucleic acid construct of embodiment 171, wherein the CAV VP2 protein, or the functional fragment thereof, comprises the amino acid sequence I⁹⁴CNCGQFRKH¹⁰³.
173. The nucleic acid construct of embodiment 171, wherein the CAV VP2 protein, or the functional fragment thereof, comprises the amino acid sequence WX₇HX₃CXCX₅H.
174. The nucleic acid construct of embodiment 171 or 172, wherein the CAV VP2 protein, or the functional fragment thereof, comprises the amino acid sequence WLRECSRSHAKICNCGQFRKH.
175. The nucleic acid construct of any of embodiments 172-174, wherein the cysteine and histidine residues of the amino acid sequence form a metal ion coordination site (e.g., a Zn2+ coordination site).
176. The nucleic acid construct of any of the preceding embodiments, comprising a nucleic acid sequence encoding a CAV Apoptin protein, or a functional fragment or variant thereof.
177. A host cell (e.g., an avian cell, e.g., an MDCC cell, e.g., an MDCC-MSB1 cell) comprising the nucleic acid construct of embodiment 21a and a genetic element of any of the preceding embodiments.
178. A CAVector comprising:
   a) a proteinaceous exterior comprising a CAV VP1 molecule;
   b) a genetic element comprising: (i) a promoter element, (ii) a nucleic acid sequence encoding an exogenous effector, and (iii) a protein binding sequence that specifically binds the CAV VP1 molecule.
179. The CAVector of embodiment 78, wherein the genetic element is a genetic element according to any of embodiments 1-125.
180. The CAVector of embodiment 78, wherein the protein binding sequence comprises the nucleic acid sequence AGCCCTGAAAAGGGGGGGGGGCTAAAGCCCCCCCCCCTTAAACCCCCCCCTGGGGGGGATT CCCCCCCAGACCCCCCCTTTATATAGCACTCAATAAACGCAGAAAATAGATTTATCGCACTA TC (SEQ ID NO: 17), or a nucleic acid sequence having at least 75%, 80%, 85%, 90%, 95%, 96%, 97%, 98%, or 99% sequence identity thereto, or a reverse complement thereof.
181. A CAVector comprising:
a) a genetic element of any of embodiments 1-125, and
b) a proteinaceous exterior, e.g., a proteinaceous exterior comprising a CAV VP1 molecule.
182. A CAVector comprising:
a) a genetic element of any of embodiments 1-125, and
b) a capsid, e.g., a capsid comprising a CAV VP1 molecule.
183. The CAVector of any of the preceding embodiments, wherein the genetic element comprises a CAV UTR, e.g., a CAV 5′ UTR (e.g., as listed in any of Tables 1A, 1B, or 2-13), or a nucleic acid sequence having at least 75%, 80%, 85%, 90%, 95%, 96%, 97%, 98%, or 99% sequence identity thereto.
184. A complex comprising:
   a CAV VP1 molecule bound to a genetic element,
   wherein the genetic element comprises: (i) a promoter element, (ii) a nucleic acid sequence encoding an exogenous effector, and (iii) a protein binding sequence.
185. A complex comprising:
   a genetic element according to any of the preceding embodiments, and
   a capsid protein (e.g., a CAV VP1 molecule) bound to the genetic element.
186. The complex of embodiment 184 or 185, wherein the genetic element is a genetic element according to any of the preceding embodiments; and/or
wherein the complex is in a cell-free system.
187. The complex of any of embodiments 184-186, wherein the protein binding sequence comprises the nucleic acid sequence AGCCCTGAAAAGGGGGGGGGGCTAAAGCCCCCCCCCCTTAAACCCCCCCCTGGGGGGGATT CCCCCCCAGACCCCCCCTTTATATAGCACTCAATAAACGCAGAAAATAGATTTATCGCACTA TC (SEQ ID NO: 17), or a nucleic acid sequence having at least 75%, 80%, 85%, 90%, 95%, 96%, 97%, 98%, or 99% sequence identity thereto, or a reverse complement thereof.
188. The complex of any of embodiments 184-187, wherein the genetic element comprises a CAV UTR, e.g., a CAV 5′ UTR (e.g., as listed in any of Tables 1A, 1B, or 2-13), or a nucleic acid sequence having at least 75%, 80%, 85%, 90%, 95%, 96%, 97%, 98%, or 99% sequence identity thereto.
189. A method of delivering an exogenous effector to a target cell (e.g., a vertebrate cell, e.g., a mammalian cell, e.g., a human cell), the method comprising introducing into the cell a CAVector of any of embodiments 178-183.
190. The method of embodiment 189, wherein the method does not comprise administering an inhibitor of endocytosis, e.g., a dynamin inhibitor, e.g., Dynasore.
191. The method of embodiment 189 or 190, wherein the method does not comprise administering an inhibitor of endosome acidification, e.g., Bafilomycin A1 (BafA1) or chloroquine.
192. The method of any of embodiments 189-191, wherein the target cell takes up the CAVector by endocytosis.
193. A method of delivering an exogenous effector to a target cell (e.g., a vertebrate cell, e.g., a mammalian cell, e.g., a human cell), the method comprising introducing into the cell a CAVector, e.g., a CAVector as described herein, wherein the cell is not contacted with an inhibitor of endocytosis (e.g., a dynamin inhibitor, e.g., Dynasore).
194. A method of delivering an exogenous effector to a target cell (e.g., a vertebrate cell, e.g., a mammalian cell, e.g., a human cell), the method comprising introducing into the cell a CAVector, e.g., a CAVector as described herein, wherein the cell is not contacted with an inhibitor of endosome acidification, e.g., Bafilomycin A1 (BafA1) or chloroquine.
195. A method of delivering an exogenous effector to a target cell (e.g., a vertebrate cell, e.g., a mammalian cell, e.g., a human cell), the method comprising:
(a) assessing the target cell, or a subject comprising the target cell, for the presence of an unwanted immune response to CAV, e.g., an anti-CAV antibody, e.g., a CAV neutralizing antibody; and
(b) introducing into the cell a CAVector of any of embodiments 178-183.
196. A method of selecting a subject for receiving a CAVector, the method comprising assessing the subject for the presence of an unwanted immune response to CAV, e.g., an anti-CAV antibody, e.g., a CAV neutralizing antibody.
197. A method of modulating a biological activity in a subject in need thereof, the method comprising introducing into the subject a CAVector of any of embodiments 178-183, e.g., wherein the disease or disorder is cancer.
198. The method of embodiment 197, wherein the method does not comprise administering an inhibitor of endocytosis, e.g., a dynamin inhibitor, e.g., Dynasore.
199. The method of embodiment 197 or 198, wherein the method does not comprise administering an inhibitor of endosome acidification, e.g., Bafilomycin A1 (BafA1) or chloroquine.
200. The method of any of embodiments 197-199, wherein the target cell takes up the CAVector by endocytosis.
201. A method of modulating a biological activity in a subject in need thereof, the method comprising administering to the subject a CAVector, e.g., a CAVector as described herein, wherein the subject is not administered an inhibitor of endocytosis (e.g., a dynamin inhibitor, e.g., Dynasore).
202. A method of modulating a biological activity in a subject in need thereof, the method comprising administering to the subject a CAVector, e.g., a CAVector as described herein, wherein the subject is not administered an inhibitor of endosome acidification, e.g., Bafilomycin A1 (BafA1) or chloroquine.
203. A method of treating a disease or disorder in a subject in need thereof, the method comprising administering to the subject a CAVector of any of embodiments 178-183, e.g., wherein the disease or disorder is cancer.
204. The method of embodiment 203, wherein the method does not comprise administering an inhibitor of endocytosis, e.g., a dynamin inhibitor, e.g., Dynasore.
205. The method of embodiment 203 or 204, wherein the method does not comprise administering an inhibitor of endosome acidification, e.g., Bafilomycin A1 (BafA1) or chloroquine.
206. The method of any of embodiments 203-205, wherein the target cell takes up the CAVector by endocytosis.
207. A method of treating a disease or disorder in a subject in need thereof, the method comprising administering to the subject a CAVector, e.g., a CAVector as described herein, wherein the subject is not administered an inhibitor of endocytosis (e.g., a dynamin inhibitor, e.g., Dynasore).
208. A method of treating a disease or disorder in a subject in need thereof, the method comprising administering to the subject a CAVector, e.g., a CAVector as described herein, wherein the subject is not administered an inhibitor of endosome acidification, e.g., Bafilomycin A1 (BafA1) or chloroquine.
209. A method of treating a disease or disorder in a subject in need thereof, the method comprising:
(a) assessing the subject for the presence of an unwanted immune response to CAV, e.g., an anti-CAV antibody, e.g., a CAV neutralizing antibody; and
(b) administering to the subject a CAVector of any of embodiments 178-183.
210. The method of embodiment 209, wherein the method does not comprise administering an inhibitor of endocytosis, e.g., a dynamin inhibitor, e.g., Dynasore.
211. The method of embodiment 209 or 210, wherein the method does not comprise administering an inhibitor of endosome acidification, e.g., Bafilomycin A1 (BafA1) or chloroquine.
212. The method of any of embodiments 209-211, wherein the target cell takes up the CAVector by endocytosis.
213. A method of vaccinating a subject in need thereof, the method comprising administering to the subject a CAVector of any of embodiments 178-283, wherein the exogenous effector comprises an antigen from an infectious agent (e.g., a virus or bacteria).
214. The method of embodiment 213, wherein the method does not comprise administering an inhibitor of endocytosis, e.g., a dynamin inhibitor, e.g., Dynasore.
215. The method of embodiment 213 or 214, wherein the method does not comprise administering an inhibitor of endosome acidification, e.g., Bafilomycin A1 (BafA1) or chloroquine.
216. The method of any of embodiments 213-215, wherein the target cell takes up the CAVector by endocytosis.
217. A method of vaccinating a subject in need thereof, the method comprising administering to the subject a CAVector, e.g., a CAVector as described herein, wherein the subject is not administered an inhibitor of endocytosis (e.g., a dynamin inhibitor, e.g., Dynasore), and wherein the exogenous effector comprises an antigen from an infectious agent (e.g., a virus or bacteria).
218. A method of vaccinating a subject in need thereof, the method comprising administering to the subject a CAVector, e.g., a CAVector as described herein, wherein the subject is not administered an inhibitor of endosome acidification, e.g., Bafilomycin A1 (BafA1) or chloroquine, and wherein the exogenous effector comprises an antigen from an infectious agent (e.g., a virus or bacteria).
219. The method of any of embodiments 189-218, wherein the target cell is a lymphoid cell (e.g., a B cell or T cell), an epithelial cell, a lung cell, or a fibroblast.
220. The method of any of embodiments 189-219, wherein the target cell is a human cell.
221. The method of any of embodiments 189-219, wherein the target cell is a cell from an animal (e.g., an agricultural animal, e.g., a cow, sheep, pig, goat, horse, bison, or camel).
222. The method of embodiment 221, wherein the animal is an avian animal (e.g., a turkey, chicken, quail, emu, or ostrich).
223. The method of embodiment any of embodiments 189-222, wherein the target cell is in vivo or in vitro.
224. The method of any of embodiments 189-223, wherein the CAVector is delivered at a MOI of about 1-10 (e.g., about 2-4, e.g., about 3), 10-50, 50-100, 100-500, 500-1000, 1000-5000, 5000-10,000, 10,000-50,000, or 50,000-100,000.
225. The genetic element, nucleic acid construct, CAVector, or method of any of the preceding embodiments, wherein if the exogenous effector is replaced with nano-luciferase, the CAVector can deliver nano-luciferase to a plurality of target cells in vitro, resulting in luminescence of at least about 10⁴, 10⁵, 10⁶, or 10⁷, or about 10⁴-10⁵, 10⁵-10⁶, or 10⁶-10⁷ e.g., in an assay of Example 5.
226. The genetic element, nucleic acid construct, CAVector, or method of any of the preceding embodiments, which can bind to human cells (e.g., Raji cells), e.g., at a vg relative to binding to MDCC cells of at least 10%, 20%, 30%, or 40%, e.g., in an assay of Example 3.
227. The genetic element, nucleic acid construct, CAVector, or method of any of the preceding embodiments, wherein the genetic element produces at least 2, 3, 4, 5, 10, 20, 30, 40, 50, 100, 500, 1000, 10,000-fold more copies of the exogenous effector than a control vector.
228. The genetic element, nucleic acid construct, CAVector, or method of any of the preceding embodiments, wherein the control vector comprises a wild-type CAV genome (e.g., as described herein, e.g., as listed in any of Tables 1A, 1B, or 17).
229. The genetic element, nucleic acid construct, CAVector, or method of any of the preceding embodiments, wherein the control vector comprises a viral genome other than CAV (e.g., an AAV genome or a lentivirus genome).
230. The genetic element, nucleic acid construct, CAVector, or method of embodiment 229, wherein the control vector is identical to the genetic element except lacking any CAV sequence.
231. The genetic element, nucleic acid construct, CAVector, or method of any of the preceding embodiments, wherein the genetic element produces at least 100, 200, 300, 400, 500, 1000, 5000, 10,000, 50,000, 100,000, 1,000,000, 10,000,000, 50,000,000, or 100,000,000 copies of the exogenous effector per cell.
232. The genetic element, nucleic acid construct, CAVector, or method of any of the preceding embodiments, wherein the exogenous effector has the same sequence as, or a sequence having at least 75%, 80%, 85%, 90%, 95%, 96%, 97%, 98%, or 99% sequence identity to, a corresponding molecule endogenous to the target cell, and wherein the level of exogenous effector expressed in the target cell is at least 2, 3, 4, 5, 6, 7, 8, 9, 10, 15, 20, 25, 30, 40, 50, 60, 70, 80, 90, 100, 500, 1000, 5000, 10,000, 50,000, or 100,000-fold greater than the amount of the corresponding endogenous molecule.
233. A method of making a CAVector, comprising:
   a) providing a host cell comprising a genetic element of any of the preceding embodiments, and
   b) incubating the host cell under conditions suitable for enclosure of the genetic element in a proteinaceous exterior (e.g., a proteinaceous exterior comprising a CAV VP1 molecule),
   thereby making the CAVector.
234. The method of embodiment 233, wherein the host cell releases CAVector into supernatant (e.g., wherein the host cell cell secretes the CAVector into supernatant and/or wherein the host cell is lysed, e.g., in SDS, e.g., 0.5% SDS).
235. The method of embodiment 233 or 234, which comprises harvesting the CAVector from supernatant from the host cell (e.g., supernatant secreted from the host cell and/or supernatant obtained from lysis of the host cell).
236. The method of any of embodiments 233-235, which comprises harvesting the CAVector from lysate from the host cell.
237. The method of embodiment 235 or 236, wherein the the supernatant or lysate are treated with a nuclease (e.g., benzonase).
238. The method of any of embodiments 235-237, wherein the supernatant or lysate is filtered (e.g., through a 0.45 μm filter.
239. The method of any of embodiments 235-238, wherein the supernatant or lysate is ultracentrifuged, e.g., through a sucrose cushion (e.g., a 20% sucrose cushion).
240. The method of any of embodiments 235-239, wherein the supernatant or lysate is run through a CsCl gradient.
241. The method of any of embodiments 235-240, wherein the supernatant or lysate is fractionate and/or dialyzed.
242. The method of any of embodiments 235-241, wherein the host cell further comprises one or more additional nucleic acids encoding one or more additional ORFs (e.g., one or more additional CAV ORFs, e.g., one or more of CAV VP1, VP2, or Apoptin).
243. A host cell (e.g., a vertebrate cell, e.g., (i) a mammalian cell, e.g., a human cell; or (ii) an avian cell, e.g., a chicken cell) comprising a genetic element of any of the preceding embodiments, or a nucleic acid construct comprising the genetic element.
244. The host cell of embodiment 243, which further comprises a CAV VP1 molecule or a nucleic acid encoding the CAV VP1 molecule.
245. A host cell comprising a CAVector of any of embodiments 178-183.
246. A host cell comprising:
   a) a CAV VP1 molecule, or a nucleic acid encoding the CAV VP1 molecule; and
   b) a genetic element or a nucleic acid construct comprising the genetic element, wherein the genetic element comprises (i) a promoter element, (ii) a nucleic acid sequence encoding an exogenous effector, and (iii) a protein binding sequence, e.g., wherein the genetic element is a genetic element according to any of the preceding embodiments.
247. The host cell of embodiment 246, wherein the protein binding sequence comprises the nucleic acid sequence AGCCCTGAAAAGGGGGGGGGGCTAAAGCCCCCCCCCCTTAAACCCCCCCCTGGGGGGGATT CCCCCCCAGACCCCCCCTTTATATAGCACTCAATAAACGCAGAAAATAGATTTATCGCACTA TC (SEQ ID NO: 17), or a nucleic acid sequence having at least 75%, 80%, 85%, 90%, 95%, 96%, 97%, 98%, or 99% sequence identity thereto, or a reverse complement thereof.
248. The complex of any of embodiments 184-188, wherein the complex is in vitro, e.g., wherein the complex is in a substantially cell-free composition.
249. The complex of any of embodiments 184-188 or 248, wherein the complex is in a cell, e.g., a host cell, e.g., a helper cell, e.g., in the nucleus of the cell.
250. The complex of any of embodiments 184-188, 248, or 249, wherein the CAV VP1 molecule is part of a proteinaceous exterior.
251. The complex of any of embodiments 184-188 or 248-250, wherein the genetic element is undergoing replication.
252. The complex of any of embodiments 184-188 or 248-251, wherein the complex is in an CAVector.
253. A method of making a host cell of any of embodiments 243-252, comprising introducing the genetic element into a cell, e.g., wherein introducing the genetic element into the cell comprises introducing a nucleic acid construct comprising the genetic element into the cell.
254. The method of embodiment 253, which further comprises introducing into the cell a nucleic acid encoding a VP1 molecule.
255. The genetic element, nucleic acid construct, CAVector, complex, method, or host cell of any of the preceding embodiments, wherein the protein binding sequence specifically binds a CAV capsid polypeptide (e.g., a CAV VP1 molecule), e.g., with an affinity/specificity of less than about 10 μM (e.g., less than about 10, 9, 8, 7, 6, 5, 4, 3, 2, or 1 μM, e.g., less than about 900, 800, 700, 600, 500, 400, 300, 200, 100, 90, 80, 70, 60, 50, 40, 30, 20, or 10 nM).
256. The genetic element, nucleic acid construct, CAVector, complex, method, or host cell of any of the preceding embodiments, wherein the genetic element is capable of being packaged (e.g., specifically packaged) by a CAV VP1 molecule.
257. The genetic element, nucleic acid construct, CAVector, complex, method, or host cell of any of the preceding embodiments, wherein the exogenous effector is:
   (a) codon optimized for expression in a human cell,
   (b) a human polypeptide or nucleic acid,
   (c) binds a human polypeptide or nucleic acid, or
   (d) has an activity in a human cell, e.g., modulates (e.g., increases or decreases) the activity and/or level of a human gene in the human cell).
258. The genetic element, nucleic acid construct, CAVector, complex, method, or host cell of any of the preceding embodiments, wherein the proteinaceous exterior comprises a CAV VP1 molecule.
259. The genetic element, nucleic acid construct, CAVector, complex, method, or host cell of any of the preceding embodiments, wherein at least 60% (e.g., at least 60%, 65%, 70%, 75%, 80%, 85%, 90%, 95%, 96%, 97%, 98%, 99%, or 100%) of protein in the proteinaceous exterior comprises a CAV VP1 molecule.
260. The genetic element, nucleic acid construct, CAVector, complex, method, or host cell of any of the preceding embodiments, wherein the genetic element or nucleic acid construct comprises at least 10, 20, 30, 40, 50, 60, 70, 80, 90, 100, 200, 300, 400, 500, 600, 700, 800, 900, 1000, 1500, 2000, 2100, 2200, 2300, 2310, 2311, or 2312 contiguous nucleotides of a wild-type CAV genome sequence (e.g., as described herein, e.g., in any of Tables 1A, 1B, or 17), or a nucleic acid sequence having at least 75%, 80%, 85%, 90%, 95%, 96%, 97%, 98%, or 99% sequence identity thereto.
261. The genetic element, nucleic acid construct, CAVector, complex, method, or host cell of any of the preceding embodiments, wherein the genetic element or nucleic acid construct comprises between 1-10, 10-20, 20-30, 30-40, 40-50, 50-60, 60-70, 70-80, 80-90, 90-100, 100-200, 200-300, 300-400, 400-500, 500-600, 600-700, 700-800, 800-900, 900-1000, 1000-1500, 1500-2000, 2000-2100, 2100-2200, 2200-2300, 2300-2310, or 2310-2313 contiguous nucleotides of a wild-type CAV genome sequence (e.g., as described herein, e.g., in any of Tables 1A, 1B, or 17), or a nucleic acid sequence having at least 75%, 80%, 85%, 90%, 95%, 96%, 97%, 98%, or 99% sequence identity thereto.
262. The genetic element, nucleic acid construct, CAVector, complex, method, or host cell of any of the preceding embodiments, wherein the genetic element or nucleic acid construct comprises a deletion (e.g., of at least 10, 20, 30, 40, 50, 60, 70, 80, 90, 100, 200, 300, 400, 500, 600, 700, 800, 900, 1000, 1500, or 2000 nucleotides) relative to a wild-type CAV genome sequence, e.g., as described herein.
263. The genetic element, nucleic acid construct, CAVector, complex, method, or host cell of any of the preceding embodiments, wherein the genetic element or nucleic acid construct comprises a CAV packaging signal, e.g., as described herein, e.g., comprising the nucleic acid sequence AGCCCTGAAAAGGGGGGGGGGCTAAAGCCCCCCCCCCTTAAACCCCCCCCTGGGGGGGATT CCCCCCCAGACCCCCCCTTTATATAGCACTCAATAAACGCAGAAAATAGATTTATCGCACTA TC (SEQ ID NO: 17), or a nucleic acid sequence having at least 75%, 80%, 85%, 90%, 95%, 96%, 97%, 98%, or 99% sequence identity thereto, or a reverse complement thereof.
264. The genetic element, nucleic acid construct, CAVector, complex, method, or host cell of any of the preceding embodiments, wherein the effector comprises a miRNA.
265. The genetic element, nucleic acid construct, CAVector, complex, method, or host cell of any of the preceding embodiments, wherein the effector, e.g., miRNA, targets a host gene, e.g., modulates expression of the gene, e.g., increases or decreases expression of the gene.
266. The genetic element, nucleic acid construct, CAVector, complex, method, or host cell of any of the preceding embodiments, wherein the effector comprises an miRNA, and decreases expression of a host gene.
267. The genetic element, nucleic acid construct, CAVector, complex, method, or host cell of any of the preceding embodiments, wherein the effector comprises a nucleic acid sequence about 20-200, 30-180, 40-160, 50-140, or 60-120 nucleotides in length.
268. The genetic element, nucleic acid construct, CAVector, complex, method, or host cell of any of the preceding embodiments, wherein the nucleic acid sequence encoding the effector is about 20-200, 30-180, 40-160, 50-140, or 60-120 nucleotides in length.
269. The genetic element, nucleic acid construct, CAVector, complex, method, or host cell of any of the preceding embodiments, wherein the sequence encoding the effector has a size of at least about 100 nucleotides.
270. The genetic element, nucleic acid construct, CAVector, complex, method, or host cell of any of the preceding embodiments, wherein the sequence encoding the effector has a size of about 100 to about 5000 nucleotides.
271. The genetic element, nucleic acid construct, CAVector, complex, method, or host cell of any of the preceding embodiments, wherein the sequence encoding the effector has a size of about 100-200, 200-300, 300-400, 400-500, 500-600, 600-700, 700-800, 800-900, 900-1000, 1000-1500, or 1500-2000 nucleotides.
272. The genetic element, nucleic acid construct, CAVector, complex, method, or host cell of any of the preceding embodiments, wherein the genetic element is single-stranded.
273. The genetic element, nucleic acid construct, CAVector, complex, method, or host cell of any of the preceding embodiments, wherein the genetic element is circular.
274. The genetic element, nucleic acid construct, CAVector, complex, method, or host cell of any of the preceding embodiments, wherein the genetic element is DNA, e.g., single-stranded DNA.
275. The genetic element, nucleic acid construct, CAVector, complex, method, or host cell of any of the preceding embodiments, wherein the CAVector is contacted to a cell in vitro, ex vivo, or in vivo.
276. The genetic element, nucleic acid construct, CAVector, complex, method, or host cell of any of the preceding embodiments, wherein the CAVector is replication-deficient.
277. The genetic element, nucleic acid construct, CAVector, complex, method, or host cell of any of the preceding embodiments, wherein the CAVector is administered to a subject intravenously, intraperitoneally, intramuscularly, or subretinally.
278. The genetic element, nucleic acid construct, CAVector, complex, method, or host cell of any of the preceding embodiments, wherein the CAVector is formulated for intravenous, intraperitoneal, intramuscular, or subretinal administration.
279. The genetic element, nucleic acid construct, CAVector, complex, method, or host cell of any of the preceding embodiments, wherein the CAVector is delivered to a cell or tissue in a subject (e.g., a mammalian subject, e.g., a human subject).
280. The genetic element, nucleic acid construct, CAVector, complex, method, or host cell of embodiment 279, wherein the cell or tissue is selected from blood, liver, spleen, lung, heart, ovary, muscle, brain, kidney, and/or retina.
281. The genetic element, nucleic acid construct, CAVector, complex, method, or host cell of any of the preceding embodiments, wherein the genetic element, nucleic acid construct, or CAVector induces a neutralizing antibody response less than that of an equivalent quantity of an adeno-associated virus (AAV, e.g., AAV2) or an AAV vector, e.g., according to a method of Example 12.
282. The genetic element, nucleic acid construct, CAVector, complex, method, or host cell of any of the preceding embodiments, wherein the genetic element, nucleic acid construct, or CAVector, when administered to a test subject (e.g., a mouse) intramuscularly, induces a neutralizing antibody response having a 50% geometric mean neutralizing reciprocal titer (50% GMT) less than the 50% GMT induced by an equivalent quantity of an adeno-associated virus (AAV, e.g., AAV2) or an AAV vector, when administered to a test subject intramuscularly, e.g., according to a method of Example 12.
283. The genetic element, nucleic acid construct, CAVector, complex, method, or host cell of any of the preceding embodiments, wherein the genetic element, nucleic acid construct, or CAVector, when administered to a test subject (e.g., a mouse) intramuscularly, induces a neutralizing antibody response having a 50% geometric mean neutralizing reciprocal titer (50% GMT) less than about 320, 321, 322, 323, 324, 325, 330, 340, 350, 360, 370, 380, 390, 400, 450, 500, 550, 600, 610, 620, 630, 635, 636, 637, 638, 639, or 640.
284. The genetic element, nucleic acid construct, CAVector, complex, method, or host cell of any of the preceding embodiments, wherein the genetic element, nucleic acid construct, or CAVector, when administered to a test subject (e.g., a mouse) intramuscularly, induces a neutralizing antibody response having a 50% geometric mean neutralizing reciprocal titer (50% GMT) less than about 160, 170, 180, 190, 200, 250, 300, 310, or 320.
285. The genetic element, nucleic acid construct, CAVector, complex, method, or host cell of any of the preceding embodiments, wherein the genetic element, nucleic acid construct, or CAVector, when administered to a test subject (e.g., a mouse) intravenously or intraperitoneally, induces a neutralizing antibody response having a 50% geometric mean neutralizing reciprocal titer (50% GMT) less than or equal to about 20, 21, 22, 23, 24, 25, 30, 40, 50, 60, 70, 80, 90, 100, 110, 120, 130, 140, 150, 151, 152, 153, 154, 155, 156, 157, 158, 159, or 160.
286. The genetic element, nucleic acid construct, CAVector, complex, method, or host cell of any of the preceding embodiments, wherein the CAVector is administered to the subject in need thereof intravenously, intraperitoneally, or intramuscularly.
287. The genetic element, nucleic acid construct, CAVector, complex, method, or host cell of any of the preceding embodiments, wherein the CAVector is assembled in vitro.
288. The genetic element, nucleic acid construct, CAVector, complex, method, or host cell of embodiment 287, wherein the in vitro assembly comprises enclosing a genetic element (e.g., a CAVector genetic element as described herein) within a proteinaceous exterior comprising a VP1 molecule.
289. The genetic element, nucleic acid construct, CAVector, complex, method, or host cell of embodiment 288, wherein the in vitro assembly further comprises contacting the VP1 molecule with the genetic element.
290. The genetic element, nucleic acid construct, CAVector, complex, method, or host cell of any of the preceding embodiments, the wherein the genetic element, nucleic acid construct, or CAVector induces a neutralizing antibody response sufficiently low as to be suitable for administration in multiple doses (e.g., doses administered separately, e.g., as described herein).
291. A method of delivering an effector or payload (e.g., an endogenous or exogenous effector) to a cell, tissue, or subject, the method comprising administering to the cell, tissue, or subject an effective amount of a CAVector (e.g., a synthetic CAVector, e.g., as described herein),
wherein the CAVector comprises a nucleic acid sequence encoding the effector or payload, and
wherein the CAVector, when administered to a test subject (e.g., a mouse) intramuscularly, induces a lower neutralizing antibody response than induced by an equivalent quantity of an adeno-associated virus (AAV, e.g., AAV2) or an AAV vector, when introduced into a test subject intramuscularly, e.g., according to a method of Example 12.
292. A method of delivering an effector or payload (e.g., an endogenous or exogenous effector) to a cell, tissue, or subject, the method comprising administering to the cell, tissue, or subject an effective amount of a CAVector (e.g., a synthetic CAVector, e.g., as described herein),
wherein the CAVector comprises a nucleic acid sequence encoding the effector or payload, and
wherein the CAVector, when administered to a test subject (e.g., a mouse) intramuscularly, induces a neutralizing antibody response having a 50% geometric mean neutralizing reciprocal titer (50% GMT) less than the 50% GMT induced by an equivalent quantity of an adeno-associated virus (AAV, e.g., AAV2) or an AAV vector, when introduced into a test subject intramuscularly, e.g., according to a method of Example 12.
293. A method of delivering an effector or payload (e.g., an endogenous or exogenous effector) to a cell, tissue, or subject in need thereof, the method comprising administering to the cell, tissue, or subject an effective amount of a CAVector (e.g., a synthetic CAVector, e.g., as described herein),
wherein the CAVector comprises a nucleic acid sequence encoding the effector or payload, and
wherein the CAVector, when introduced into a test subject (e.g., a mouse) intramuscularly, induces a neutralizing antibody response having a 50% geometric mean neutralizing reciprocal titer (50% GMT) less than about 320, 321, 322, 323, 324, 325, 330, 340, 350, 360, 370, 380, 390, 400, 450, 500, 550, 600, 610, 620, 630, 635, 636, 637, 638, 639, or 640.
294. A method of delivering an effector or payload (e.g., an endogenous or exogenous effector) to a cell, tissue, or subject in need thereof, the method comprising administering to the cell, tissue, or subject an effective amount of a CAVector (e.g., a synthetic CAVector, e.g., as described herein),
wherein the CAVector comprises a nucleic acid sequence encoding the effector or payload, and
wherein the CAVector, when introduced into a test subject (e.g., a mouse) intramuscularly, induces a neutralizing antibody response having a 50% geometric mean neutralizing reciprocal titer (50% GMT) less than about 160, 170, 180, 190, 200, 250, 300, 310, or 320.
295. A method of delivering an effector or payload (e.g., an endogenous or exogenous effector) to a cell, tissue, or subject in need thereof, the method comprising administering to the cell, tissue, or subject an effective amount of a CAVector (e.g., a synthetic CAVector, e.g., as described herein),
wherein the CAVector comprises a nucleic acid sequence encoding the effector or payload, and
wherein the CAVector, when introduced into a test subject (e.g., a mouse) intravenously or intraperitoneally, induces a neutralizing antibody response having a 50% geometric mean neutralizing reciprocal titer (50% GMT) less than or equal to about 20, 21, 22, 23, 24, 25, 30, 40, 50, 60, 70, 80, 90, 100, 110, 120, 130, 140, 150, 151, 152, 153, 154, 155, 156, 157, 158, 159, or 160.
296. The method of any of embodiments 291-295, wherein the CAVector is administered to the subject in need thereof intravenously, intraperitoneally, or intramuscularly.
297. The method of any of embodiments 291-296, wherein the level of neutralizing antibodies induced by the administration is determined according to a method of Example 12.
298. The method of any of embodiments 291-296, wherein the level of neutralizing antibodies induced by the administration is determined according to a method comprising assessing a level of neutralizing antibodies in a sample (e.g., a serum sample) obtained from a subject administered the CAVector.
299. The method of embodiment 298, wherein the assessing comprises contacting the sample with a test cell in vitro and a test CAVector comprising a genetic element encoding a reporter (e.g., a fluorescent or luminescent reporter, e.g., nano-luciferase), and measuring a level or activity of the reporter in the test cell compared to an otherwise similar test cell contacted with the test CAVector in the absence of the sample.
300. The method of any of embodiments 291-299, wherein the CAVector induces a neutralizing antibody response sufficiently low as to be suitable for administration in multiple doses (e.g., doses administered separately, e.g., as described herein).
301. A pharmaceutical composition comprising the genetic element, nucleic acid construct, CAVector, complex, method, or host cell of any of the preceding embodiments, and a pharmaceutically acceptable carrier and/or excipient.
302. The pharmaceutical composition of embodiment 301, wherein the pH of the pharmaceutical composition is about 5.0-5.5, 5.5-6.0, 6.0-6.5, 6.5-7.0, 7.0-7.4, 7.4-7.6, or 7.6-8.0.
303. The pharmaceutical composition of any of embodiments 301-302, wherein the composition is at a temperature between 25-30° C., 30-35° C., 35-40° C., 40-45° C., 45-50° C., 50-55° C., 55-60° C., 60-65° C., or 65-70° C.
304. The pharmaceutical composition of any of embodiments 301-303, wherein the composition is at a temperature between 1-5° C., 5-10° C., 10-15° C., 15-20° C., or 20-25° C.
305. The pharmaceutical composition of any of embodiments 301-304, wherein the composition is at a temperature of about 4° C.
306. A method of storing a composition comprising a CAVector, the method comprising maintaining a composition comprising a CAVector (e.g., a CAVector as described herein) at a temperature between 1-5° C., 5-10° C., 10-15° C., 15-20° C., or 20-25° C. for a period of at least about 1 week, 2 weeks, 3 weeks, 4 weeks, 2 months, 3 months, 4 months, 5 months, 6 months, 7 months, 8 months, 9 months, 10 months, 11 months, 1 year, or 2 years, or a period of about 1-2 weeks, 2-3 weeks, 3-4 weeks, 1-2 months, 2-3 months, 3-4 months, 4-5 months, 5-6 months, 6-7 months, 7-8 months, 8-9 months, 9-10 months, 10-11 months, 11-12 months, 12-18 months, 18-24 months, or 2-3 years.
307. The method of embodiment 306, wherein the composition is maintained at a temperature between 1-5° C. (e.g., about 4° C.).
308. The method of embodiment 306 or 307, wherein the composition is maintained at the temperature for at least 6 months.
309. A method of cooling a composition comprising a CAVector, the method comprising lowering the temperature of a composition comprising a CAVector (e.g., a CAVector as described herein) to about 1, 2, 3, 4, 5, 6, 7, 8, 9, or 10° C., or to between 1-5° C. (e.g., about 4° C.).
310. The method of embodiment 309, wherein the temperature of the composition prior to the lowering step is at least about 10, 15, 20, 25, 30, 35, 37, or 40° C., or between 10-15° C., 15-20° C., 20-25° C., 25-30° C., 30-35° C., 35-37° C., or 37-40° C. (e.g., about 25° C.).
311. A method of heating a composition comprising a CAVector, the method comprising raising the temperature of a composition comprising a CAVector (e.g., a CAVector as described herein) to about 20, 21, 22, 23, 24, 25, 26, 27, 28, 29, or 30° C., or to between 20-25° C. or 25-30° C. (e.g., about 25° C.).
312. The method of embodiment 311, wherein the temperature of the composition prior to the raising step is less than about 1, 2, 3, 4, 5, 6, 7, 8, 9, or 10° C., or between 1-5° C. or 5-10° C. (e.g., about 4° C.).
313. A method of heating a composition comprising a CAVector, the method comprising raising the temperature of a composition comprising a CAVector (e.g., a CAVector as described herein) to about 35, 36, 37, 38, 39, or 40° C., or to between 30-35° C. or 35-40° C. (e.g., about 37° C.).
314. The method of any one of embodiments 306-313, wherein the composition is a pharmaceutical composition of any of embodiments 301-305.

Other features, objects, and advantages of the invention will be apparent from the description and drawings, and from the claims.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In addition, the materials, methods, and examples are illustrative only and not intended to be limiting.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a series of diagrams showing that only cells transfected with in vitro circularized (IVC) CAV DNA were able to produce increasing titers of CAV genomes over repeated passages, as a function of passage number (P0, P1, P2, and P4). In contrast, p637 negative control samples (consisting of wild-type CAV with a deletion in VP1) and plasmid CAV samples showed decreasing titers over repeated passages. Passages P1 and P2 were analyzed by western blotting and only showed expression of CAV VP2 and VP3 (a.k.a. apoptin) in IVC CAV samples. Passage P3 was carried out but not analyzed.

FIG. 2 is a series of diagrams showing total DNA copies of constructs found in cells exposed to passage P2 lysates from cells receiving IVC CAV DNA versus negative control plasmid p637. The graph shows qPCR results confirming that P3 material (Output) increased CAV DNA copies compared to P2 lysate (Input) when derived from cells that were transfected by IVC CAV DNA, but not by negative control plasmid p637.

FIG. 3 is a graph showing qPCR analysis of lysate from passage P3. Lysate from P3 cells was subjected to isopycnic centrifugation using CsCl and the resulting fractions analyzed by qPCR. CAV DNA is detected at a peak density of 1.32 g/mL.

FIG. 4 is a graph showing CAV copy numbers at various times post-infection. Following isolation by isopycnic centrifugation, purified CAV (labeled as IVC) or mock-infected cell lysate (Neg), was dialyzed, diluted with culture medium as indicated and added to fresh MDCC-MSB1 cells. Samples were taken daily, and CAV copy numbers were quantified by qPCR.

FIG. 5 is a series of electron micrographs showing CAV particles rescued from synthetic genomic DNA. Scale bars indicate the magnification of each image. Arrows indicate some of the CAV particles visible in the image on the left. Particles were measured to be approximately 24 nm in diameter.

FIG. 6A is a diagram showing schematics of exemplary CAV tandem constructs. pRTx-966 includes (i) repeats, promoter, ORFs, and hairpin, and (ii) repeats, promoter, ORFs, and hairpin. pRTx-1113 includes (i) truncated repeats, promoter, ORFs, and hairpin, and (ii) repeats, promoter, ORFs, and hairpin. pRTx-1114 includes (i) further truncated repeats, promoter, ORFs, and hairpin, and (ii) repeats, promoter, ORFs, and hairpin. pRTx-1115 includes (i) the promoter, ORFs, and hairpin, and (ii) repeats, promoter, ORFs, and hairpin. pRTx-1116 includes (i) ORFs, and hairpin, and (ii) repeats, promoter, ORFs, and hairpin. pRTx-1117 includes (i) a truncated ORF region comprising only a 3′ fragment of the region, and hairpin, and (ii) repeats, promoter, ORFs, and hairpin. pRTx-1118 includes (i) repeats, promoter, ORFs, and hairpin, and (ii) repeats, promoter, and ORFs. pRTx-1119 includes (i) repeats, promoter, ORFs, and hairpin, and (ii) repeats, promoter, and a truncated ORF region comprising only a 5′ fragment of the region. pRTx-1120 includes (i) repeats, promoter, ORFs, and hairpin, and (ii) repeats. pRTx-1118 includes (i) repeats, promoter, ORFs, and hairpin, and (ii) a truncated repeats region.

FIG. 6B is a graph showing CAV qPCR on cell suspension transfected with the CAV tandem constructs shown in FIG. 6A. 200 ul of cell suspension was collected at P0 day 2, P0 day 3, and P1 day 2. Viral DNA was extracted, and CAV genomes were quantified by qPCR.

FIG. 6C is a diagram showing a Southern blot for samples from cells transfected with the CAV tandem constructs shown in FIG. 6A. Samples were collected on P1 day 2. Cell pellets were lysed from a 10 ml culture. DNA was extracted and digested with enzymes that cut backbone (B) or non-replicated plasmid (D, DpnI).

FIG. 7 is a graph showing binding of CAV to human cell lines. The y-axis indicates CAV viral genomes (vg) normalized to the MDCC cell control. Each circle represents a biological replicate. Error bars represent standard deviation. As shown, of the human cell lines, CAV bound most strongly to Raji cells, followed by EKVX cells, MRC5 cells, and MCF7 cells.

FIGS. 8A-8B are a series of diagrams showing the genome organization of CAV and a set of exemplary CAVectors. (A) A linearized representation of the WT CAV genome drawn to scale, including the 5′ UTR, VP2 open reading frame, Apoptin open reading frame, VP1 open reading frame, and 3′ UTR. The CAV genome is 2.3 kb in length. (B) Linearized representations of the various CAVector constructs generated and tested. For each CAVector, a Nano-luciferase (nLuc) reporter cassette was inserted into the CAV genome, replacing a fragment of the genome equal in length to the reporter cassette, as shown. The Nano-luciferase reporter comprises an SV40 promoter, the nano-luciferase ORF, and an SV40 terminator sequence. The sequences for the exemplary CAVectors shown in this figure are listed in Tables 2-9.

FIGS. 9A-9C are a series of diagrams showing transduction of MDCC cells with CAVector supernatant. (A) Schematic of transduction assay. In brief, 1e5 cells were incubated with CAVector supernatant for 30 minutes, followed by three washes and the day 0 luminescence measurement. After 24 hours, cells were washed again three times and the day 1 luminescence measurement was obtained. After another 24 hours, cells were washed again three times and the day 2 luminescence measurement was obtained. (B) Negative control transductions with CAVector+pRTX-637. As shown, no luminescence was detected at any of Days 0, 1, or 2 in the negative control samples. (C) Transductions with CAVectors produced in cells co-transfected with pRTX-966 (the full tandem CAV construct). Significance was calculated using a two-way ANOVA with multiple comparisons (*=p<0.05, **=p<0.01, ***=p<0.001). The nLuc4, nLuc5, and nLuc7 CAVectors showed significant increase of luminescence on day 2. The nLuc6 CAVector showed significant increase of luminescence starting on day 1.

FIGS. 10A-10B are a series of diagrams showing purification of CAVectors from supernatant and analysis thereof in a normalized transduction assay. (A) DNase protection assay on purified CAVector particles. Purified particles were treated with or without DNase and then genomes were quantified by qPCR using an nluc probe. (B) Transduction assay with normalized CAVector genomes. CAVectors nLuc4, nLuc5, nLuc6, and nLuc7 showed increased nano-luciferase luminescence at day 2.

FIGS. 11A-11B are a series of graphs showing transduction of human cells by CAVectors. (A) CAVector transduction of Jurkat cells at an MOI of 3. (B) CAVector transduction of Raji cells at an MOI of 3. Significance was calculated using a two-way ANOVA with multiple comparisons (*=p<0.05, **=p<0.01, ***=p<0.001).

FIG. 12 is a table showing the titer, endotoxin levels, and BCA levels for cells producing either CAVectors carrying an nLuc transgene, wild-type CAV (negative control), or AAV2 (positive control).

FIG. 13A-13C are a series of diagrams showing that CAVectors are neutralized by VP1-neutralizing antibodies. (A) Neutralization assay with WT CAV. (B) Neutralizing antibodies in chicken sera specifically recognize VP1. (C) Neutralization of CAVector with chicken serum but not IVIG.

FIG. 14 is a graph showing plasmid containing a full WT CAV genome (pCAV) rescuing Nluc6 CAVector production compared to a sample using a CAV genome in which VP1 was deleted. Addition of VP1-neutralizing antibodies prevented this rescue.

FIGS. 15A-15C are a series of diagrams showing the plasmid maps for exemplary tandem constructs each comprising CAV and/or CAVector genetic element regions arranged in tandem. Each construct includes an Ampicillin resistance cassette and an origin of replication. FIG. 15A shows the plasmid map for pCAV-nLuc6_CAV, the sequence of which is listed in annotated form in Table 14. This construct includes a CAVector genetic element sequence comprising, in order from 5′ to 3′, CAV repeats, inactive CAV VP-encoding sequences, an SV40p_nLuc insert, and CAV hairpin sequences. The CAVector genetic element sequence is positioned 5′ relative to a wild-type CAV genetic element sequence comprising, in order from 5′ to 3′, CAV repeats, CAV VP-encoding sequences, and CAV hairpin sequences. FIG. 15B shows the plasmid map for pCAV-nLuc6_CAVΔ3NCR, the sequence of which is listed in annotated form in Table 15. This construct includes the same nLuc6 CAVector genetic element sequence positioned 5′ relative to a truncated wild-type CAV genetic element sequence comprising only CAV repeats and CAV VP-encoding sequences. FIG. 15C shows the plasmid map for pCAV-nLuc6_CAVΔPromΔORFsΔ3NCR (the latter portion of which is diagrammed as “CAVΔΔΔ”), the sequence of which is listed in annotated form in Table 16. This construct includes the same nLuc6 CAVector genetic element sequence positioned 5′ relative to a truncated wild-type CAV genetic element sequence comprising only CAV repeats.

FIGS. 16A-16D are a series of graphs showing levels of nLuc copies or wild-type CAV genome copies detected in the indicated tissues of mice administered either wild-type CAV, a CAVector encoding nano-luciferase, or an AAV2 encoding nano-luciferase.

FIGS. 17A-17C are a series of diagrams showing the results of in vitro neutralization assays for CAVector or AAV2, as indicated. FIG. 17A shows neutralization of CAVector in mice administered either nLuc CAVector, wild-type CAV, or AAV2-nLuc intramuscularly, or neutralization of CAVector by chicken serum. FIG. 17B shows neutralization of AAV2 in mice administered either nLuc CAVector, wild-type CAV, or AAV2-nLuc intramuscularly, or neutralization of CAVector by chicken serum. FIG. 17C shows the 50% GMT (which stands for geometric mean neutralizing reciprocal titer) for CAV (middle column) or AAV2 (right column) in mice receiving the indicated vectors.

FIG. 18 is a series of diagrams showing assembly of CAV VP1 proteins into virus-like particle (VLP) structures in vitro.

FIG. 19A-19D are a series of graphs showing that CAVectors have CAV-like density and are neutralized by immune serum but not by human serum samples. (A) Density of CAV wildtype and vector particles following ultracentrifugation in a cesium chloride linear gradient and DNase-resistant qPCR of CAV and nanoluciferase (Nluc) amplicons. (B) After dialysis, gradient fractions shown in panel A were assayed for transduction of MDCC-MSB1 cells within minutes of adding vector to cells (day 0) or after 48 hours (day 2). (−) indicates a negative control. (C) Neutralization of CAVector by chicken immune serum is readily observed, while neutralization by human IVIG and individual human serum samples is undetectable except for a weak signal in one sample (donor 38). (D) AAV2-nluc is neutralized by IVIG and 3 of 10 individual human serum samples, but not chicken serum.

FIG. 20 is a series of graphs showing detection of CAVector transgene nLuc in mouse tissues by qPCR 3 weeks after intramuscular injection. Detection of CAVector transgene nLuc in mouse tissues by qPCR 3 weeks after injection. Each symbol represents one mouse. Horizontal line is the geometric mean of each value and error bars represent the geometric standard deviation. Mice were administered the indicated samples by various routes (SR, IV, IP, IM) and their tissues were collected 3 weeks later.

FIG. 21A-21C is a series of diagrams showing that entry of CAVector into a cell is a dynamin- and pH-dependent process. (A) Illustration of common viral entry pathways denoting inhibitors that act at different steps. (B) MDCC-MSB1 cells were pretreated with inhibitors or DMSO control at the indicated concentrations prior to infection. Cells were then transduced in the presence of inhibitor and luminescence was read 20 hours later. (C) For comparison, inhibition of AAV2-nluc entry was similarly studied using Expi293 cells pretreated with inhibitors or DMSO control. Each symbol represents one of two biological replicates. PM: plasma membrane.

FIG. 22A-22B are graphs showing that CAVector retains transduction potency at up to 65° C. and in storage at 4° C. (A) CAVector nLuc 6 was incubated at the indicated temperature for 15 minutes and residual transduction potency was measured by luminescence assay 36 hours after the addition of heat-treated virus to MDCC-MSB1 cells. Each symbol represents one sample. A neutralization sample (NAb) was included as a negative control, as well as a no-vector control (−). (B) A CAVector suspension purified by isopycnic CsCl centrifugation and dialysis was stored for 6 months at 4° C. in buffered saline and assayed for transduction activity. Transduction assays at 0, 3, and 6 months are shown in chronological order, including an uninfected negative control, at the indicated MOIs.

FIG. 23A-23C are a series of diagrams showing the recovery of CAVector using a tandem plasmid. FIG. 23A is a diagram depicting the tandem plasmid. FIG. 23B is a graph depicting quantification of vector genomes following DNase treatment. FIG. 23C is a graph showing an increase of luminescence demonstrating the vector particles are capable of transduction.

The following detailed description of the embodiments of the invention will be better understood when read in conjunction with the appended drawings. For the purpose of illustrating the invention, there are shown in the drawings embodiments that are presently exemplified. It should be understood, however, that the invention is not limited to the precise arrangement and instrumentalities of the embodiments shown in the drawings.

### DETAILED DESCRIPTION OF THE INVENTION

### Definitions

The present invention will be described with respect to particular embodiments and with reference to certain figures but the invention is not limited thereto but only by the claims. Terms as set forth hereinafter are generally to be understood in their common sense unless indicated otherwise.

Where the term “comprising” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is to be understood to preferably also disclose a group which consists only of these embodiments.

Where an indefinite or definite article is used when referring to a singular noun, e.g. “a”, “an” or “the”, this includes a plural of that noun unless something else is specifically stated.

The wording “compound, composition, product, etc. for treating, modulating, etc.” is to be understood to refer a compound, composition, product, etc. per se which is suitable for the indicated purposes of treating, modulating, etc. The wording “compound, composition, product, etc. for treating, modulating, etc.” additionally discloses that, as an embodiment, such compound, composition, product, etc. is for use in treating, modulating, etc.

The wording “compound, composition, product, etc. for use in . . . ”, “use of a compound, composition, product, etc in the manufacture of a medicament, pharmaceutical composition, veterinary composition, diagnostic composition, etc. for . . . ”, or “compound, composition, product, etc. for use as a medicament . . . ” indicates that such compounds, compositions, products, etc. are to be used in therapeutic methods which may be practiced on the human or animal body. They are considered as an equivalent disclosure of embodiments and claims pertaining to methods of treatment, etc. If an embodiment or a claim thus refers to “a compound for use in treating a human or animal being suspected to suffer from a disease”, this is considered to be also a disclosure of a “use of a compound in the manufacture of a medicament for treating a human or animal being suspected to suffer from a disease” or a “method of treatment by administering a compound to a human or animal being suspected to suffer from a disease”. The wording “compound, composition, product, etc. for treating, modulating, etc.” is to be understood to refer a compound, composition, product, etc. per se which is suitable for the indicated purposes of treating, modulating, etc.

If hereinafter examples of a term, value, number, etc. are provided in parentheses, this is to be understood as an indication that the examples mentioned in the parentheses can constitute an embodiment. In some instances, if it is stated that “in embodiments, the nucleic acid molecule comprises a nucleic acid sequence having at least about 70%, 75%, 80%, 85%, 90%, 95%, 96%, 97%, 98%, 99%, or 100% sequence identity to the CAV VP1-encoding nucleotide sequence of Table 1A,” then some embodiments relate to nucleic acid molecules comprising a nucleic acid sequence having at least about 70%, 75%, 80%, 85%, 90%, 95%, 96%, 97%, 98%, 99%, or 100% sequence identity to the VP1-encoding nucleic acid sequence as listed in Table 1A.

The term “amplification,” as used herein, refers to replication of a nucleic acid molecule or a portion thereof, to produce one or more additional copies of the nucleic acid molecule or a portion thereof (e.g., a genetic element or a genetic element region). In some embodiments, amplification results in partial replication of a nucleic acid sequence. In some embodiments, amplification occurs via rolling circle replication.

As used herein, the term “CAVector” refers to a vehicle comprising a genetic element, e.g., a circular DNA, enclosed in a proteinaceous exterior, e.g, the genetic element is substantially protected from digestion with DNAse I by a proteinaceous exterior, and one or both of the genetic element and the proteinaceous exterior comprise a CAV component (for example, a fragment or homolog of a wild-type CAV sequence, e.g., as described herein). A “synthetic CAVector,” as used herein, generally refers to a CAVector that is not naturally occurring, e.g., has a sequence that is different relative to a wild-type virus (e.g., a wild-type CAV as described herein). In some embodiments, the synthetic CAVector is engineered or recombinant, e.g., comprises a genetic element that comprises a difference or modification relative to a wild-type viral genome (e.g., a wild-type CAV genome as described herein). In some embodiments, enclosed within a proteinaceous exterior encompasses 100% coverage by a proteinaceous exterior, as well as less than 100% coverage, e.g., 95%, 90%, 85%, 80%, 70%, 60%, 50% or less. For example, gaps or discontinuities (e.g., that render the proteinaceous exterior permeable to water, ions, peptides, or small molecules) may be present in the proteinaceous exterior, so long as the genetic element is retained in the proteinaceous exterior or protected from digestion with DNAse I, e.g., prior to entry into a host cell. In some embodiments, the CAVector is purified, e.g., it is separated from its original source and/or substantially free (>50%, >60%, >70%, >80%, >90%) of other components. In some embodiments, the CAVector is capable of introducing the genetic element into a target cell (e.g., via infection). In some embodiments, the CAVector is an infective synthetic CAV viral particle. A “CAV component,” as used herein, generally refers to a polypeptide or nucleic acid molecule comprising an activity of a polypeptide or nucleic acid molecule, respectively, encoded by or comprised by a CAV genome, and/or comprising a sequence having at least 50%, 60%, 70%, 75%, 80%, 85%, 90%, 95%, 96%, 97%, 98%, 99%, or 100% sequence identity to a polypeptide encoded by a CAV genome, e.g., a wild-type CAV genome (e.g., as described herein, e.g., as listed in Table 1A, 1B, or 17) or a nucleic acid element comprised in a CAV genome, respectively, or a functional fragment thereof.

As used herein, the term “antibody molecule” refers to a protein, e.g., an immunoglobulin chain or fragment thereof, comprising at least one immunoglobulin variable domain sequence. The term “antibody molecule” encompasses full-length antibodies and antibody fragments (e.g., scFvs). In some embodiments, an antibody molecule is a multispecific antibody molecule, e.g., the antibody molecule comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In embodiments, the multispecific antibody molecule is a bispecific antibody molecule. A bispecific antibody molecule is generally characterized by a first immunoglobulin variable domain sequence which has binding specificity for a first epitope and a second immunoglobulin variable domain sequence that has binding specificity for a second epitope.

As used herein, a “downstream replication-facilitating sequence” (dRFS) refers to a fragment of the sequence of a genetic element (e.g., as described herein), that, when positioned downstream of a genetic element sequence (e.g., the genetic element is 5′ relative to the dRFS), increases replication of the genetic element sequence compared to an otherwise similar genetic element sequence in the absence of the dRFS. Generally, the resultant replicated strand is a functional genetic element that can be enclosed in a proteinaceous exterior to form an CAVector (e.g., as described herein). In some embodiments, a dRFS comprises a displacement site for a Rep protein (e.g., an CAV Rep protein). In some embodiments, a dRFS comprises an CAV 3′ UTR sequence or a fragment thereof, or a sequence having at least 70%, 75%, 80%, 85%, 90%, 95%, 96%, 97%, 98%, or 99% sequence identity thereto). In some embodiments, a dRFS comprises a 5′ UTR (e.g., comprising a hairpin loop). In some embodiments, a dRFS comprises an origin of replication.

As used herein, an “upstream replication-facilitating sequence” (uRFS) refers to a fragment of the sequence of a genetic element (e.g., as described herein), that, when positioned upstream of a genetic element sequence (e.g., the genetic element is 3′ relative to the uRFS) increases replication of the genetic element sequence compared to an otherwise similar genetic element sequence in the absence of the uRFS. Generally, the resultant replicated strand is a functional genetic element that can be enclosed in a proteinaceous exterior to form an CAVector (e.g., as described herein). In some embodiments, an uRFS comprises a binding and/or recognition site for a Rep protein (e.g., an CAV Rep protein). In some embodiments, an uRFS comprises an CAV 5′ UTR sequence or a fragment thereof, or a sequence having at least 70%, 75%, 80%, 85%, 90%, 95%, 96%, 97%, 98%, or 99% sequence identity thereto). In some embodiments, an uRFS comprises a 5′ UTR (e.g., comprising a hairpin loop). In some embodiments, an uRFS comprises an origin of replication.

As used herein, a nucleic acid “encoding” refers to a nucleic acid sequence encoding an amino acid sequence or a functional polynucleotide (e.g., a non-coding RNA, e.g., an siRNA or miRNA).

An “exogenous” agent (e.g., an effector, a nucleic acid (e.g., RNA), a gene, payload, protein) as used herein refers to an agent that is either not comprised by, or not encoded by, a corresponding wild-type virus, e.g., a wild-type CAV, e.g., as described herein. In some embodiments, the exogenous agent does not naturally exist, such as a protein or nucleic acid that has a sequence that is altered (e.g., by insertion, deletion, or substitution) relative to a naturally occurring protein or nucleic acid. In some embodiments, the exogenous agent does not naturally exist in the host cell. In some embodiments, the exogenous agent exists naturally in the host cell but is exogenous to the virus. In some embodiments, the exogenous agent exists naturally in the host cell, but is not present at a desired level or at a desired time. In some embodiments, the exogenous agent does not naturally exist in the target cell. In some embodiments, the exogenous agent exists naturally in the target cell but is exogenous to the virus. In some embodiments, the exogenous agent exists naturally in the target cell, but is not present at a desired level or at a desired time. In embodiments, introduction of the exogenous agent into a target cell results in an unnatural level of the agent in the target cell (e.g., a level greater than the endogenous levels of the agent in the cell).

A “heterologous” agent or element (e.g., an effector, a nucleic acid sequence, an amino acid sequence), as used herein with respect to another agent or element (e.g., an effector, a nucleic acid sequence, an amino acid sequence), refers to agents or elements that are not naturally found together, e.g., in a wild-type virus, e.g., a CAV. In some embodiments, a heterologous nucleic acid sequence may be present in the same nucleic acid as a naturally occurring nucleic acid sequence (e.g., a sequence that is naturally occurring in the CAV). In some embodiments, a heterologous agent or element is exogenous relative to an CAV from which other (e.g., the remainder of) elements of the CAVector are based.

As used herein, the term “genetic element” refers to a nucleic acid molecule that is or can be enclosed within (e.g, protected from DNAse I digestion by) a proteinaceous exterior, e.g., to form a CAVector as described herein. It is understood that the genetic element can be produced as naked DNA and optionally further assembled into a proteinaceous exterior. It is also understood that a CAVector can insert its genetic element into a cell, resulting in the genetic element being present in the cell and the proteinaceous exterior not necessarily entering the cell. In some embodiments, the genetic element comprises a nucleic acid sequence encoding an effector (e.g., an exogenous effector). In some embodiments, the genetic element is single-stranded. In some embodiments the genetic element is circular. In some embodiments, the genetic element comprises one or more sequences of a CAV (e.g., as described herein), e.g., a packaging signal from a CAV as described herein.

As used herein, “genetic element construct” refers to a nucleic acid construct (e.g., a plasmid, bacmid, cosmid, or minicircle) comprising at least one (e.g., two) genetic element sequence(s), or fragment thereof. In some embodiments, a tandem construct as described herein is a genetic element construct comprising two or more genetic element sequences, or fragments thereof, arranged in tandem (e.g., as described herein). In some embodiments, a genetic element construct comprises at least one full length genetic element sequence. In some embodiments, a genetic element comprises a full length genetic element sequence and a partial genetic element sequence. In some embodiments, a genetic element comprises two or more partial genetic element sequences (e.g., in 5′ to 3′ order, a 5′-truncated genetic element sequence arranged in tandem with a 3′-truncated genetic element sequence.

The term “genetic element region,” as used herein, refers to a region of a construct that comprises the sequence of a genetic element. In some embodiments, the genetic element region comprises a sequence having sufficient identity to a wild-type CAV sequence, or a fragment thereof, to be enclosed by a proteinaceous exterior, thereby forming a CAVector (e.g., a sequence having at least 70%, 75%, 80%, 85%, 90%, 95%, 96%, 97%, 98%, 99%, or 100% sequence identity to the wild-type CAV sequence or fragment thereof). In embodiments, the genetic element region comprises a protein binding sequence, e.g., as described herein (e.g., a 5′ UTR, 3′ UTR, and/or a GC-rich region as described herein, or a sequence having at least 70%, 75%, 80%, 85%, 90%, 95%, 96%, 97%, 98%, 99%, or 100% sequence identity thereto). In some embodiments, the genetic element region can undergo rolling circle replication. In some embodiments, the genetic element region comprises an uRFS. In some embodiments, the genetic element region comprises a dRFS. In some embodiments, the genetic element comprises a Rep protein binding site. In some embodiments, the genetic element comprises a Rep protein displacement site. In some embodiments, the construct comprising a genetic element region is not enclosed in a proteinaceous exterior, but a genetic element produced from the construct can be enclosed in a proteinaceous exterior. In some embodiments, the construct comprising the genetic element region further comprises a second uRFS or a second dRFS. In some embodiments, the construct comprising the genetic element region further comprises a vector backbone.

As used herein with reference to a VP1-, VP2-, or Apoptin-coding gene, or a fragment thereof, “nonfunctional” refers to a gene or fragment thereof that does not produce a protein, or that produces a protein that lacks at least one activity (e.g., all activities) compared to its wild-type counterpart. In some embodiments, a nonfunctional gene comprises a premature stop codon. In some embodiments, a nonfunctional gene comprises a frameshift mutation. In some embodiments, a nonfunctional gene lacks a start codon. In some embodiments, the activity is a binding activity or an enzymatic activity.

As used herein, a “functional fragment” of a full-length protein or polypeptide refers to a polypeptide having one or more activities (e.g., all activities) of the full-length protein or polypeptide, and which lacks at least one amino acid (e.g., at least 1, 2, 3, 4, 5, 10, 15, 20, 25, 30, 40, 50, 60, 70, 80, 90, 100, 125, 150, 175, 200, 250, 300, 400, 500, 600, 700, 800, 900, or 1000 amino acids) relative to the full-length protein or polypeptide. In some embodiments, the activity is a binding activity or an enzymatic activity. As used herein, a “functional fragment” of a full-length nucleic acid sequence that binds a protein refers to a nucleic acid sequence that binds to at least one protein bound by the full-length nucleic acid sequence. In some embodiments, the full-length nucleic acid sequence comprises a 5′ UTR sequence, e.g., as described herein.

As used herein, the term “promoter element” refers to a regulatory nucleic acid sequence comprising a sequence having the functionality of a promoter. In some embodiments, the promoter element comprises a promoter as described herein. In some embodiments, the promoter element binds to and/or recruits an RNA polymerase molecule, e.g., such that the RNA polymerase can transcribe an RNA molecule from a nucleic acid sequence downstream of the promoter element. In some embodiments, the promoter element comprises a constitutive promoter, a cell-specific promoter, or a tissue-specific promoter, e.g., as described herein. In some embodiments, the promoter element comprises an inducible promoter, e.g., as described herein.

As used herein, the term “VP1 molecule” refers to a polypeptide having an activity and/or a structural feature of a CAV VP1 protein (e.g., a CAV VP1 protein as described herein, or a functional fragment thereof. A VP1 molecule may, in some instances, comprise one or more of (e.g., 1, 2, 3 or 4 of): a first region comprising. In some instances, the VP1 molecule comprises, in N-terminal to C-terminal order, the first, second, third, and fourth regions. A VP1 molecule may, in some instances, comprise a polypeptide encoded by a CAV VP1 nucleic acid. A VP1 molecule may, in some instances, further comprise a heterologous sequence, e.g., from a CAV VP1 protein, e.g., as described herein. In some embodiments, a VP1 molecule is encoded by a CAV genome (e.g., a wild-type CAV genome, e.g., as described herein). In some embodiments, a VP1 molecule is a polypeptide encoded by a CAV VP1 nucleic acid (e.g., a VP1 gene, e.g., as described herein). In some embodiments, a VP1 molecule is a splice variant or comprises a post-translational modification.

As used herein, the term “VP2 molecule” refers to a polypeptide having an activity and/or a structural feature of a CAV VP2 protein (e.g., a CAV VP2 protein as described herein, or a functional fragment thereof. In some embodiments, a VP2 molecule is encoded by a CAV genome (e.g., a wild-type CAV genome, e.g., as described herein). In some embodiments, a VP2 molecule is a polypeptide encoded by a CAV VP2 nucleic acid (e.g., a VP2 gene, e.g., as described herein). In some embodiments, a VP2 molecule is a splice variant or comprises a post-translational modification.

As used herein, the term “Apoptin molecule” refers to a polypeptide having an activity and/or a structural feature of a CAV Apoptin protein (e.g., a CAV Apoptin protein as described herein, or a functional fragment thereof. In some embodiments, an Apoptin molecule is encoded by a CAV genome (e.g., a wild-type CAV genome, e.g., as described herein). In some embodiments, an Apoptin molecule is a polypeptide encoded by a CAV Apoptin nucleic acid (e.g., an Apoptin gene). In some embodiments, an Apoptin molecule is a splice variant or comprises a post-translational modification.

As used herein, the term “CAV capsid polypeptide” refers to a polypeptide present in the capsid of a wild-type CAV, or a polypeptide having an activity and/or a structural feature of said polypeptide. In some embodiments, the CAV capsid polypeptide is a VP1 molecule.

As used herein, the term “VP1 nucleic acid” refers to a nucleic acid that encodes a VP1 molecule, or the reverse complement thereof. The nucleic acid may be single stranded or double stranded. In some embodiments, the VP1 nucleic acid comprises a CAV VP1 gene, e.g., as described herein. A “VP1 gene” generally refers to a nucleic acid sequence encoding a wild-type VP1 molecule, or the reverse complement thereof. In some embodiments, a VP1 gene comprises a sense strand. In some embodiments, a VP1 gene comprises an antisense strand. In some embodiments, a VP1 gene is double-stranded.

As used herein, the term “VP2 nucleic acid” refers to a nucleic acid that encodes a VP2 molecule, or the reverse complement thereof. The nucleic acid may be single stranded or double stranded. In some embodiments, the VP2 nucleic acid comprises a CAV VP2 gene, e.g., as described herein. A “VP2 gene” generally refers to a nucleic acid sequence encoding a wild-type VP2 molecule, or the reverse complement thereof. In some embodiments, a VP2 gene comprises a sense strand. In some embodiments, a VP2 gene comprises an antisense strand. In some embodiments, a VP2 gene is double-stranded.

As used herein, the term “Apoptin nucleic acid” refers to a nucleic acid that encodes a Apoptin molecule, or the reverse complement thereof. The nucleic acid may be single stranded or double stranded. In some embodiments, the Apoptin nucleic acid comprises a CAV Apoptin gene, e.g., as described herein. An “Apoptin gene” generally refers to a nucleic acid sequence encoding a wild-type Apoptin molecule, or the reverse complement thereof. In some embodiments, an Apoptin gene comprises a sense strand. In some embodiments, an Apoptin gene comprises an antisense strand. In some embodiments, an Apoptin gene is double-stranded.

As used herein, the term “CAV genome sequence” refers to a nucleic acid sequence comprising a full-length genome sequence from a wild-type CAV, e.g., as described herein, or a sequence having at least 70%, 75%, 80%, 85%, 90%, 95%, 96%, 97%, 98%, 99%, or 100% sequence identity thereto. In some embodiments, a CAV genome comprises a CAV genome sequence as described herein (e.g., a wild-type CAV genome sequence, e.g., as listed in any of Tables 1A and 1B or Table 17).

As used herein, the term “CAV UTR” refers to a nucleic acid sequence comprising an untranslated region (UTR) sequence (e.g., the sequence of a 5′ UTR or a 3′ UTR) from a CAV (e.g., a wild-type CAV, e.g., as described herein, e.g., as listed in Table 1A, 1B, or 17), or a sequence having at least 70%, 75%, 80%, 85%, 90%, 95%, 96%, 97%, 98%, 99%, or 100% identity thereto.

As used herein, the term “proteinaceous exterior” refers to an exterior component that is predominantly (e.g., >50%, >60%, >70%, >80%, >90%) protein. In some embodiments, a proteinaceous exterior encloses a nucleic acid molecule, e.g., a genetic element, e.g., as described herein. In some embodiments, the proteinaceous exterior comprises a capsid. In some embodiments, the proteinaceous exterior forms the capsid, or a portion thereof, of a viral particle.

As used herein, the term “protein binding sequence” refers to a nucleic acid sequence capable of binding to (e.g., specifically binding to) a protein, e.g., a component of a proteinaceous exterior, e.g., as described herein. In some embodiments, the protein binding sequence binds to the component of the proteinaceous exterior with sufficient affinity to promote enclosure of the genetic element comprising the protein binding sequence within the proteinaceous exterior. In some embodiments, the protein binding sequence binds directly to the proteinaceous exterior protein. In some embodiments, the protein binding sequence binds indirectly to the proteinaceous exterior protein (e.g., the protein binding sequence binds to a protein, nucleic acid molecule, or other moiety associated with the proteinaceous exterior protein).

As used herein, the term “regulatory nucleic acid” refers to a nucleic acid sequence that modifies expression, e.g., transcription and/or translation, of a DNA sequence that encodes an expression product. In embodiments, the expression product comprises RNA or protein.

As used herein, the term “regulatory sequence” refers to a nucleic acid sequence that modifies transcription of a target gene product. In some embodiments, the regulatory sequence is a promoter or an enhancer.

As used herein, the term “Rep” or “replication protein” refers to a protein, e.g., a viral protein, that promotes viral genome replication. In some embodiments, the replication protein is an CAV Rep protein.

As used herein, the term “Rep binding site” refers to a nucleic acid sequence within a nucleic acid molecule that is recognized and bound by a Rep protein (e.g., an CAV Rep protein). In some embodiments, a Rep binding site comprises a 5′ UTR (e.g., comprising a hairpin loop). In some embodiments, a Rep binding site comprises an origin of replication (ORI).

As used herein, the term “Rep displacement site” refers to a nucleic acid sequence within a nucleic acid molecule that is capable of causing a Rep protein (e.g., an CAV Rep protein) associated with (e.g., bound to) the nucleic acid molecule to release the nucleic acid molecule upon reaching the Rep displacement site. In some embodiments, a Rep displacement site comprises a 5′ UTR (e.g., comprising a hairpin loop). In some embodiments, a Rep displacement site comprises an origin of replication (ORI).

As used herein, the term “specifically binds” refers to a first molecule (e.g., a capsid protein, e.g., a VP1 protein) that binds to a second molecule (e.g., a nucleic acid comprising a protein binding sequence, e.g., a packaging signal) more strongly than to a nonspecific control molecule (e.g., a nucleic acid lacking the protein binding sequence). In some embodiments, the first molecule shows a detectable level of binding to the nonspecific control molecule. In some embodiments, the K_{D} of the first molecule for the second molecule is lower than the K_{D} of the first molecule for the nonspecific control molecule by a factor of 5, 10, 20, 50, or 100.

As used herein, a “substantially non-pathogenic” organism, particle, or component, refers to an organism, particle (e.g., a virus or a CAVector, e.g., as described herein), or component thereof that does not cause or induce an unacceptable disease or pathogenic condition, e.g., in a host organism, e.g., a mammal, e.g., a human. In some embodiments, administration of a CAVector to a subject can result in minor reactions or side effects that are acceptable as part of standard of care.

As used herein, the term “non-pathogenic” refers to an organism or component thereof that does not cause or induce a unacceptable disease or pathogenic condition, e.g., in a host organism, e.g., a mammal, e.g., a human.

As used herein, a “substantially non-integrating” genetic element refers to a genetic element, e.g., a genetic element in a virus or CAVector, e.g., as described herein, wherein less than about 0.01%, 0.05%, 0.1%, 0.5%, or 1% of the genetic element that enter into a host cell (e.g., a eukaryotic cell) or organism (e.g., a mammal, e.g., a human) integrate into the genome. In some embodiments the genetic element does not detectably integrate into the genome of, e.g., a host cell. In some embodiments, integration of the genetic element into the genome can be detected using techniques as described herein, e.g., nucleic acid sequencing, PCR detection and/or nucleic acid hybridization. In some embodiments, integration frequency is determined by quantitative gel purification assay of genomic DNA separated from free vector, e.g., as described in Wang et al. (2004, Gene Therapy 11: 711-721, incorporated herein by reference in its entirety).

A “subsequence” as used herein refers to a nucleic acid sequence or an amino acid sequence that is comprised in a larger nucleic acid sequence or amino acid sequence, respectively. In some instances, a subsequence may comprise a domain or functional fragment of the larger sequence. In some instances, the subsequence may comprise a fragment of the larger sequence capable of forming secondary and/or tertiary structures when isolated from the larger sequence similar to the secondary and/or tertiary structures formed by the subsequence when present with the remainder of the larger sequence. In some instances, a subsequence can be replaced by another sequence (e.g., a subsequence comprising an exogenous sequence or a sequence heterologous to the remainder of the larger sequence, e.g., a corresponding subsequence from a different CAV).

This invention relates generally to CAVectors, e.g., synthetic CAVectors, and uses thereof. The present disclosure provides CAVectors, compositions comprising CAVectors, and methods of making or using CAVectors. CAVectors are generally useful as delivery vehicles, e.g., for delivering a therapeutic agent to a eukaryotic cell. Generally, an CAVector will include a genetic element comprising a nucleic acid sequence (e.g., encoding an effector, e.g., an exogenous effector or an endogenous effector) enclosed within a proteinaceous exterior. A CAVector may include one or more deletions of sequences (e.g., regions or domains as described herein) relative to a CAV sequence (e.g., as described herein). CAVectors can be used as a vehicle for delivering the genetic element, or an effector encoded therein (e.g., a polypeptide or nucleic acid effector, e.g., as described herein), into eukaryotic cells, e.g., to treat a disease or disorder in a subject comprising the cells.

### TABLE OF CONTENTS

I. Compositions and Methods for Making CAVectors
   A. Components and Assembly of CAVectors
      i. VP1 molecules for assembly of CAVectors
      ii. VP2 molecules for assembly of CAVectors
   B. Genetic Element Constructs
      i. Plasmids
      ii. Circular genetic element constructs
      iii. In vitro circularization
      iv. Cis/trans constructs
      v. Expression cassettes
      vi. Design and production of a genetic element construct
   C. Effectors
   D. Host Cells
      i. Introduction of genetic elements into host cells
      ii. Methods for providing CAV protein(s) in cis or trans
      iii. Helpers
      iv. Exemplary cell types
   E. Culture Conditions
   F. Harvest and Purification
II. CAVectors
   A. CAVs
   B. VP1 molecules
   C. VP2 molecules
   D. Genetic elements
   E. Protein binding sequences
   F. 5′ UTR Regions
   G. GC-rich regions
   H. Effectors
   I. Regulatory Sequences
   J. Replication Proteins
   K. Other Sequences
   L. Proteinaceous exterior
III. Genetic element constructs
IV. Compositions
V. Host cells
VI. Methods of use
VII. Administration/Delivery

### I. Compositions and Methods for Making CAVectors

The present disclosure provides, in some aspects, genetic element constructs that can be used for producing CAVectors, e.g., as described herein.

Components and Assembly of CAVectors

The compositions and methods herein can be used to produce CAVectors. As described herein, an CAVector generally comprises a genetic element (e.g., a single-stranded, circular DNA molecule, e.g., comprising a 5′ UTR region as described herein) enclosed within a proteinaceous exterior (e.g., comprising a polypeptide encoded by an CAV VP1 nucleic acid, e.g., as described herein). In some embodiments, the genetic element comprises one or more sequences encoding CAV ORFs (e.g., one or more of an CAV VP1, VP2, and/or Apoptin). As used herein, an CAV ORF or ORF molecule (e.g., an CAV VP1, VP2, and/or Apoptin) includes a polypeptide comprising an amino acid sequence having at least 70%, 75%, 80%, 85%, 90%, 95%, 96%, 97%, 98%, 99%, or 100% sequence identity to a corresponding CAV ORF sequence, e.g., as described herein). In embodiments, the genetic element comprises a sequence encoding an CAV VP1, or a splice variant or functional fragment thereof (e.g., a jelly-roll region, e.g., as described herein). In some embodiments, the proteinaceous exterior comprises a polypeptide encoded by an CAV VP1 nucleic acid (e.g., an CAV VP1 molecule or a splice variant or functional fragment thereof).

In some embodiments, a CAVector is assembled by enclosing a genetic element (e.g., as described herein) within a proteinaceous exterior (e.g., as described herein). In some embodiments, the genetic element is enclosed within the proteinaceous exterior in a host cell (e.g., as described herein). In some embodiments, the host cell expresses one or more polypeptides comprised in the proteinaceous exterior (e.g., a polypeptide encoded by a CAV VP1 nucleic acid, e.g., an VP1 molecule). For example, in some embodiments, the host cell comprises a nucleic acid sequence encoding a CAV VP1 molecule, e.g., a splice variant or a functional fragment of a CAV VP1 polypeptide (e.g., a wild-type CAV VP1 protein or a polypeptide encoded by a wild-type CAV VP1 nucleic acid, e.g., as described herein). In embodiments, the nucleic acid sequence encoding the CAV VP1 molecule is comprised in a genetic element construct (e.g., a plasmid, viral vector, virus, minicircle, bacmid, or artificial chromosome) comprised in the host cell. In embodiments, the nucleic acid sequence encoding the CAV VP1 molecule is integrated into the genome of the host cell.

In some embodiments, the host cell comprises the genetic element and/or a genetic element construct comprising the sequence of the genetic element. In some embodiments, the genetic element construct is selected from a plasmid, viral nucleic acid, minicircle, bacmid, or artificial chromosome. In some embodiments, the genetic element is excised from the genetic element construct and, optionally, converted from a double-stranded form to a single-stranded form (e.g., by denaturation). In some embodiments, the genetic element is generated by a polymerase based on a template sequence in the genetic element construct. In some embodiments, the polymerase produces a single-stranded copy of the genetic element sequence, which can optionally be circularized to form a genetic element as described herein. In other embodiments, the genetic element construct is a double-stranded minicircle produced by circularizing the nucleic acid sequence of the genetic element in vitro. In embodiments, the in vitro-circularized (IVC) minicircle is introduced into the host cell, where it is converted to a single-stranded genetic element suitable for enclosure in a proteinaceous exterior, as described herein.

VP1 Molecules, e.g., for Assembly of CAVectors

A CAVector can be made, for example, by enclosing a genetic element within a proteinaceous exterior. The proteinaceous exterior of a CAVector generally comprises a polypeptide encoded by an CAV VP1 nucleic acid (e.g., an CAV VP1 molecule or a splice variant or functional fragment thereof, e.g., as described herein). In embodiments, the proteinaceous exterior comprises one or both of a CAV VP1 arginine-rich region and/or jelly-roll region. In some embodiments, the proteinaceous exterior comprises an CAV VP1 jelly-roll region (e.g., as described herein). In some embodiments, the proteinaceous exterior comprises an CAV VP1 arginine-rich region (e.g., as described herein).

In some embodiments, the CAVector comprises a VP1 molecule and/or a nucleic acid encoding a VP1 molecule. Generally, a VP1 molecule comprises a polypeptide having the structural features and/or activity of an CAV VP1 protein (e.g., an CAV VP1 protein as described herein), or a functional fragment thereof. In some embodiments, the VP1 molecule comprises a truncation relative to an CAV VP1 protein (e.g., an CAV VP1 protein as described herein). In some embodiments, the VP1 molecule is truncated by at least 10, 20, 30, 40, 50, 60, 70, 80, 90, 100, 150, 200, 250, 300, 350, 400, 450, 500, 550, 600, 650, or 700 amino acids of the CAV VP1 protein. In some embodiments, an VP1 molecule comprises an amino acid sequence having at least 75%, 80%, 85%, 90%, 95%, 96%, 97%, 98%, or 99% sequence identity to a CAV VP1 protein, e.g., as described herein. A VP1 molecule can generally bind to a nucleic acid molecule, such as DNA (e.g., a genetic element, e.g., as described herein), e.g., at a protein binding sequence therein.

Without wishing to be bound by theory, a VP1 molecule may be capable of binding to other VP1 molecules, e.g., to form a proteinaceous exterior (e.g., as described herein). Such a VP1 molecule may be described as having the capacity to form a capsid. In some embodiments, the proteinaceous exterior may enclose a nucleic acid molecule (e.g., a genetic element as described herein, e.g., produced using a tandem construct as described herein). In some embodiments, a plurality of VP1 molecules may form a multimer, e.g., to produce a proteinaceous exterior. In some embodiments, the multimer may be a homomultimer. In other embodiments, the multimer may be a heteromultimer.

Other CAV Polypeptides, e.g., for Assembly of CAVectors

In some embodiments, producing an CAVector using the compositions or methods described herein may involve expression of a CAV VP2 molecule (e.g., as described herein), or a splice variant or functional fragment thereof. In some embodiments, the CAVector comprises a VP2 molecule, or a splice variant or functional fragment thereof, and/or a nucleic acid encoding a VP2 molecule, or a splice variant or functional fragment thereof. In some embodiments, the CAVector does not comprise a VP2 molecule, or a splice variant or functional fragment thereof, and/or a nucleic acid encoding a VP2 molecule, or a splice variant or functional fragment thereof. In some embodiments, producing the CAVector comprises expression of a VP2 molecule, or a splice variant or functional fragment thereof, but the VP2 molecule is not incorporated into the CAVector.

In some embodiments, producing an CAVector using the compositions or methods described herein may involve expression of a CAV Apoptin molecule (e.g., as described herein), or a splice variant or functional fragment thereof. In some embodiments, the CAVector comprises a Apoptin molecule, or a splice variant or functional fragment thereof, and/or a nucleic acid encoding a Apoptin molecule, or a splice variant or functional fragment thereof. In some embodiments, the CAVector does not comprise a Apoptin molecule, or a splice variant or functional fragment thereof, and/or a nucleic acid encoding a Apoptin molecule, or a splice variant or functional fragment thereof. In some embodiments, producing the CAVector comprises expression of a Apoptin molecule, or a splice variant or functional fragment thereof, but the Apoptin molecule is not incorporated into the CAVector.

Genetic Element Constructs, e.g., for Assembly of CAVectors

The genetic element of a CAVector as described herein may be produced from a genetic element construct that comprises a genetic element region and optionally other sequence such as vector backbone. Generally, the genetic element construct comprises a CAV 5′ UTR (e.g., as described herein). A genetic element construct may be any genetic element construct suitable for delivery of the sequence of the genetic element into a host cell in which the genetic element can be enclosed within a proteinaceous exterior. In some embodiments, the genetic element construct comprises a promoter. In some embodiments, the genetic element construct is a linear nucleic acid molecule. In some embodiments, the genetic element construct is a circular nucleic acid molecule (e.g., a plasmid, bacmid, or a minicircle, e.g., as described herein). In some embodiments, the genetic element construct comprises baculovirus sequences (e.g., such that an insect cell comprising the genetic element construct can produce a baculovirus comprising the genetic element sequence of the genetic element construct, or a fragment thereof). The genetic element construct may, in some embodiments, be double-stranded. In other embodiments, the genetic element is single-stranded. In some embodiments, the genetic element construct comprises DNA. In some embodiments, the genetic element construct comprises RNA. In some embodiments, the genetic element construct comprises one or more modified nucleotides.

In some embodiments, the genetic element construct comprises one copy of the genetic element sequence. In some embodiments, the genetic element comprises a plurality of copies of the genetic element sequence (e.g., two copies of the genetic element sequence). In some embodiments, the genetic comprises one full-length copy of the genetic element sequence and at least one partial genetic element sequence. In some embodiments, two copies of the genetic element sequence (e.g., the full length and/or partial genetic element sequences) are positioned in tandem within the genetic element construct (e.g., as described herein).

In some aspects, the present disclosure provides a method for replication and propagation of the CAVector as described herein (e.g., in a cell culture system), which may comprise one or more of the following steps: (a) introducing (e.g., transfecting) a genetic element (e.g., linearized) into a cell line sensitive to CAVector infection; (b) harvesting the cells and optionally isolating cells showing the presence of the genetic element; (c) culturing the cells obtained in step (b) (e.g., for at least three days, such as at least one week or longer), depending on experimental conditions and gene expression; and (d) harvesting the cells of step (c), e.g., as described herein.

Plasmids

In some embodiments, the genetic element construct is a plasmid. The plasmid will generally comprise the sequence of a genetic element as described herein as well as an origin of replication suitable for replication in a host cell (e.g., a bacterial origin of replication for replication in bacterial cells) and a selectable marker (e.g., an antibiotic resistance gene). In some embodiments, the sequence of the genetic element can be excised from the plasmid. In some embodiments, the plasmid is capable of replication in a bacterial cell. In some embodiments, the plasmid is capable of replication in a mammalian cell (e.g., a human cell). In some embodiments, a plasmid is at least 300, 400, 500, 600, 700, 800, 900, 1000, 2000, 3000, 4000, or 5000 bp in length. In some embodiments, the plasmid is less than 600, 700, 800, 900, 1000, 2000, 3000, 4000, 5000, 6000, 7000, 8000, 9000, or 10,000 bp in length. In some embodiments, the plasmid has a length between 300-400, 400-500, 500-600, 600-700, 700-800, 800-900, 900-1000, 1000-1500, 1500-2000, 2000-2500, 2500-3000, 3000-4000, or 4000-5000 bp. In some embodiments, the genetic element can be excised from a plasmid (e.g., by in vitro circularization), for example, to form a minicircle, e.g., as described herein. In embodiments, excision of the genetic element separates the genetic element sequence from the plasmid backbone (e.g., separates the genetic element from a bacterial backbone).

Circular Genetic Element Constructs

In some embodiments, the genetic element construct is a circular genetic element construct, e.g., lacking a backbone (e.g., lacking a bacterial origin of replication and/or selectable marker). In embodiments, the genetic element is a double-stranded circular genetic element construct. In embodiments, the double-stranded circular genetic element construct is produced by in vitro circularization (IVC), e.g., as described herein. In embodiments, the double-stranded circular genetic element construct can be introduced into a host cell, in which it can be converted into or used as a template for generating single-stranded circular genetic elements, e.g., as described herein. In some embodiments, the circular genetic element construct does not comprise a plasmid backbone or a functional fragment thereof. In some embodiments, the circular genetic element construct is at least 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, or 4500 bp in length. In some embodiments, the circular genetic element construct is less than 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5500, or 6000 bp in length. In some embodiments, the circular genetic element construct is between 2000-2100, 2100-2200, 2200-2300, 2300-2400, 2400-2500, 2500-2600, 2600-2700, 2700-2800, 2800-2900, 2900-3000, 3000-3100, 3100-3200, 3200-3300, 3300-3400, 3400-3500, 3500-3600, 3600-3700, 3700-3800, 3800-3900, 3900-4000, 4000-4100, 4100-4200, 4200-4300, 4300-4400, or 4400-4500 bp in length. In some embodiments, the circular genetic element construct is a minicircle.

In Vitro Circularization

In some instances, the genetic element to be packaged into a proteinaceous exterior is a single stranded circular DNA. The genetic element may, in some instances, be introduced into a host cell via a genetic element construct having a form other than a single stranded circular DNA. For example, the genetic element construct may be a double-stranded circular DNA. The double-stranded circular DNA may then be converted into a single-stranded circular DNA in the host cell (e.g., a host cell comprising a suitable enzyme for rolling circle replication, e.g., a CAV Rep protein). In some embodiments, the double-stranded circular DNA is produced by in vitro circularization (IVC), e.g., as described herein.

Generally, in vitro circularized DNA constructs can be produced by digesting a plasmid comprising the sequence of a genetic element to be packaged, such that the genetic element sequence is excised as a linear DNA molecule. The resultant linear DNA can then be ligated, e.g., using a DNA ligase, to form a double-stranded circular DNA. In some instances, a double-stranded circular DNA produced by in vitro circularization can undergo rolling circle replication, e.g., as described herein. Without wishing to be bound by theory, it is contemplated that in vitro circularization results in a double-stranded DNA construct that can undergo rolling circle replication without further modification, thereby being capable of producing single-stranded circular DNA of a suitable size to be packaged into an CAVector, e.g., as described herein. In some embodiments, the double-stranded DNA construct is smaller than a plasmid (e.g., a bacterial plasmid). In some embodiments, the double-stranded DNA construct is excised from a plasmid (e.g., a bacterial plasmid) and then circularized, e.g., by in vitro circularization.

Cis/Trans Constructs

In some embodiments, a genetic element construct as described herein comprises one or more sequences encoding one or more CAV ORFs, e.g., proteinaceous exterior components (e.g., polypeptides encoded by one or more of an CAV VP1, VP2, and/or Apoptin nucleic acid, e.g., as described herein). For example, the genetic element construct may comprise a nucleic acid sequence encoding a CAV VP1 molecule. Such genetic element constructs can be suitable for introducing the genetic element and the CAV ORF(s) into a host cell in cis. In other embodiments, a genetic element construct as described herein does not comprise sequences encoding one or more CAV ORFs, e.g., proteinaceous exterior components (e.g., polypeptides encoded by a CAV VP1 nucleic acid, e.g., as described herein). For example, the genetic element construct may not comprise a nucleic acid sequence encoding a CAV VP1 molecule. Such genetic element constructs can be suitable for introducing the genetic element into a host cell, with the one or more CAV ORFs to be provided in trans (e.g., via introduction of a second genetic element construct encoding one or more of the CAV ORFs, or via a CAV ORF cassette integrated into the genome of the host cell).

In some embodiments, the genetic element construct comprises a sequence encoding an CAV VP1 molecule, or a splice variant or functional fragment thereof (e.g., a jelly-roll region, e.g., as described herein). In embodiments, the portion of the genetic element that does not comprise the sequence of the genetic element comprises the sequence encoding the CAV VP1 molecule, or splice variant or functional fragment thereof (e.g., in a cassette comprising a promoter and the sequence encoding the CAV VP1 molecule, or splice variant or functional fragment thereof). In further embodiments, the portion of the construct comprising the sequence of the genetic element comprises a sequence encoding a CAV VP1 molecule, or a splice variant or functional fragment thereof (e.g., a jelly-roll region, e.g., as described herein). In embodiments, enclosure of such a genetic element in a proteinaceous exterior (e.g., as described herein) produces a replication-component CAVector (e.g., a CAVector that upon infecting a cell, enables the cell to produce additional copies of the CAVector without introducing further genetic element constructs, e.g., encoding one or more CAV ORFs as described herein, into the cell).

In other embodiments, the genetic element does not comprise a sequence encoding a CAV VP1 molecule, or a splice variant or functional fragment thereof (e.g., a jelly-roll region, e.g., as described herein). In embodiments, enclosure of such a genetic element in a proteinaceous exterior (e.g., as described herein) produces a replication-incompetent CAVector (e.g., a CAVector that, upon infecting a cell, does not enable the infected cell to produce additional CAVectors, e.g., in the absence of one or more additional constructs, e.g., encoding one or more CAV ORFs as described herein).

Expression Cassettes

In some embodiments, a genetic element construct comprises one or more cassettes for expression of a polypeptide or noncoding RNA (e.g., a miRNA or an siRNA). In some embodiments, the genetic element construct comprises a cassette for expression of an effector (e.g., an exogenous or endogenous effector), e.g., a polypeptide or noncoding RNA, as described herein. In some embodiments, the genetic element construct comprises a cassette for expression of a CAV protein (e.g., a CAV VP1, VP2, and/or Apoptin, or a functional fragment thereof). The expression cassettes may, in some embodiments, be located within the genetic element sequence. In embodiments, an expression cassette for an effector is located within the genetic element sequence. In embodiments, an expression cassette for a CAV protein is located within the genetic element sequence. In other embodiments, the expression cassettes are located at a position within the genetic element construct outside of the sequence of the genetic element (e.g., in the backbone). In embodiments, an expression cassette for a CAV protein is located at a position within the genetic element construct outside of the sequence of the genetic element (e.g., in the backbone).

A polypeptide expression cassette generally comprises a promoter and a coding sequence encoding a polypeptide, e.g., an effector (e.g., an exogenous or endogenous effector as described herein) or a CAV protein (e.g., a sequence encoding a CAV VP1, VP2, and/or Apoptin, or a functional fragment thereof). Exemplary promoters that can be included in an polypeptide expression cassette (e.g., to drive expression of the polypeptide) include, without limitation, constitutive promoters (e.g., CMV, RSV, PGK, EF1a, or SV40), cell or tissue-specific promoters (e.g., skeletal α-actin promoter, myosin light chain 2A promoter, dystrophin promoter, muscle creatine kinase promoter, liver albumin promoter, hepatitis B virus core promoter, osteocalcin promoter, bone sialoprotein promoter, CD2 promoter, immunoglobulin heavy chain promoter, T cell receptor a chain promoter, neuron-specific enolase (NSE) promoter, or neurofilament light-chain promoter), and inducible promoters (e.g., zinc-inducible sheep metallothionine (MT) promoter; the dexamethasone (Dex)-inducible mouse mammary tumor virus (MMTV) promoter; the T7 polymerase promoter system, tetracycline-repressible system, tetracycline-inducible system, RU486-inducible system, rapamycin-inducible system), e.g., as described herein. In some embodiments, the expression cassette further comprises an enhancer, e.g., as described herein.

Design and Production of a Genetic Element Construct

Various methods are available for synthesizing a genetic element construct. For instance, the genetic element construct sequence may be divided into smaller overlapping pieces (e.g., in the range of about 100 bp to about 10 kb segments or individual ORFs) that are easier to synthesize. These DNA segments are synthesized from a set of overlapping single-stranded oligonucleotides. The resulting overlapping synthons are then assembled into larger pieces of DNA, e.g., the genetic element construct. The segments or ORFs may be assembled into the genetic element construct, e.g., by in vitro recombination or unique restriction sites at 5′ and 3′ ends to enable ligation.

The genetic element construct can be synthesized with a design algorithm that parses the construct sequence into oligo-length fragments, creating suitable design conditions for synthesis that take into account the complexity of the sequence space. Oligos are then chemically synthesized on semiconductor-based, high-density chips, where over 200,000 individual oligos are synthesized per chip. The oligos are assembled with an assembly techniques, such as BioFab®, to build longer DNA segments from the smaller oligos. This is done in a parallel fashion, so hundreds to thousands of synthetic DNA segments are built at one time.

Each genetic element construct or segment of the genetic element construct may be sequence verified. In some embodiments, high-throughput sequencing of RNA or DNA can take place using AnyDot.chips (Genovoxx, Germany), which allows for the monitoring of biological processes (e.g., miRNA expression or allele variability (SNP detection). Other high-throughput sequencing systems include those disclosed in Venter, J., et al. Science 16 Feb. 2001; Adams, M. et al, Science 24 Mar. 2000; and M. J, Levene, et al. Science 299:682-686, January 2003; as well as US Publication Application No. 20030044781 and 2006/0078937. Overall such systems involve sequencing a target nucleic acid molecule having a plurality of bases by the temporal addition of bases via a polymerization reaction that is measured on a molecule of nucleic acid, i.e., the activity of a nucleic acid polymerizing enzyme on the template nucleic acid molecule to be sequenced is followed in real time. In some embodiments, shotgun sequencing is performed.

A genetic element construct can be designed such that factors for replicating or packaging may be supplied in cis or in trans, relative to the genetic element. For example, when supplied in cis, the genetic element may comprise one or more genes encoding a CAV VP1, VP2, and/or Apoptin, e.g., as described herein. In some embodiments, replication and/or packaging signals can be incorporated into a genetic element, for example, to induce amplification and/or encapsulation. In some embodiments, an effector is inserted into a specific site in the genome. In some embodiments, one or more viral ORFs are replaced with an effector.

In another example, when replication or packaging factors are supplied in trans, the genetic element may lack genes encoding one or more of a CAV VP1, VP2, and/or Apoptin, e.g., as described herein; this protein or proteins may be supplied, e.g., by another nucleic acid. In some embodiments, minimal cis signals (e.g., 5′ UTR, 3′ UTR, and/or GC-rich region) are present in the genetic element. In some embodiments, the genetic element does not encode replication or packaging factors (e.g., replicase and/or capsid proteins). Such factors may, in some embodiments, be supplied by one or more nucleic acids (e.g., a viral nucleic acid, a plasmid, or a nucleic acid integrated into the host cell genome). In some embodiments, the second nucleic acid expresses proteins and/or RNAs sufficient to induce amplification and/or packaging, but may lack its own packaging signals. In some embodiments, the genetic element and the second nucleic acid are introduced into the host cell (e.g., concurrently or separately), resulting in amplification and/or packaging of the genetic element but not of the second nucleic acid.

In some embodiments, the genetic element construct may be designed using computer-aided design tools.

General methods of making nucleic acid molecules such as genetic element constructs are described in, for example, Khudyakov & Fields, Artificial DNA: Methods and Applications, CRC Press (2002); in Zhao, Synthetic Biology: Tools and Applications, (First Edition), Academic Press (2013); and Egli & Herdewijn, Chemistry and Biology of Artificial Nucleic Acids, (First Edition), Wiley-VCH (2012).

Rolling Circle Amplification

Without wishing to be bound by theory, rolling circle amplification may occur via Rep protein binding to a Rep binding site (e.g., comprising a 5′ UTR, e.g., comprising a hairpin loop and/or an origin of replication, e.g., as described herein) positioned 5′ relative to (or within the 5′ region of) a genetic element region. The Rep protein may then proceed through the genetic element region, resulting in the synthesis of the genetic element. The released genetic element may then be circularized and then enclosed within a proteinaceous exterior to form an CAVector.

Tandem Constructs

In some embodiments, the genetic element construct is a tandem construct. Tandem constructs as described herein generally comprise a first genetic element region, which, when not connected to the remainder of the genetic element construct and/or converted to a circular, single-stranded DNA molecule, can be enclosed within a proteinaceous exterior, thereby producing a CAVector. The tandem constructs may further comprise a second genetic element region, or a portion thereof. In some embodiments, the tandem constructs described herein can be used to produce a genetic element suitable for enclosure in a proteinaceous exterior (e.g., comprising a polypeptide encoded by an VP1 nucleic acid), e.g., by rolling circle amplification. In some embodiments, a genetic element suitable for enclosure in the proteinaceous exterior is produced via rolling circle amplification of the first genetic element region.

In some embodiments, a tandem construct is a genetic element construct comprising a first copy of a genetic element sequence (e.g., a genetic element region) and at least a portion of a second copy of a genetic element sequence (e.g., comprising an uRFS or a dRFS). In some embodiments, the second copy comprises the full sequence of the genetic element. In some embodiments, the second copy comprises a partial sequence of the genetic element (e.g., at least 1%, 2%, 3%, 4%, 5%, 10%, 15%, 20%, 25%, 30%, 40%, 50%, 60%, 70%, 80%, 90%, or 95% of the genetic element sequence, e.g., from the 5′ end or the 3′ end of the genetic element sequence). In some embodiments, the genetic element sequence of the first copy and the genetic element sequence of the second copy are, or are derived from, the same genetic element sequence (e.g., the same CAV sequence). In some embodiments, the genetic element sequence of the first copy and the genetic element sequence of the second copy are, or are derived from, different genetic element sequences (e.g., sequences from different CAVs). In some embodiments, the first copy of the genetic element sequence and the second copy of the genetic element sequence are positioned adjacent to each other on the genetic element construct. In other embodiments, the first copy of the genetic element sequence and the second copy of the genetic element sequence may be separated, e.g., by a spacer region. In some embodiments, the second copy of the genetic element sequence or portion thereof (e.g., comprising an uRFS) is positioned 5′ relative to the first copy of the genetic element sequence. In some embodiments, the second copy of the genetic element sequence or portion thereof (e.g., comprising a dRFS) is positioned 3′ relative to the first copy of the genetic element sequence.

### Effectors

The compositions and methods described herein can be used to produce a genetic element of an CAVector comprising a sequence encoding an effector (e.g., an exogenous effector or an endogenous effector), e.g., as described herein. The effector may be, in some instances, an endogenous effector or an exogenous effector. In some embodiments, the effector is a therapeutic effector. In some embodiments, the effector comprises a polypeptide (e.g., a therapeutic polypeptide or peptide, e.g., as described herein). In some embodiments, the effector comprises a non-coding RNA (e.g., an miRNA, siRNA, shRNA, mRNA, lncRNA, RNA, DNA, antisense RNA, or gRNA). In some embodiments, the effector comprises a regulatory nucleic acid, e.g., as described herein.

In some embodiments, the effector-encoding sequence may be inserted into the genetic element e.g., at a non-coding region, e.g., a noncoding region disposed 3′ of the open reading frames and 5′ of the GC-rich region of the genetic element, in the 5′ noncoding region upstream of the TATA box, in the 5′ UTR, in the 3′ noncoding region downstream of the poly-A signal, or upstream of the GC-rich region. In some embodiments, the effector-encoding sequence may be inserted into the genetic element, e.g., in a coding sequence (e.g., in a sequence encoding a CAV VP1, VP2, and/or Apoptin, e.g., as described herein). In some embodiments, the effector-encoding sequence replaces all or a part of the open reading frame. In some embodiments, the genetic element comprises a regulatory sequence (e.g., a promoter or enhancer, e.g., as described herein) operably linked to the effector-encoding sequence.

In some embodiments, the genetic element comprising the effector is produced from a genetic element construct (e.g., a tandem construct) as described herein, e.g., by rolling circle replication of a genetic element sequence disposed thereon. In some embodiments, the genetic element construct comprises exactly one copy of the effector-encoding sequence. In some embodiments, the genetic element construct comprises two or more (e.g., 2, 3, 4, 5, 6, 7, 8, 9, 10, or more) copies of the effector-encoding sequence. In some embodiments, the genetic element construct comprises one full-length copy of the effector-encoding sequence and at least one (e.g., 1, 2, 3, 4, 5, 6, 7, 8, 9, or more) partial copies of the effector-encoding sequence (e.g., partial copies comprising a 5′ truncation or 3′ truncation of the effector-encoding sequence).

### Host Cells

The CAVectors described herein can be produced, for example, in a host cell. Generally, a host cell is provided that comprises a CAVector genetic element and the components of an CAVector proteinaceous exterior (e.g., a polypeptide encoded by an CAV VP1 nucleic acid or an CAV VP1 molecule). The host cell is then incubated under conditions suitable for enclosure of the genetic element within the proteinaceous exterior (e.g., culture conditions as described herein). In some embodiments, the host cell is further incubated under conditions suitable for release of the CAVector from the host cell, e.g., into the surrounding supernatant. In some embodiments, the host cell is lysed for harvest of CAVectors from the cell lysate. In some embodiments, an CAVector may be introduced to a host cell line grown to a high cell density.

Introduction of Genetic Elements into Host Cells

A genetic element or genetic element construct may be introduced into a host cell. In some embodiments, the genetic element itself is introduced into the host cell. In some embodiments, a genetic element construct comprising the sequence of the genetic element (e.g., as described herein) is introduced into the host cell. A genetic element or genetic element construct can be introduced into a host cell, for example, using methods known in the art. For example, a genetic element or genetic element construct can be introduced into a host cell by transfection (e.g., stable transfection or transient transfection). In embodiments, the genetic element or genetic element construct is introduced into the host cell by lipofectamine transfection. In embodiments, the genetic element or genetic element construct is introduced into the host cell by calcium phosphate transfection. In some embodiments, the genetic element or genetic element construct is introduced into the host cell by electroporation. In some embodiments, the genetic element or genetic element construct is introduced into the host cell using a gene gun. In some embodiments, the genetic element or genetic element construct is introduced into the host cell by nucleofection. In some embodiments, the genetic element or genetic element construct is introduced into the host cell by PEI transfection. In some embodiments, the genetic element or genetic element construct is introduced into the host cell by contacting the host cell with a viral vector comprising the genetic element or genetic element construct. In some embodiments, the genetic element is introduced into the host cell by contacting the host cell with a CAVector comprising the genetic element

In embodiments, the genetic element construct is capable of replication once introduced into the host cell. In embodiments, the genetic element can be produced from the genetic element construct once introduced into the host cell. In some embodiments, the genetic element is produced in the host cell by a polymerase, e.g., using the genetic element construct as a template.

In some embodiments, the genetic elements or vectors comprising the genetic elements are introduced (e.g., transfected) into cell lines that express a viral polymerase protein in order to achieve expression of the CAVector. To this end, cell lines that express a CAVector polymerase protein may be utilized as appropriate host cells. Host cells may be similarly engineered to provide other viral functions or additional functions.

To prepare the CAVector disclosed herein, a genetic element construct may be used to transfect cells that provide CAVector proteins and functions required for replication and production. Alternatively, cells may be transfected with a second construct (e.g., a virus) providing CAVector proteins and functions before, during, or after transfection by the genetic element or vector comprising the genetic element disclosed herein. In some embodiments, the second construct may be useful to complement production of an incomplete viral particle. The second construct (e.g., virus) may have a conditional growth defect, such as host range restriction or temperature sensitivity, e.g., which allows the subsequent selection of transfectant viruses. In some embodiments, the second construct may provide one or more replication proteins utilized by the host cells to achieve expression of the CAVector. In some embodiments, the host cells may be transfected with vectors encoding viral proteins such as the one or more replication proteins. In some embodiments, the second construct comprises an antiviral sensitivity.

The genetic element or vector comprising the genetic element disclosed herein can, in some instances, be replicated and produced into CAVectors using techniques known in the art. For example, various viral culture methods are described, e.g., in U.S. Pat. Nos. 4,650,764; 5,166,057; 5,854,037; European Patent Publication EP 0702085A1; U.S. patent application Ser. No. 09/152,845; International Patent Publications PCT WO97/12032; WO96/34625; European Patent Publication EP-A780475; WO 99/02657; WO 98/53078; WO 98/02530; WO 99/15672; WO 98/13501; WO 97/06270; and EPO 780 47SA1, each of which is incorporated by reference herein in its entirety.

Methods for Providing CAV Protein(s) in Cis or Trans

In some embodiments (e.g., cis embodiments described herein), the genetic element construct further comprises one or more expression cassettes comprising a coding sequence for an CAV ORF (e.g., a CAV VP1, VP2, and/or Apoptin, or a functional fragment thereof). In embodiments, the genetic element construct comprises an expression cassette comprising a coding sequence for an CAV VP1, or a splice variant or functional fragment thereof. Such genetic element constructs, which comprise expression cassettes for the effector as well as the one or more CAV ORFs, may be introduced into host cells. Host cells comprising such genetic element constructs may, in some instances, be capable of producing the genetic elements and components for proteinaceous exteriors, and for enclosure of the genetic elements within proteinaceous exteriors, without requiring additional genetic element constructs or integration of expression cassettes into the host cell genome. In other words, such genetic element constructs may be used for cis CAVector production methods in host cells, e.g., as described herein.

In some embodiments (e.g., trans embodiments described herein), the genetic element does not comprise an expression cassette comprising a coding sequence for one or more CAV ORFs (e.g., an CAV VP1, VP2, and/or Apoptin, or a functional fragment thereof). In embodiments, the genetic element construct does not comprise an expression cassette comprising a coding sequence for an CAV VP1, or a splice variant or functional fragment thereof. Such genetic element constructs, which comprise expression cassettes for the effector but lack expression cassettes for one or more CAV ORFs (e.g., CAV VP1 or a splice variant or functional fragment thereof), may be introduced into host cells. Host cells comprising such genetic element constructs may, in some instances, require additional genetic element constructs or integration of expression cassettes into the host cell genome for production of one or more components of the CAVector (e.g., the proteinaceous exterior proteins). In some embodiments, host cells comprising such genetic element constructs are incapable of enclosure of the genetic elements within proteinaceous exteriors in the absence of an additional nucleic construct encoding an CAV VP1 molecule. In other words, such genetic element constructs may be used for trans CAVector production methods in host cells, e.g., as described herein.

Helpers

In some embodiments, a helper construct is introduced into a host cell (e.g., a host cell comprising a genetic element construct or a genetic element as described herein). In some embodiments, the helper construct is introduced into the host cell prior to introduction of the genetic element construct. In some embodiments, the helper construct is introduced into the host cell concurrently with the introduction of the genetic element construct. In some embodiments, the helper construct is introduced into the host cell after introduction of the genetic element construct. In some embodiments, the helper construct is introduced into the host cell via a helper virus comprising the helper construct.

Exemplary Cell Types

Exemplary host cells suitable for production of CAVectors include, without limitation, eukaryotic cells (e.g., avian cells, mammalian cells, and insect cells). In some embodiments, the host cell is a human cell or cell line. In some embodiments, the host cell is an avian cell or cell line (e.g., a chicken cell or cell line, e.g., MDCC-MSB1 cells; or a duck cell or cell line, e.g., EB66 cells or AGE.CR cells). In some embodiments, the cell is an immune cell or cell line, e.g., a T cell or cell line, a cancer cell line, a hepatic cell or cell line, a neuron, a glial cell, a skin cell, an epithelial cell, a mesenchymal cell, a blood cell, an endothelial cell, an eye cell, a gastrointestinal cell, a progenitor cell, a precursor cell, a stem cell, a lung cell, a cardiac cell, or a muscle cell. In some embodiments, the host cell is an animal cell (e.g., a mouse cell, rat cell, rabbit cell, hamster cell, or insect cell).

In some embodiments, the host cell is a lymphoid cell. In some embodiments, the host cell is a T cell or an immortalized T cell. In embodiments, the host cell is a Jurkat cell. In embodiments, the host cell is a MOLT-4 cell. In some embodiments, the host cell is a B cell or an immortalized B cell. In embodiments, the host cell is a Raji cell.

In some embodiments, the host cell comprises a genetic element construct, e.g., a tandem construct (e.g., as described herein).

In embodiments, the host cell is a Raji cell, EKVX cell, MRC5 cell, or MCF7 cell. In embodiments, the host cell is a HEK293T cell, HEK293F cell, A549 cell, Jurkat cell, Chang cell, HeLa cell Phoenix cell, MRC-5 cell, NCI-H292 cell, or Wi38 cell. In some embodiments, the host cell is a non-human primate cell (e.g., a Vero cell, CV-1 cell, or LLCMK2 cell). In some embodiments, the host cell is a murine cell (e.g., a McCoy cell). In some embodiments, the host cell is a hamster cell (e.g., a CHO cell or BHK 21 cell). In some embodiments, the host cell is a MARC-145, MDBK, RK-13, or EEL cell. In some embodiments, the host cell is an epithelial cell (e.g., a cell line of epithelial lineage).

In some embodiments, the CAVector is cultivated in continuous animal cell line (e.g., immortalized cell lines that can be serially propagated). According to one embodiment of the invention, the cell lines may include porcine cell lines. The cell lines envisaged in the context of the present invention include immortalised porcine cell lines such as, but not limited to the porcine kidney epithelial cell lines PK-15 and SK, the monomyeloid cell line 3D4/31 and the testicular cell line ST.

### Culture Conditions

Host cells comprising a genetic element and components of a proteinaceous exterior can be incubated under conditions suitable for enclosure of the genetic element within the proteinaceous exterior, thereby producing an CAVector. Suitable culture conditions include those described. In some embodiments, the host cells are incubated in liquid media (e.g., RPMI (e.g., supplemented with FBS, e.g., 10% FBS), EX-CELL EBx GRO-I serume-free medium (e.g., supplemented with 1-glutamine), HyClone CDM4Avian medium (e.g., supplemented with 1-glutamine), Optipro SFM, Grace's Supplemented (TNM-FH), IPL-41, TC-100, Schneider's Drosophila, SF-900 II SFM, or EXPRESS-FIVE™ SFM). In some embodiments, the host cells are incubated in adherent culture. In some embodiments, the host cells are incubated in suspension culture. In some embodiments, the host cells are incubated in a tube, bottle, microcarrier, or flask. In some embodiments, the host cells are incubated in a dish or well (e.g., a well on a plate). In some embodiments, the host cells are incubated under conditions suitable for proliferation of the host cells. In some embodiments, the host cells are incubated under conditions suitable for the host cells to release CAVectors produced therein into the surrounding supernatant.

The production of CAVector-containing cell cultures according to the present invention can be carried out in different scales (e.g., in flasks, roller bottles or bioreactors). The media used for the cultivation of the cells to be infected generally comprise the standard nutrients required for cell viability, but may also comprise additional nutrients dependent on the cell type. Optionally, the medium can be protein-free and/or serum-free. Depending on the cell type the cells can be cultured in suspension or on a substrate. In some embodiments, different media is used for growth of the host cells and for production of CAVectors.

### Harvest and Purification

CAVectors produced by host cells can be harvested, e.g., according to methods known in the art. For example, CAVectors released into the surrounding supernatant by host cells in culture can be harvested from the supernatant. In some embodiments, the supernatant is separated from the host cells to obtain the CAVectors. In some embodiments, the host cells are lysed before or during harvest. In some embodiments, the CAVectors are harvested from the host cell lysates. In some embodiments, the CAVectors are harvested from both the host cell lysates and the supernatant. In some embodiments, the purification and isolation of CAVectors is performed according to known methods in virus production, for example, as described in Rinaldi, et al., DNA Vaccines: Methods and Protocols (Methods in Molecular Biology), 3rd ed. 2014, Humana Press (incorporated herein by reference in its entirety). In some embodiments, the CAVector may be harvested and/or purified by separation of solutes based on biophysical properties, e.g., size, density, charge. In one embodiment, CAVectors are purified from cells or cell culture by density gradient centrifugation and/or ultracentrifugation, e.g., sucrose density gradient centrifugation or cesium chloride density gradient centrifugation. For example, a CAVector preparation is enriched or purified by (a) optionally lysing host cells from the host cell culture, (b) optionally eliminating cellular debris from the host cell culture to generate a soluble fraction of the cell culture, (c) performing density gradient centrifugation and/or ultracentrifugation on the soluble fraction, and (d) collecting the enriched CAVector preparation from the density gradient. Other steps, such as sucrose cushion centrifugation/ultracentrifugation, ion exchange chromatography, and/or tangential flow filtration, may be performed prior to formulation with a pharmaceutical excipient.

Harvested CAVectors can be purified and/or enriched, e.g., to produce a CAVector preparation. In some embodiments, the harvested CAVectors are isolated from other constituents or contaminants present in the harvest solution, e.g., using methods known in the art for purifying viral particles (e.g., purification by sedimentation, chromatography, and/or ultrafiltration). In some embodiments, the purification steps comprise removing one or more of serum, host cell DNA, host cell proteins, particles lacking the genetic element, and/or phenol red from the preparation. In some embodiments, the harvested CAVectors are enriched relative to other constituents or contaminants present in the harvest solution, e.g., using methods known in the art for enriching viral particles.

In some embodiments, the resultant preparation or a pharmaceutical composition comprising the preparation will be stable over an acceptable period of time and temperature, and/or be compatible with the desired route of administration and/or any devices this route of administration will require, e.g., needles or syringes.

### II. CAVectors

In some aspects, the invention described herein comprises compositions and methods of using and making CAVectors, CAVector preparations, and therapeutic compositions. In some embodiments, the CAVectors are made using a genetic element construct as described herein. In some embodiments, the CAVector comprises one or more nucleic acids or polypeptides comprising a sequence, structure, and/or function that is based on a CAV (e.g., a CAV as described herein), or fragments or portions thereof, or other substantially non-pathogenic virus, e.g., a symbiotic virus, commensal virus, native virus. In some embodiments, a CAV-based CAVector comprises at least one element exogenous to that CAV, e.g., an exogenous effector or a nucleic acid sequence encoding an exogenous effector disposed within a genetic element of the CAVector. In some embodiments, a CAV-based CAVector comprises at least one element heterologous to another element from that CAV, e.g., an effector-encoding nucleic acid sequence that is heterologous to another linked nucleic acid sequence, such as a promoter element. In some embodiments, an CAVector comprises a genetic element (e.g., circular DNA, e.g., single stranded DNA), which comprise at least one element that is heterologous relative to the remainder of the genetic element and/or the proteinaceous exterior (e.g., an exogenous element encoding an effector, e.g., as described herein). A CAVector may be a delivery vehicle (e.g., a substantially non-pathogenic delivery vehicle) for a payload into a host, e.g., a human. In some embodiments, the CAVector is capable of replicating in a eukaryotic cell, e.g., a mammalian cell, e.g., a human cell. In some embodiments, the CAVector is substantially non-pathogenic and/or substantially non-integrating in the mammalian (e.g., human) cell. In some embodiments, the CAVector is replication-deficient. In some embodiments, the CAVector is replication-competent.

In an aspect, the invention includes a CAVector comprising (i) a genetic element comprising a promoter element, a sequence encoding an effector, (e.g., an endogenous effector or an exogenous effector, e.g., a payload), and a protein binding sequence (e.g., an exterior protein binding sequence, e.g., a packaging signal), wherein the genetic element is a single-stranded DNA, and has one or both of the following properties: is circular and/or integrates into the genome of a eukaryotic cell at a frequency of less than about 0.001%, 0.005%, 0.01%, 0.05%, 0.1%, 0.5%, 1%, 1.5%, or 2% of the genetic element that enters the cell; and (ii) a proteinaceous exterior; wherein the genetic element is enclosed within the proteinaceous exterior; and wherein the CAVector is capable of delivering the genetic element into a eukaryotic cell.

In some embodiments, the protein binding sequence comprises a packaging signal, e.g., suitable to permit packaging of the genetic element into a proteinaceous exterior comprising a CAV VP1 molecule. In embodiments, the protein binding sequence (e.g., the packaging signal) comprises the nucleic acid sequence AGCCCTGAAAAGGGGGGGGGGCTAAAGCCCCCCCCCCTTAAACCCCCCCCTGGGGGGGATT CCCCCCCAGACCCCCCCTTTATATAGCACTCAATAAACGCAGAAAATAGATTTATCGCACTA TC (SEQ ID NO: 17), or a nucleic acid sequence having at least 75%, 80%, 85%, 90%, 95%, 96%, 97%, 98%, or 99% sequence identity thereto, or a reverse complement thereof.

In some embodiments of the CAVector described herein, the genetic element integrates at a frequency of less than about 0.001%, 0.005%, 0.01%, 0.05%, 0.1%, 0.5%, 1%, 1.5%, or 2% of the genetic element that enters a cell. In some embodiments, less than about 0.01%, 0.05%, 0.1%, 0.5%, 1%, 2%, 3%, 4%, or 5% of the genetic elements from a plurality of the CAVectors administered to a subject will integrate into the genome of one or more host cells in the subject. In some embodiments, the genetic elements of a population of CAVectors, e.g., as described herein, integrate into the genome of a host cell at a frequency less than that of a comparable population of AAV viruses, e.g., at about a 50%, 60%, 70%, 75%, 80%, 85%, 90%, 95%, 100%, or more lower frequency than the comparable population of AAV viruses.

In an aspect, the invention includes a CAVector comprising: (i) a genetic element comprising a promoter element and a sequence encoding an effector (e.g., an endogenous effector or an exogenous effector, e.g., a payload), and a protein binding sequence (e.g., an exterior protein binding sequence), wherein the genetic element has at least 75% (e.g., at least 75, 76, 77, 78, 79, 80, 90, 91, 92, 93, 94, 95, 96, 97, 98, 99, or 100%) sequence identity to a wild-type CAV sequence, e.g., a wild-type CAV sequence as described herein; and (ii) a proteinaceous exterior; wherein the genetic element is enclosed within the proteinaceous exterior; and wherein the CAVector is capable of delivering the genetic element into a eukaryotic cell.

In one aspect, the invention includes an CAVector comprising:
a) a genetic element comprising (i) a sequence encoding an exterior protein (e.g., a non-pathogenic exterior protein), (ii) an exterior protein binding sequence that binds the genetic element to the non-pathogenic exterior protein, and (iii) a sequence encoding an effector (e.g., an endogenous or exogenous effector); and
b) a proteinaceous exterior that is associated with, e.g., envelops, encapsidates, or encloses, the genetic element.

In some embodiments, the CAVector includes sequences or expression products from (or having >70%, 75%, 80%, 85%, 90%, 95%, 97%, 98%, 99%, 100% homology to) a non-enveloped, circular, single-stranded DNA virus. Animal circular single-stranded DNA viruses generally refer to a subgroup of single strand DNA (ssDNA) viruses, which infect eukaryotic non-plant hosts, and have a circular genome. Thus, animal circular ssDNA viruses are distinguishable from ssDNA viruses that infect prokaryotes (i.e. Microviridae and Inoviridae) and from ssDNA viruses that infect plants (i.e. Geminiviridae and Nanoviridae). They are also distinguishable from linear ssDNA viruses that infect non-plant eukaryotes (i.e. Parvoviridiae).

In some embodiments, the CAVector modulates a host cellular function, e.g., transiently or long term. In certain embodiments, the cellular function is stably altered, such as a modulation that persists for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween. In certain embodiments, the cellular function is transiently altered, e.g., such as a modulation that persists for no more than about 30 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time therebetween.

In some embodiments, the genetic element comprises a promoter element. In embodiments, the promoter element is selected from an RNA polymerase II-dependent promoter, an RNA polymerase III-dependent promoter, a PGK promoter, a CMV promoter, an EF-1α promoter, an SV40 promoter, a CAGG promoter, or a UBC promoter, TTV viral promoters, Tissue specific, U6 (pollIII), minimal CMV promoter with upstream DNA binding sites for activator proteins (TetR-VP16, Gal4-VP16, dCas9-VP16, etc). In embodiments, the promoter element comprises a TATA box. In embodiments, the promoter element is endogenous to a wild-type CAV, e.g., as described herein.

In some embodiments, the genetic element comprises one or more of the following characteristics: single-stranded, circular, negative strand, and/or DNA. In embodiments, the genetic element comprises an episome. In some embodiments, the portions of the genetic element excluding the effector have a combined size of about 1-5 kb (e.g., about 2.8-4 kb, about 2.8-3.2 kb, about 3.6-3.9 kb, or about 2.8-2.9 kb), less than about 5 kb (e.g., less than about 2.9 kb, 3.2 kb, 3.6 kb, 3.9 kb, or 4 kb), or at least 100 nucleotides (e.g., at least 1 kb). In certain embodiments, the portions of the genetic element excluding the effector have a combined size of about 0.5-1 kb, 1-1.5 kb, 1.5-2 kb, 2-2.5 kb, 2.5-3 kb, or 3-3.5 kb.

The CAVectors, compositions comprising CAVectors, methods using such CAVectors, etc., as described herein are, in some instances, based in part on the examples which illustrate how different effectors, for example miRNAs (e.g. against IFN or miR-625), shRNA, etc and protein binding sequences, for example DNA sequences that bind to capsid protein such as Q99153, are combined with proteinaceious exteriors, for example a capsid disclosed in Arch Virol (2007) 152: 1961-1975, to produce CAVectors which can then be used to deliver an effector to cells (e.g., animal cells, e.g., human cells or non-human animal cells such as pig or mouse cells). In embodiments, the effector can silence expression of a factor such as an interferon. The examples further describe how CAVectors can be made by inserting effectors into sequences derived, e.g., from an CAV. It is on the basis of these examples that the description hereinafter contemplates various variations of the specific findings and combinations considered in the examples. For example, the skilled person will understand from the examples that the specific miRNAs are used just as an example of an effector and that other effectors may be, e.g., other regulatory nucleic acids or therapeutic peptides. Similarly, the specific capsids used in the examples may be replaced by substantially non-pathogenic proteins described hereinafter. The specific CAV sequences described in the examples may also be replaced by the CAV sequences described hereinafter. These considerations similarly apply to protein binding sequences, regulatory sequences such as promoters, and the like. Independent thereof, the person skilled in the art will in particular consider such embodiments which are closely related to the examples.

In some embodiments, a CAVector, or the genetic element comprised in the CAVector, is introduced into a cell (e.g., a human cell). In some embodiments, the effector (e.g., an RNA, e.g., an miRNA), e.g., encoded by the genetic element of an CAVector, is expressed in a cell (e.g., a human cell), e.g., once the CAVector or the genetic element has been introduced into the cell. In embodiments, introduction of the CAVector, or genetic element comprised therein, into a cell modulates (e.g., increases or decreases) the level of a target molecule (e.g., a target nucleic acid, e.g., RNA, or a target polypeptide) in the cell, e.g., by altering the expression level of the target molecule by the cell. In embodiments, introduction of the CAVector, or genetic element comprised therein, decreases level of interferon produced by the cell. In embodiments, introduction of the CAVector, or genetic element comprised therein, into a cell modulates (e.g., increases or decreases) a function of the cell. In embodiments, introduction of the CAVector, or genetic element comprised therein, into a cell modulates (e.g., increases or decreases) the viability of the cell. In embodiments, introduction of the CAVector, or genetic element comprised therein, into a cell decreases viability of a cell (e.g., a cancer cell).

In some embodiments, a CAVector (e.g., a synthetic CAVector) described herein induces an antibody prevalence of less than 70% (e.g., less than about 60%, 50%, 40%, 30%, 20%, or 10% antibody prevalence). In embodiments, antibody prevalence is determined according to methods known in the art. In embodiments, antibody prevalence is determined by detecting antibodies against an CAV (e.g., as described herein), or an CAVector based thereon, in a biological sample, e.g., according to the anti-TTV antibody detection method described in Tsuda et al. (1999; J. Virol. Methods 77: 199-206; incorporated herein by reference) and/or the method for determining anti-TTV IgG seroprevalence described in Kakkola et al. (2008; Virology 382: 182-189; incorporated herein by reference). Antibodies against an CAV or an CAVector based thereon can also be detected by methods in the art for detecting anti-viral antibodies, e.g., methods of detecting anti-AAV antibodies, e.g., as described in Calcedo et al. (2013; Front. Immunol. 4(341): 1-7; incorporated herein by reference).

In some embodiments, a replication deficient, replication defective, or replication incompetent genetic element does not encode all of the necessary machinery or components required for replication of the genetic element. In some embodiments, a replication defective genetic element does not encode a replication factor. In some embodiments, a replication defective genetic element does not encode one or more ORFs (e.g., CAV VP1, VP2, and/or Apoptin, e.g., as described herein). In some embodiments, the machinery or components not encoded by the genetic element may be provided in trans (e.g., using a virus or plasmid, or encoded in a nucleic acid also comprised by the host cell and/or surrounding medium, e.g., integrated into the genome of the host cell), e.g., such that the replication deficient, replication defective, or replication incompetent genetic element can undergo replication in the presence of the machinery or components provided in trans.

In some embodiments, a packaging deficient, packaging defective, or packaging incompetent genetic element cannot be packaged into a proteinaceous exterior (e.g., wherein the proteinaceous exterior comprises a capsid or a portion thereof, e.g., comprising a polypeptide encoded by a CAV VP1 nucleic acid, e.g., as described herein). In some embodiments, a packaging deficient genetic element is packaged into a proteinaceous exterior at an efficiency less than 10% (e.g., less than 10%, 9%, 8%, 7%, 6%, 5%, 4%, 3%, 2%, 1%, 0.5%, 0.1%, 0.01%, or 0.001%) compared to a wild-type CAV (e.g., as described herein). In some embodiments, the packaging defective genetic element cannot be packaged into a proteinaceous exterior even in the presence of factors (e.g., VP1, VP2, and/or Apoptin) that would permit packaging of the genetic element of a wild-type CAV (e.g., as described herein). In some embodiments, a packaging deficient genetic element is packaged into a proteinaceous exterior at an efficiency less than 10% (e.g., less than 10%, 9%, 8%, 7%, 6%, 5%, 4%, 3%, 2%, 1%, 0.5%, 0.1%, 0.01%, or 0.001%) compared to a wild-type CAV (e.g., as described herein), even in the presence of factors (e.g., CAV VP1, VP2, and/or Apoptin) that would permit packaging of the genetic element of a wild-type CAV (e.g., as described herein).

In some embodiments, a packaging competent genetic element can be packaged into a proteinaceous exterior (e.g., wherein the proteinaceous exterior comprises a capsid or a portion thereof, e.g., comprising a polypeptide encoded by an VP1 nucleic acid, e.g., as described herein). In some embodiments, a packaging competent genetic element is packaged into a proteinaceous exterior at an efficiency of at least 20% (e.g., at least 20%, 30%, 40%, 50%, 60%, 70%, 80%, 85%, 90%, 95%, 96%, 97%, 98%, 99%, 100%, or higher) compared to a wild-type CAV (e.g., as described herein). In some embodiments, the packaging competent genetic element can be packaged into a proteinaceous exterior in the presence of factors (e.g., VP1, VP2, and/or Apoptin) that would permit packaging of the genetic element of a wild-type CAV (e.g., as described herein). In some embodiments, a packaging competent genetic element is packaged into a proteinaceous exterior at an efficiency of at least 20% (e.g., at least 20%, 30%, 40%, 50%, 60%, 70%, 80%, 85%, 90%, 95%, 96%, 97%, 98%, 99%, 100%, or higher) compared to a wild-type CAV (e.g., as described herein) in the presence of factors (e.g., VP1, VP2, and/or Apoptin) that would permit packaging of the genetic element of a wild-type CAV (e.g., as described herein).

### Chicken Anemia Viruses (CAV)

In some embodiments, a CAVector, e.g., as described herein, comprises sequences or expression products derived from or similar to a wild-type chicken anemia virus (CAV). In some embodiments, a CAVector includes one or more sequences or expression products that are exogenous relative to a wild-type CAV. In some embodiments, a CAVector includes one or more sequences or expression products that are endogenous relative to the wild-type CAV. In some embodiments, a CAVector includes one or more sequences or expression products that are heterologous relative to one or more other sequences or expression products in the CAVector. CAVs generally have single-stranded circular DNA genomes with negative polarity. CAVs have not generally been linked to any human disease, nor are thought to infect human cells (see, e.g., Shulman and Davidson 2017, Ann. Rev. Virol. 4: 159-180; and Fatoba and Adeleke 2019, Acta Virologica 63: 19-25; each of which is incorporated by reference herein in their entirety).

In some embodiments, the genetic element comprises a nucleotide sequence encoding an amino acid sequence or a functional fragment thereof or a sequence having at least about 60%, 70% 80%, 85%, 90% 95%, 96%, 97%, 98%, 99%, or 100% sequence identity to any one of the amino acid sequences described herein, e.g., a CAV polypeptide sequence.

In some embodiments, an CAVector as described herein comprises one or more nucleic acid molecules (e.g., a genetic element as described herein) comprising a sequence having at least about 70%, 75%, 80%, 85%, 90%, 95%, 96%, 97%, 98%, 99%, or 100% sequence identity to a CAV nucleic acid sequence, e.g., as described herein, or a fragment thereof.

In some embodiments, a CAVector as described herein comprises one or more nucleic acid molecules (e.g., a genetic element as described herein) comprising a sequence having at least about 70%, 75%, 80%, 85%, 90%, 95%, 96%, 97%, 98%, 99%, or 100% sequence identity to one or more of a 5′ UTR, repeat region, CAAT signal, TATA box, VP2 gene, Apoptin gene, VP1, 3′ UTR, GC-rich region, polyA signal sequence, or any combination thereof, of a CAV, e.g., as described herein. In some embodiments, the nucleic acid molecule comprises a sequence encoding a capsid protein, e.g., a VP1, VP2, and/or Apoptin sequence of any of the CAVs described herein. In embodiments, the nucleic acid molecule comprises a sequence encoding a capsid protein comprising an amino acid sequence having at least about 70%, 75%, 80%, 85%, 90%, 95%, 96%, 97%, 98%, 99%, or 100% sequence identity to a CAV VP1 protein (or a splice variant or functional fragment thereof) or a polypeptide encoded by an CAV VP1 nucleic acid.

In embodiments, the nucleic acid molecule comprises a nucleic acid sequence having at least about 70%, 75%, 80%, 85%, 90%, 95%, 96%, 97%, 98%, 99%, or 100% sequence identity to the full nucleic acid sequence of Table 1A, or a contiguous sequence of 100-200, 200-300, 300-400, 400-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2000-2100, 2100-2200, 2200-2300, or 2300-2319 nucleotides therein. In embodiments, the nucleic acid molecule comprises a nucleic acid sequence having at least about 70%, 75%, 80%, 85%, 90%, 95%, 96%, 97%, 98%, 99%, or 100% sequence identity to the 5′ UTR nucleotide sequence of Table 1A. In embodiments, the nucleic acid molecule comprises a nucleic acid sequence having at least about 70%, 75%, 80%, 85%, 90%, 95%, 96%, 97%, 98%, 99%, or 100% sequence identity to the repeat region nucleotide sequence of Table 1A. In embodiments, the nucleic acid molecule comprises a nucleic acid sequence having at least about 70%, 75%, 80%, 85%, 90%, 95%, 96%, 97%, 98%, 99%, or 100% sequence identity to the CAAT signal nucleotide sequence of Table 1A. In embodiments, the nucleic acid molecule comprises a nucleic acid sequence having at least about 70%, 75%, 80%, 85%, 90%, 95%, 96%, 97%, 98%, 99%, or 100% sequence identity to the TATA box nucleotide sequence of Table 1A. In embodiments, the nucleic acid molecule comprises a nucleic acid sequence having at least about 70%, 75%, 80%, 85%, 90%, 95%, 96%, 97%, 98%, 99%, or 100% sequence identity to the VP2 nucleotide sequence of Table 1A. In embodiments, the nucleic acid molecule comprises a nucleic acid sequence having at least about 70%, 75%, 80%, 85%, 90%, 95%, 96%, 97%, 98%, 99%, or 100% sequence identity to the Apoptin nucleotide sequence of Table 1A. In embodiments, the nucleic acid molecule comprises a nucleic acid sequence having at least about 70%, 75%, 80%, 85%, 90%, 95%, 96%, 97%, 98%, 99%, or 100% sequence identity to the VP1 nucleotide sequence of Table 1A. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the VP2 nucleotide sequence of Table 1A. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the Apoptin nucleotide sequence of Table 1A. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the VP1 nucleotide sequence of Table 1A. In embodiments, the nucleic acid molecule comprises a nucleic acid sequence having at least about 70%, 75%, 80%, 85%, 90%, 95%, 96%, 97%, 98%, 99%, or 100% sequence identity to the 3′ UTR nucleotide sequence of Table 1A. In embodiments, the nucleic acid molecule comprises a nucleic acid sequence having at least about 70%, 75%, 80%, 85%, 90%, 95%, 96%, 97%, 98%, 99%, or 100% sequence identity to the GC-rich nucleotide sequence of Table 1A. In embodiments, the nucleic acid molecule comprises a nucleic acid sequence having at least about 70%, 75%, 80%, 85%, 90%, 95%, 96%, 97%, 98%, 99%, or 100% sequence identity to the polyA signal nucleotide sequence of Table 1A.

In embodiments, the nucleic acid molecule comprises a nucleic acid sequence having at least about 70%, 75%, 80%, 85%, 90%, 95%, 96%, 97%, 98%, 99%, or 100% sequence identity to the full nucleic acid sequence of Table 1B, or a contiguous sequence of 100-200, 200-300, 300-400, 400-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2000-2100, 2100-2200, 2200-2300, or 2300-2319 nucleotides therein. In embodiments, the nucleic acid molecule comprises a nucleic acid sequence having at least about 70%, 75%, 80%, 85%, 90%, 95%, 96%, 97%, 98%, 99%, or 100% sequence identity to the 5′ UTR nucleotide sequence of Table 1B. In embodiments, the nucleic acid molecule comprises a nucleic acid sequence having at least about 70%, 75%, 80%, 85%, 90%, 95%, 96%, 97%, 98%, 99%, or 100% sequence identity to the VP2 nucleotide sequence of Table 1B. In embodiments, the nucleic acid molecule comprises a nucleic acid sequence having at least about 70%, 75%, 80%, 85%, 90%, 95%, 96%, 97%, 98%, 99%, or 100% sequence identity to the Apoptin nucleotide sequence of Table 1B. In embodiments, the nucleic acid molecule comprises a nucleic acid sequence having at least about 70%, 75%, 80%, 85%, 90%, 95%, 96%, 97%, 98%, 99%, or 100% sequence identity to the VP1 nucleotide sequence of Table 1B. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the VP2 nucleotide sequence of Table 1B. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the Apoptin nucleotide sequence of Table 1B. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the VP1 nucleotide sequence of Table 1B. In embodiments, the nucleic acid molecule comprises a nucleic acid sequence having at least about 70%, 75%, 80%, 85%, 90%, 95%, 96%, 97%, 98%, 99%, or 100% sequence identity to the 3′ UTR nucleotide sequence of Table 1B.

In embodiments, the nucleic acid molecule comprises a nucleic acid sequence having at least about 70%, 75%, 80%, 85%, 90%, 95%, 96%, 97%, 98%, 99%, or 100% sequence identity to the full nucleic acid sequence of Table 2, or a contiguous sequence of 100-200, 200-300, 300-400, 400-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2000-2100, 2100-2200, 2200-2300, or 2300-2319 nucleotides therein. In embodiments, the nucleic acid molecule comprises a nucleic acid sequence having at least about 70%, 75%, 80%, 85%, 90%, 95%, 96%, 97%, 98%, 99%, or 100% sequence identity to nucleotides 1-6 of the full nucleic acid sequence of Table 2. In embodiments, the nucleic acid molecule comprises a nucleic acid sequence having at least about 70%, 75%, 80%, 85%, 90%, 95%, 96%, 97%, 98%, 99%, or 100% sequence identity to nucleotides 995-2319 of the full nucleic acid sequence of Table 2. In embodiments, the nucleic acid molecule does not comprise a nucleic acid sequence having at least about 70%, 75%, 80%, 85%, 90%, 95%, 96%, 97%, 98%, 99%, or 100% sequence identity to nucleotides 7-994 of the full nucleic acid sequence of Table 1B, or a contiguous sequence of between 10-20, 20-30, 30-40, 40-50, 50-60, 60-70, 70-80, 80-90, 100-150, 150-200, 200-300, 300-400, 400-500, 500-600, 600-700, 700-800, 800-900, 900-950, or 950-987 nucleotides therein. In embodiments, the nucleic acid molecule comprises a nucleic acid sequence having at least about 70%, 75%, 80%, 85%, 90%, 95%, 96%, 97%, 98%, 99%, or 100% sequence identity to the nLuc insert nucleotide sequence of Table 2. In embodiments, the nucleic acid molecule comprises a nucleic acid sequence having at least about 70%, 75%, 80%, 85%, 90%, 95%, 96%, 97%, 98%, 99%, or 100% sequence identity to the 5′ UTR nucleotide sequence of Table 2. In embodiments, the nucleic acid molecule comprises a nucleic acid sequence having at least about 70%, 75%, 80%, 85%, 90%, 95%, 96%, 97%, 98%, 99%, or 100% sequence identity to the VP2 nucleotide sequence of Table 2. In embodiments, the nucleic acid molecule comprises a nucleic acid sequence having at least about 70%, 75%, 80%, 85%, 90%, 95%, 96%, 97%, 98%, 99%, or 100% sequence identity to the Apoptin nucleotide sequence of Table 2. In embodiments, the nucleic acid molecule comprises a nucleic acid sequence having at least about 70%, 75%, 80%, 85%, 90%, 95%, 96%, 97%, 98%, 99%, or 100% sequence identity to the VP1 nucleotide sequence of Table 2. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the VP2 nucleotide sequence of Table 2. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the Apoptin nucleotide sequence of Table 2. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the VP1 nucleotide sequence of Table 2. In embodiments, the nucleic acid molecule comprises a nucleic acid sequence having at least about 70%, 75%, 80%, 85%, 90%, 95%, 96%, 97%, 98%, 99%, or 100% sequence identity to the 3′ UTR nucleotide sequence of Table 2.

In embodiments, the nucleic acid molecule comprises a nucleic acid sequence having at least about 70%, 75%, 80%, 85%, 90%, 95%, 96%, 97%, 98%, 99%, or 100% sequence identity to the full nucleic acid sequence of Table 3, or a contiguous sequence of 100-200, 200-300, 300-400, 400-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2000-2100, 2100-2200, 2200-2300, or 2300-2319 nucleotides therein. In embodiments, the nucleic acid molecule comprises a nucleic acid sequence having at least about 70%, 75%, 80%, 85%, 90%, 95%, 96%, 97%, 98%, 99%, or 100% sequence identity to nucleotides 1-206 of the full nucleic acid sequence of Table 3. In embodiments, the nucleic acid molecule comprises a nucleic acid sequence having at least about 70%, 75%, 80%, 85%, 90%, 95%, 96%, 97%, 98%, 99%, or 100% sequence identity to nucleotides 1195-2319 of the full nucleic acid sequence of Table 3. In embodiments, the nucleic acid molecule does not comprise a nucleic acid sequence having at least about 70%, 75%, 80%, 85%, 90%, 95%, 96%, 97%, 98%, 99%, or 100% sequence identity to nucleotides 207-1194 of the full nucleic acid sequence of Table 1B, or a contiguous sequence of between 10-20, 20-30, 30-40, 40-50, 50-60, 60-70, 70-80, 80-90, 100-150, 150-200, 200-300, 300-400, 400-500, 500-600, 600-700, 700-800, 800-900, 900-950, or 950-987 nucleotides therein. In embodiments, the nucleic acid molecule comprises a nucleic acid sequence having at least about 70%, 75%, 80%, 85%, 90%, 95%, 96%, 97%, 98%, 99%, or 100% sequence identity to the nLuc insert nucleotide sequence of Table 3. In embodiments, the nucleic acid molecule comprises a nucleic acid sequence having at least about 70%, 75%, 80%, 85%, 90%, 95%, 96%, 97%, 98%, 99%, or 100% sequence identity to the 5′ UTR nucleotide sequence of Table 3. In embodiments, the nucleic acid molecule comprises a nucleic acid sequence having at least about 70%, 75%, 80%, 85%, 90%, 95%, 96%, 97%, 98%, 99%, or 100% sequence identity to the VP2 nucleotide sequence of Table 3. In embodiments, the nucleic acid molecule comprises a nucleic acid sequence having at least about 70%, 75%, 80%, 85%, 90%, 95%, 96%, 97%, 98%, 99%, or 100% sequence identity to the Apoptin nucleotide sequence of Table 3. In embodiments, the nucleic acid molecule comprises a nucleic acid sequence having at least about 70%, 75%, 80%, 85%, 90%, 95%, 96%, 97%, 98%, 99%, or 100% sequence identity to the VP1 nucleotide sequence of Table 3. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the VP2 nucleotide sequence of Table 3. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the Apoptin nucleotide sequence of Table 3. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the VP1 nucleotide sequence of Table 3. In embodiments, the nucleic acid molecule comprises a nucleic acid sequence having at least about 70%, 75%, 80%, 85%, 90%, 95%, 96%, 97%, 98%, 99%, or 100% sequence identity to the 3′ UTR nucleotide sequence of Table 3.

In embodiments, the nucleic acid molecule comprises a nucleic acid sequence having at least about 70%, 75%, 80%, 85%, 90%, 95%, 96%, 97%, 98%, 99%, or 100% sequence identity to the full nucleic acid sequence of Table 4, or a contiguous sequence of 100-200, 200-300, 300-400, 400-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2000-2100, 2100-2200, 2200-2300, or 2300-2319 nucleotides therein. In embodiments, the nucleic acid molecule comprises a nucleic acid sequence having at least about 70%, 75%, 80%, 85%, 90%, 95%, 96%, 97%, 98%, 99%, or 100% sequence identity to nucleotides 1-406 of the full nucleic acid sequence of Table 4. In embodiments, the nucleic acid molecule comprises a nucleic acid sequence having at least about 70%, 75%, 80%, 85%, 90%, 95%, 96%, 97%, 98%, 99%, or 100% sequence identity to nucleotides 1395-2319 of the full nucleic acid sequence of Table 4. In embodiments, the nucleic acid molecule does not comprise a nucleic acid sequence having at least about 70%, 75%, 80%, 85%, 90%, 95%, 96%, 97%, 98%, 99%, or 100% sequence identity to nucleotides 407-1394 of the full nucleic acid sequence of Table 1B, or a contiguous sequence of between 10-20, 20-30, 30-40, 40-50, 50-60, 60-70, 70-80, 80-90, 100-150, 150-200, 200-300, 300-400, 400-500, 500-600, 600-700, 700-800, 800-900, 900-950, or 950-987 nucleotides therein. In embodiments, the nucleic acid molecule comprises a nucleic acid sequence having at least about 70%, 75%, 80%, 85%, 90%, 95%, 96%, 97%, 98%, 99%, or 100% sequence identity to the nLuc insert nucleotide sequence of Table 4. In embodiments, the nucleic acid molecule comprises a nucleic acid sequence having at least about 70%, 75%, 80%, 85%, 90%, 95%, 96%, 97%, 98%, 99%, or 100% sequence identity to the 5′ UTR nucleotide sequence of Table 4. In embodiments, the nucleic acid molecule comprises a nucleic acid sequence having at least about 70%, 75%, 80%, 85%, 90%, 95%, 96%, 97%, 98%, 99%, or 100% sequence identity to the VP2 nucleotide sequence of Table 4. In embodiments, the nucleic acid molecule comprises a nucleic acid sequence having at least about 70%, 75%, 80%, 85%, 90%, 95%, 96%, 97%, 98%, 99%, or 100% sequence identity to the Apoptin nucleotide sequence of Table 4. In embodiments, the nucleic acid molecule comprises a nucleic acid sequence having at least about 70%, 75%, 80%, 85%, 90%, 95%, 96%, 97%, 98%, 99%, or 100% sequence identity to the VP1 nucleotide sequence of Table 4. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the VP2 nucleotide sequence of Table 4. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the Apoptin nucleotide sequence of Table 4. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the VP1 nucleotide sequence of Table 4. In embodiments, the nucleic acid molecule comprises a nucleic acid sequence having at least about 70%, 75%, 80%, 85%, 90%, 95%, 96%, 97%, 98%, 99%, or 100% sequence identity to the 3′ UTR nucleotide sequence of Table 4.

In embodiments, the nucleic acid molecule comprises a nucleic acid sequence having at least about 70%, 75%, 80%, 85%, 90%, 95%, 96%, 97%, 98%, 99%, or 100% sequence identity to the full nucleic acid sequence of Table 5, or a contiguous sequence of 100-200, 200-300, 300-400, 400-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2000-2100, 2100-2200, 2200-2300, or 2300-2319 nucleotides therein. In embodiments, the nucleic acid molecule comprises a nucleic acid sequence having at least about 70%, 75%, 80%, 85%, 90%, 95%, 96%, 97%, 98%, 99%, or 100% sequence identity to nucleotides 1-606 of the full nucleic acid sequence of Table 5. In embodiments, the nucleic acid molecule comprises a nucleic acid sequence having at least about 70%, 75%, 80%, 85%, 90%, 95%, 96%, 97%, 98%, 99%, or 100% sequence identity to nucleotides 1595-2319 of the full nucleic acid sequence of Table 5. In embodiments, the nucleic acid molecule does not comprise a nucleic acid sequence having at least about 70%, 75%, 80%, 85%, 90%, 95%, 96%, 97%, 98%, 99%, or 100% sequence identity to nucleotides 607-1594 of the full nucleic acid sequence of Table 1B, or a contiguous sequence of between 10-20, 20-30, 30-40, 40-50, 50-60, 60-70, 70-80, 80-90, 100-150, 150-200, 200-300, 300-400, 400-500, 500-600, 600-700, 700-800, 800-900, 900-950, or 950-987 nucleotides therein. In embodiments, the nucleic acid molecule comprises a nucleic acid sequence having at least about 70%, 75%, 80%, 85%, 90%, 95%, 96%, 97%, 98%, 99%, or 100% sequence identity to the nLuc insert nucleotide sequence of Table 5. In embodiments, the nucleic acid molecule comprises a nucleic acid sequence having at least about 70%, 75%, 80%, 85%, 90%, 95%, 96%, 97%, 98%, 99%, or 100% sequence identity to the 5′ UTR nucleotide sequence of Table 5. In embodiments, the nucleic acid molecule comprises a nucleic acid sequence having at least about 70%, 75%, 80%, 85%, 90%, 95%, 96%, 97%, 98%, 99%, or 100% sequence identity to the VP2 nucleotide sequence of Table 5. In embodiments, the nucleic acid molecule comprises a nucleic acid sequence having at least about 70%, 75%, 80%, 85%, 90%, 95%, 96%, 97%, 98%, 99%, or 100% sequence identity to the Apoptin nucleotide sequence of Table 5. In embodiments, the nucleic acid molecule comprises a nucleic acid sequence having at least about 70%, 75%, 80%, 85%, 90%, 95%, 96%, 97%, 98%, 99%, or 100% sequence identity to the VP1 nucleotide sequence of Table 5. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the VP2 nucleotide sequence of Table 5. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the Apoptin nucleotide sequence of Table 5. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the VP1 nucleotide sequence of Table 5. In embodiments, the nucleic acid molecule comprises a nucleic acid sequence having at least about 70%, 75%, 80%, 85%, 90%, 95%, 96%, 97%, 98%, 99%, or 100% sequence identity to the 3′ UTR nucleotide sequence of Table 5.

In embodiments, the nucleic acid molecule comprises a nucleic acid sequence having at least about 70%, 75%, 80%, 85%, 90%, 95%, 96%, 97%, 98%, 99%, or 100% sequence identity to the full nucleic acid sequence of Table 6, or a contiguous sequence of 100-200, 200-300, 300-400, 400-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2000-2100, 2100-2200, 2200-2300, or 2300-2319 nucleotides therein. In embodiments, the nucleic acid molecule comprises a nucleic acid sequence having at least about 70%, 75%, 80%, 85%, 90%, 95%, 96%, 97%, 98%, 99%, or 100% sequence identity to nucleotides 1-806 of the full nucleic acid sequence of Table 6. In embodiments, the nucleic acid molecule comprises a nucleic acid sequence having at least about 70%, 75%, 80%, 85%, 90%, 95%, 96%, 97%, 98%, 99%, or 100% sequence identity to nucleotides 1795-2319 of the full nucleic acid sequence of Table 6. In embodiments, the nucleic acid molecule does not comprise a nucleic acid sequence having at least about 70%, 75%, 80%, 85%, 90%, 95%, 96%, 97%, 98%, 99%, or 100% sequence identity to nucleotides 807-1794 of the full nucleic acid sequence of Table 1B, or a contiguous sequence of between 10-20, 20-30, 30-40, 40-50, 50-60, 60-70, 70-80, 80-90, 100-150, 150-200, 200-300, 300-400, 400-500, 500-600, 600-700, 700-800, 800-900, 900-950, or 950-987 nucleotides therein. In embodiments, the nucleic acid molecule comprises a nucleic acid sequence having at least about 70%, 75%, 80%, 85%, 90%, 95%, 96%, 97%, 98%, 99%, or 100% sequence identity to the nLuc insert nucleotide sequence of Table 6. In embodiments, the nucleic acid molecule comprises a nucleic acid sequence having at least about 70%, 75%, 80%, 85%, 90%, 95%, 96%, 97%, 98%, 99%, or 100% sequence identity to the 5′ UTR nucleotide sequence of Table 6. In embodiments, the nucleic acid molecule comprises a nucleic acid sequence having at least about 70%, 75%, 80%, 85%, 90%, 95%, 96%, 97%, 98%, 99%, or 100% sequence identity to the VP2 nucleotide sequence of Table 6. In embodiments, the nucleic acid molecule comprises a nucleic acid sequence having at least about 70%, 75%, 80%, 85%, 90%, 95%, 96%, 97%, 98%, 99%, or 100% sequence identity to the Apoptin nucleotide sequence of Table 6. In embodiments, the nucleic acid molecule comprises a nucleic acid sequence having at least about 70%, 75%, 80%, 85%, 90%, 95%, 96%, 97%, 98%, 99%, or 100% sequence identity to the VP1 nucleotide sequence of Table 6. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the VP2 nucleotide sequence of Table 6. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the Apoptin nucleotide sequence of Table 6. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the VP1 nucleotide sequence of Table 6. In embodiments, the nucleic acid molecule comprises a nucleic acid sequence having at least about 70%, 75%, 80%, 85%, 90%, 95%, 96%, 97%, 98%, 99%, or 100% sequence identity to the 3′ UTR nucleotide sequence of Table 6.

In embodiments, the nucleic acid molecule comprises a nucleic acid sequence having at least about 70%, 75%, 80%, 85%, 90%, 95%, 96%, 97%, 98%, 99%, or 100% sequence identity to the full nucleic acid sequence of Table 7, or a contiguous sequence of 100-200, 200-300, 300-400, 400-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2000-2100, 2100-2200, 2200-2300, or 2300-2319 nucleotides therein. In embodiments, the nucleic acid molecule comprises a nucleic acid sequence having at least about 70%, 75%, 80%, 85%, 90%, 95%, 96%, 97%, 98%, 99%, or 100% sequence identity to nucleotides 1-1006 of the full nucleic acid sequence of Table 7. In embodiments, the nucleic acid molecule comprises a nucleic acid sequence having at least about 70%, 75%, 80%, 85%, 90%, 95%, 96%, 97%, 98%, 99%, or 100% sequence identity to nucleotides 1995-2319 of the full nucleic acid sequence of Table 7. In embodiments, the nucleic acid molecule does not comprise a nucleic acid sequence having at least about 70%, 75%, 80%, 85%, 90%, 95%, 96%, 97%, 98%, 99%, or 100% sequence identity to nucleotides 1007-1994 of the full nucleic acid sequence of Table 1B, or a contiguous sequence of between 10-20, 20-30, 30-40, 40-50, 50-60, 60-70, 70-80, 80-90, 100-150, 150-200, 200-300, 300-400, 400-500, 500-600, 600-700, 700-800, 800-900, 900-950, or 950-987 nucleotides therein. In embodiments, the nucleic acid molecule comprises a nucleic acid sequence having at least about 70%, 75%, 80%, 85%, 90%, 95%, 96%, 97%, 98%, 99%, or 100% sequence identity to the nLuc insert nucleotide sequence of Table 7. In embodiments, the nucleic acid molecule comprises a nucleic acid sequence having at least about 70%, 75%, 80%, 85%, 90%, 95%, 96%, 97%, 98%, 99%, or 100% sequence identity to the 5′ UTR nucleotide sequence of Table 7. In embodiments, the nucleic acid molecule comprises a nucleic acid sequence having at least about 70%, 75%, 80%, 85%, 90%, 95%, 96%, 97%, 98%, 99%, or 100% sequence identity to the VP2 nucleotide sequence of Table 7. In embodiments, the nucleic acid molecule comprises a nucleic acid sequence having at least about 70%, 75%, 80%, 85%, 90%, 95%, 96%, 97%, 98%, 99%, or 100% sequence identity to the Apoptin nucleotide sequence of Table 7. In embodiments, the nucleic acid molecule comprises a nucleic acid sequence having at least about 70%, 75%, 80%, 85%, 90%, 95%, 96%, 97%, 98%, 99%, or 100% sequence identity to the VP1 nucleotide sequence of Table 7. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the VP2 nucleotide sequence of Table 7. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the Apoptin nucleotide sequence of Table 7. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the VP1 nucleotide sequence of Table 7. In embodiments, the nucleic acid molecule comprises a nucleic acid sequence having at least about 70%, 75%, 80%, 85%, 90%, 95%, 96%, 97%, 98%, 99%, or 100% sequence identity to the 3′ UTR nucleotide sequence of Table 7.

In embodiments, the nucleic acid molecule comprises a nucleic acid sequence having at least about 70%, 75%, 80%, 85%, 90%, 95%, 96%, 97%, 98%, 99%, or 100% sequence identity to the full nucleic acid sequence of Table 8, or a contiguous sequence of 100-200, 200-300, 300-400, 400-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2000-2100, 2100-2200, 2200-2300, or 2300-2319 nucleotides therein. In embodiments, the nucleic acid molecule comprises a nucleic acid sequence having at least about 70%, 75%, 80%, 85%, 90%, 95%, 96%, 97%, 98%, 99%, or 100% sequence identity to nucleotides 1-1206 of the full nucleic acid sequence of Table 8. In embodiments, the nucleic acid molecule comprises a nucleic acid sequence having at least about 70%, 75%, 80%, 85%, 90%, 95%, 96%, 97%, 98%, 99%, or 100% sequence identity to nucleotides 2195-2319 of the full nucleic acid sequence of Table 8. In embodiments, the nucleic acid molecule does not comprise a nucleic acid sequence having at least about 70%, 75%, 80%, 85%, 90%, 95%, 96%, 97%, 98%, 99%, or 100% sequence identity to nucleotides 1207-2194 of the full nucleic acid sequence of Table 1B, or a contiguous sequence of between 10-20, 20-30, 30-40, 40-50, 50-60, 60-70, 70-80, 80-90, 100-150, 150-200, 200-300, 300-400, 400-500, 500-600, 600-700, 700-800, 800-900, 900-950, or 950-987 nucleotides therein. In embodiments, the nucleic acid molecule comprises a nucleic acid sequence having at least about 70%, 75%, 80%, 85%, 90%, 95%, 96%, 97%, 98%, 99%, or 100% sequence identity to the nLuc insert nucleotide sequence of Table 8. In embodiments, the nucleic acid molecule comprises a nucleic acid sequence having at least about 70%, 75%, 80%, 85%, 90%, 95%, 96%, 97%, 98%, 99%, or 100% sequence identity to the 5′ UTR nucleotide sequence of Table 8. In embodiments, the nucleic acid molecule comprises a nucleic acid sequence having at least about 70%, 75%, 80%, 85%, 90%, 95%, 96%, 97%, 98%, 99%, or 100% sequence identity to the VP2 nucleotide sequence of Table 8. In embodiments, the nucleic acid molecule comprises a nucleic acid sequence having at least about 70%, 75%, 80%, 85%, 90%, 95%, 96%, 97%, 98%, 99%, or 100% sequence identity to the Apoptin nucleotide sequence of Table 8. In embodiments, the nucleic acid molecule comprises a nucleic acid sequence having at least about 70%, 75%, 80%, 85%, 90%, 95%, 96%, 97%, 98%, 99%, or 100% sequence identity to the VP1 nucleotide sequence of Table 8. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the VP2 nucleotide sequence of Table 8. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the Apoptin nucleotide sequence of Table 8. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the VP1 nucleotide sequence of Table 8. In embodiments, the nucleic acid molecule comprises a nucleic acid sequence having at least about 70%, 75%, 80%, 85%, 90%, 95%, 96%, 97%, 98%, 99%, or 100% sequence identity to the 3′ UTR nucleotide sequence of Table 8.

In embodiments, the nucleic acid molecule comprises a nucleic acid sequence having at least about 70%, 75%, 80%, 85%, 90%, 95%, 96%, 97%, 98%, 99%, or 100% sequence identity to the full nucleic acid sequence of Table 9, or a contiguous sequence of 100-200, 200-300, 300-400, 400-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2000-2100, 2100-2200, 2200-2300, or 2300-2319 nucleotides therein. In embodiments, the nucleic acid molecule comprises a nucleic acid sequence having at least about 70%, 75%, 80%, 85%, 90%, 95%, 96%, 97%, 98%, 99%, or 100% sequence identity to nucleotides 1-1271 of the full nucleic acid sequence of Table 9. In embodiments, the nucleic acid molecule does not comprise a nucleic acid sequence having at least about 70%, 75%, 80%, 85%, 90%, 95%, 96%, 97%, 98%, 99%, or 100% sequence identity to nucleotides 1272-2319 of the full nucleic acid sequence of Table 1B, or a contiguous sequence of between 10-20, 20-30, 30-40, 40-50, 50-60, 60-70, 70-80, 80-90, 100-150, 150-200, 200-300, 300-400, 400-500, 500-600, 600-700, 700-800, 800-900, 900-950, or 950-987 nucleotides therein. In embodiments, the nucleic acid molecule comprises a nucleic acid sequence having at least about 70%, 75%, 80%, 85%, 90%, 95%, 96%, 97%, 98%, 99%, or 100% sequence identity to the nLuc insert nucleotide sequence of Table 9. In embodiments, the nucleic acid molecule comprises a nucleic acid sequence having at least about 70%, 75%, 80%, 85%, 90%, 95%, 96%, 97%, 98%, 99%, or 100% sequence identity to the 5′ UTR nucleotide sequence of Table 9. In embodiments, the nucleic acid molecule comprises a nucleic acid sequence having at least about 70%, 75%, 80%, 85%, 90%, 95%, 96%, 97%, 98%, 99%, or 100% sequence identity to the VP2 nucleotide sequence of Table 9. In embodiments, the nucleic acid molecule comprises a nucleic acid sequence having at least about 70%, 75%, 80%, 85%, 90%, 95%, 96%, 97%, 98%, 99%, or 100% sequence identity to the Apoptin nucleotide sequence of Table 9. In embodiments, the nucleic acid molecule comprises a nucleic acid sequence having at least about 70%, 75%, 80%, 85%, 90%, 95%, 96%, 97%, 98%, 99%, or 100% sequence identity to the VP1 nucleotide sequence of Table 9. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the VP2 nucleotide sequence of Table 9. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the Apoptin nucleotide sequence of Table 9. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the VP1 nucleotide sequence of Table 9. In embodiments, the nucleic acid molecule comprises a nucleic acid sequence having at least about 70%, 75%, 80%, 85%, 90%, 95%, 96%, 97%, 98%, 99%, or 100% sequence identity to the 3′ UTR nucleotide sequence of Table 9.

In embodiments, the nucleic acid molecule comprises a nucleic acid sequence having at least about 70%, 75%, 80%, 85%, 90%, 95%, 96%, 97%, 98%, 99%, or 100% sequence identity to the full nucleic acid sequence of Table 10, or a contiguous sequence of 100-200, 200-300, 300-400, 400-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2000-2100, 2100-2200, 2200-2300, or 2300-2319 nucleotides therein. In embodiments, the nucleic acid molecule comprises a nucleic acid sequence having at least about 70%, 75%, 80%, 85%, 90%, 95%, 96%, 97%, 98%, 99%, or 100% sequence identity to nucleotides 1-546 of the full nucleic acid sequence of Table 10. In embodiments, the nucleic acid molecule comprises a nucleic acid sequence having at least about 70%, 75%, 80%, 85%, 90%, 95%, 96%, 97%, 98%, 99%, or 100% sequence identity to nucleotides 2133-2319 of the full nucleic acid sequence of Table 10. In embodiments, the nucleic acid molecule does not comprise a nucleic acid sequence having at least about 70%, 75%, 80%, 85%, 90%, 95%, 96%, 97%, 98%, 99%, or 100% sequence identity to nucleotides 547-2134 of the full nucleic acid sequence of Table 1B, or a contiguous sequence of between 10-20, 20-30, 30-40, 40-50, 50-60, 60-70, 70-80, 80-90, 100-150, 150-200, 200-300, 300-400, 400-500, 500-600, 600-700, 700-800, 800-900, 900-950, 950-1000, 1000-1100, 1100-1200, 1200-1300, 1300-1400, 1400-1500, or 1500-1587 nucleotides therein. In embodiments, the nucleic acid molecule comprises a nucleic acid sequence having at least about 70%, 75%, 80%, 85%, 90%, 95%, 96%, 97%, 98%, 99%, or 100% sequence identity to the iCRE insert nucleotide sequence of Table 10. In embodiments, the nucleic acid molecule comprises a nucleic acid sequence having at least about 70%, 75%, 80%, 85%, 90%, 95%, 96%, 97%, 98%, 99%, or 100% sequence identity to the 5′ UTR nucleotide sequence of Table 10. In embodiments, the nucleic acid molecule comprises a nucleic acid sequence having at least about 70%, 75%, 80%, 85%, 90%, 95%, 96%, 97%, 98%, 99%, or 100% sequence identity to the VP2 nucleotide sequence of Table 10. In embodiments, the nucleic acid molecule comprises a nucleic acid sequence having at least about 70%, 75%, 80%, 85%, 90%, 95%, 96%, 97%, 98%, 99%, or 100% sequence identity to the Apoptin nucleotide sequence of Table 10. In embodiments, the nucleic acid molecule comprises a nucleic acid sequence having at least about 70%, 75%, 80%, 85%, 90%, 95%, 96%, 97%, 98%, 99%, or 100% sequence identity to the VP1 nucleotide sequence of Table 10. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the VP2 nucleotide sequence of Table 10. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the Apoptin nucleotide sequence of Table 10. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the VP1 nucleotide sequence of Table 10. In embodiments, the nucleic acid molecule comprises a nucleic acid sequence having at least about 70%, 75%, 80%, 85%, 90%, 95%, 96%, 97%, 98%, 99%, or 100% sequence identity to the 3′ UTR nucleotide sequence of Table 10.

In embodiments, the nucleic acid molecule comprises a nucleic acid sequence having at least about 70%, 75%, 80%, 85%, 90%, 95%, 96%, 97%, 98%, 99%, or 100% sequence identity to the full nucleic acid sequence of Table 11, or a contiguous sequence of 100-200, 200-300, 300-400, 400-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2000-2100, 2100-2200, 2200-2300, or 2300-2319 nucleotides therein. In embodiments, the nucleic acid molecule comprises a nucleic acid sequence having at least about 70%, 75%, 80%, 85%, 90%, 95%, 96%, 97%, 98%, 99%, or 100% sequence identity to nucleotides 1-606 of the full nucleic acid sequence of Table 11. In embodiments, the nucleic acid molecule comprises a nucleic acid sequence having at least about 70%, 75%, 80%, 85%, 90%, 95%, 96%, 97%, 98%, 99%, or 100% sequence identity to nucleotides 1798-2319 of the full nucleic acid sequence of Table 11. In embodiments, the nucleic acid molecule does not comprise a nucleic acid sequence having at least about 70%, 75%, 80%, 85%, 90%, 95%, 96%, 97%, 98%, 99%, or 100% sequence identity to nucleotides 607-1797 of the full nucleic acid sequence of Table 1B, or a contiguous sequence of between 10-20, 20-30, 30-40, 40-50, 50-60, 60-70, 70-80, 80-90, 100-150, 150-200, 200-300, 300-400, 400-500, 500-600, 600-700, 700-800, 800-900, 900-950, 950-1000, 1000-1100, or 1100-1190 nucleotides therein. In embodiments, the nucleic acid molecule comprises a nucleic acid sequence having at least about 70%, 75%, 80%, 85%, 90%, 95%, 96%, 97%, 98%, 99%, or 100% sequence identity to the GFP insert nucleotide sequence of Table 11. In embodiments, the nucleic acid molecule comprises a nucleic acid sequence having at least about 70%, 75%, 80%, 85%, 90%, 95%, 96%, 97%, 98%, 99%, or 100% sequence identity to the 5′ UTR nucleotide sequence of Table 11. In embodiments, the nucleic acid molecule comprises a nucleic acid sequence having at least about 70%, 75%, 80%, 85%, 90%, 95%, 96%, 97%, 98%, 99%, or 100% sequence identity to the VP2 nucleotide sequence of Table 11. In embodiments, the nucleic acid molecule comprises a nucleic acid sequence having at least about 70%, 75%, 80%, 85%, 90%, 95%, 96%, 97%, 98%, 99%, or 100% sequence identity to the Apoptin nucleotide sequence of Table 11. In embodiments, the nucleic acid molecule comprises a nucleic acid sequence having at least about 70%, 75%, 80%, 85%, 90%, 95%, 96%, 97%, 98%, 99%, or 100% sequence identity to the VP1 nucleotide sequence of Table 11. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the VP2 nucleotide sequence of Table 11. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the Apoptin nucleotide sequence of Table 11. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the VP1 nucleotide sequence of Table 11. In embodiments, the nucleic acid molecule comprises a nucleic acid sequence having at least about 70%, 75%, 80%, 85%, 90%, 95%, 96%, 97%, 98%, 99%, or 100% sequence identity to the 3′ UTR nucleotide sequence of Table 11.

In embodiments, the nucleic acid molecule comprises a nucleic acid sequence having at least about 70%, 75%, 80%, 85%, 90%, 95%, 96%, 97%, 98%, 99%, or 100% sequence identity to the full nucleic acid sequence of Table 12, or a contiguous sequence of 100-200, 200-300, 300-400, 400-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2000-2100, 2100-2200, 2200-2300, or 2300-2319 nucleotides therein. In embodiments, the nucleic acid molecule comprises a nucleic acid sequence having at least about 70%, 75%, 80%, 85%, 90%, 95%, 96%, 97%, 98%, 99%, or 100% sequence identity to nucleotides 1-606 of the full nucleic acid sequence of Table 12. In embodiments, the nucleic acid molecule comprises a nucleic acid sequence having at least about 70%, 75%, 80%, 85%, 90%, 95%, 96%, 97%, 98%, 99%, or 100% sequence identity to nucleotides 2015-2319 of the full nucleic acid sequence of Table 12. In embodiments, the nucleic acid molecule does not comprise a nucleic acid sequence having at least about 70%, 75%, 80%, 85%, 90%, 95%, 96%, 97%, 98%, 99%, or 100% sequence identity to nucleotides 607-2014 of the full nucleic acid sequence of Table 1B, or a contiguous sequence of between 10-20, 20-30, 30-40, 40-50, 50-60, 60-70, 70-80, 80-90, 100-150, 150-200, 200-300, 300-400, 400-500, 500-600, 600-700, 700-800, 800-900, 900-950, 950-1000, 1000-1100, 1100-1200, 1200-1300, 1300-1400, or 1400-1407 nucleotides therein. In embodiments, the nucleic acid molecule comprises a nucleic acid sequence having at least about 70%, 75%, 80%, 85%, 90%, 95%, 96%, 97%, 98%, 99%, or 100% sequence identity to the Glue insert nucleotide sequence of Table 12. In embodiments, the nucleic acid molecule comprises a nucleic acid sequence having at least about 70%, 75%, 80%, 85%, 90%, 95%, 96%, 97%, 98%, 99%, or 100% sequence identity to the 5′ UTR nucleotide sequence of Table 12. In embodiments, the nucleic acid molecule comprises a nucleic acid sequence having at least about 70%, 75%, 80%, 85%, 90%, 95%, 96%, 97%, 98%, 99%, or 100% sequence identity to the VP2 nucleotide sequence of Table 12. In embodiments, the nucleic acid molecule comprises a nucleic acid sequence having at least about 70%, 75%, 80%, 85%, 90%, 95%, 96%, 97%, 98%, 99%, or 100% sequence identity to the Apoptin nucleotide sequence of Table 12. In embodiments, the nucleic acid molecule comprises a nucleic acid sequence having at least about 70%, 75%, 80%, 85%, 90%, 95%, 96%, 97%, 98%, 99%, or 100% sequence identity to the VP1 nucleotide sequence of Table 12. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the VP2 nucleotide sequence of Table 12. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the Apoptin nucleotide sequence of Table 12. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the VP1 nucleotide sequence of Table 12. In embodiments, the nucleic acid molecule comprises a nucleic acid sequence having at least about 70%, 75%, 80%, 85%, 90%, 95%, 96%, 97%, 98%, 99%, or 100% sequence identity to the 3′ UTR nucleotide sequence of Table 12.

In embodiments, the nucleic acid molecule comprises a nucleic acid sequence having at least about 70%, 75%, 80%, 85%, 90%, 95%, 96%, 97%, 98%, 99%, or 100% sequence identity to the full nucleic acid sequence of Table 13, or a contiguous sequence of 100-200, 200-300, 300-400, 400-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2000-2100, 2100-2200, 2200-2300, or 2300-2319 nucleotides therein. In embodiments, the nucleic acid molecule comprises a nucleic acid sequence having at least about 70%, 75%, 80%, 85%, 90%, 95%, 96%, 97%, 98%, 99%, or 100% sequence identity to nucleotides 1-606 of the full nucleic acid sequence of Table 13. In embodiments, the nucleic acid molecule comprises a nucleic acid sequence having at least about 70%, 75%, 80%, 85%, 90%, 95%, 96%, 97%, 98%, 99%, or 100% sequence identity to nucleotides 1790-2319 of the full nucleic acid sequence of Table 13. In embodiments, the nucleic acid molecule does not comprise a nucleic acid sequence having at least about 70%, 75%, 80%, 85%, 90%, 95%, 96%, 97%, 98%, 99%, or 100% sequence identity to nucleotides 607-1789 of the full nucleic acid sequence of Table 1B, or a contiguous sequence of between 10-20, 20-30, 30-40, 40-50, 50-60, 60-70, 70-80, 80-90, 100-150, 150-200, 200-300, 300-400, 400-500, 500-600, 600-700, 700-800, 800-900, 900-950, 950-1000, 1000-1100, or 1100-1182 nucleotides therein. In embodiments, the nucleic acid molecule comprises a nucleic acid sequence having at least about 70%, 75%, 80%, 85%, 90%, 95%, 96%, 97%, 98%, 99%, or 100% sequence identity to the mCherry insert nucleotide sequence of Table 13. In embodiments, the nucleic acid molecule comprises a nucleic acid sequence having at least about 70%, 75%, 80%, 85%, 90%, 95%, 96%, 97%, 98%, 99%, or 100% sequence identity to the 5′ UTR nucleotide sequence of Table 13. In embodiments, the nucleic acid molecule comprises a nucleic acid sequence having at least about 70%, 75%, 80%, 85%, 90%, 95%, 96%, 97%, 98%, 99%, or 100% sequence identity to the VP2 nucleotide sequence of Table 13. In embodiments, the nucleic acid molecule comprises a nucleic acid sequence having at least about 70%, 75%, 80%, 85%, 90%, 95%, 96%, 97%, 98%, 99%, or 100% sequence identity to the Apoptin nucleotide sequence of Table 13. In embodiments, the nucleic acid molecule comprises a nucleic acid sequence having at least about 70%, 75%, 80%, 85%, 90%, 95%, 96%, 97%, 98%, 99%, or 100% sequence identity to the VP1 nucleotide sequence of Table 13. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the VP2 nucleotide sequence of Table 13. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the Apoptin nucleotide sequence of Table 13. In embodiments, the nucleic acid molecule comprises a nucleic acid sequence encoding a polypeptide having at least about 70%, 75%, 80%, 85%, 90%, 95%, 96%, 97%, 98%, 99%, or 100% amino acid sequence identity to a polypeptide encoded by the VP1 nucleotide sequence of Table 13. In embodiments, the nucleic acid molecule comprises a nucleic acid sequence having at least about 70%, 75%, 80%, 85%, 90%, 95%, 96%, 97%, 98%, 99%, or 100% sequence identity to the 3′ UTR nucleotide sequence of Table 13.

In some embodiments, the genetic element comprises a nucleotide sequence encoding an amino acid sequence or a functional fragment thereof or a sequence having at least about 60%, 70% 80%, 85%, 90% 95%, 96%, 97%, 98%, 99%, or 100% sequence identity to any one of the amino acid sequences described herein, e.g., a CAV amino acid sequence, e.g., as listed in or encoded by a sequence listed in any of Tables 1-17.

In some embodiments, a CAVector as described herein comprises one or more nucleic acid molecules (e.g., a genetic element as described herein) comprising a sequence having at least about 70%, 75%, 80%, 85%, 90%, 95%, 96%, 97%, 98%, 99%, or 100% sequence identity to a CAV sequence, e.g., as described herein, or a fragment thereof. In embodiments, the CAVector comprises a nucleic acid sequence selected from a sequence as shown in any of Tables 1-17, or a sequence having at least 70%, 75%, 80%, 85%, 90%, 95%, 96%, 97%, 98%, 99%, or 100% sequence identity thereto. In embodiments, the CAVector comprises a polypeptide comprising an amino acid sequence encoded by a sequence as shown in any of Tables 1-17, or a sequence having at least 70%, 75%, 80%, 85%, 90%, 95%, 96%, 97%, 98%, 99%, or 100% sequence identity thereto.

In some embodiments, a CAVector as described herein comprises one or more nucleic acid molecules (e.g., a genetic element as described herein) comprising a sequence having at least about 70%, 75%, 80%, 85%, 90%, 95%, 96%, 97%, 98%, 99%, or 100% sequence identity to one or more of a 5′ UTR, repeat region, CAAT signal, TATA box, VP2 gene, Apoptin gene, VP1, 3′ UTR, GC-rich region, polyA signal sequence, or any combination thereof, of any of the CAVs described herein. In some embodiments, the nucleic acid molecule comprises a sequence encoding a capsid protein (e.g., a VP1 molecule), a VP2 molecule, and/or a Apoptin molecule of any of the CAVs described herein. In embodiments, the nucleic acid molecule comprises a sequence encoding a capsid protein comprising an amino acid sequence having at least about 70%, 75%, 80%, 85%, 90%, 95%, 96%, 97%, 98%, 99%, or 100% sequence identity to a CAV VP1 molecule (e.g., an VP1 amino acid sequence as shown in any of Tables 1-17, or an VP1 amino acid sequence encoded by a nucleic acid sequence as shown in any of Tables 1-17).

In some embodiments, a CAVector comprises a genetic element comprising a nucleic acid sequence encoding a polypeptide having at least about 70%, 75%, 80%, 85%, 90%, 95%, 96%, 97%, 98%, 99%, or 100% sequence identity to a VP1 molecule encoded by a CAV genome sequence listed in any of Tables 1-17. In some embodiments, a CAVector comprises a genetic element comprising a nucleic acid sequence encoding a polypeptide having at least about 70%, 75%, 80%, 85%, 90%, 95%, 96%, 97%, 98%, 99%, or 100% sequence identity to a VP2 molecule encoded by a CAV genome sequence listed in any of Tables 1-17. In some embodiments, a CAVector comprises a genetic element comprising a nucleic acid sequence encoding a polypeptide having at least about 70%, 75%, 80%, 85%, 90%, 95%, 96%, 97%, 98%, 99%, or 100% sequence identity to an Apoptin molecule encoded by a CAV genome sequence listed in any of Tables 1-17.

In some embodiments, a CAVector comprises a genetic element that does not comprise a nucleic acid sequence encoding a polypeptide having at least about 70%, 75%, 80%, 85%, 90%, 95%, 96%, 97%, 98%, 99%, or 100% sequence identity to a VP1 molecule encoded by a CAV genome sequence listed in any of Tables 1-17. In some embodiments, a CAVector comprises a genetic element that does not comprise a nucleic acid sequence encoding a polypeptide having at least about 70%, 75%, 80%, 85%, 90%, 95%, 96%, 97%, 98%, 99%, or 100% sequence identity to a VP2 molecule encoded by a CAV genome sequence listed in any of Tables 1-17. In some embodiments, a CAVector comprises a genetic element that does not comprise a nucleic acid sequence encoding a polypeptide having at least about 70%, 75%, 80%, 85%, 90%, 95%, 96%, 97%, 98%, 99%, or 100% sequence identity to an Apoptin molecule encoded by a CAV genome sequence listed in any of Tables 1-17.

TABLE 1A
Exemplary chicken anemia virus (CAV) nucleic acid sequence

Name CAV isolate Cuxhaven 1
Genus/Clade Gyrovirus
Accession Number M55918
Full Sequence: 2313 bp

| | |
|---|---|
| CGAGTGGTTA CTATTCCATC ACCATTCTAG CCTGTACACA GAAAGTCAAG ATGGACGAAT | 60 |
| CGCTCGACTT CGCTCGCGAT TCGTCGAAGG CGGGGGGCCG GAGGCCCCCC GGTGGCCCCC | 120 |
| CTCCAACGAG TGGAGCACGT ACAGGGGGGT ACGTCATCCG TACAGGGGGG TACGTCATCC | 180 |
| GTACAGGGGG GTACGTCACA AAGAGGCGTT CCCGTACAGG GGGGTACGTC ACGCGTACAG | 240 |
| GGGGGTACGT CACAGCCAAT CAAAAGCTGC CACGTTGCGA AAGTGACGTT TCGAAAATGG | 300 |
| GCGGCGCAAG CCTCTCTATA TATTGAGCGC ACATACCGGT CGGCAGTAGG TATACGCAAG | 360 |
| GCGGTCCGGG TGGATGCACG GGAACGGCGG ACAACCGGCC GCTGGGGGCA GTGAATCGGC | 420 |
| GCTTAGCCGA GAGGGGCAAC CTGGGCCCAG CGGAGCCGCG CAGGGGCAAG TAATTTCAAA | 480 |
| TGAACGCTCT CCAAGAAGAT ACTCCACCCG GACCATCAAC GGTGTTCAGG CCACCAACAA | 540 |
| GTTCACGGCC GTTGGAAACC CCTCACTGCA GAGAGATCCG GATTGGTATC GCTGGAATTA | 600 |
| CAATCACTCT ATCGCTGTGT GGCTGCGCGA ATGCTCGCGC TCCCACGCTA AGATCTGCAA | 660 |
| CTGCGGACAA TTCAGAAAGC ACTGGTTTCA AGAATGTGCC GGACTTGAGG ACCGATCAAC | 720 |
| CCAAGCCTCC CTCGAAGAAG CGATCCTGCG ACCCCTCCGA GTACAGGGTA AGCGAGCTAA | 780 |
| AAGAAAGCTT GATTACCACT ACTCCCAGCC GACCCCGAAC CGCAAAAAGG CGTATAAGAC | 840 |
| TGTAAGATGG CAAGACGAGC TCGCAGACCG AGAGGCCGAT TTTACTCCTT CAGAAGAGGA | 900 |
| CGGTGGCACC ACCTCAAGCG ACTTCGACGA AGATATAAAT TTCGACATCG GAGGAGACAG | 960 |
| CGGTATCGTA GACGAGCTTT TAGGAAGGCC TTTCACAACC CCCGCCCCGG TACGTATAGT | 1020 |
| GTGAGGCTGC CGAACCCCCA ATCTACTATG ACTATCCGCT TCCAAGGGGT CATCTTTCTC | 1080 |
| ACGGAAGGAC TCATTCTGCC TAAAAACAGC ACAGCGGGGG GCTATGCAGA CCACATGTAC | 1140 |
| GGGGCGAGAG TCGCCAAGAT CTCTGTGAAC CTGAAAGAGT TCCTGCTAGC CTCAATGAAC | 1200 |
| CTGACATACG TGAGCAAAAT CGGAGGCCCC ATCGCCGGTG AGTTGATTGC GGACGGGTCT | 1260 |
| AAATCACAAG CCGCGGACAA TTGGCCTAAT TGCTGGCTGC CGCTAGATAA TAACGTGCCC | 1320 |
| TCCGCTACAC CATCGGCATG GTGGAGATGG GCCTTAATGA TGATGCAGCC CACGGACTCT | 1380 |
| TGCCGGTTCT TTAATCACCC AAAGCAGATG ACCCTGCAAG ACATGGGTCG CATGTTTGGG | 1440 |
| GGCTGGCACC TGTTCCGACA CATTGAAACC CGCTTTCAGC TCCTTGCCAC TAAGAATGAG | 1500 |
| GGATCCTTCA GCCCCGTGGC GAGTCTTCTC TCCCAGGGAG AGTACCTCAC GCGTCGGGAC | 1560 |
| GATGTTAAGT ACAGCAGCGA TCACCAGAAC CGGTGGCAAA AAGGCGGACA ACCGATGACG | 1620 |
| GGGGGCATTG CTTATGCGAC CGGGAAAATG AGACCCGACG AGCAACAGTA CCCTGCTATG | 1680 |
| CCCCCAGACC CCCCGATCAT CACCGCTACT ACAGCGCAAG GCACGCAAGT CCGCTGCATG | 1740 |
| AATAGCACGC AAGCTTGGTG GTCATGGGAC ACATATATGA GCTTTGCAAC ACTCACAGCA | 1800 |
| CTCGGTGCAC AATGGTCTTT TCCTCCAGGG CAACGTTCAG TTTCTAGACG GTCCTTCAAC | 1860 |
| CACCACAAGG CGAGAGGAGC CGGGGACCCC AAGGGCCAGA GATGGCACAC GCTGGTGCCG | 1920 |
| CTCGGCACGG AGACCATCAC CGACAGCTAC ATGTCAGCAC CCGCATCAGA GCTGGACACT | 1980 |
| AATTTCTTTA CGCTTTACGT AGCGCAAGGC ACAAATAAGT CGCAACAGTA CAAGTTCGGC | 2040 |
| ACAGCTACAT ACGCGCTAAA GGAGCCGGTA ATGAAGAGCG ATGCATGGGC AGTGGTACGC | 2100 |
| GTCCAGTCGG TCTGGCAGCT GGGTAACAGG CAGAGGCCAT ACCCATGGGA CGTCAACTGG | 2160 |
| GCGAACAGCA CCATGTACTG GGGGACGCAG CCCTGAAAAG GGGGGGGGGC TAAAGCCCCC | 2220 |
| CCCCCTTAAA CCCCCCCCTG GGGGGGATTC CCCCCCAGAC CCCCCCTTTA TATAGCACTC | 2280 |
| AATAAACGCA GAAAATAGAT TTATCGCACT ATC | 2313 |
| (SEQ ID NO: 1) | |

| Putative Domain | Base range |
|---|---|
| 5′ UTR | 1-374 |
| Repeat Region | 138-254 |
| CAAT Signal | 255-260 |
| TATA Box | 317-322 |
| VP2 | 374-1024 |
| Apoptin | 480-845 |
| VP1 | 847-2196 |
| 3′ UTR | 2197-2313 |
| GC-Rich Region | 2200-2266 |
| PolyA Signal Sequence | 2281-2286 |

TABLE 1B
Alternate exemplary chicken anemia virus (CAV) nucleic acid sequence

Name CAV isolate Cuxhaven 1
Genus/Clade Gyrovirus
Accession Number M55918
Full Sequence: 2319 bp

| | |
|---|---|
| GAATTCCGAG TGGTTACTAT TCCATCACCA TTCTAGCCTG TACACAGAAA GTCAAGATGG | 60 |
| ACGAATCGCT CGACTTCGCT CGCGATTCGT CGAAGGCGGG GGGCCGGAGG CCCCCCGGTG | 120 |
| GCCCCCCTCC AACGAGTGGA GCACGTACAG GGGGGTACGT CATCCGTACA GGGGGGTACG | 180 |
| TCATCCGTAC AGGGGGGTAC GTCACAAAGA GGCGTTCCCG TACAGGGGGG TACGTCACGC | 240 |
| GTACAGGGGG GTACGTCACA GCCAATCAAA AGCTGCCACG TTGCGAAAGT GACGTTTCGA | 300 |
| AAATGGGCGG CGCAAGCCTC TCTATATATT GAGCGCACAT ACCGGTCGGC AGTAGGTATA | 360 |
| CGCAAGGCGG TCCGGGTGGA TGCACGGGAA CGGCGGACAA CCGGCCGCTG GGGGCAGTGA | 420 |
| ATCGGCGCTT AGCCGAGAGG GGCAACCTGG GCCCAGCGGA GCCGCGCAGG GGCAAGTAAT | 480 |
| TTCAAATGAA CGCTCTCCAA GAAGATACTC CACCCGGACC ATCAACGGTG TTCAGGCCAC | 540 |
| CAACAAGTTC ACGGCCGTTG GAAACCCCTC ACTGCAGAGA GATCCGGATT GGTATCGCTG | 600 |
| GAATTACAAT CACTCTATCG CTGTGTGGCT GCGCGAATGC TCGCGCTCCC ACGCTAAGAT | 660 |
| CTGCAACTGC GGACAATTCA GAAAGCACTG GTTTCAAGAA TGTGCCGGAC TTGAGGACCG | 720 |
| ATCAACCCAA GCCTCCCTCG AAGAAGCGAT CCTGCGACCC CTCCGAGTAC AGGGTAAGCG | 780 |
| AGCTAAAAGA AAGCTTGATT ACCACTACTC CCAGCCGACC CCGAACCGCA AAAAGGCGTA | 840 |
| TAAGACTGTA AGATGGCAAG ACGAGCTCGC AGACCGAGAG GCCGATTTTA CTCCTTCAGA | 900 |
| AGAGGACGGT GGCACCACCT CAAGCGACTT CGACGAAGAT ATAAATTTCG ACATCGGAGG | 960 |
| AGACAGCGGT ATCGTAGACG AGCTTTTAGG AAGGCCTTTC ACAACCCCCG CCCCGGTACG | 1020 |
| TATAGTGTGA GGCTGCCGAA CCCCCAATCT ACTATGACTA TCCGCTTCCA AGGGGTCATC | 1080 |
| TTTCTCACGG AAGGACTCAT TCTGCCTAAA AACAGCACAG CGGGGGGCTA TGCAGACCAC | 1140 |
| ATGTACGGGG CGAGAGTCGC CAAGATCTCT GTGAACCTGA AAGAGTTCCT GCTAGCCTCA | 1200 |
| ATGAACCTGA CATACGTGAG CAAAATCGGA GGCCCCATCG CCGGTGAGTT GATTGCGGAC | 1260 |
| GGGTCTAAAT CACAAGCCGC GGACAATTGG CCTAATTGCT GGCTGCCGCT AGATAATAAC | 1320 |
| GTGCCCTCCG CTACACCATC GGCATGGTGG AGATGGGCCT TAATGATGAT GCAGCCCACG | 1380 |
| GACTCTTGCC GGTTCTTTAA TCACCCAAAG CAGATGACCC TGCAAGACAT GGGTCGCATG | 1440 |
| TTTGGGGGCT GGCACCTGTT CCGACACATT GAAACCCGCT TTCAGCTCCT TGCCACTAAG | 1500 |
| AATGAGGGAT CCTTCAGCCC CGTGGCGAGT CTTCTCTCCC AGGGAGAGTA CCTCACGCGT | 1560 |
| CGGGACGATG TTAAGTACAG CAGCGATCAC CAGAACCGGT GGCAAAAAGG CGGACAACCG | 1620 |
| ATGACGGGGG GCATTGCTTA TGCGACCGGG AAAATGAGAC CCGACGAGCA ACAGTACCCT | 1680 |
| GCTATGCCCC CAGACCCCCC GATCATCACC GCTACTACAG CGCAAGGCAC GCAAGTCCGC | 1740 |
| TGCATGAATA GCACGCAAGC TTGGTGGTCA TGGGACACAT ATATGAGCTT TGCAACACTC | 1800 |
| ACAGCACTCG GTGCACAATG GTCTTTTCCT CCAGGGCAAC GTTCAGTTTC TAGACGGTCC | 1860 |
| TTCAACCACC ACAAGGCGAG AGGAGCCGGG GACCCCAAGG GCCAGAGATG GCACACGCTG | 1920 |
| GTGCCGCTCG GCACGGAGAC CATCACCGAC AGCTACATGT CAGCACCCGC ATCAGAGCTG | 1980 |
| GACACTAATT TCTTTACGCT TTACGTAGCG CAAGGCACAA ATAAGTCGCA ACAGTACAAG | 2040 |
| TTCGGCACAG CTACATACGC GCTAAAGGAG CCGGTAATGA AGAGCGATGC ATGGGCAGTG | 2100 |
| GTACGCGTCC AGTCGGTCTG GCAGCTGGGT AACAGGCAGA GGCCATACCC ATGGGACGTC | 2160 |
| AACTGGGCGA ACAGCACCAT GTACTGGGGG ACGCAGCCCT GAAAAGGGGG GGGGGCTAAA | 2220 |
| GCCCCCCCCC CTTAAACCCC CCCCTGGGGG GGATTCCCCC CCAGACCCCC CCTTTATATA | 2280 |
| GCACTCAATA AACGCAGAAA ATAGATTTAT CGCACTATC | 2319 |
| (SEQ ID NO: 100) | |

| Annotations: | |
|---|---|
| Putative Domain | Base range |
| 5′ UTR | 1-379 |
| VP2 | 380-1030 |
| Apoptin | 485-851 |
| VP1 | 853-2202 |
| 3′ UTR | 2203-2319 |

TABLE 2
CAV-nLuc1 nucleic acid sequence

Name CAV-nLuc1 mutant
Genus/Clade Gyrovirus
Full Sequence: 2319 bp

| | |
|---|---|
| GAATTCCTGT GGAATGTGTG TCAGTTAGGG TGTGGAAAGT CCCCAGGCTC CCCAGCAGGC | 60 |
| AGAAGTATGC AAAGCATGCA TCTCAATTAG TCAGCAACCA GGTGTGGAAA GTCCCCAGGC | 120 |
| TCCCCAGCAG GCAGAAGTAT GCAAAGCATG CATCTCAATT AGTCAGCAAC CATAGTCCCG | 180 |
| CCCCTAACTC CGCCCATCCC GCCCCTAACT CCGCCCAGTT CCGCCCATTC TCCGCCCCAT | 240 |
| GGCTGACTAA TTTTTTTTAT TTATGCAGAG GCCGAGGCCG CCTCTGCCTC TGAGCTATTC | 300 |
| CAGAAGTAGT GAGGAGGCTT TTTTGGAGGC CTAGGCTTTT GCAAAAAGCT GCCACCATGG | 360 |
| TCTTCACACT CGAAGATTTC GTTGGGGACT GGCGACAGAC AGCCGGCTAC AACCTGGACC | 420 |
| AAGTCCTTGA ACAGGGAGGT GTGTCCAGTT TGTTTCAGAA TCTCGGGGTG TCCGTAACTC | 480 |
| CGATCCAAAG GATTGTCCTG AGCGGTGAAA ATGGGCTGAA GATCGACATC CATGTCATCA | 540 |
| TCCCGTATGA AGGTCTGAGC GGCGACCAAA TGGGCCAGAT CGAAAAAATT TTTAAGGTGG | 600 |
| TGTACCCTGT GGATGATCAT CACTTTAAGG TGATCCTGCA CTATGGCACA CTGGTAATCG | 660 |
| ACGGGGTTAC GCCGAACATG ATCGACTATT TCGGACGGCC GTATGAAGGC ATCGCCGTGT | 720 |
| TCGACGGCAA AAAGATCACT GTAACAGGGA CCCTGTGGAA CGGCAACAAA ATTATCGACG | 780 |
| AGCGCCTGAT CAACCCCGAC GGCTCCCTGC TGTTCCGAGT AACCATCAAC GGAGTGACCG | 840 |
| GCTGGCGGCT GTGCGAACGC ATTCTGGCGT AATAAGATAC ATTGATGAGT TTGGACAAAC | 900 |
| CACAACTAGA ATGCAGTGAA AAAAATGCTT TATTTGTGAA ATTTGTGATG CTATTGCTTT | 960 |
| ATTTGTAACC ATTATAAGCT GCAATAAACA AGTTCCTTTC ACAACCCCCG CCCCGGTACG | 1020 |
| TATAGTGTGA GGCTGCCGAA CCCCCAATCT ACTATGACTA TCCGCTTCCA AGGGGTCATC | 1080 |
| TTTCTCACGG AAGGACTCAT TCTGCCTAAA AACAGCACAG CGGGGGGCTA TGCAGACCAC | 1140 |
| ATGTACGGGG CGAGAGTCGC CAAGATCTCT GTGAACCTGA AAGAGTTCCT GCTAGCCTCA | 1200 |
| ATGAACCTGA CATACGTGAG CAAAATCGGA GGCCCCATCG CCGGTGAGTT GATTGCGGAC | 1260 |
| GGGTCTAAAT CACAAGCCGC GGACAATTGG CCTAATTGCT GGCTGCCGCT AGATAATAAC | 1320 |
| GTGCCCTCCG CTACACCATC GGCATGGTGG AGATGGGCCT TAATGATGAT GCAGCCCACG | 1380 |
| GACTCTTGCC GGTTCTTTAA TCACCCAAAG CAGATGACCC TGCAAGACAT GGGTCGCATG | 1440 |
| TTTGGGGGCT GGCACCTGTT CCGACACATT GAAACCCGCT TTCAGCTCCT TGCCACTAAG | 1500 |
| AATGAGGGAT CCTTCAGCCC CGTGGCGAGT CTTCTCTCCC AGGGAGAGTA CCTCACGCGT | 1560 |
| CGGGACGATG TTAAGTACAG CAGCGATCAC CAGAACCGGT GGCAAAAAGG CGGACAACCG | 1620 |
| ATGACGGGGG GCATTGCTTA TGCGACCGGG AAAATGAGAC CCGACGAGCA ACAGTACCCT | 1680 |
| GCTATGCCCC CAGACCCCCC GATCATCACC GCTACTACAG CGCAAGGCAC GCAAGTCCGC | 1740 |
| TGCATGAATA GCACGCAAGC TTGGTGGTCA TGGGACACAT ATATGAGCTT TGCAACACTC | 1800 |
| ACAGCACTCG GTGCACAATG GTCTTTTCCT CCAGGGCAAC GTTCAGTTTC TAGACGGTCC | 1860 |
| TTCAACCACC ACAAGGCGAG AGGAGCCGGG GACCCCAAGG GCCAGAGATG GCACACGCTG | 1920 |
| GTGCCGCTCG GCACGGAGAC CATCACCGAC AGCTACATGT CAGCACCCGC ATCAGAGCTG | 1980 |
| GACACTAATT TCTTTACGCT TTACGTAGCG CAAGGCACAA ATAAGTCGCA ACAGTACAAG | 2040 |
| TTCGGCACAG CTACATACGC GCTAAAGGAG CCGGTAATGA AGAGCGATGC ATGGGCAGTG | 2100 |
| GTACGCGTCC AGTCGGTCTG GCAGCTGGGT AACAGGCAGA GGCCATACCC ATGGGACGTC | 2160 |
| AACTGGGCGA ACAGCACCAT GTACTGGGGG ACGCAGCCCT GAAAAGGGGG GGGGGCTAAA | 2220 |
| GCCCCCCCCC CTTAAACCCC CCCCTGGGGG GGATTCCCCC CCAGACCCCC CCTTTATATA | 2280 |
| GCACTCAATA AACGCAGAAA ATAGATTTAT CGCACTATC | 2319 |
| (SEQ ID NO: 2) | |

| Annotations: | |
|---|---|
| Putative Domain | Base range |
| nLuc insert | 7-994 |
| 5′ UTR | 1-6 |
| VP2 | 995-1030 |
| Apoptin | N/A |
| VP1 | 995-2202 |
| 3′ UTR | 2203-2319 |

TABLE 3
CAV-nLuc2 nucleic acid sequence

Name CAV-nLuc2 mutant
Genus/Clade Gyrovirus
Full Sequence: 2319 bp

| | |
|---|---|
| GAATTCCGAG TGGTTACTAT TCCATCACCA TTCTAGCCTG TACACAGAAA GTCAAGATGG | 60 |
| ACGAATCGCT CGACTTCGCT CGCGATTCGT CGAAGGCGGG GGGCCGGAGG CCCCCCGGTG | 120 |
| GCCCCCCTCC AACGAGTGGA GCACGTACAG GGGGGTACGT CATCCGTACA GGGGGGTACG | 180 |
| TCATCCGTAC AGGGGGGTAC GTCACACTGT GGAATGTGTG TCAGTTAGGG TGTGGAAAGT | 240 |
| CCCCAGGCTC CCCAGCAGGC AGAAGTATGC AAAGCATGCA TCTCAATTAG TCAGCAACCA | 300 |
| GGTGTGGAAA GTCCCCAGGC TCCCCAGCAG GCAGAAGTAT GCAAAGCATG CATCTCAATT | 360 |
| AGTCAGCAAC CATAGTCCCG CCCCTAACTC CGCCCATCCC GCCCCTAACT CCGCCCAGTT | 420 |
| CCGCCCATTC TCCGCCCCAT GGCTGACTAA TTTTTTTTAT TTATGCAGAG GCCGAGGCCG | 480 |
| CCTCTGCCTC TGAGCTATTC CAGAAGTAGT GAGGAGGCTT TTTTGGAGGC CTAGGCTTTT | 540 |
| GCAAAAAGCT GCCACCATGG TCTTCACACT CGAAGATTTC GTTGGGGACT GGCGACAGAC | 600 |
| AGCCGGCTAC AACCTGGACC AAGTCCTTGA ACAGGGAGGT GTGTCCAGTT TGTTTCAGAA | 660 |
| TCTCGGGGTG TCCGTAACTC CGATCCAAAG GATTGTCCTG AGCGGTGAAA ATGGGCTGAA | 720 |
| GATCGACATC CATGTCATCA TCCCGTATGA AGGTCTGAGC GGCGACCAAA TGGGCCAGAT | 780 |
| CGAAAAAATT TTTAAGGTGG TGTACCCTGT GGATGATCAT CACTTTAAGG TGATCCTGCA | 840 |
| CTATGGCACA CTGGTAATCG ACGGGGTTAC GCCGAACATG ATCGACTATT TCGGACGGCC | 900 |
| GTATGAAGGC ATCGCCGTGT TCGACGGCAA AAAGATCACT GTAACAGGGA CCCTGTGGAA | 960 |
| CGGCAACAAA ATTATCGACG AGCGCCTGAT CAACCCCGAC GGCTCCCTGC TGTTCCGAGT | 1020 |
| AACCATCAAC GGAGTGACCG GCTGGCGGCT GTGCGAACGC ATTCTGGCGT AATAAGATAC | 1080 |
| ATTGATGAGT TTGGACAAAC CACAACTAGA ATGCAGTGAA AAAAATGCTT TATTTGTGAA | 1140 |
| ATTTGTGATG CTATTGCTTT ATTTGTAACC ATTATAAGCT GCAATAAACA AGTTGCCTCA | 1200 |
| ATGAACCTGA CATACGTGAG CAAAATCGGA GGCCCCATCG CCGGTGAGTT GATTGCGGAC | 1260 |
| GGGTCTAAAT CACAAGCCGC GGACAATTGG CCTAATTGCT GGCTGCCGCT AGATAATAAC | 1320 |
| GTGCCCTCCG CTACACCATC GGCATGGTGG AGATGGGCCT TAATGATGAT GCAGCCCACG | 1380 |
| GACTCTTGCC GGTTCTTTAA TCACCCAAAG CAGATGACCC TGCAAGACAT GGGTCGCATG | 1440 |
| TTTGGGGGCT GGCACCTGTT CCGACACATT GAAACCCGCT TTCAGCTCCT TGCCACTAAG | 1500 |
| AATGAGGGAT CCTTCAGCCC CGTGGCGAGT CTTCTCTCCC AGGGAGAGTA CCTCACGCGT | 1560 |
| CGGGACGATG TTAAGTACAG CAGCGATCAC CAGAACCGGT GGCAAAAAGG CGGACAACCG | 1620 |
| ATGACGGGGG GCATTGCTTA TGCGACCGGG AAAATGAGAC CCGACGAGCA ACAGTACCCT | 1680 |
| GCTATGCCCC CAGACCCCCC GATCATCACC GCTACTACAG CGCAAGGCAC GCAAGTCCGC | 1740 |
| TGCATGAATA GCACGCAAGC TTGGTGGTCA TGGGACACAT ATATGAGCTT TGCAACACTC | 1800 |
| ACAGCACTCG GTGCACAATG GTCTTTTCCT CCAGGGCAAC GTTCAGTTTC TAGACGGTCC | 1860 |
| TTCAACCACC ACAAGGCGAG AGGAGCCGGG GACCCCAAGG GCCAGAGATG GCACACGCTG | 1920 |
| GTGCCGCTCG GCACGGAGAC CATCACCGAC AGCTACATGT CAGCACCCGC ATCAGAGCTG | 1980 |
| GACACTAATT TCTTTACGCT TTACGTAGCG CAAGGCACAA ATAAGTCGCA ACAGTACAAG | 2040 |
| TTCGGCACAG CTACATACGC GCTAAAGGAG CCGGTAATGA AGAGCGATGC ATGGGCAGTG | 2100 |
| GTACGCGTCC AGTCGGTCTG GCAGCTGGGT AACAGGCAGA GGCCATACCC ATGGGACGTC | 2160 |
| AACTGGGCGA ACAGCACCAT GTACTGGGGG ACGCAGCCCT GAAAAGGGGG GGGGGCTAAA | 2220 |
| GCCCCCCCCC CTTAAACCCC CCCCTGGGGG GGATTCCCCC CCAGACCCCC | 2280 |
| GCACTCAATA AACGCAGAAA ATAGATTTAT CGCACTATCC CTTTATATA | 2319 |
| (SEQ ID NO: 3) | |

| Annotations: | |
|---|---|
| Putative Domain | Base range |
| nLuc insert | 207-1194 |
| 5′ UTR | 1-206 |
| VP2 | N/A |
| Apoptin | N/A |
| VP1 | 1194-2202 |
| 3′ UTR | 2203-2319 |

TABLE 4
CAV-nLuc3 nucleic acid sequence

Name CAV-nLuc3 mutant
Genus/Clade Gyrovirus
Full Sequence: 2319 bp

| | |
|---|---|
| GAATTCCGAG TGGTTACTAT TCCATCACCA TTCTAGCCTG TACACAGAAA GTCAAGATGG | 60 |
| ACGAATCGCT CGACTTCGCT CGCGATTCGT CGAAGGCGGG GGGCCGGAGG CCCCCCGGTG | 120 |
| GCCCCCCTCC AACGAGTGGA GCACGTACAG GGGGGTACGT CATCCGTACA GGGGGGTACG | 180 |
| TCATCCGTAC AGGGGGGTAC GTCACAAAGA GGCGTTCCCG TACAGGGGGG TACGTCACGC | 240 |
| GTACAGGGGG GTACGTCACA GCCAATCAAA AGCTGCCACG TTGCGAAAGT GACGTTTCGA | 300 |
| AAATGGGCGG CGCAAGCCTC TCTATATATT GAGCGCACAT ACCGGTCGGC AGTAGGTATA | 360 |
| CGCAAGGCGG TCCGGGTGGT TGCACGGGAA CGGCGGACAA CCGGCCCTGT GGAATGTGTG | 420 |
| TCAGTTAGGG TGTGGAAAGT CCCCAGGCTC CCCAGCAGGC AGAAGTATGC AAAGCATGCA | 480 |
| TCTCAATTAG TCAGCAACCA GGTGTGGAAA GTCCCCAGGC TCCCCAGCAG GCAGAAGTAT | 540 |
| GCAAAGCATG CATCTCAATT AGTCAGCAAC CATAGTCCCG CCCCTAACTC CGCCCATCCC | 600 |
| GCCCCTAACT CCGCCCAGTT CCGCCCATTC TCCGCCCCAT GGCTGACTAA TTTTTTTTAT | 660 |
| TTATGCAGAG GCCGAGGCCG CCTCTGCCTC TGAGCTATTC CAGAAGTAGT GAGGAGGCTT | 720 |
| TTTTGGAGGC CTAGGCTTTT GCAAAAAGCT GCCACCATGG TCTTCACACT CGAAGATTTC | 780 |
| GTTGGGGACT GGCGACAGAC AGCCGGCTAC AACCTGGACC AAGTCCTTGA ACAGGGAGGT | 840 |
| GTGTCCAGTT TGTTTCAGAA TCTCGGGGTG TCCGTAACTC CGATCCAAAG GATTGTCCTG | 900 |
| AGCGGTGAAA ATGGGCTGAA GATCGACATC CATGTCATCA TCCCGTATGA AGGTCTGAGC | 960 |
| GGCGACCAAA TGGGCCAGAT CGAAAAAATT TTTAAGGTGG TGTACCCTGT GGATGATCAT | 1020 |
| CACTTTAAGG TGATCCTGCA CTATGGCACA CTGGTAATCG ACGGGGTTAC GCCGAACATG | 1080 |
| ATCGACTATT TCGGACGGCC GTATGAAGGC ATCGCCGTGT TCGACGGCAA AAAGATCACT | 1140 |
| GTAACAGGGA CCCTGTGGAA CGGCAACAAA ATTATCGACG AGCGCCTGAT CAACCCCGAC | 1200 |
| GGCTCCCTGC TGTTCCGAGT AACCATCAAC GGAGTGACCG GCTGGCGGCT GTGCGAACGC | 1260 |
| ATTCTGGCGT AATAAGATAC ATTGATGAGT TTGGACAAAC CACAACTAGA ATGCAGTGAA | 1320 |
| AAAAATGCTT TATTTGTGAA ATTTGTGATG CTATTGCTTT ATTTGTAACC ATTATAAGCT | 1380 |
| GCAATAAACA AGTTCTTTAA TCACCCAAAG CAGATGACCC TGCAAGACAT GGGTCGCATG | 1440 |
| TTTGGGGGCT GGCACCTGTT CCGACACATT GAAACCCGCT TTCAGCTCCT TGCCACTAAG | 1500 |
| AATGAGGGAT CCTTCAGCCC CGTGGCGAGT CTTCTCTCCC AGGGAGAGTA CCTCACGCGT | 1560 |
| CGGGACGATG TTAAGTACAG CAGCGATCAC CAGAACCGGT GGCAAAAAGG CGGACAACCG | 1620 |
| ATGACGGGGG GCATTGCTTA TGCGACCGGG AAAATGAGAC CCGACGAGCA ACAGTACCCT | 1680 |
| GCTATGCCCC CAGACCCCCC GATCATCACC GCTACTACAG CGCAAGGCAC GCAAGTCCGC | 1740 |
| TGCATGAATA GCACGCAAGC TTGGTGGTCA TGGGACACAT ATATGAGCTT TGCAACACTC | 1800 |
| ACAGCACTCG GTGCACAATG GTCTTTTCCT CCAGGGCAAC GTTCAGTTTC TAGACGGTCC | 1860 |
| TTCAACCACC ACAAGGCGAG AGGAGCCGGG GACCCCAAGG GCCAGAGATG GCACACGCTG | 1920 |
| GTGCCGCTCG GCACGGAGAC CATCACCGAC AGCTACATGT CAGCACCCGC ATCAGAGCTG | 1980 |
| GACACTAATT TCTTTACGCT TTACGTAGCG CAAGGCACAA ATAAGTCGCA ACAGTACAAG | 2040 |
| TTCGGCACAG CTACATACGC GCTAAAGGAG CCGGTAATGA AGAGCGATGC ATGGGCAGTG | 2100 |
| GTACGCGTCC AGTCGGTCTG GCAGCTGGGT AACAGGCAGA GGCCATACCC ATGGGACGTC | 2160 |
| AACTGGGCGA ACAGCACCAT GTACTGGGGG ACGCAGCCCT GAAAAGGGGG GGGGGCTAAA | 2220 |
| GCCCCCCCCC CTTAAACCCC CCCCTGGGGG GGATTCCCCC CCAGACCCCC CCTTTATATA | 2280 |
| GCACTCAATA AACGCAGAAA ATAGATTTAT CGCACTATC | 2319 |
| (SEQ ID NO: 4) | |

| Annotations: | |
|---|---|
| Putative Domain | Base range |
| nLuc insert | 407-1394 |
| 5′ UTR | 1-379 |
| VP2 | 380-406 |
| Apoptin | N/A |
| VP1 | 1394-2202 |
| 3′ UTR | 2203-2319 |

TABLE 5
CAV-nLuc4 nucleic acid sequence

Name CAV-nLuc4 mutant
Genus/Clade Gyrovirus
Full Sequence: 2319 bp

| | |
|---|---|
| GAATTCCGAG TGGTTACTAT TCCATCACCA TTCTAGCCTG TACACAGAAA GTCAAGATGG | 60 |
| ACGAATCGCT CGACTTCGCT CGCGATTCGT CGAAGGCGGG GGGCCGGAGG CCCCCCGGTG | 120 |
| GCCCCCCTCC AACGAGTGGA GCACGTACAG GGGGGTACGT CATCCGTACA GGGGGGTACG | 180 |
| TCATCCGTAC AGGGGGGTAC GTCACAAAGA GGCGTTCCCG TACAGGGGGG TACGTCACGC | 240 |
| GTACAGGGGG GTACGTCACA GCCAATCAAA AGCTGCCACG TTGCGAAAGT GACGTTTCGA | 300 |
| AAATGGGCGG CGCAAGCCTC TCTATATATT GAGCGCACAT ACCGGTCGGC AGTAGGTATA | 360 |
| CGCAAGGCGG TCCGGGTGGT TGCACGGGAA CGGCGGACAA CCGGCCGCTG GGGGCAGTGA | 420 |
| ATCGGCGCTT AGCCGAGAGG GGCAACCTGG GCCCAGCGGA GCCGCGCAGG GGCAAGTAAT | 480 |
| TTCAATTGAA CGCTCTCCAA GAAGATACTC CACCCGGACC ATCAACGGTG TTCAGGCCAC | 540 |
| CAACAAGTTC ACGGCCGTTG GAAACCCCTC ACTGCAGAGA GATCCGGATT GGTATCGCTG | 600 |
| GAATTACTGT GGAATGTGTG TCAGTTAGGG TGTGGAAAGT CCCCAGGCTC CCCAGCAGGC | 660 |
| AGAAGTATGC AAAGCATGCA TCTCAATTAG TCAGCAACCA GGTGTGGAAA GTCCCCAGGC | 720 |
| TCCCCAGCAG GCAGAAGTAT GCAAAGCATG CATCTCAATT AGTCAGCAAC CATAGTCCCG | 780 |
| CCCCTAACTC CGCCCATCCC GCCCCTAACT CCGCCCAGTT CCGCCCATTC TCCGCCCCAT | 840 |
| GGCTGACTAA TTTTTTTTAT TTATGCAGAG GCCGAGGCCG CCTCTGCCTC TGAGCTATTC | 900 |
| CAGAAGTAGT GAGGAGGCTT TTTTGGAGGC CTAGGCTTTT GCAAAAAGCT GCCACCATGG | 960 |
| TCTTCACACT CGAAGATTTC GTTGGGGACT GGCGACAGAC AGCCGGCTAC AACCTGGACC | 1020 |
| AAGTCCTTGA ACAGGGAGGT GTGTCCAGTT TGTTTCAGAA TCTCGGGGTG TCCGTAACTC | 1080 |
| CGATCCAAAG GATTGTCCTG AGCGGTGAAA ATGGGCTGAA GATCGACATC CATGTCATCA | 1140 |
| TCCCGTATGA AGGTCTGAGC GGCGACCAAA TGGGCCAGAT CGAAAAAATT TTTAAGGTGG | 1200 |
| TGTACCCTGT GGATGATCAT CACTTTAAGG TGATCCTGCA CTATGGCACA CTGGTAATCG | 1260 |
| ACGGGGTTAC GCCGAACATG ATCGACTATT TCGGACGGCC GTATGAAGGC ATCGCCGTGT | 1320 |
| TCGACGGCAA AAAGATCACT GTAACAGGGA CCCTGTGGAA CGGCAACAAA ATTATCGACG | 1380 |
| AGCGCCTGAT CAACCCCGAC GGCTCCCTGC TGTTCCGAGT AACCATCAAC GGAGTGACCG | 1440 |
| GCTGGCGGCT GTGCGAACGC ATTCTGGCGT AATAAGATAC ATTGATGAGT TTGGACAAAC | 1500 |
| CACAACTAGA ATGCAGTGAA AAAAATGCTT TATTTGTGAA ATTTGTGATG CTATTGCTTT | 1560 |
| ATTTGTAACC ATTATAAGCT GCAATAAACA AGTTACCGGT GGCAAAAAGG CGGACAACCG | 1620 |
| ATGACGGGGG GCATTGCTTA TGCGACCGGG AAAATGAGAC CCGACGAGCA ACAGTACCCT | 1680 |
| GCTATGCCCC CAGACCCCCC GATCATCACC GCTACTACAG CGCAAGGCAC GCAAGTCCGC | 1740 |
| TGCATGAATA GCACGCAAGC TTGGTGGTCA TGGGACACAT ATATGAGCTT TGCAACACTC | 1800 |
| ACAGCACTCG GTGCACAATG GTCTTTTCCT CCAGGGCAAC GTTCAGTTTC TAGACGGTCC | 1860 |
| TTCAACCACC ACAAGGCGAG AGGAGCCGGG GACCCCAAGG GCCAGAGATG GCACACGCTG | 1920 |
| GTGCCGCTCG GCACGGAGAC CATCACCGAC AGCTACATGT CAGCACCCGC ATCAGAGCTG | 1980 |
| GACACTAATT TCTTTACGCT TTACGTAGCG CAAGGCACAA ATAAGTCGCA ACAGTACAAG | 2040 |
| TTCGGCACAG CTACATACGC GCTAAAGGAG CCGGTAATGA AGAGCGATGC ATGGGCAGTG | 2100 |
| GTACGCGTCC AGTCGGTCTG GCAGCTGGGT AACAGGCAGA GGCCATACCC ATGGGACGTC | 2160 |
| AACTGGGCGA ACAGCACCAT GTACTGGGGG ACGCAGCCCT GAAAAGGGGG GGGGGCTAAA | 2220 |
| GCCCCCCCCC CTTAAACCCC CCCCTGGGGG GGATTCCCCC CCAGACCCCC CCTTTATATA | 2280 |
| GCACTCAATA AACGCAGAAA ATAGATTTAT CGCACTATC | 2319 |
| (SEQ ID NO: 5) | |

| Annotations: | |
|---|---|
| Putative Domain | Base range |
| nLuc insert | 607-1594 |
| 5′ UTR | 1-379 |
| VP2 | 380-606 |
| Apoptin | 486-606 |
| VP1 | 1595-2202 |
| 3′ UTR | 2203-2319 |

TABLE 6
CAV-nLuc5 nucleic acid sequence

Name CAV-nLuc5 mutant
Genus/Clade Gyrovirus
Full Sequence: 2319 bp

| | |
|---|---|
| GAATTCCGAG TGGTTACTAT TCCATCACCA TTCTAGCCTG TACACAGAAA GTCAAGATGG | 60 |
| ACGAATCGCT CGACTTCGCT CGCGATTCGT CGAAGGCGGG GGGCCGGAGG CCCCCCGGTG | 120 |
| GCCCCCCTCC AACGAGTGGA GCACGTACAG GGGGGTACGT CATCCGTACA GGGGGGTACG | 180 |
| TCATCCGTAC AGGGGGGTAC GTCACAAAGA GGCGTTCCCG TACAGGGGGG TACGTCACGC | 240 |
| GTACAGGGGG GTACGTCACA GCCAATCAAA AGCTGCCACG TTGCGAAAGT GACGTTTCGA | 300 |
| AAATGGGCGG CGCAAGCCTC TCTATATATT GAGCGCACAT ACCGGTCGGC AGTAGGTATA | 360 |
| CGCAAGGCGG TCCGGGTGGT TGCACGGGAA CGGCGGACAA CCGGCCGCTG GGGGCAGTGA | 420 |
| ATCGGCGCTT AGCCGAGAGG GGCAACCTGG GCCCAGCGGA GCCGCGCAGG GGCAAGTAAT | 480 |
| TTCAATTGAA CGCTCTCCAA GAAGATACTC CACCCGGACC ATCAACGGTG TTCAGGCCAC | 540 |
| CAACAAGTTC ACGGCCGTTG GAAACCCCTC ACTGCAGAGA GATCCGGATT GGTATCGCTG | 600 |
| GAATTACAAT CACTCTATCG CTGTGTGGCT GCGCGAATGC TCGCGCTCCC ACGCTAAGAT | 660 |
| CTGCAACTGC GGACAATTCA GAAAGCACTG GTTTCAAGAA TGTGCCGGAC TTGAGGACCG | 720 |
| ATCAACCCAA GCCTCCCTCG AAGAAGCGAT CCTGCGACCC CTCCGAGTAC AGGGTAAGCG | 780 |
| AGCTAAAAGA AAGCTTGATT ACCACTCTGT GGAATGTGTG TCAGTTAGGG TGTGGAAAGT | 840 |
| CCCCAGGCTC CCCAGCAGGC AGAAGTATGC AAAGCATGCA TCTCAATTAG TCAGCAACCA | 900 |
| GGTGTGGAAA GTCCCCAGGC TCCCCAGCAG GCAGAAGTAT GCAAAGCATG CATCTCAATT | 960 |
| AGTCAGCAAC CATAGTCCCG CCCCTAACTC CGCCCATCCC GCCCCTAACT CCGCCCAGTT | 1020 |
| CCGCCCATTC TCCGCCCCAT GGCTGACTAA TTTTTTTTAT TTATGCAGAG GCCGAGGCCG | 1080 |
| CCTCTGCCTC TGAGCTATTC CAGAAGTAGT GAGGAGGCTT TTTTGGAGGC CTAGGCTTTT | 1140 |
| GCAAAAAGCT GCCACCATGG TCTTCACACT CGAAGATTTC GTTGGGGACT GGCGACAGAC | 1200 |
| AGCCGGCTAC AACCTGGACC AAGTCCTTGA ACAGGGAGGT GTGTCCAGTT TGTTTCAGAA | 1260 |
| TCTCGGGGTG TCCGTAACTC CGATCCAAAG GATTGTCCTG AGCGGTGAAA ATGGGCTGAA | 1320 |
| GATCGACATC CATGTCATCA TCCCGTATGA AGGTCTGAGC GGCGACCAAA TGGGCCAGAT | 1380 |
| CGAAAAAATT TTTAAGGTGG TGTACCCTGT GGATGATCAT CACTTTAAGG TGATCCTGCA | 1440 |
| CTATGGCACA CTGGTAATCG ACGGGGTTAC GCCGAACATG ATCGACTATT TCGGACGGCC | 1500 |
| GTATGAAGGC ATCGCCGTGT TCGACGGCAA AAAGATCACT GTAACAGGGA CCCTGTGGAA | 1560 |
| CGGCAACAAA ATTATCGACG AGCGCCTGAT CAACCCCGAC GGCTCCCTGC TGTTCCGAGT | 1620 |
| AACCATCAAC GGAGTGACCG GCTGGCGGCT GTGCGAACGC ATTCTGGCGT AATAAGATAC | 1680 |
| ATTGATGAGT TTGGACAAAC CACAACTAGA ATGCAGTGAA AAAAATGCTT TATTTGTGAA | 1740 |
| ATTTGTGATG CTATTGCTTT ATTTGTAACC ATTATAAGCT GCAATAAACA AGTTACACTC | 1800 |
| ACAGCACTCG GTGCACAATG GTCTTTTCCT CCAGGGCAAC GTTCAGTTTC TAGACGGTCC | 1860 |
| TTCAACCACC ACAAGGCGAG AGGAGCCGGG GACCCCAAGG GCCAGAGATG GCACACGCTG | 1920 |
| GTGCCGCTCG GCACGGAGAC CATCACCGAC AGCTACATGT CAGCACCCGC ATCAGAGCTG | 1980 |
| GACACTAATT TCTTTACGCT TTACGTAGCG CAAGGCACAA ATAAGTCGCA ACAGTACAAG | 2040 |
| TTCGGCACAG CTACATACGC GCTAAAGGAG CCGGTAATGA AGAGCGATGC ATGGGCAGTG | 2100 |
| GTACGCGTCC AGTCGGTCTG GCAGCTGGGT AACAGGCAGA GGCCATACCC ATGGGACGTC | 2160 |
| AACTGGGCGA ACAGCACCAT GTACTGGGGG ACGCAGCCCT GAAAAGGGGG GGGGGCTAAA | 2220 |
| GCCCCCCCCC CTTAAACCCC CCCCTGGGGG GGATTCCCCC CCAGACCCCC CCTTTATATA | 2280 |
| GCACTCAATA AACGCAGAAA ATAGATTTAT CGCACTATC | 2319 |
| (SEQ ID NO: 6) | |

| Annotations: | |
|---|---|
| Putative Domain | Base range |
| nLuc insert | 807-1794 |
| 5′ UTR | 1-379 |
| VP2 | 380-806 |
| Apoptin | 486-806 |
| VP1 | 1795-2202 |
| 3′ UTR | 2203-2319 |

TABLE 7
CAV-nLuc6 nucleic acid sequence

Name CAV-nLuc6 mutant
Genus/Clade Gyrovirus
Full Sequence: 2319 bp

| | |
|---|---|
| GAATTCCGAG TGGTTACTAT TCCATCACCA TTCTAGCCTG TACACAGAAA GTCAAGATGG | 60 |
| ACGAATCGCT CGACTTCGCT CGCGATTCGT CGAAGGCGGG GGGCCGGAGG CCCCCCGGTG | 120 |
| GCCCCCCTCC AACGAGTGGA GCACGTACAG GGGGGTACGT CATCCGTACA GGGGGGTACG | 180 |
| TCATCCGTAC AGGGGGGTAC GTCACAAAGA GGCGTTCCCG TACAGGGGGG TACGTCACGC | 240 |
| GTACAGGGGG GTACGTCACA GCCAATCAAA AGCTGCCACG TTGCGAAAGT GACGTTTCGA | 300 |
| AAATGGGCGG CGCAAGCCTC TCTATATATT GAGCGCACAT ACCGGTCGGC AGTAGGTATA | 360 |
| CGCAAGGCGG TCCGGGTGGT TGCACGGGAA CGGCGGACAA CCGGCCGCTG GGGGCAGTGA | 420 |
| ATCGGCGCTT AGCCGAGAGG GGCAACCTGG GCCCAGCGGA GCCGCGCAGG GGCAAGTAAT | 480 |
| TTCAATTGAA CGCTCTCCAA GAAGATACTC CACCCGGACC ATCAACGGTG TTCAGGCCAC | 540 |
| CAACAAGTTC ACGGCCGTTG GAAACCCCTC ACTGCAGAGA GATCCGGATT GGTATCGCTG | 600 |
| GAATTACAAT CACTCTATCG CTGTGTGGCT GCGCGAATGC TCGCGCTCCC ACGCTAAGAT | 660 |
| CTGCAACTGC GGACAATTCA GAAAGCACTG GTTTCAAGAA TGTGCCGGAC TTGAGGACCG | 720 |
| ATCAACCCAA GCCTCCCTCG AAGAAGCGAT CCTGCGACCC CTCCGAGTAC AGGGTAAGCG | 780 |
| AGCTAAAAGA AAGCTTGATT ACCACTACTC CCAGCCGACC CCGAACCGCA AAAAGGCGTA | 840 |
| TAAGACTGTA AGTTGGCAAG ACGAGCTCGC AGACCGAGAG GCCGATTTTA CTCCTTCAGA | 900 |
| AGAGGACGGT GGCACCACCT CAAGCGACTT CGACGAAGAT ATAAATTTCG ACATCGGAGG | 960 |
| AGACAGCGGT ATCGTAGACG AGCTTTTAGG AAGGCCTTTC ACAACCCTGT GGAATGTGTG | 1020 |
| TCAGTTAGGG TGTGGAAAGT CCCCAGGCTC CCCAGCAGGC AGAAGTATGC AAAGCATGCA | 1080 |
| TCTCAATTAG TCAGCAACCA GGTGTGGAAA GTCCCCAGGC TCCCCAGCAG GCAGAAGTAT | 1140 |
| GCAAAGCATG CATCTCAATT AGTCAGCAAC CATAGTCCCG CCCCTAACTC CGCCCATCCC | 1200 |
| GCCCCTAACT CCGCCCAGTT CCGCCCATTC TCCGCCCCAT GGCTGACTAA TTTTTTTTAT | 1260 |
| TTATGCAGAG GCCGAGGCCG CCTCTGCCTC TGAGCTATTC CAGAAGTAGT GAGGAGGCTT | 1320 |
| TTTTGGAGGC CTAGGCTTTT GCAAAAAGCT GCCACCATGG TCTTCACACT CGAAGATTTC | 1380 |
| GTTGGGGACT GGCGACAGAC AGCCGGCTAC AACCTGGACC AAGTCCTTGA ACAGGGAGGT | 1440 |
| GTGTCCAGTT TGTTTCAGAA TCTCGGGGTG TCCGTAACTC CGATCCAAAG GATTGTCCTG | 1500 |
| AGCGGTGAAA ATGGGCTGAA GATCGACATC CATGTCATCA TCCCGTATGA AGGTCTGAGC | 1560 |
| GGCGACCAAA TGGGCCAGAT CGAAAAAATT TTTAAGGTGG TGTACCCTGT GGATGATCAT | 1620 |
| CACTTTAAGG TGATCCTGCA CTATGGCACA CTGGTAATCG ACGGGGTTAC GCCGAACATG | 1680 |
| ATCGACTATT TCGGACGGCC GTATGAAGGC ATCGCCGTGT TCGACGGCAA AAAGATCACT | 1740 |
| GTAACAGGGA CCCTGTGGAA CGGCAACAAA ATTATCGACG AGCGCCTGAT CAACCCCGAC | 1800 |
| GGCTCCCTGC TGTTCCGAGT AACCATCAAC GGAGTGACCG GCTGGCGGCT GTGCGAACGC | 1860 |
| ATTCTGGCGT AATAAGATAC ATTGATGAGT TTGGACAAAC CACAACTAGA ATGCAGTGAA | 1920 |
| AAAAATGCTT TATTTGTGAA ATTTGTGATG CTATTGCTTT ATTTGTAACC ATTATAAGCT | 1980 |
| GCAATAAACA AGTTTACGCT TTACGTAGCG CAAGGCACAA ATAAGTCGCA ACAGTACAAG | 2040 |
| TTCGGCACAG CTACATACGC GCTAAAGGAG CCGGTAATGA AGAGCGATGC ATGGGCAGTG | 2100 |
| GTACGCGTCC AGTCGGTCTG GCAGCTGGGT AACAGGCAGA GGCCATACCC ATGGGACGTC | 2160 |
| AACTGGGCGA ACAGCACCAT GTACTGGGGG ACGCAGCCCT GAAAAGGGGG GGGGGCTAAA | 2220 |
| GCCCCCCCCC CTTAAACCCC CCCCTGGGGG GGATTCCCCC CCAGACCCCC CCTTTATATA | 2280 |
| GCACTCAATA AACGCAGAAA ATAGATTTAT CGCACTATC | 2319 |
| (SEQ ID NO: 7) | |

| Annotations: | |
|---|---|
| Putative Domain | Base range |
| nLuc insert | 1007-1994 |
| 5′ UTR | 1-379 |
| VP2 | 380-1006 |
| Apoptin | 486-851 |
| VP1 | 853-1006; 1995-2202 |
| 3′ UTR | 2203-2319 |

TABLE 8
CAV-nLuc7 nucleic acid sequence

Name CAV-nLuc7 mutant
Genus/Clade Gyrovirus
Full Sequence: 2319 bp

| | |
|---|---|
| GAATTCCGAG TGGTTACTAT TCCATCACCA TTCTAGCCTG TACACAGAAA GTCAAGATGG | 60 |
| ACGAATCGCT CGACTTCGCT CGCGATTCGT CGAAGGCGGG GGGCCGGAGG CCCCCCGGTG | 120 |
| GCCCCCCTCC AACGAGTGGA GCACGTACAG GGGGGTACGT CATCCGTACA GGGGGGTACG | 180 |
| TCATCCGTAC AGGGGGGTAC GTCACAAAGA GGCGTTCCCG TACAGGGGGG TACGTCACGC | 240 |
| GTACAGGGGG GTACGTCACA GCCAATCAAA AGCTGCCACG TTGCGAAAGT GACGTTTCGA | 300 |
| AAATGGGCGG CGCAAGCCTC TCTATATATT GAGCGCACAT ACCGGTCGGC AGTAGGTATA | 360 |
| CGCAAGGCGG TCCGGGTGGT TGCACGGGAA CGGCGGACAA CCGGCCGCTG GGGGCAGTGA | 420 |
| ATCGGCGCTT AGCCGAGAGG GGCAACCTGG GCCCAGCGGA GCCGCGCAGG GGCAAGTAAT | 480 |
| TTCAATTGAA CGCTCTCCAA GAAGATACTC CACCCGGACC ATCAACGGTG TTCAGGCCAC | 540 |
| CAACAAGTTC ACGGCCGTTG GAAACCCCTC ACTGCAGAGA GATCCGGATT GGTATCGCTG | 600 |
| GAATTACAAT CACTCTATCG CTGTGTGGCT GCGCGAATGC TCGCGCTCCC ACGCTAAGAT | 660 |
| CTGCAACTGC GGACAATTCA GAAAGCACTG GTTTCAAGAA TGTGCCGGAC TTGAGGACCG | 720 |
| ATCAACCCAA GCCTCCCTCG AAGAAGCGAT CCTGCGACCC CTCCGAGTAC AGGGTAAGCG | 780 |
| AGCTAAAAGA AAGCTTGATT ACCACTACTC CCAGCCGACC CCGAACCGCA AAAAGGCGTA | 840 |
| TAAGACTGTA AGTTGGCAAG ACGAGCTCGC AGACCGAGAG GCCGATTTTA CTCCTTCAGA | 900 |
| AGAGGACGGT GGCACCACCT CAAGCGACTT CGACGAAGAT ATAAATTTCG ACATCGGAGG | 960 |
| AGACAGCGGT ATCGTAGACG AGCTTTTAGG AAGGCCTTTC ACAACCCCCG CCCCGGTACG | 1020 |
| TATAGTGTGA GGCTGCCGAA CCCCCAATCT ACTATGACTA TCCGCTTCCA AGGGGTCATC | 1080 |
| TTTCTCACGG AAGGACTCAT TCTGCCTAAA AACAGCACAG CGGGGGGCTA TGCAGACCAC | 1140 |
| ATGTACGGGG CGAGAGTCGC CAAGATCTCT GTGAACCTGA AAGAGTTCCT GCTAGCCTCA | 1200 |
| ATGAACCTGT GGAATGTGTG TCAGTTAGGG TGTGGAAAGT CCCCAGGCTC CCCAGCAGGC | 1260 |
| AGAAGTATGC AAAGCATGCA TCTCAATTAG TCAGCAACCA GGTGTGGAAA GTCCCCAGGC | 1320 |
| TCCCCAGCAG GCAGAAGTAT GCAAAGCATG CATCTCAATT AGTCAGCAAC CATAGTCCCG | 1380 |
| CCCCTAACTC CGCCCATCCC GCCCCTAACT CCGCCCAGTT CCGCCCATTC TCCGCCCCAT | 1440 |
| GGCTGACTAA TTTTTTTTAT TTATGCAGAG GCCGAGGCCG CCTCTGCCTC TGAGCTATTC | 1500 |
| CAGAAGTAGT GAGGAGGCTT TTTTGGAGGC CTAGGCTTTT GCAAAAAGCT GCCACCATGG | 1560 |
| TCTTCACACT CGAAGATTTC GTTGGGGACT GGCGACAGAC AGCCGGCTAC AACCTGGACC | 1620 |
| AAGTCCTTGA ACAGGGAGGT GTGTCCAGTT TGTTTCAGAA TCTCGGGGTG TCCGTAACTC | 1680 |
| CGATCCAAAG GATTGTCCTG AGCGGTGAAA ATGGGCTGAA GATCGACATC CATGTCATCA | 1740 |
| TCCCGTATGA AGGTCTGAGC GGCGACCAAA TGGGCCAGAT CGAAAAAATT TTTAAGGTGG | 1800 |
| TGTACCCTGT GGATGATCAT CACTTTAAGG TGATCCTGCA CTATGGCACA CTGGTAATCG | 1860 |
| ACGGGGTTAC GCCGAACATG ATCGACTATT TCGGACGGCC GTATGAAGGC ATCGCCGTGT | 1920 |
| TCGACGGCAA AAAGATCACT GTAACAGGGA CCCTGTGGAA CGGCAACAAA ATTATCGACG | 1980 |
| AGCGCCTGAT CAACCCCGAC GGCTCCCTGC TGTTCCGAGT AACCATCAAC GGAGTGACCG | 2040 |
| GCTGGCGGCT GTGCGAACGC ATTCTGGCGT AATAAGATAC ATTGATGAGT TTGGACAAAC | 2100 |
| CACAACTAGA ATGCAGTGAA AAAAATGCTT TATTTGTGAA ATTTGTGATG CTATTGCTTT | 2160 |
| ATTTGTAACC ATTATAAGCT GCAATAAACA AGTTAGCCCT GAAAAGGGGG GGGGGCTAAA | 2220 |
| GCCCCCCCCC CTTAAACCCC CCCCTGGGGG GGATTCCCCC CCAGACCCCC CCTTTATATA | 2280 |
| GCACTCAATA AACGCAGAAA ATAGATTTAT CGCACTATC | 2319 |
| (SEQ ID NO: 8) | |

| Annotations: | |
|---|---|
| Putative Domain | Base range |
| nLuc insert | 1207-2194 |
| 5′ UTR | 1-379 |
| VP2 | 380-1030 |
| Apoptin | 486-851 |
| VP1 | 853-1206; 2195-2202 |
| 3′ UTR | 2203-2319 |

TABLE 9
CAV-nLuc8 nucleic acid sequence

Name CAV-nLuc8 mutant
Genus/Clade Gyrovirus
Full Sequence: 2319 bp

| | |
|---|---|
| GAATTCCGAG TGGTTACTAT TCCATCACCA TTCTAGCCTG TACACAGAAA GTCAAGATGG | 60 |
| ACGAATCGCT CGACTTCGCT CGCGATTCGT CGAAGGCGGG GGGCCGGAGG CCCCCCGGTG | 120 |
| GCCCCCCTCC AACGAGTGGA GCACGTACAG GGGGGTACGT CATCCGTACA GGGGGGTACG | 180 |
| TCATCCGTAC AGGGGGGTAC GTCACAAAGA GGCGTTCCCG TACAGGGGGG TACGTCACGC | 240 |
| GTACAGGGGG GTACGTCACA GCCAATCAAA AGCTGCCACG TTGCGAAAGT GACGTTTCGA | 300 |
| AAATGGGCGG CGCAAGCCTC TCTATATATT GAGCGCACAT ACCGGTCGGC AGTAGGTATA | 360 |
| CGCAAGGCGG TCCGGGTGGA TGCACGGGAA CGGCGGACAA CCGGCCGCTG GGGGCAGTGA | 420 |
| ATCGGCGCTT AGCCGAGAGG GGCAACCTGG GCCCAGCGGA GCCGCGCAGG GGCAAGTAAT | 480 |
| TTCAAATGAA CGCTCTCCAA GAAGATACTC CACCCGGACC ATCAACGGTG TTCAGGCCAC | 540 |
| CAACAAGTTC ACGGCCGTTG GAAACCCCTC ACTGCAGAGA GATCCGGATT GGTATCGCTG | 600 |
| GAATTACAAT CACTCTATCG CTGTGTGGCT GCGCGAATGC TCGCGCTCCC ACGCTAAGAT | 660 |
| CTGCAACTGC GGACAATTCA GAAAGCACTG GTTTCAAGAA TGTGCCGGAC TTGAGGACCG | 720 |
| ATCAACCCAA GCCTCCCTCG AAGAAGCGAT CCTGCGACCC CTCCGAGTAC AGGGTAAGCG | 780 |
| AGCTAAAAGA AAGCTTGATT ACCACTACTC CCAGCCGACC CCGAACCGCA AAAAGGCGTA | 840 |
| TAAGACTGTA AGATGGCAAG ACGAGCTCGC AGACCGAGAG GCCGATTTTA CTCCTTCAGA | 900 |
| AGAGGACGGT GGCACCACCT CAAGCGACTT CGACGAAGAT ATAAATTTCG ACATCGGAGG | 960 |
| AGACAGCGGT ATCGTAGACG AGCTTTTAGG AAGGCCTTTC ACAACCCCCG CCCCGGTACG | 1020 |
| TATAGTGTGA GGCTGCCGAA CCCCCAATCT ACTATGACTA TCCGCTTCCA AGGGGTCATC | 1080 |
| TTTCTCACGG AAGGACTCAT TCTGCCTAAA AACAGCACAG CGGGGGGCTA TGCAGACCAC | 1140 |
| ATGTACGGGG CGAGAGTCGC CAAGATCTCT GTGAACCTGA AAGAGTTCCT GCTAGCCTCA | 1200 |
| ATGAACCTGA CATACGTGAG CAAAATCGGA GGCCCCATCG CCGGTGAGTT GATTGCGGAC | 1260 |
| GGGTCTAAAT CCTGTGGAAT GTGTGTCAGT TAGGGTGTGG AAAGTCCCCA GGCTCCCCAG | 1380 |
| CAGGCAGAAG TATGCAAAGC ATGCATCTCA ATTAGTCAGC AACCAGGTGT GGAAAGTCCC | 1440 |
| CAGGCTCCCC AGCAGGCAGA AGTATGCAAA GCATGCATCT CAATTAGTCA GCAACCATAG | 1500 |
| TCCCGCCCCT AACTCCGCCC ATCCCGCCCC TAACTCCGCC CAGTTCCGCC CATTCTCCGC | 1560 |
| CCCATGGCTG ACTAATTTTT TTTATTTATG CAGAGGCCGA GGCCGCCTCT GCCTCTGAGC | 1620 |
| TATTCCAGAA GTAGTGAGGA GGCTTTTTTG GAGGCCTAGG CTTTTGCAAA AAGCTGCCAC | 1680 |
| CATGGTCTTC ACACTCGAAG ATTTCGTTGG GGACTGGCGA CAGACAGCCG GCTACAACCT | 1740 |
| GGACCAAGTC CTTGAACAGG GAGGTGTGTC CAGTTTGTTT CAGAATCTCG GGGTGTCCGT | 1800 |
| AACTCCGATC CAAAGGATTG TCCTGAGCGG TGAAAATGGG CTGAAGATCG ACATCCATGT | 1860 |
| CATCATCCCG TATGAAGGTC TGAGCGGCGA CCAAATGGGC CAGATCGAAA AAATTTTTAA | 1920 |
| GGTGGTGTAC CCTGTGGATG ATCATCACTT TAAGGTGATC CTGCACTATG GCACACTGGT | 1980 |
| AATCGACGGG GTTACGCCGA ACATGATCGA CTATTTCGGA CGGCCGTATG AAGGCATCGC | 2040 |
| CGTGTTCGAC GGCAAAAAGA TCACTGTAAC AGGGACCCTG TGGAACGGCA ACAAAATTAT | 2100 |
| CGACGAGCGC CTGATCAACC CCGACGGCTC CCTGCTGTTC CGAGTAACCA TCAACGGAGT | 2160 |
| GACCGGCTGG CGGCTGTGCG AACGCATTCT GGCGTAATAA GATACATTGA TGAGTTTGGA | 2220 |
| CAAACCACAA CTAGAATGCA GTGAAAAAAA TGCTTTATTT GTGAAATTTG TGATGCTATT | 2280 |
| GCTTTATTTG TAACCATTAT AAGCTGCAAT AAACAAGTT | 2319 |

| Annotations: | |
|---|---|
| Putative Domain | Base range |
| nLuc insert | 1272-2319 |
| 5′ UTR | 1-379 |
| VP2 | 380-1030 |
| Apoptin | 486-851 |
| VP1 | 853-1271 |
| 3′ UTR | N/A |

TABLE 10
CRE-CA Vector nucleic acid sequence

| | |
|---|---|
| Name | CRE-CA Vector |
| Genus/Clade | Gyrovirus |

Full Sequence: 2319 bp

| | |
|---|---|
| GAATTCCGAG TGGTTACTAT TCCATCACCA TTCTAGCCTG TACACAGAAA GTCAAGATGG | 60 |
| ACGAATCGCT CGACTTCGCT CGCGATTCGT CGAAGGCGGG GGGCCGGAGG CCCCCCGGTG | 120 |
| GCCCCCCTCC AACGAGTGGA GCACGTACAG GGGGGTACGT CATCCGTACA GGGGGGTACG | 180 |
| TCATCCGTAC AGGGGGGTAC GTCACAAAGA GGCGTTCCCG TACAGGGGGG TACGTCACGC | 240 |
| GTACAGGGGG GTACGTCACA GCCAATCAAA AGCTGCCACG TTGCGAAAGT GACGTTTCGA | 300 |
| AAATGGGCGG CGCAAGCCTC TCTATATATT GAGCGCACAT ACCGGTCGGC AGTAGGTATA | 360 |
| CGCAAGGCGG TCCGGGTGGT TGCACGGGAA CGGCGGACAA CCGGCCGCTG GGGGCAGTGA | 420 |
| ATCGGCGCTT AGCCGAGAGG GGCAACCTGG GCCCAGCGGA GCCGCGCAGG GGCAAGTAAT | 480 |
| TTCAATTGAA CGCTCTCCAA GAAGATACTC CACCCGGACC ATCAACGGTG TTCAGGCCAC | 540 |
| CAACAACTGT GGAATGTGTG TCAGTTAGGG TGTGGAAAGT CCCCAGGCTC CCCAGCAGGC | 660 |
| AGAAGTATGC AAAGCATGCA TCTCAATTAG TCAGCAACCA GGTGTGGAAA GTCCCCAGGC | 720 |
| TCCCCAGCAG GCAGAAGTAT GCAAAGCATG CATCTCAATT AGTCAGCAAC CATAGTCCCG | 780 |
| CCCCTAACTC CGCCCATCCC GCCCCTAACT CCGCCCAGTT CCGCCCATTC TCCGCCCCAT | 840 |
| GGCTGACTAA TTTTTTTTAT TTATGCAGAG GCCGAGGCCG CCTCTGCCTC TGAGCTATTC | 900 |
| CAGAAGTAGT GAGGAGGCTT TTTTGGAGGC CTAGGCTTTT GCAAAAAGCT GCCACCATGG | 960 |
| TGCCCAAGAA GAAGAGGAAA GTCTCCAACC TGCTGACTGT GCACCAAAAC CTGCCTGCCC | 1020 |
| TCCCTGTGGA TGCCACCTCT GATGAAGTCA GGAAGAACCT GATGGACATG TTCAGGGACA | 1080 |
| GGCAGGCCTT CTCTGAACAC ACCTGGAAGA TGCTCCTGTC TGTGTGCAGA TCCTGGGCTG | 1140 |
| CCTGGTGCAA GCTGAACAAC AGGAAATGGT TCCCTGCTGA ACCTGAGGAT GTGAGGGACT | 1200 |
| ACCTCCTGTA CCTGCAAGCC AGAGGCCTGG CTGTGAAAAC CATCCAACAG CACCTGGGCC | 1260 |
| AGCTCAACAT GCTGCACAGG AGATCTGGCC TGCCTCGCCC TTCTGACTCC AATGCTGTGT | 1320 |
| CCCTGGTGAT GAGGAGAATC AGAAAGGAGA ATGTGGATGC TGGGGAGAGA GCCAAGCAGG | 1380 |
| CCCTGGCCTT TGAACGCACT GACTTTGACC AAGTCAGATC CCTGATGGAG AACTCTGACA | 1440 |
| GATGCCAGGA CATCAGGAAC CTGGCCTTCC TGGGCATTGC CTACAACACC CTGCTGCGCA | 1500 |
| TTGCCGAAAT TGCCAGAATC AGAGTGAAGG ACATCTCCCG CACCGATGGT GGGAGAATGC | 1560 |
| TGATCCACAT TGGCAGGACC AAGACCCTGG TGTCCACAGC TGGTGTGGAG AAGGCCCTGT | 1620 |
| CCCTGGGGGT TACCAAGCTG GTGGAGAGAT GGATCTCTGT GTCTGGTGTG GCTGATGACC | 1680 |
| CCAACAACTA CCTGTTCTGC CGGGTCAGAA AGAATGGTGT GGCTGCCCCT TCTGCCACCT | 1740 |
| CCCAACTGTC CACCCGCGCC CTGGAAGGGA TCTTTGAGGC CACCCACCGC CTGATCTATG | 1800 |
| GTGCCAAGGA TGACTCTGGG CAGAGATACC TGGCCTGGTC TGGCCACTCT GCCAGAGTGG | 1860 |
| GTGCTGCCAG GGACATGGCC AGGGCTGGTG TGTCCATCCC TGAAATCATG CAGGCTGGTG | 1920 |
| GCTGGACCAA TGTGAACATA GTGATGAACT ACATCAGAAA CCTGGACTCT GAGACTGGGG | 1980 |
| CCATGGTGAG GCTGCTCGAG GATGGGGACT GATAAGATAC ATTGATGAGT TTGGACAAAC | 2040 |
| CACAACTAGA ATGCAGTGAA AAAAATGCTT TATTTGTGAA ATTTGTGATG CTATTGCTTT | 2100 |
| ATTTGTAACC ATTATAAGCT GCAATAAACA AGTTGGCAGA GGCCATACCC ATGGGACGTC | 2160 |
| AACTGGGCGA ACAGCACCAT GTACTGGGGG ACGCAGCCCT GAAAAGGGGG GGGGGCTAAA | 2220 |
| GCCCCCCCCC CTTAAACCCC CCCCTGGGGG GGATTCCCCC CCAGACCCCC CCTTTATATA | 2280 |
| GCACTCAATA AACGCAGAAA ATAGATTTAT CGCACTATC | 2319 |

(SEQ ID NO: 10)
Annotations:

| Putative Domain | Base range |
|---|---|
| iCRE insert | 547-2134 |
| 5′ UTR | 1-379 |
| VP2 | 380-546 |
| Apoptin | 486-546 |
| VP1 | 2135-2202 |
| 3′ UTR | 2203-2319 |

TABLE 11
GFP-CA Vector nucleic acid sequence

| | |
|---|---|
| Name | GFP-CA Vector |
| Genus/Clade | Gyrovirus |
| Full Sequence: 2319 bp | |

| | |
|---|---|
| GAATTCCGAG TGGTTACTAT TCCATCACCA TTCTAGCCTG TACACAGAAA GTCAAGATGG | 60 |
| ACGAATCGCT CGACTTCGCT CGCGATTCGT CGAAGGCGGG GGGCCGGAGG CCCCCCGGTG | 120 |
| GCCCCCCTCC AACGAGTGGA GCACGTACAG GGGGGTACGT CATCCGTACA GGGGGGTACG | 180 |
| TCATCCGTAC AGGGGGGTAC GTCACAAAGA GGCGTTCCCG TACAGGGGGG TACGTCACGC | 240 |
| GTACAGGGGG GTACGTCACA GCCAATCAAA AGCTGCCACG TTGCGAAAGT GACGTTTCGA | 300 |
| AAATGGGCGG CGCAAGCCTC TCTATATATT GAGCGCACAT ACCGGTCGGC AGTAGGTATA | 360 |
| CGCAAGGCGG TCCGGGTGGT TGCACGGGAA CGGCGGACAA CCGGCCGCTG GGGGCAGTGA | 420 |
| ATCGGCGCTT AGCCGAGAGG GGCAACCTGG GCCCAGCGGA GCCGCGCAGG GGCAAGTAAT | 480 |
| TTCAATTGAA CGCTCTCCAA GAAGATACTC CACCCGGACC ATCAACGGTG TTCAGGCCAC | 540 |
| CAACAAGTTC ACGGCCGTTG GAAACCCCTC ACTGCAGAGA GATCCGGATT GGTATCGCTG | 600 |
| GAATTACTGT GGAATGTGTG TCAGTTAGGG TGTGGAAAGT CCCCAGGCTC CCCAGCAGGC | 660 |
| AGAAGTATGC AAAGCATGCA TCTCAATTAG TCAGCAACCA GGTGTGGAAA GTCCCCAGGC | 720 |
| TCCCCAGCAG GCAGAAGTAT GCAAAGCATG CATCTCAATT AGTCAGCAAC CATAGTCCCG | 780 |
| CCCCTAACTC CGCCCATCCC GCCCCTAACT CCGCCCAGTT CCGCCCATTC TCCGCCCCAT | 840 |
| GGCTGACTAA TTTTTTTTAT TTATGCAGAG GCCGAGGCCG CCTCTGCCTC TGAGCTATTC | 900 |
| CAGAAGTAGT GAGGAGGCTT TTTTGGAGGC CTAGGCTTTT GCAAAAAGCT GCCACCATGG | 960 |
| TGAGCAAGGG CGAGGAGCTG TTCACCGGGG TGGTGCCCAT CCTGGTCGAG CTGGACGGCG | 1020 |
| ACGTAAACGG CCACAAGTTC AGCGTGTCCG GCGAGGGCGA GGGCGATGCC ACCTACGGCA | 1080 |
| AGCTGACCCT GAAGTTCATC TGCACCACCG GCAAGCTGCC CGTGCCCTGG CCCACCCTCG | 1140 |
| TGACCACCCT GACCTACGGC GTGCAGTGCT TCAGCCGCTA CCCCGACCAC ATGAAGCAGC | 1200 |
| ACGACTTCTT CAAGTCCGCC ATGCCCGAAG GCTACGTCCA GGAGCGCACC ATCTTCTTCA | 1260 |
| AGGACGACGG CAACTACAAG ACCCGCGCCG AGGTGAAGTT CGAGGGCGAC ACCCTGGTGA | 1320 |
| ACCGCATCGA GCTGAAGGGC ATCGACTTCA AGGAGGACGG CAACATCCTG GGGCACAAGC | 1380 |
| TGGAGTACAA CTACAACAGC CACAACGTCT ATATCATGGC CGACAAGCAG AAGAACGGCA | 1440 |
| TCAAGGTGAA CTTCAAGATC CGCCACAACA TCGAGGACGG CAGCGTGCAG CTCGCCGACC | 1500 |
| ACTACCAGCA GAACACCCCC ATCGGCGACG GCCCCGTGCT GCTGCCCGAC AACCACTACC | 1560 |
| TGAGCACCCA GTCCGCCCTG AGCAAAGACC CCAACGAGAA GCGCGATCAC ATGGTCCTGC | 1620 |
| TGGAGTTCGT GACCGCCGCC GGGATCACTC TCGGCATGGA CGAGCTGTAC AAGTAATAAG | 1680 |
| ATACATTGAT GAGTTTGGAC AAACCACAAC TAGAATGCAG TGAAAAAAAT GCTTTATTTG | 1740 |
| TGAAATTTGT GATGCTATTG CTTTATTTGT AACCATTATA AGCTGCAATA AACAAGTTTC | 1800 |
| ACAGCACTCG GTGCACAATG GTCTTTTCCT CCAGGGCAAC GTTCAGTTTC TAGACGGTCC | 1860 |
| TTCAACCACC ACAAGGCGAG AGGAGCCGGG GACCCCAAGG GCCAGAGATG GCACACGCTG | 1920 |
| GTGCCGCTCG GCACGGAGAC CATCACCGAC AGCTACATGT CAGCACCCGC ATCAGAGCTG | 1980 |
| GACACTAATT TCTTTACGCT TTACGTAGCG CAAGGCACAA ATAAGTCGCA ACAGTACAAG | 2040 |
| TTCGGCACAG CTACATACGC GCTAAAGGAG CCGGTAATGA AGAGCGATGC ATGGGCAGTG | 2100 |
| GTACGCGTCC AGTCGGTCTG GCAGCTGGGT AACAGGCAGA GGCCATACCC ATGGGACGTC | 2160 |
| AACTGGGCGA ACAGCACCAT GTACTGGGGG ACGCAGCCCT GAAAAGGGGG GGGGGCTAAA | 2220 |
| GCCCCCCCCC CTTAAACCCC CCCCTGGGGG GGATTCCCCC CCAGACCCCC CCTTTATATA | 2280 |
| GCACTCAATA AACGCAGAAA ATAGATTTAT CGCACTATC | 2319 |

(SEQ ID NO: 11)
Annotations:

| Putative Domain | Base range |
|---|---|
| GFP insert | 607-1797 |
| 5′ UTR | 1-379 |
| VP2 | 380-606 |
| Apoptin | 486-606 |
| VP1 | 1798-2202 |
| 3′ UTR | 2203-2319 |

TABLE 12
Gluc-CA Vector nucleic acid sequence

| | |
|---|---|
| Name | Gluc-CAVector |
| Genus/Clade | Gyrovirus |

Full Sequence: 2319 bp

| | |
|---|---|
| GAATTCCGAG TGGTTACTAT TCCATCACCA TTCTAGCCTG TACACAGAAA CTCAAGATGG | 60 |
| ACGAATCGCT CGACTTCGCT CGCGATTCGT CGAAGGCGGG GGGCCGGAGG CCCCCCGGTG | 120 |
| GCCCCCCTCC AACGAGTGGA GCACGTACAG GGGGGTACGT CATCCGTACA GGGGGGTACG | 180 |
| TCATCCGTAC AGGGGGGTAC GTCACAAAGA GGCGTTCCCG TACAGGGGGG TACGTCACGC | 240 |
| GTACAGGGGG GTACGTCACA GCCAATCAAA AGCTGCCACG TTGCGAAAGT GACGTTTCGA | 300 |
| AAATGGGCGG CGCAAGCCTC TCTATATATT GAGCGCACAT ACCGGTCGGC AGTAGGTATA | 360 |
| CGCAAGGCGG TCCGGGTGGT TGCACGGGAA CGGCGGACAA CCGGCCGCTG GGGGCAGTGA | 420 |
| ATCGGCGCTT AGCCGAGAGG GGCAACCTGG GCCCAGCGGA GCCGCGCAGG GGCAAGTAAT | 480 |
| TTCAATTGAA CGCTCTCCAA GAAGATACTC CACCCGGACC ATCAACGGTG TTCAGGCCAC | 540 |
| CAACAAGTTC ACGGCCGTTG GAAACCCCTC ACTGCAGAGA GATCCGGATT GGTATCGCTG | 600 |
| GAATTACTGT GGAATGTGTG TCAGTTAGGG TGTGGAAAGT CCCCAGGCTC CCCAGCAGGC | 660 |
| AGAAGTATGC AAAGCATGCA TCTCAATTAG TCAGCAACCA GGTGTGGAAA GTCCCCAGGC | 720 |
| TCCCCAGCAG GCAGAAGTAT GCAAAGCATG CATCTCAATT AGTCAGCAAC CATAGTCCCG | 780 |
| CCCCTAACTC CGCCCATCCC GCCCCTAACT CCGCCCAGTT CCGCCCATTC TCCGCCCCAT | 840 |
| GGCTGACTAA TTTTTTTTAT TTATGCAGAG GCCGAGGCCG CCTCTGCCTC TGAGCTATTC | 900 |
| CAGAAGTAGT GAGGAGGCTT TTTTGGAGGC CTAGGCTTTT GCAAAAAGCT GCCACCATGG | 960 |
| CGTCTAAAGT GTATGACCCA GAGCAGAGAA AACGAATGAT CACCGGCCCC CAATGGTGGG | 1020 |
| CTCGGTGCAA GCAGATGAAT GTGCTTGATA GCTTTATAAA CTACTATGAT AGTGAGAAAC | 1080 |
| ACGCAGAAAA TGCCGTGATA TTCTTGCATG GGAACGCAGC ATCTTCTTAC CTTTGGAGGC | 1140 |
| ATGTTGTTCC ACATATCGAA CCAGTAGCCA GGTGCATCAT CCCAGATTTG ATTGGTATGG | 1200 |
| GGAAATCTGG CAAGAGTGGC AACGGTAGTT ATCGACTGCT TGACCATTAT AAGTATCTGA | 1260 |
| CCGCCTGGTT CGAGCTCCTT AACCTTCCAA AAAAAATAAT TTTTGTGGGC CATGACTGGG | 1320 |
| GCGCATGTCT TGCATTCCAC TACAGTTATG AACATCAAGA TAAGATTAAG GCCATAGTAC | 1380 |
| ACGCAGAGTC CGTGGTTGAC GTTATCGAGT CTTGGGATGA ATGGCCTGAT ATTGAAGAGG | 1440 |
| ACATAGCACT TATTAAGAGC GAGGAGGGAG AGAAGATGGT TCTTGAAAAC AACTTTTTTG | 1500 |
| TCGAAACCAT GTTGCCAAGC AAGATAATGC GAAAGCTGGA ACCAGAGGAA TTCGCAGCCT | 1560 |
| ATCTTGAGCC GTTCAAGGAG AAAGGGGAGG TAAGACGCCC GACATTGTCA TGGCCTCGAG | 1620 |
| AAATCCCCCT CGTCAAAGGT GGTAAACCAG ATGTGGTTCA AATTGTCAGG AACTATAACG | 1680 |
| CCTACCTCAG GGCGTCAGAT GACCTCCCCA AAATGTTTAT AGAGTCTGAT CCGGGATTTT | 1740 |
| TCTCTAATGC CATAGTAGAG GGTGCTAAGA AGTTCCCTAA CACTGAATTT GTAAAAGTTA | 1800 |
| AAGGGCTCCA TTTCTCACAG GAAGATGCGC CAGATGAAAT GGGCAAATAC ATTAAAAGTT | 1860 |
| TCGTAGAGCG GGTACTGAAA AATGAGCAGT AATAAGATAC ATTGATGAGT TTGGACAAAC | 1920 |
| CACAACTAGA ATGCAGTGAA AAAAATGCTT TATTTGTGAA ATTTGTGATG CTATTGCTTT | 1980 |
| ATTTGTAACC ATTATAAGCT GCAATAAACA AGTTGCACAA ATAAGTCGCA ACAGTACAAG | 2040 |
| TTCGGCACAG CTACATACGC GCTAAAGGAG CCGGTAATGA AGAGCGATGC ATGGGCAGTG | 2100 |
| GTACGCGTCC AGTCGGTCTG GCAGCTGGGT AACAGGCAGA GGCCATACCC ATGGGACGTC | 2160 |
| AACTGGGCGA ACAGCACCAT GTACTGGGGG ACGCAGCCCT GAAAAGGGGG GGGGGCTAAA | 2220 |
| GCCCCCCCCC CTTAAACCCC CCCCTGGGGG GGATTCCCCC CCAGACCCCC CCTTTATATA | 2280 |
| GCACTCAATA AACGCAGAAA ATAGATTTAT CGCACTATC | 2319 |

(SEQ ID NO: 12)
Annotations:

| Putative Domain | Base range |
|---|---|
| Gluc insert | 607-2014 |
| 5′ UTR | 1-379 |
| VP2 | 380-606 |
| Apoptin | 486-606 |
| VP1 | 2015-2202 |
| 3′ UTR | 2203-2319 |

TABLE 13
mCherry-CA Vector nucleic acid sequence

| | |
|---|---|
| Name | mCherry-CA Vector |
| Genus/Clade | Gyrovirus |

Full Sequence: 2319 bp

| | |
|---|---|
| GAATTCCGAG TGGTTACTAT TCCATCACCA TTCTAGCCTG TACACAGAAA GTCAAGATGG | 60 |
| ACGAATCGCT CGACTTCGCT CGCGATTCGT CGAAGGCGGG GGGCCGGAGG CCCCCCGGTG | 120 |
| GCCCCCCTCC AACGAGTGGA GCACGTACAG GGGGGTACGT CATCCGTACA GGGGGGTACG | 180 |
| TCATCCGTAC AGGGGGGTAC GTCACAAAGA GGCGTTCCCG TACAGGGGGG TACGTCACGC | 240 |
| GTACAGGGGG GTACGTCACA GCCAATCAAA AGCTGCCACG TTGCGAAAGT GACGTTTCGA | 300 |
| AAATGGGCGG CGCAAGCCTC TCTATATATT GAGCGCACAT ACCGGTCGGC AGTAGGTATA | 360 |
| CGCAAGGCGG TCCGGGTGGT TGCACGGGAA CGGCGGACAA CCGGCCGCTG GGGGCAGTGA | 420 |
| ATCGGCGCTT AGCCGAGAGG GGCAACCTGG GCCCAGCGGA GCCGCGCAGG GGCAAGTAAT | 480 |
| TTCAATTGAA CGCTCTCCAA GAAGATACTC CACCCGGACC ATCAACGGTG TTCAGGCCAC | 540 |
| CAACAAGTTC ACGGCCGTTG GAAACCCCTC ACTGCAGAGA GATCCGGATT GGTATCGCTG | 600 |
| GAATTACTGT GGAATGTGTG TCAGTTAGGG TGTGGAAAGT CCCCAGGCTC CCCAGCAGGC | 660 |
| AGAAGTATGC AAAGCATGCA TCTCAATTAG TCAGCAACCA GGTGTGGAAA GTCCCCAGGC | 720 |
| TCCCCAGCAG GCAGAAGTAT GCAAAGCATG CATCTCAATT AGTCAGCAAC CATAGTCCCG | 780 |
| CCCCTAACTC CGCCCATCCC GCCCCTAACT CCGCCCAGTT CCGCCCATTC TCCGCCCCAT | 840 |
| GGCTGACTAA TTTTTTTTAT TTATGCAGAG GCCGAGGCCG CCTCTGCCTC TGAGCTATTC | 900 |
| CAGAAGTAGT GAGGAGGCTT TTTTGGAGGC CTAGGCTTTT GCAAAAAGCT GCCACCATGG | 960 |
| TGAGCAAGGG CGAGGAGGAT AACATGGCCA TCATCAAGGA GTTCATGCGC TTCAAGGTGC | 1020 |
| ACATGGAGGG CTCCGTGAAC GGCCACGAGT TCGAGATCGA GGGCGAGGGC GAGGGCCGCC | 1080 |
| CCTACGAGGG CACCCAGACC GCCAAGCTGA AGGTGACCAA GGGTGGCCCC CTGCCCTTCG | 1140 |
| CCTGGGACAT CCTGTCCCCT CAGTTCATGT ACGGCTCCAA GGCCTACGTG AAGCACCCCG | 1200 |
| CCGACATCCC CGACTACTTG AAGCTGTCCT TCCCCGAGGG CTTCAAGTGG GAGCGCGTGA | 1260 |
| TGAACTTCGA GGACGGCGGC GTGGTGACCG TGACCCAGGA CTCCTCCCTG CAGGACGGCG | 1320 |
| AGTTCATCTA CAAGGTGAAG CTGCGCGGCA CCAACTTCCC CTCCGACGGC CCCGTAATGC | 1380 |
| AGAAGAAGAC CATGGGCTGG GAGGCCTCCT CCGAGCGGAT GTACCCCGAG GACGGCGCCC | 1440 |
| TGAAGGGCGA GATCAAGCAG AGGCTGAAGC TGAAGGACGG CGGCCACTAC GACGCTGAGG | 1500 |
| TCAAGACCAC CTACAAGGCC AAGAAGCCCG TGCAGCTGCC CGGCGCCTAC AACGTCAACA | 1560 |
| TCAAGTTGGA CATCACCTCC CACAACGAGG ACTACACCAT CGTGGAACAG TACGAACGCG | 1620 |
| CCGAGGGCCG CCACTCCACC GGCGGCATGG ACGAGCTGTA CAAGTAGTAA GATACATTGA | 1680 |
| TGAGTTTGGA CAAACCACAA CTAGAATGCA GTGAAAAAAA TGCTTTATTT GTGAAATTTG | 1740 |
| TGATGCTATT GCTTTATTTG TAACCATTAT AAGCTGCAAT AAACAAGTTT TGCAACACTC | 1800 |
| ACAGCACTCG GTGCACAATG GTCTTTTCCT CCAGGGCAAC GTTCAGTTTC TAGACGGTCC | 1860 |
| TTCAACCACC ACAAGGCGAG AGGAGCCGGG GACCCCAAGG GCCAGAGATG GCACACGCTG | 1920 |
| GTGCCGCTCG GCACGGAGAC CATCACCGAC AGCTACATGT CAGCACCCGC ATCAGAGCTG | 1980 |
| GACACTAATT TCTTTACGCT TTACGTAGCG CAAGGCACAA ATAAGTCGCA ACAGTACAAG | 2040 |
| TTCGGCACAG CTACATACGC GCTAAAGGAG CCGGTAATGA AGAGCGATGC ATGGGCAGTG | 2100 |
| GTACGCGTCC AGTCGGTCTG GCAGCTGGGT AACAGGCAGA GGCCATACCC ATGGGACGTC | 2160 |
| AACTGGGCGA ACAGCACCAT GTACTGGGGG ACGCAGCCCT GAAAAGGGGG GGGGGCTAAA | 2220 |
| GCCCCCCCCC CTTAAACCCC CCCCTGGGGG GGATTCCCCC CCAGACCCCC CCTTTATATA | 2280 |
| GCACTCAATA AACGCAGAAA ATAGATTTAT CGCACTATC | 2319 |

(SEQ ID NO: 13)
Annotations:

| Putative Domain | Base range |
|---|---|
| mCherry insert | 607-1789 |
| 5′ UTR | 1-379 |
| VP2 | 380-606 |
| Apoptin | 486-606 |
| VP1 | 1790-2202 |
| 3′ UTR | 2203-2319 |

In embodiments, the chimeric CAVector comprises a plurality of polypeptides (e.g., CAV VP1, VP2, and/or Apoptin) comprising sequences from a plurality of different CAVs (e.g., as described herein). For example, a chimeric CAVector may comprise an VP1 molecule from one CAV (e.g., a VP1 molecule, or an VP1 molecule having at least 75%, 80%, 85%, 90%, 95%, 96%, 97%, 98%, or 99% amino acid sequence identity thereto) and a VP2 molecule from or having similarity to a different CAV. In another example, a chimeric CAVector may comprise a first VP1 molecule from or similar to one CAV and a second VP1 molecule from or similar to a different CAV.

In some embodiments, the CAVector comprises a chimeric polypeptide (e.g., CAV VP1, VP2, and/or Apoptin), e.g., comprising at least one portion from one CAV (e.g., as described herein) and at least one portion from a different CAV (e.g., as described herein). In embodiments, the CAVector comprises a chimeric VP1 molecule comprising at least one portion of an VP1 molecule from one CAV (e.g., as described herein), or an VP1 molecule having at least 75%, 80%, 85%, 90%, 95%, 96%, 97%, 98%, or 99% amino acid sequence identity thereto, and at least one portion of an VP1 molecule from a different CAV (e.g., as described herein), or an VP1 molecule having at least 75%, 80%, 85%, 90%, 95%, 96%, 97%, 98%, or 99% amino acid sequence identity thereto. In embodiments, the chimeric VP1 molecule comprises an VP1 jelly-roll domain from one CAV, or a sequence having at least 75%, 80%, 85%, 90%, 95%, 96%, 97%, 98%, or 99% sequence identity thereto, and an VP1 amino acid subsequence (e.g., as described herein) from a different CAV, or a sequence having at least 75%, 80%, 85%, 90%, 95%, 96%, 97%, 98%, or 99% sequence identity thereto. In embodiments, the chimeric VP1 molecule comprises an VP1 arginine-rich region from one CAV, or a sequence having at least 75%, 80%, 85%, 90%, 95%, 96%, 97%, 98%, or 99% sequence identity thereto, and an VP1 amino acid subsequence (e.g., as described herein) from a different CAV, or a sequence having at least 75%, 80%, 85%, 90%, 95%, 96%, 97%, 98%, or 99% sequence identity thereto.

In embodiments, the CAVector comprises a chimeric VP2 molecule comprising at least one portion of a VP2 molecule from one CAV (e.g., as described herein), or a VP2 molecule having at least 75%, 80%, 85%, 90%, 95%, 96%, 97%, 98%, or 99% amino acid sequence identity thereto, and at least one portion of a VP2 molecule from a different CAV (e.g., as described herein), or a VP2 molecule having at least 75%, 80%, 85%, 90%, 95%, 96%, 97%, 98%, or 99% amino acid sequence identity thereto. In embodiments, the CAVector comprises a chimeric Apoptin molecule comprising at least one portion of an Apoptin molecule from one CAV (e.g., as described herein), or an Apoptin molecule having at least 75%, 80%, 85%, 90%, 95%, 96%, 97%, 98%, or 99% amino acid sequence identity thereto, and at least one portion of an Apoptin molecule from a different CAV (e.g., as described herein), or an Apoptin molecule having at least 75%, 80%, 85%, 90%, 95%, 96%, 97%, 98%, or 99% amino acid sequence identity thereto.

In some embodiments, a CAVector can be produced using a tandem construct, e.g., as described herein. Non-limiting examples of CAV tandem construct sequences are provided in Tables 14-16 below. In some embodiments, a genetic element construct (e.g., a tandem construct) described herein comprises one or more of a 5′ UTR, repeat region, CAAT signal, TATA box, VP2-encoding sequence, Apoptin-encoding sequence, VP1-encoding sequence, 3′ UTR, GC-rich region, or polyA signal sequence from a CAV genetic element sequence listed in any of Tables 14-16. In some embodiments, a genetic element construct (e.g., a tandem construct) described herein comprises one or more of a VP1 fragment (e.g., a start-less VP1 fragment), VP2 fragment (e.g., start-less VP2 fragment), and/or Apoptin fragment (e.g., start-less Apoptin fragment) from a CAV genetic element sequence listed in any of Tables 14-16. In some embodiments, a genetic element construct (e.g., a tandem construct) described herein comprises one or more of a promoter (e.g., an SV40 promoter), an SV40 polyA sequence, a hairpin region, an origin of replication (e.g., a pUC origin), or a resistance gene (e.g., an AmpR gene) from a tandem construct sequence listed in any of Tables 14-16.

TABLE 14
Exemplary CAV tandem plasmid pCAV-nLuc6_CAV

Sequence: 6794 bp
GAATTCCGAGTGGTTACTATTCCATCACCATTCTAGCCTGTACACAGAAAGTCAAGATG
GACGAATCGCTCGACTTCGCTCGCGATTCGTCGAAGGCGGGGGGCCGGAGGCCCCCCGG
TGGCCCCCCTCCAACGAGTGGAGCACGTACAGGGGGGTACGTCATCCGTACAGGGGGGT
ACGTCATCCGTACAGGGGGGTACGTCACAAAGAGGCGTTCCCGTACAGGGGGGTACGTC
ACGCGTACAGGGGGGTACGTCACAGCCAATCAAAAGCTGCCACGTTGCGAAAGTGACGT
TTCGAAAATGGGCGGCGCAAGCCTCTCTATATATTGAGCGCACATACCGGTCGGCAGTA
GGTATACGCAAGGCGGTCCGGGTGGTTGCACGGGAACGGCGGACAACCGGCCGCTGGGG
GCAGTGAATCGGCGCTTAGCCGAGAGGGGCAACCTGGGCCCAGCGGAGCCGCGCAGGGG
CAAGTAATTTCAATTGAACGCTCTCCAAGAAGATACTCCACCCGGACCATCAACGGTGT
TCAGGCCACCAACAAGTTCACGGCCGTTGGAAACCCCTCACTGCAGAGAGATCCGGATT
GGTATCGCTGGAATTACAATCACTCTATCGCTGTGTGGCTGCGCGAATGCTCGCGCTCC
CACGCTAAGATCTGCAACTGCGGACAATTCAGAAAGCACTGGTTTCAAGAATGTGCCGG
ACTTGAGGACCGATCAACCCAAGCCTCCCTCGAAGAAGCGATCCTGCGACCCCTCCGAG
TACAGGGTAAGCGAGCTAAAAGAAAGCTTGATTACCACTACTCCCAGCCGACCCCGAAC
CGCAAAAAGGCGTATAAGACTGTAAGTTGGCAAGACGAGCTCGCAGACCGAGAGGCCGA
TTTTACTCCTTCAGAAGAGGACGGTGGCACCACCTCAAGCGACTTCGACGAAGATATAA
ATTTCGACATCGGAGGAGACAGCGGTATCGTAGACGAGCTTTTAGGAAGGCCTTTCACA
ACCCTGTGGAATGTGTGTCAGTTAGGGTGTGGAAAGTCCCCAGGCTCCCCAGCAGGCAG
AAGTATGCAAAGCATGCATCTCAATTAGTCAGCAACCAGGTGTGGAAAGTCCCCAGGCT
CCCCAGCAGGCAGAAGTATGCAAAGCATGCATCTCAATTAGTCAGCAACCATAGTCCCG
CCCCTAACTCCGCCCATCCCGCCCCTAACTCCGCCCAGTTCCGCCCATTCTCCGCCCCA
TGGCTGACTAATTTTTTTTATTTATGCAGAGGCCGAGGCCGCCTCTGCCTCTGAGCTAT
TCCAGAAGTAGTGAGGAGGCTTTTTTGGAGGCCTAGGCTTTTGCAAAAAGCTGCCACCA
TGGTCTTCACACTCGAAGATTTCGTTGGGGACTGGCGACAGACAGCCGGCTACAACCTG
GACCAAGTCCTTGAACAGGGAGGTGTGTCCAGTTTGTTTCAGAATCTCGGGGTGTCCGT
AACTCCGATCCAAAGGATTGTCCTGAGCGGTGAAAATGGGCTGAAGATCGACATCCATG
TCATCATCCCGTATGAAGGTCTGAGCGGCGACCAAATGGGCCAGATCGAAAAAATTTTT
AAGGTGGTGTACCCTGTGGATGATCATCACTTTAAGGTGATCCTGCACTATGGCACACT
GGTAATCGACGGGGTTACGCCGAACATGATCGACTATTTCGGACGGCCGTATGAAGGCA
TCGCCGTGTTCGACGGCAAAAAGATCACTGTAACAGGGACCCTGTGGAACGGCAACAAA
ATTATCGACGAGCGCCTGATCAACCCCGACGGCTCCCTGCTGTTCCGAGTAACCATCAA
CGGAGTGACCGGCTGGCGGCTGTGCGAACGCATTCTGGCGTAATAAGATACATTGATGA
GTTTGGACAAACCACAACTAGAATGCAGTGAAAAAAATGCTTTATTTGTGAAATTTGTG
ATGCTATTGCTTTATTTGTAACCATTATAAGCTGCAATAAACAAGTTTACGCTTTACGT
AGCGCAAGGCACAAATAAGTCGCAACAGTACAAGTTCGGCACAGCTACATACGCGCTAA
AGGAGCCGGTAATGAAGAGCGATGCATGGGCAGTGGTACGCGTCCAGTCGGTCTGGCAG
CTGGGTAACAGGCAGAGGCCATACCCATGGGACGTCAACTGGGCGAACAGCACCATGTA
CTGGGGGACGCAGCCCTGAAAAGGGGGGGGGGCTAAAGCCCCCCCCCCTTAAACCCCCC
CCTGGGGGGGATTCCCCCCCAGACCCCCCCTTTATATAGCACTCAATAAACGCAGAAAA
TAGATTTATCGCACTATCGAATTCCGAGTGGTTACTATTCCATCACCATTCTAGCCTGT
ACACAGAAAGTCAAGATGGACGAATCGCTCGACTTCGCTCGCGATTCGTCGAAGGCGGG
GGGCCGGAGGCCCCCCGGTGGCCCCCCTCCAACGAGTGGAGCACGTACAGGGGGGTACG
TCATCCGTACAGGGGGGTACGTCATCCGTACAGGGGGGTACGTCACAAAGAGGCGTTCC
CGTACAGGGGGGTACGTCACGCGTACAGGGGGGTACGTCACAGCCAATCAAAAGCTGCC
ACGTTGCGAAAGTGACGTTTCGAAAATGGGCGGCGCAAGCCTCTCTATATATTGAGCGC
ACATACCGGTCGGCAGTAGGTATACGCAAGGCGGTCCGGGTGGATGCACGGGAACGGCG
GACAACCGGCCGCTGGGGGCAGTGAATCGGCGCTTAGCCGAGAGGGGCAACCTGGGCCC
AGCGGAGCCGCGCAGGGGCAAGTAATTTCAAATGAACGCTCTCCAAGAAGATACTCCAC
CCGGACCATCAACGGTGTTCAGGCCACCAACAAGTTCACGGCCGTTGGAAACCCCTCAC
TGCAGAGAGATCCGGATTGGTATCGCTGGAATTACAATCACTCTATCGCTGTGTGGCTG
CGCGAATGCTCGCGCTCCCACGCTAAGATCTGCAACTGCGGACAATTCAGAAAGCACTG
GTTTCAAGAATGTGCCGGACTTGAGGACCGATCAACCCAAGCCTCCCTCGAAGAAGCGA
TCCTGCGACCCCTCCGAGTACAGGGTAAGCGAGCTAAAAGAAAGCTTGATTACCACTAC
TCCCAGCCGACCCCGAACCGCAAAAAGGCGTATAAGACTGTAAGATGGCAAGACGAGCT
CGCAGACCGAGAGGCCGATTTTACTCCTTCAGAAGAGGACGGTGGCACCACCTCAAGCG
ACTTCGACGAAGATATAAATTTCGACATCGGAGGAGACAGCGGTATCGTAGACGAGCTT
TTAGGAAGGCCTTTCACAACCCCCGCCCCGGTACGTATAGTGTGAGGCTGCCGAACCCC
CAATCTACTATGACTATCCGCTTCCAAGGGGTCATCTTTCTCACGGAAGGACTCATTCT
GCCTAAAAACAGCACAGCGGGGGGCTATGCAGACCACATGTACGGGGCGAGAGTCGCCA
AGATCTCTGTGAACCTGAAAGAGTTCCTGCTAGCCTCAATGAACCTGACATACGTGAGC
AAAATCGGAGGCCCCATCGCCGGTGAGTTGATTGCGGACGGGTCTAAATCACAAGCCGC
GGACAATTGGCCTAATTGCTGGCTGCCGCTAGATAATAACGTGCCCTCCGCTACACCAT
CGGCATGGTGGAGATGGGCCTTAATGATGATGCAGCCCACGGACTCTTGCCGGTTCTTT
AATCACCCAAAGCAGATGACCCTGCAAGACATGGGTCGCATGTTTGGGGGCTGGCACCT
GTTCCGACACATTGAAACCCGCTTTCAGCTCCTTGCCACTAAGAATGAGGGATCCTTCA
GCCCCGTGGCGAGTCTTCTCTCCCAGGGAGAGTACCTCACGCGTCGGGACGATGTTAAG
TACAGCAGCGATCACCAGAACCGGTGGCAAAAAGGCGGACAACCGATGACGGGGGGCAT
TGCTTATGCGACCGGGAAAATGAGACCCGACGAGCAACAGTACCCTGCTATGCCCCCAG
ACCCCCCGATCATCACCGCTACTACAGCGCAAGGCACGCAAGTCCGCTGCATGAATAGC
ACGCAAGCTTGGTGGTCATGGGACACATATATGAGCTTTGCAACACTCACAGCACTCGG
TGCACAATGGTCTTTTCCTCCAGGGCAACGTTCAGTTTCTAGACGGTCCTTCAACCACC
ACAAGGCGAGAGGAGCCGGGGACCCCAAGGGCCAGAGATGGCACACGCTGGTGCCGCTC
GGCACGGAGACCATCACCGACAGCTACATGTCAGCACCCGCATCAGAGCTGGACACTAA
TTTCTTTACGCTTTACGTAGCGCAAGGCACAAATAAGTCGCAACAGTACAAGTTCGGCA
CAGCTACATACGCGCTAAAGGAGCCGGTAATGAAGAGCGATGCATGGGCAGTGGTACGC
GTCCAGTCGGTCTGGCAGCTGGGTAACAGGCAGAGGCCATACCCATGGGACGTCAACTG
GGCGAACAGCACCATGTACTGGGGGACGCAGCCCTGAAAAGGGGGGGGGGCTAAAGCCC
CCCCCCCTTAAACCCCCCCCTGGGGGGGATTCCCCCCCAGACCCCCCCTTTATATAGCA
CTCAATAAACGCAGAAAATAGATTTATCGCACTATCTGAATGAGACGCACGCTTCCTCG
CTCACTGACTCGCTGCGCTCGGTCGTTCGGCTGCGGCGAGCGGTATCAGCTCACTCAAA
GGCGGTAATACGGTTATCCACAGAATCAGGGGATAACGCAGGAAAGAACATGTGAGCAA
AAGGCCAGCAAAAGGCCAGGAACCGTAAAAAGGCCGCGTTGCTGGCGTTTTTCCATAGG
CTCCGCCCCCCTGACGAGCATCACAAAAATCGACGCTCAAGTCAGAGGTGGCGAAACCC
GACAGGACTATAAAGATACCAGGCGTTTCCCCCTGGAAGCTCCCTCGTGCGCTCTCCTG
TTCCGACCCTGCCGCTTACCGGATACCTGTCCGCCTTTCTCCCTTCGGGAAGCGTGGCG
CTTTCTCATAGCTCACGCTGTAGGTATCTCAGTTCGGTGTAGGTCGTTCGCTCCAAGCT
GGGCTGTGTGCACGAACCCCCCGTTCAGCCCGACCGCTGCGCCTTATCCGGTAACTATC
GTCTTGAGTCCAACCCGGTAAGACACGACTTATCGCCACTGGCAGCAGCCACTGGTAAC
AGGATTAGCAGAGCGAGGTATGTAGGCGGTGCTACAGAGTTCTTGAAGTGGTGGCCTAA
CTACGGCTACACTAGAAGAACAGTATTTGGTATCTGCGCTCTGCTGAAGCCAGTTACCT
TCGGAAAAAGAGTTGGTAGCTCTTGATCCGGCAAACAAACCACCGCTGGTAGCGGTGGT
TTTTTTGTTTGCAAGCAGCAGATTACGCGCAGAAAAAAAGGATCTCAAGAAGATCCTTT
GATCTTTTCTACGGGGTCTGACGCTCAGTGGAACGAAAACTCACGTTAAGGGATTTTGG
TCATGAGATTATCAAAAAGGATCTTCACCTAGATCCTTTTAAATTAAAAATGAAGTTTT
AAATCAATCTAAAGTATATATGAGTAAACTTGGTCTGACAGTTACCAATGCTTAATCAG
TGAGGCACCTATCTCAGCGATCTGTCTATTTCGTTCATCCATAGTTGCCTGACTCCCCG
TCGTGTAGATAACTACGATACGGGAGGGCTTACCATCTGGCCCCAGTGCTGCAATGATA
CCGCGAGACCCACGCTCACCGGCTCCAGATTTATCAGCAATAAACCAGCCAGCCGGAAG
GGCCGAGCGCAGAAGTGGTCCTGCAACTTTATCCGCCTCCATCCAGTCTATTAATTGTT
GCCGGGAAGCTAGAGTAAGTAGTTCGCCAGTTAATAGTTTGCGCAACGTTGTTGCCATT
GCTACAGGCATCGTGGTGTCACGCTCGTCGTTTGGTATGGCTTCATTCAGCTCCGGTTC
CCAACGATCAAGGCGAGTTACATGATCCCCCATGTTGTGCAAAAAAGCGGTTAGCTCCT
TCGGTCCTCCGATCGTTGTCAGAAGTAAGTTGGCCGCAGTGTTATCACTCATGGTTATG
GCAGCACTGCATAATTCTCTTACTGTCATGCCATCCGTAAGATGCTTTTCTGTGACTGG
TGAGTACTCAACCAAGTCATTCTGAGAATAGTGTATGCGGCGACCGAGTTGCTCTTGCC
CGGCGTCAATACGGGATAATACCGCGCCACATAGCAGAACTTTAAAAGTGCTCATCATT
GGAAAACGTTCTTCGGGGCGAAAACTCTCAAGGATCTTACCGCTGTTGAGATCCAGTTC
GATGTAACCCACTCGTGCACCCAACTGATCTTCAGCATCTTTTACTTTCACCAGCGTTT
CTGGGTGAGCAAAAACAGGAAGGCAAAATGCCGCAAAAAAGGGAATAAGGGCGACACGG
AAATGTTGAATACTCATACTCTTCCTTTTTCAATATTATTGAAGCATTTATCAGGGTTA
TTGTCTCATGAGCGGATACATATTTGAATGTATTTAGAAAAATAAACAAATAGGGGTTC
CGCGCACATTTCCCCGAAAAGTGCCACCTGACGTCGACGGATCGGGAGATCTCCCGATC
CCCTATGGTGCACTCTCAGTACAATCTGCTCTGATGCCGCATAGTTAAGCCAGTATCTG
CTCCCTGCTTGTGTGTTGGAGGTCGCTGAGTAGTGCGCGAGCAAAATTTAAGCTACAAC
AAGGCAAGGCTTGACCGACAATTCTCTGGCTAACTAGAGAACCCACTGCTTACTAGGCG
TCTCAGAAT
(SEQ ID NO: 14)
Annotations:

| Name | Start | End |
|---|---|---|
| CA Vector-nLuc6 | 1 | 2319 |
| CAV repeat region | | 260 |
| Start-less VP2 fragment | 380 | 1006 |
| Start-less VP3 (Apoptin) | 486 | 851 |
| Start-less VP1 upstream | 853 | 1006 |
| fragment | | |
| SV40 promoter | 1007 | 1350 |
| NanoLuc | 1357 | 1872 |
| SV40 poly(A) | 1873 | 1994 |
| Downstream VP1 fragment | 1995 | 2202 |
| Hairpin Region | 2206 | 2272 |
| CAV (wt) | 2320 | 4638 |
| CAV repeat region | 2463 | 2579 |
| VP2 | 2699 | 3349 |
| VP3 (Apoptin) | 2805 | 3170 |
| VP1 | 3172 | 4521 |
| Hairpin Region | 4525 | 4591 |
| pUC origin | 4770 | 5443 |
| AmpR Gene | 5588 | 6547 |

TABLE 15
Exemplary CAV tandem plasmid pCAV-nLuc6_CAVA3NCR

Sequence: 6673 bp
GAATTCCGAGTGGTTACTATTCCATCACCATTCTAGCCTGTACACAGAAAGTCAAGATG
GACGAATCGCTCGACTTCGCTCGCGATTCGTCGAAGGCGGGGGGCCGGAGGCCCCCCGG
TGGCCCCCCTCCAACGAGTGGAGCACGTACAGGGGGGTACGTCATCCGTACAGGGGGGT
ACGTCATCCGTACAGGGGGGTACGTCACAAAGAGGCGTTCCCGTACAGGGGGGTACGTC
ACGCGTACAGGGGGGTACGTCACAGCCAATCAAAAGCTGCCACGTTGCGAAAGTGACGT
TTCGAAAATGGGCGGCGCAAGCCTCTCTATATATTGAGCGCACATACCGGTCGGCAGTA
GGTATACGCAAGGCGGTCCGGGTGGTTGCACGGGAACGGCGGACAACCGGCCGCTGGGG
GCAGTGAATCGGCGCTTAGCCGAGAGGGGCAACCTGGGCCCAGCGGAGCCGCGCAGGGG
CAAGTAATTTCAATTGAACGCTCTCCAAGAAGATACTCCACCCGGACCATCAACGGTGT
TCAGGCCACCAACAAGTTCACGGCCGTTGGAAACCCCTCACTGCAGAGAGATCCGGATT
GGTATCGCTGGAATTACAATCACTCTATCGCTGTGTGGCTGCGCGAATGCTCGCGCTCC
CACGCTAAGATCTGCAACTGCGGACAATTCAGAAAGCACTGGTTTCAAGAATGTGCCGG
ACTTGAGGACCGATCAACCCAAGCCTCCCTCGAAGAAGCGATCCTGCGACCCCTCCGAG
TACAGGGTAAGCGAGCTAAAAGAAAGCTTGATTACCACTACTCCCAGCCGACCCCGAAC
CGCAAAAAGGCGTATAAGACTGTAAGTTGGCAAGACGAGCTCGCAGACCGAGAGGCCGA
TTTTACTCCTTCAGAAGAGGACGGTGGCACCACCTCAAGCGACTTCGACGAAGATATAA
ATTTCGACATCGGAGGAGACAGCGGTATCGTAGACGAGCTTTTAGGAAGGCCTTTCACA
ACCCTGTGGAATGTGTGTCAGTTAGGGTGTGGAAAGTCCCCAGGCTCCCCAGCAGGCAG
AAGTATGCAAAGCATGCATCTCAATTAGTCAGCAACCAGGTGTGGAAAGTCCCCAGGCT
CCCCAGCAGGCAGAAGTATGCAAAGCATGCATCTCAATTAGTCAGCAACCATAGTCCCG
CCCCTAACTCCGCCCATCCCGCCCCTAACTCCGCCCAGTTCCGCCCATTCTCCGCCCCA
TGGCTGACTAATTTTTTTTATTTATGCAGAGGCCGAGGCCGCCTCTGCCTCTGAGCTAT
TCCAGAAGTAGTGAGGAGGCTTTTTTGGAGGCCTAGGCTTTTGCAAAAAGCTGCCACCA
TGGTCTTCACACTCGAAGATTTCGTTGGGGACTGGCGACAGACAGCCGGCTACAACCTG
GACCAAGTCCTTGAACAGGGAGGTGTGTCCAGTTTGTTTCAGAATCTCGGGGTGTCCGT
AACTCCGATCCAAAGGATTGTCCTGAGCGGTGAAAATGGGCTGAAGATCGACATCCATG
TCATCATCCCGTATGAAGGTCTGAGCGGCGACCAAATGGGCCAGATCGAAAAAATTTTT
AAGGTGGTGTACCCTGTGGATGATCATCACTTTAAGGTGATCCTGCACTATGGCACACT
GGTAATCGACGGGGTTACGCCGAACATGATCGACTATTTCGGACGGCCGTATGAAGGCA
TCGCCGTGTTCGACGGCAAAAAGATCACTGTAACAGGGACCCTGTGGAACGGCAACAAA
ATTATCGACGAGCGCCTGATCAACCCCGACGGCTCCCTGCTGTTCCGAGTAACCATCAA
CGGAGTGACCGGCTGGCGGCTGTGCGAACGCATTCTGGCGTAATAAGATACATTGATGA
GTTTGGACAAACCACAACTAGAATGCAGTGAAAAAAATGCTTTATTTGTGAAATTTGTG
ATGCTATTGCTTTATTTGTAACCATTATAAGCTGCAATAAACAAGTTTACGCTTTACGT
AGCGCAAGGCACAAATAAGTCGCAACAGTACAAGTTCGGCACAGCTACATACGCGCTAA
AGGAGCCGGTAATGAAGAGCGATGCATGGGCAGTGGTACGCGTCCAGTCGGTCTGGCAG
CTGGGTAACAGGCAGAGGCCATACCCATGGGACGTCAACTGGGCGAACAGCACCATGTA
CTGGGGGACGCAGCCCTGAAAAGGGGGGGGGGCTAAAGCCCCCCCCCCTTAAACCCCCC
CCTGGGGGGGATTCCCCCCCAGACCCCCCCTTTATATAGCACTCAATAAACGCAGAAAA
TAGATTTATCGCACTATCGAATTCCGAGTGGTTACTATTCCATCACCATTCTAGCCTGT
ACACAGAAAGTCAAGATGGACGAATCGCTCGACTTCGCTCGCGATTCGTCGAAGGCGGG
GGGCCGGAGGCCCCCCGGTGGCCCCCCTCCAACGAGTGGAGCACGTACAGGGGGGTACG
TCATCCGTACAGGGGGGTACGTCATCCGTACAGGGGGGTACGTCACAAAGAGGCGTTCC
CGTACAGGGGGGTACGTCACGCGTACAGGGGGGTACGTCACAGCCAATCAAAAGCTGCC
ACGTTGCGAAAGTGACGTTTCGAAAATGGGCGGCGCAAGCCTCTCTATATATTGAGCGC
ACATACCGGTCGGCAGTAGGTATACGCAAGGCGGTCCGGGTGGATGCACGGGAACGGCG
GACAACCGGCCGCTGGGGGCAGTGAATCGGCGCTTAGCCGAGAGGGGCAACCTGGGCCC
AGCGGAGCCGCGCAGGGGCAAGTAATTTCAAATGAACGCTCTCCAAGAAGATACTCCAC
CCGGACCATCAACGGTGTTCAGGCCACCAACAAGTTCACGGCCGTTGGAAACCCCTCAC
TGCAGAGAGATCCGGATTGGTATCGCTGGAATTACAATCACTCTATCGCTGTGTGGCTG
CGCGAATGCTCGCGCTCCCACGCTAAGATCTGCAACTGCGGACAATTCAGAAAGCACTG
GTTTCAAGAATGTGCCGGACTTGAGGACCGATCAACCCAAGCCTCCCTCGAAGAAGCGA
TCCTGCGACCCCTCCGAGTACAGGGTAAGCGAGCTAAAAGAAAGCTTGATTACCACTAC
TCCCAGCCGACCCCGAACCGCAAAAAGGCGTATAAGACTGTAAGATGGCAAGACGAGCT
CGCAGACCGAGAGGCCGATTTTACTCCTTCAGAAGAGGACGGTGGCACCACCTCAAGCG
ACTTCGACGAAGATATAAATTTCGACATCGGAGGAGACAGCGGTATCGTAGACGAGCTT
TTAGGAAGGCCTTTCACAACCCCCGCCCCGGTACGTATAGTGTGAGGCTGCCGAACCCC
CAATCTACTATGACTATCCGCTTCCAAGGGGTCATCTTTCTCACGGAAGGACTCATTCT
GCCTAAAAACAGCACAGCGGGGGGCTATGCAGACCACATGTACGGGGCGAGAGTCGCCA
AGATCTCTGTGAACCTGAAAGAGTTCCTGCTAGCCTCAATGAACCTGACATACGTGAGC
AAAATCGGAGGCCCCATCGCCGGTGAGTTGATTGCGGACGGGTCTAAATCACAAGCCGC
GGACAATTGGCCTAATTGCTGGCTGCCGCTAGATAATAACGTGCCCTCCGCTACACCAT
CGGCATGGTGGAGATGGGCCTTAATGATGATGCAGCCCACGGACTCTTGCCGGTTCTTT
AATCACCCAAAGCAGATGACCCTGCAAGACATGGGTCGCATGTTTGGGGGCTGGCACCT
GTTCCGACACATTGAAACCCGCTTTCAGCTCCTTGCCACTAAGAATGAGGGATCCTTCA
GCCCCGTGGCGAGTCTTCTCTCCCAGGGAGAGTACCTCACGCGTCGGGACGATGTTAAG
TACAGCAGCGATCACCAGAACCGGTGGCAAAAAGGCGGACAACCGATGACGGGGGGCAT
TGCTTATGCGACCGGGAAAATGAGACCCGACGAGCAACAGTACCCTGCTATGCCCCCAG
ACCCCCCGATCATCACCGCTACTACAGCGCAAGGCACGCAAGTCCGCTGCATGAATAGC
ACGCAAGCTTGGTGGTCATGGGACACATATATGAGCTTTGCAACACTCACAGCACTCGG
TGCACAATGGTCTTTTCCTCCAGGGCAACGTTCAGTTTCTAGACGGTCCTTCAACCACC
ACAAGGCGAGAGGAGCCGGGGACCCCAAGGGCCAGAGATGGCACACGCTGGTGCCGCTC
GGCACGGAGACCATCACCGACAGCTACATGTCAGCACCCGCATCAGAGCTGGACACTAA
TTTCTTTACGCTTTACGTAGCGCAAGGCACAAATAAGTCGCAACAGTACAAGTTCGGCA
CAGCTACATACGCGCTAAAGGAGCCGGTAATGAAGAGCGATGCATGGGCAGTGGTACGC
GTCCAGTCGGTCTGGCAGCTGGGTAACAGGCAGAGGCCATACCCATGGGACGTCAACTG
GGCGAACAGCACCATGTACTGGGGGACGCAGCCCTGATGAATGAGACGCACGCTTCCTC
GCTCACTGACTCGCTGCGCTCGGTCGTTCGGCTGCGGCGAGCGGTATCAGCTCACTCAA
AGGCGGTAATACGGTTATCCACAGAATCAGGGGATAACGCAGGAAAGAACATGTGAGCA
AAAGGCCAGCAAAAGGCCAGGAACCGTAAAAAGGCCGCGTTGCTGGCGTTTTTCCATAG
GCTCCGCCCCCCTGACGAGCATCACAAAAATCGACGCTCAAGTCAGAGGTGGCGAAACC
CGACAGGACTATAAAGATACCAGGCGTTTCCCCCTGGAAGCTCCCTCGTGCGCTCTCCT
GTTCCGACCCTGCCGCTTACCGGATACCTGTCCGCCTTTCTCCCTTCGGGAAGCGTGGC
GCTTTCTCATAGCTCACGCTGTAGGTATCTCAGTTCGGTGTAGGTCGTTCGCTCCAAGC
TGGGCTGTGTGCACGAACCCCCCGTTCAGCCCGACCGCTGCGCCTTATCCGGTAACTAT
CGTCTTGAGTCCAACCCGGTAAGACACGACTTATCGCCACTGGCAGCAGCCACTGGTAA
CAGGATTAGCAGAGCGAGGTATGTAGGCGGTGCTACAGAGTTCTTGAAGTGGTGGCCTA
ACTACGGCTACACTAGAAGAACAGTATTTGGTATCTGCGCTCTGCTGAAGCCAGTTACC
TTCGGAAAAAGAGTTGGTAGCTCTTGATCCGGCAAACAAACCACCGCTGGTAGCGGTGG
TTTTTTTGTTTGCAAGCAGCAGATTACGCGCAGAAAAAAAGGATCTCAAGAAGATCCTT
TGATCTTTTCTACGGGGTCTGACGCTCAGTGGAACGAAAACTCACGTTAAGGGATTTTG
GTCATGAGATTATCAAAAAGGATCTTCACCTAGATCCTTTTAAATTAAAAATGAAGTTT
TAAATCAATCTAAAGTATATATGAGTAAACTTGGTCTGACAGTTACCAATGCTTAATCA
GTGAGGCACCTATCTCAGCGATCTGTCTATTTCGTTCATCCATAGTTGCCTGACTCCCC
GTCGTGTAGATAACTACGATACGGGAGGGCTTACCATCTGGCCCCAGTGCTGCAATGAT
ACCGCGAGACCCACGCTCACCGGCTCCAGATTTATCAGCAATAAACCAGCCAGCCGGAA
GGGCCGAGCGCAGAAGTGGTCCTGCAACTTTATCCGCCTCCATCCAGTCTATTAATTGT
TGCCGGGAAGCTAGAGTAAGTAGTTCGCCAGTTAATAGTTTGCGCAACGTTGTTGCCAT
TGCTACAGGCATCGTGGTGTCACGCTCGTCGTTTGGTATGGCTTCATTCAGCTCCGGTT
CCCAACGATCAAGGCGAGTTACATGATCCCCCATGTTGTGCAAAAAAGCGGTTAGCTCC
TTCGGTCCTCCGATCGTTGTCAGAAGTAAGTTGGCCGCAGTGTTATCACTCATGGTTAT
GGCAGCACTGCATAATTCTCTTACTGTCATGCCATCCGTAAGATGCTTTTCTGTGACTG
GTGAGTACTCAACCAAGTCATTCTGAGAATAGTGTATGCGGCGACCGAGTTGCTCTTGC
CCGGCGTCAATACGGGATAATACCGCGCCACATAGCAGAACTTTAAAAGTGCTCATCAT
TGGAAAACGTTCTTCGGGGCGAAAACTCTCAAGGATCTTACCGCTGTTGAGATCCAGTT
CGATGTAACCCACTCGTGCACCCAACTGATCTTCAGCATCTTTTACTTTCACCAGCGTT
TCTGGGTGAGCAAAAACAGGAAGGCAAAATGCCGCAAAAAAGGGAATAAGGGCGACACG
GAAATGTTGAATACTCATACTCTTCCTTTTTCAATATTATTGAAGCATTTATCAGGGTT
ATTGTCTCATGAGCGGATACATATTTGAATGTATTTAGAAAAATAAACAAATAGGGGTT
CCGCGCACATTTCCCCGAAAAGTGCCACCTGACGTCGACGGATCGGGAGATCTCCCGAT
CCCCTATGGTGCACTCTCAGTACAATCTGCTCTGATGCCGCATAGTTAAGCCAGTATCT
GCTCCCTGCTTGTGTGTTGGAGGTCGCTGAGTAGTGCGCGAGCAAAATTTAAGCTACAA
CAAGGCAAGGCTTGACCGACAATTCTCTGGCTAACTAGAGAACCCACTGCTTACTAGGC
GTCTCA (SEQ ID NO: 15)
Annotations:

| Name | Start | End |
|---|---|---|
| CA Vector-nLuc6 | 1 | 2319 |
| CAV repeat region | 144 | 260 |
| Start-less VP2 fragment | 380 | 1006 |
| Start-less VP3 (Apoptin) | 486 | 851 |
| Start-less VP1 upstream | 853 | 1006 |
| fragment | | |
| SV40 promoter | 1007 | 1350 |
| NanoLuc | 1357 | 1872 |
| SV40 poly(A) | 1873 | 1994 |
| Downstream VP1 fragment | 1995 | 2202 |
| Hairpin Region | 2206 | 2272 |
| CAVA3NCR | 2320 | 4521 |
| CAV repeat region | 2463 | 2579 |
| VP2 | 2699 | 3349 |
| VP3 (Apoptin) | 2805 | 3170 |
| VP1 | 3172 | 4521 |
| pUC origin | 4653 | 5326 |
| AmpR Gene | 5471 | 6430 |

TABLE 16
Exemplary CAV tandem plasmid pCAV-nLuc6_CAVAPromAORFA3NCR

Sequence: 4793 bp
GAATTCCGAGTGGTTACTATTCCATCACCATTCTAGCCTGTACACAGAAAGTCAAGATG
GACGAATCGCTCGACTTCGCTCGCGATTCGTCGAAGGCGGGGGGCCGGAGGCCCCCCGG
TGGCCCCCCTCCAACGAGTGGAGCACGTACAGGGGGGTACGTCATCCGTACAGGGGGGT
ACGTCATCCGTACAGGGGGGTACGTCACAAAGAGGCGTTCCCGTACAGGGGGGTACGTC
ACGCGTACAGGGGGGTACGTCACAGCCAATCAAAAGCTGCCACGTTGCGAAAGTGACGT
TTCGAAAATGGGCGGCGCAAGCCTCTCTATATATTGAGCGCACATACCGGTCGGCAGTA
GGTATACGCAAGGCGGTCCGGGTGGTTGCACGGGAACGGCGGACAACCGGCCGCTGGGG
GCAGTGAATCGGCGCTTAGCCGAGAGGGGCAACCTGGGCCCAGCGGAGCCGCGCAGGGG
CAAGTAATTTCAATTGAACGCTCTCCAAGAAGATACTCCACCCGGACCATCAACGGTGT
TCAGGCCACCAACAAGTTCACGGCCGTTGGAAACCCCTCACTGCAGAGAGATCCGGATT
GGTATCGCTGGAATTACAATCACTCTATCGCTGTGTGGCTGCGCGAATGCTCGCGCTCC
CACGCTAAGATCTGCAACTGCGGACAATTCAGAAAGCACTGGTTTCAAGAATGTGCCGG
ACTTGAGGACCGATCAACCCAAGCCTCCCTCGAAGAAGCGATCCTGCGACCCCTCCGAG
TACAGGGTAAGCGAGCTAAAAGAAAGCTTGATTACCACTACTCCCAGCCGACCCCGAAC
CGCAAAAAGGCGTATAAGACTGTAAGTTGGCAAGACGAGCTCGCAGACCGAGAGGCCGA
TTTTACTCCTTCAGAAGAGGACGGTGGCACCACCTCAAGCGACTTCGACGAAGATATAA
ATTTCGACATCGGAGGAGACAGCGGTATCGTAGACGAGCTTTTAGGAAGGCCTTTCACA
ACCCTGTGGAATGTGTGTCAGTTAGGGTGTGGAAAGTCCCCAGGCTCCCCAGCAGGCAG
AAGTATGCAAAGCATGCATCTCAATTAGTCAGCAACCAGGTGTGGAAAGTCCCCAGGCT
CCCCAGCAGGCAGAAGTATGCAAAGCATGCATCTCAATTAGTCAGCAACCATAGTCCCG
CCCCTAACTCCGCCCATCCCGCCCCTAACTCCGCCCAGTTCCGCCCATTCTCCGCCCCA
TGGCTGACTAATTTTTTTTATTTATGCAGAGGCCGAGGCCGCCTCTGCCTCTGAGCTAT
TCCAGAAGTAGTGAGGAGGCTTTTTTGGAGGCCTAGGCTTTTGCAAAAAGCTGCCACCA
TGGTCTTCACACTCGAAGATTTCGTTGGGGACTGGCGACAGACAGCCGGCTACAACCTG
GACCAAGTCCTTGAACAGGGAGGTGTGTCCAGTTTGTTTCAGAATCTCGGGGTGTCCGT
AACTCCGATCCAAAGGATTGTCCTGAGCGGTGAAAATGGGCTGAAGATCGACATCCATG
TCATCATCCCGTATGAAGGTCTGAGCGGCGACCAAATGGGCCAGATCGAAAAAATTTTT
AAGGTGGTGTACCCTGTGGATGATCATCACTTTAAGGTGATCCTGCACTATGGCACACT
GGTAATCGACGGGGTTACGCCGAACATGATCGACTATTTCGGACGGCCGTATGAAGGCA
TCGCCGTGTTCGACGGCAAAAAGATCACTGTAACAGGGACCCTGTGGAACGGCAACAAA
ATTATCGACGAGCGCCTGATCAACCCCGACGGCTCCCTGCTGTTCCGAGTAACCATCAA
CGGAGTGACCGGCTGGCGGCTGTGCGAACGCATTCTGGCGTAATAAGATACATTGATGA
GTTTGGACAAACCACAACTAGAATGCAGTGAAAAAAATGCTTTATTTGTGAAATTTGTG
ATGCTATTGCTTTATTTGTAACCATTATAAGCTGCAATAAACAAGTTTACGCTTTACGT
AGCGCAAGGCACAAATAAGTCGCAACAGTACAAGTTCGGCACAGCTACATACGCGCTAA
AGGAGCCGGTAATGAAGAGCGATGCATGGGCAGTGGTACGCGTCCAGTCGGTCTGGCAG
CTGGGTAACAGGCAGAGGCCATACCCATGGGACGTCAACTGGGCGAACAGCACCATGTA
CTGGGGGACGCAGCCCTGAAAAGGGGGGGGGGCTAAAGCCCCCCCCCCTTAAACCCCCC
CCTGGGGGGGATTCCCCCCCAGACCCCCCCTTTATATAGCACTCAATAAACGCAGAAAA
TAGATTTATCGCACTATCGAATTCCGAGTGGTTACTATTCCATCACCATTCTAGCCTGT
ACACAGAAAGTCAAGATGGACGAATCGCTCGACTTCGCTCGCGATTCGTCGAAGGCGGG
GGGCCGGAGGCCCCCCGGTGGCCCCCCTCCAACGAGTGGAGCACGTACAGGGGGGTACG
TCATCCGTACAGGGGGGTACGTCATCCGTACAGGGGGGTACGTCACAAAGAGGCGTTCC
CGTACAGGGGGGTACGTCACGCGTACAGGGGGGTACGTCACAGCCAATCAAAAGCTGCC
ACGTTGCGAAAGTGACGTTTCGAAAATGGGCGGCGCAAGCCTCTCTGAATGAGACGCAC
GCTTCCTCGCTCACTGACTCGCTGCGCTCGGTCGTTCGGCTGCGGCGAGCGGTATCAGC
TCACTCAAAGGCGGTAATACGGTTATCCACAGAATCAGGGGATAACGCAGGAAAGAACA
TGTGAGCAAAAGGCCAGCAAAAGGCCAGGAACCGTAAAAAGGCCGCGTTGCTGGCGTTT
TTCCATAGGCTCCGCCCCCCTGACGAGCATCACAAAAATCGACGCTCAAGTCAGAGGTG
GCGAAACCCGACAGGACTATAAAGATACCAGGCGTTTCCCCCTGGAAGCTCCCTCGTGC
GCTCTCCTGTTCCGACCCTGCCGCTTACCGGATACCTGTCCGCCTTTCTCCCTTCGGGA
AGCGTGGCGCTTTCTCATAGCTCACGCTGTAGGTATCTCAGTTCGGTGTAGGTCGTTCG
CTCCAAGCTGGGCTGTGTGCACGAACCCCCCGTTCAGCCCGACCGCTGCGCCTTATCCG
GTAACTATCGTCTTGAGTCCAACCCGGTAAGACACGACTTATCGCCACTGGCAGCAGCC
ACTGGTAACAGGATTAGCAGAGCGAGGTATGTAGGCGGTGCTACAGAGTTCTTGAAGTG
GTGGCCTAACTACGGCTACACTAGAAGAACAGTATTTGGTATCTGCGCTCTGCTGAAGC
CAGTTACCTTCGGAAAAAGAGTTGGTAGCTCTTGATCCGGCAAACAAACCACCGCTGGT
AGCGGTGGTTTTTTTGTTTGCAAGCAGCAGATTACGCGCAGAAAAAAAGGATCTCAAGA
AGATCCTTTGATCTTTTCTACGGGGTCTGACGCTCAGTGGAACGAAAACTCACGTTAAG
GGATTTTGGTCATGAGATTATCAAAAAGGATCTTCACCTAGATCCTTTTAAATTAAAAA
TGAAGTTTTAAATCAATCTAAAGTATATATGAGTAAACTTGGTCTGACAGTTACCAATG
CTTAATCAGTGAGGCACCTATCTCAGCGATCTGTCTATTTCGTTCATCCATAGTTGCCT
GACTCCCCGTCGTGTAGATAACTACGATACGGGAGGGCTTACCATCTGGCCCCAGTGCT
GCAATGATACCGCGAGACCCACGCTCACCGGCTCCAGATTTATCAGCAATAAACCAGCC
AGCCGGAAGGGCCGAGCGCAGAAGTGGTCCTGCAACTTTATCCGCCTCCATCCAGTCTA
TTAATTGTTGCCGGGAAGCTAGAGTAAGTAGTTCGCCAGTTAATAGTTTGCGCAACGTT
GTTGCCATTGCTACAGGCATCGTGGTGTCACGCTCGTCGTTTGGTATGGCTTCATTCAG
CTCCGGTTCCCAACGATCAAGGCGAGTTACATGATCCCCCATGTTGTGCAAAAAAGCGG
TTAGCTCCTTCGGTCCTCCGATCGTTGTCAGAAGTAAGTTGGCCGCAGTGTTATCACTC
ATGGTTATGGCAGCACTGCATAATTCTCTTACTGTCATGCCATCCGTAAGATGCTTTTC
TGTGACTGGTGAGTACTCAACCAAGTCATTCTGAGAATAGTGTATGCGGCGACCGAGTT
GCTCTTGCCCGGCGTCAATACGGGATAATACCGCGCCACATAGCAGAACTTTAAAAGTG
CTCATCATTGGAAAACGTTCTTCGGGGCGAAAACTCTCAAGGATCTTACCGCTGTTGAG
ATCCAGTTCGATGTAACCCACTCGTGCACCCAACTGATCTTCAGCATCTTTTACTTTCA
CCAGCGTTTCTGGGTGAGCAAAAACAGGAAGGCAAAATGCCGCAAAAAAGGGAATAAGG
GCGACACGGAAATGTTGAATACTCATACTCTTCCTTTTTCAATATTATTGAAGCATTTA
TCAGGGTTATTGTCTCATGAGCGGATACATATTTGAATGTATTTAGAAAAATAAACAAA
TAGGGGTTCCGCGCACATTTCCCCGAAAAGTGCCACCTGACGTCGACGGATCGGGAGAT
CTCCCGATCCCCTATGGTGCACTCTCAGTACAATCTGCTCTGATGCCGCATAGTTAAGC
CAGTATCTGCTCCCTGCTTGTGTGTTGGAGGTCGCTGAGTAGTGCGCGAGCAAAATTTA
AGCTACAACAAGGCAAGGCTTGACCGACAATTCTCTGGCTAACTAGAGAACCCACTGCT
TACTAGGCGTCTCA (SEQ ID NO: 16)
Annotations:

| Name | Start | End |
|---|---|---|
| CA Vector-nLuc6 | 1 | 2319 |
| CAV repeat region | 144 | 260 |
| Start-less VP2 fragment | 380 | 1006 |
| Start-less VP3 (Apoptin) | 486 | 851 |
| Start-less VP1 upstream | 853 | 1006 |
| fragment | | |
| SV40 promoter | 1007 | 1350 |
| NanoLuc | 1357 | 1872 |
| SV40 poly(A) | 1873 | 1994 |
| Downstream VP1 fragment | 1995 | 2202 |
| Hairpin Region | 2206 | 2272 |
| CAV-APromAORFA3NCR | 2320 | 2641 |
| CAV repeat region | 2463 | 2579 |
| pUC origin | 2773 | 3446 |
| AmpR Gene | 3591 | 4550 |

Additional exemplary CAV genome sequences, for which sequences or subsequences comprised therein can be utilized in the compositions and methods described herein (e.g., to form a genetic element of an CAVector, as part of a genetic element construct for producing a CAVector, or as the genetic element sequence in a tandem construct, e.g., as described herein) are listed below in Table 17. In some embodiments, a CAVector comprises a genetic element comprising one or more of (e.g., at least 1, 2, 3, 4, 5, 6, 7, 8, 9, or 10 of) a 5′ UTR, repeat region, CAAT signal, TATA box, VP2-encoding sequence, Apoptin-encoding sequence, VP1-encoding sequence, 3′ UTR, GC-rich region, or polyA signal sequence from a CAV genome sequence listed in Table 17, or a sequence having at least about 70%, 75%, 80%, 85%, 90%, 95%, 96%, 97%, 98%, or 99% sequence identity thereto.

In some embodiments, a CAVector comprises a genetic element comprising a nucleic acid sequence encoding a polypeptide having at least about 70%, 75%, 80%, 85%, 90%, 95%, 96%, 97%, 98%, 99%, or 100% sequence identity to a VP1 molecule encoded by a CAV genome sequence listed in Table 17. In some embodiments, a CAVector comprises a genetic element comprising a nucleic acid sequence encoding a polypeptide having at least about 70%, 75%, 80%, 85%, 90%, 95%, 96%, 97%, 98%, 99%, or 100% sequence identity to a VP2 molecule encoded by a CAV genome sequence listed in Table 17. In some embodiments, a CAVector comprises a genetic element comprising a nucleic acid sequence encoding a polypeptide having at least about 70%, 75%, 80%, 85%, 90%, 95%, 96%, 97%, 98%, 99%, or 100% sequence identity to an Apoptin molecule encoded by a CAV genome sequence listed in Table 17.

In some embodiments, a CAVector comprises a genetic element that does not comprise a nucleic acid sequence encoding a polypeptide having at least about 70%, 75%, 80%, 85%, 90%, 95%, 96%, 97%, 98%, 99%, or 100% sequence identity to a VP& molecule encoded by a CAV genome sequence listed in Table 17. In some embodiments, a CAVector comprises a genetic element that does not comprise a nucleic acid sequence encoding a polypeptide having at least about 70%, 75%, 80%, 85%, 90%, 95%, 96%, 97%, 98%, 99%, or 100% sequence identity to a VP2 molecule encoded by a CAV genome sequence listed in Table 17. In some embodiments, a CAVector comprises a genetic element that does not comprise a nucleic acid sequence encoding a polypeptide having at least about 70%, 75%, 80%, 85%, 90%, 95%, 96%, 97%, 98%, 99%, or 100% sequence identity to an Apoptin molecule encoded by a CAV genome sequence listed in Table 17.

TABLE 17
Listing of Exemplary Wild-Type CAV Isolates

| # | Sequence Name | Length & Topology | Accession No(s) |
|---|---|---|---|
| 1 | 1. Chicken anemia virus | 2,298 bp circular DNA | MN103406.1 GI:1808996461 |
| | isolate GX1904B, complete | | |
| | genome | | |
| 2 | 2. Chicken anemia virus | 2,298 bp circular DNA | MN103405.1 GI:1808996453 |
| | isolate GX1805, complete | | |
| | genome | | |
| 3 | 3. Chicken anemia virus | 2,298 bp circular DNA | MN103404.1 GI:1808996446 |
| | isolate GX1905, complete | | |
| | genome | | |
| 4 | 4. Chicken anemia virus | 2,298 bp circular DNA | MN103403.1 GI:1808996438 |
| | isolate GX1904P, complete | | |
| | genome | | |
| 5 | 5. Chicken anemia virus | 2,298 bp circular DNA | MN103402.1 GI:1808996431 |
| | isolate GX1904A, complete | | |
| | genome | | |
| 6 | 6. Chicken anemia virus | 2,298 bp circular DNA | MN299317.1 GI:1785996135 |
| | isolate 1619TW, complete | | |
| | genome | | |
| 7 | 7. Chicken anemia virus | 2,298 bp circular DNA | MN299316.1 GI:1785996131 |
| | isolate 1852TW, complete | | |
| | genome | | |
| 8 | 8. Chicken anemia virus | 2,298 bp circular DNA | MN299315.1 GI:1785996127 |
| | isolate 1535TW, complete | | |
| | genome | | |
| 9 | 9. Chicken anemia virus | 2,298 bp circular DNA | MN299313.1 GI:1785996121 |
| | isolate 1777TW, complete | | |
| | genome | | |
| 10 | 10. Chicken anemia virus | 2,298 bp circular DNA | MN299312.1 GI:1785996117 |
| | isolate 1716TW, complete | | |
| | genome | | |
| 11 | 11. Chicken anemia virus | 2,298 bp circular DNA | MN299311.1 GI:1785996113 |
| | isolate 1725PT, complete | | |
| | genome | | |
| 12 | 12. Chicken anemia virus | 2,298 bp circular DNA | MN299310.1 GI:1785996109 |
| | isolate 1637TW, complete | | |
| | genome | | |
| 13 | 13. Chicken anemia virus | 2,298 bp circular DNA | MN299309.1 GI:1785996105 |
| | isolate 1510TW, complete | | |
| | genome | | |
| 14 | 14. Chicken anemia virus | 2,298 bp linear DNA | MK887171.1 GI:1777083749 |
| | isolate N1, complete | | |
| | genome | | |
| 15 | 15. Chicken anemia virus | 2,298 bp linear DNA | MK887170.1 GI:1777083745 |
| | isolate N2, complete | | |
| | genome | | |
| 16 | 16. Chicken anemia virus | 2,298 bp linear DNA | MK887169.1 GI:1777083741 |
| | isolate N3, complete | | |
| | genome | | |
| 17 | 17. Chicken anemia virus | 2,298 bp linear DNA | MK887168.1 GI:1777083737 |
| | isolate N4, complete | | |
| | genome | | |
| 18 | 18. Chicken anemia virus | 2,298 bp linear DNA | MK887167.1 GI:1777083733 |
| | isolate N5, complete | | |
| | genome | | |
| 19 | 19. Chicken anemia virus | 2,298 bp linear DNA | MK887166.1 GI:1777083729 |
| | isolate N6, complete | | |
| | genome | | |
| 20 | 20. Chicken anemia virus | 2,298 bp linear DNA | MK887165.1 GI:1777083725 |
| | isolate N7, complete | | |
| | genome | | |
| 21 | 21. Chicken anemia virus | 2,298 bp linear DNA | MK887164.1 GI:1777083721 |
| | isolate N8, complete | | |
| | genome | | |
| 22 | 22. Chicken anemia virus | 2,298 bp circular DNA | MK770259.1 GI:1755994599 |
| | isolate XH16, complete | | |
| | genome | | |
| 23 | 23. Chicken anemia virus | 1,823 bp circular DNA | MK423874.1 GI:1729913537 |
| | isolate Vietnam/VP1/18, | | |
| | complete genome | | |
| 24 | 24. Chicken anemia virus | 1,823 bp circular DNA | MK423873.1 GI:1729913533 |
| | isolate Vietnam/PT1/17, | | |
| | complete genome | | |
| 25 | 25. Chicken anemia virus | 1,823 bp circular DNA | MK423872.1 GI:1729913529 |
| | isolate Vietnam/HN3/17, | | |
| | complete genome | | |
| 26 | 26. Chicken anemia virus | 1,823 bp circular DNA | MK423871.1 GI:1729913525 |
| | isolate Vietnam/HN1/17, | | |
| | complete genome | | |
| 27 | 27. Chicken anemia virus | 1,823 bp circular DNA | MK423870.1 GI:1729913521 |
| | isolate Vietnam/HP1/17, | | |
| | complete genome | | |
| 28 | 28. Chicken anemia virus | 1,823 bp circular DNA | MK423869.1 GI:1729913517 |
| | isolate Vietnam/HB1/17, | | |
| | complete genome | | |
| 29 | 29. Chicken anemia virus | 1,823 bp circular DNA | MK423868.1 GI:1729913513 |
| | isolate Vietnam/BN1/17, | | |
| | complete genome | | |
| 30 | 30. Chicken anemia virus | 1,823 bp circular DNA | MK423867.1 GI:1729913509 |
| | isolate Vietnam/BN2/16, | | |
| | complete genome | | |
| 31 | 31. Chicken anemia virus | 1,823 bp circular DNA | MK423866.1 GI:1729913505 |
| | isolate Vietnam/HN2/16, | | |
| | complete genome | | |
| 32 | 32. Chicken anemia virus | 2,298 bp circular DNA | MK484616.1 GI:1682038571 |
| | isolate GX1810, complete | | |
| | genome | | |
| 33 | 33. Chicken anemia virus | 2,298 bp circular DNA | MK484615.1 GI:1682038567 |
| | isolate GX1804, complete | | |
| | genome | | |
| 34 | 34. Chicken anemia virus | 2,298 bp circular DNA | MK484614.1 GI:1682038563 |
| | isolate GX1801, complete | | |
| | genome | | |
| 35 | 35. Chicken anemia virus | 2,298 bp circular DNA | MK089243.1 GI:1658264359 |
| | isolate 17SY0902, complete | | |
| | genome | | |
| 36 | 36. Chicken anemia virus | 2,298 bp circular DNA | MK089242.1 GI:1658264355 |
| | isolate 17JL0314, complete | | |
| | genome | | |
| 37 | 37. Chicken anemia virus | 2,298 bp circular DNA | MK089241.1 GI:1658264351 |
| | isolate 17JL0310, complete | | |
| | genome | | |
| 38 | 38. Chicken anemia virus | 2,298 bp circular DNA | MK089240.1 GI:1658264347 |
| | isolate 17CC0509, complete | | |
| | genome | | |
| 39 | 39. Chicken anemia virus | 2,298 bp circular DNA | MK386570.1 GI:1616393965 |
| | isolate 1705PT, complete | | |
| | genome | | |
| 40 | 40. Chicken anemia virus | 2,298 bp circular DNA | MK376316.1 GI:1616393961 |
| | isolate 1636TW, complete | | |
| | genome | | |
| 41 | 41. Chicken anemia virus | 2,298 bp circular DNA | MK376315.1 GI:1616393957 |
| | isolate 1623TW, complete | | |
| | genome | | |
| 42 | 42. Chicken anemia virus | 2,298 bp circular DNA | MK360817.1 GI:1616393949 |
| | isolate 1520TW, complete | | |
| | genome | | |
| 43 | 43. Chicken anemia virus | 2,298 bp circular DNA | MK358456.1 GI:1616393943 |
| | isolate 1504TW, complete | | |
| | genome | | |
| 44 | 44. Chicken anemia virus | 2,298 bp circular DNA | MH536106.1 GI:1586080948 |
| | isolate G17.3.1, complete | | |
| | genome | | |
| 45 | 45. Chicken anemia virus | 2,298 bp circular DNA | MH536105.1 GI:1586080944 |
| | isolate G17.33.5, complete | | |
| | genome | | |
| 46 | 46. Chicken anemia virus | 2,298 bp circular DNA | MH536104.1 GI:1586080940 |
| | isolate G17.33.3, complete | | |
| | genome | | |
| 47 | 47. Chicken anemia virus | 2,298 bp circular DNA | MG827100.1 GI:1496531976 |
| | isolate CAV-SK4-2017, | | |
| | complete genome | | |
| 48 | 48. Chicken anemia virus | 2,298 bp circular DNA | MG827099.1 GI:1496531972 |
| | isolate CAV-GZ2-2016, | | |
| | complete genome | | |
| 49 | 49. Chicken anemia virus | 2,298 bp circular DNA | MG827098.1 GI:1496531968 |
| | isolate CAV-CA1-2015, | | |
| | complete genome | | |
| 50 | 50. Chicken anemia virus | 2,291 bp linear DNA | MH186142.1 GI:1493076326 |
| | strain CIAV-Shanxi7, | | |
| | complete genome | | |
| 51 | 51. Chicken anemia virus | 2,291 bp linear DNA | MH186141.1 GI:1493076321 |
| | strain CIAV-Guangdong11, | | |
| | complete genome | | |
| 52 | 52. Chicken anemia virus | 2,295 bp linear DNA | MH186140.1 GI:1493076316 |
| | strain CIAV-Anhui8, | | |
| | complete genome | | |
| 53 | 53. Chicken anemia virus | 2,293 bp linear DNA | MH186139.1 GI:1493076311 |
| | strain CIAV-Hebei2, | | |
| | complete genome | | |
| 54 | 54. Chicken anemia virus | 2,293 bp linear DNA | MH186138.1 GI:1493076306 |
| | strain CIAV-Hebei12, | | |
| | complete genome | | |
| 55 | 55. Chicken anemia virus | 2,295 bp linear DNA | MH186137.1 GI:1493076301 |
| | strain CIAV- | | |
| | Heilongjiang16, complete | | |
| | genome | | |
| 56 | 56. Chicken anemia virus | 2,298 bp circular DNA | MH001570.1 GI:1436168173 |
| | isolate CAV-EG-28, | | |
| | complete genome | | |
| 57 | 57. Chicken anemia virus | 2,298 bp circular DNA | MH001569.1 GI:1436168169 |
| | isolate CAV-EG-25, | | |
| | complete genome | | |
| 58 | 58. Chicken anemia virus | 2,298 bp circular DNA | MH001568.1 GI:1436168165 |
| | isolate CAV-EG-15, | | |
| | complete genome | | |
| 59 | 59. Chicken anemia virus | 2,298 bp circular DNA | MH001567.1 GI:1436168161 |
| | isolate CAV-EG-21, | | |
| | complete genome | | |
| 60 | 60. Chicken anemia virus | 2,298 bp circular DNA | MH001566.1 GI:1436168157 |
| | isolate CAV-EG-24, | | |
| | complete genome | | |
| 61 | 61. Chicken anemia virus | 2,298 bp circular DNA | MH001565.1 GI:1436168153 |
| | isolate CAV-EG-14, | | |
| | complete genome | | |
| 62 | 62. Chicken anemia virus | 2,298 bp circular DNA | MH001564.1 GI:1436168149 |
| | isolate CAV-EG-26, | | |
| | complete genome | | |
| 63 | 63. Chicken anemia virus | 2,298 bp circular DNA | MH001563.1 GI:1436168145 |
| | isolate CAV-EG-27, | | |
| | complete genome | | |
| 64 | 64. Chicken anemia virus | 2,298 bp circular DNA | MH001562.1 GI:1436168141 |
| | isolate CAV-EG-23, | | |
| | complete genome | | |
| 65 | 65. Chicken anemia virus | 2,298 bp circular DNA | MH001561.1 GI:1436168137 |
| | isolate CAV-EG-22, | | |
| | complete genome | | |
| 66 | 66. Chicken anemia virus | 2,298 bp circular DNA | MH001560.1 GI:1436168133 |
| | isolate CAV-EG-13, | | |
| | complete genome | | |
| 67 | 67. Chicken anemia virus | 2,298 bp circular DNA | MH001559.1 GI:1436168129 |
| | isolate CAV-EG-11, | | |
| | complete genome | | |
| 68 | 68. Chicken anemia virus | 2,298 bp circular DNA | MH001558.1 GI:1436168125 |
| | isolate CAV-EG-12, | | |
| | complete genome | | |
| 69 | 69. Chicken anemia virus | 2,298 bp circular DNA | MH001557.1 GI:1436168121 |
| | isolate CAV-EG-10, | | |
| | complete genome | | |
| 70 | 70. Chicken anemia virus | 2,298 bp circular DNA | MH001556.1 GI:1436168117 |
| | isolate CAV-EG-7, | | |
| | complete genome | | |
| 71 | 71. Chicken anemia virus | 2,298 bp circular DNA | MH001555.1 GI:1436168113 |
| | isolate CAV-EG-6, | | |
| | complete genome | | |
| 72 | 72. Chicken anemia virus | 2,298 bp circular DNA | MH001554.1 GI:1436168109 |
| | isolate CAV-EG-4, | | |
| | complete genome | | |
| 73 | 73. Chicken anemia virus | 2,298 bp circular DNA | MH001553.1 GI:1436168105 |
| | isolate CAV-EG-2, | | |
| | complete genome | | |
| 74 | 74. UNVERIFIED: Chicken | 2,293 bp linear DNA | MF614011.1 GI:1420860182 |
| | anemia virus strain | | |
| | SDAUC-Vac, complete | | |
| | genome | | |
| 75 | 75. Chicken anemia virus | 2,298 bp circular DNA | MG846491.1 GI:1360474804 |
| | strain RS/BR/15/1R, | | |
| | complete genome | | |
| 76 | 76. Chicken anemia virus | 2,298 bp circular DNA | KY486155.1 GI:1325381006 |
| | isolate LN15170, complete | | |
| | genome | | |
| 77 | 77. Chicken anemia virus | 2,298 bp circular DNA | KY486154.1 GI:1325381002 |
| | isolate LN15169, complete | | |
| | genome | | |
| 78 | 78. Chicken anemia virus | 2,298 bp circular DNA | KY486153.1 GI:1325380998 |
| | isolate JS15166, complete | | |
| | genome | | |
| 79 | 79. Chicken anemia virus | 2,298 bp circular DNA | KY486152.1 GI:1325380994 |
| | isolate JS15165, complete | | |
| | genome | | |
| 80 | 80. Chicken anemia virus | 2,298 bp circular DNA | KY486151.1 GI:1325380990 |
| | isolate NX15140, complete | | |
| | genome | | |
| 81 | 81. Chicken anemia virus | 2,298 bp circular DNA | KY486150.1 GI:1325380986 |
| | isolate NX15091, complete | | |
| | genome | | |
| 82 | 82. Chicken anemia virus | 2,298 bp circular DNA | KY486149.1 GI:1325380982 |
| | isolate JL15120, complete | | |
| | genome | | |
| 83 | 83. Chicken anemia virus | 2,298 bp circular DNA | KY486148.1 GI:1325380978 |
| | isolate JL14028, complete | | |
| | genome | | |
| 84 | 84. Chicken anemia virus | 2,298 bp circular DNA | KY486147.1 GI:1325380974 |
| | isolate JL14026, complete | | |
| | genome | | |
| 85 | 85. Chicken anemia virus | 2,298 bp circular DNA | KY486146.1 GI:1325380970 |
| | isolate JL14024, complete | | |
| | genome | | |
| 86 | 86. Chicken anemia virus | 2,298 bp circular DNA | KY486145.1 GI:1325380966 |
| | isolate JL14023, complete | | |
| | genome | | |
| 87 | 87. Chicken anemia virus | 2,298 bp circular DNA | KY486144.1 GI:1325380962 |
| | isolate HLJ15170, complete | | |
| | genome | | |
| 88 | 88. Chicken anemia virus | 2,298 bp circular DNA | KY486143.1 GI:1325380958 |
| | isolate HLJ15169, complete | | |
| | genome | | |
| 89 | 89. Chicken anemia virus | 2,298 bp circular DNA | KY486142.1 GI:1325380954 |
| | isolate HLJ15166, complete | | |
| | genome | | |
| 90 | 90. Chicken anemia virus | 2,298 bp circular DNA | KY486141.1 GI:1325380950 |
| | isolate HLJ15165, complete | | |
| | genome | | |
| 91 | 91. Chicken anemia virus | 2,298 bp circular DNA | KY486140.1 GI:1325380946 |
| | isolate HLJ15140, complete | | |
| | genome | | |
| 92 | 92. Chicken anemia virus | 2,298 bp circular DNA | KY486139.1 GI:1325380942 |
| | isolate HLJ15125, complete | | |
| | genome | | |
| 93 | 93. Chicken anemia virus | 2,298 bp circular DNA | KY486138.1 GI:1325380938 |
| | isolate HLJ15109, complete | | |
| | genome | | |
| 94 | 94. Chicken anemia virus | 2,298 bp circular DNA | KY486137.1 GI:1325380934 |
| | isolate HLJ15108, complete | | |
| | genome | | |
| 95 | 95. Chicken anemia virus | 2,298 bp circular DNA | KY486136.1 GI:1325380930 |
| | isolate HLJ14101, complete | | |
| | genome | | |
| 96 | 96. Chicken anemia virus | 2,263 bp circular DNA | KY053900.1 GI:1249091115 |
| | isolate | | |
| | CAV/NAM/TANUVAS/09, | | |
| | complete genome | | |
| 97 | 97. Chicken anemia virus | 2,298 bp circular DNA | KX447637.1 GI:1216618959 |
| | isolate LY-2, complete | | |
| | genome | | |
| 98 | 98. Chicken anemia virus | 2,298 bp circular DNA | KX447636.1 GI:1216618955 |
| | isolate LY-1, complete | | |
| | genome | | |
| 99 | 99. Chicken anemia virus | 2,298 bp circular DNA | KX447635.1 GI:1216618951 |
| | isolate HB160430, complete | | |
| | genome | | |
| 100 | 100. Chicken anemia virus | 2,298 bp circular DNA | KX447634.1 GI:1216618947 |
| | isolate BS-C2, complete | | |
| | genome | | |
| 101 | 101. Chicken anemia virus | 2,298 bp circular DNA | KX447633.1 GI:1216618943 |
| | isolate BS-C1, complete | | |
| | genome | | |
| 102 | 102. Chicken anemia virus | 2,298 bp circular DNA | KY024579.1 GI:1124881742 |
| | strain RS/BR/15, complete | | |
| | genome | | |
| 103 | 103. Chicken anemia virus | 2,298 bp circular DNA | KU645525.1 GI:1078570421 |
| | isolate CIAV-Mouse, | | |
| | complete genome | | |
| 104 | 104. Chicken anemia virus | 2,298 bp circular DNA | KU645524.1 GI:1078570417 |
| | isolate CIAV-Dog, | | |
| | complete genome | | |
| 105 | 105. Chicken anemia virus | 2,298 bp circular DNA | KU645523.1 GI:1078570413 |
| | isolate SD1505, complete | | |
| | genome | | |
| 106 | 106. Chicken anemia virus | 2,298 bp circular DNA | KU645522.1 GI:1078570409 |
| | isolate SD1518, complete | | |
| | genome | | |
| 107 | 107. Chicken anemia virus | 2,298 bp circular DNA | KU645521.1 GI:1078570405 |
| | isolate SD1514, complete | | |
| | genome | | |
| 108 | 108. Chicken anemia virus | 2,298 bp circular DNA | KU645520.1 GI:1078570401 |
| | isolate HN1405, complete | | |
| | genome | | |
| 109 | 109. Chicken anemia virus | 2,298 bp circular DNA | KU645519.1 GI:1078570397 |
| | isolate SD1508, complete | | |
| | genome | | |
| 110 | 110. Chicken anemia virus | 2,298 bp circular DNA | KU645518.1 GI:1078570393 |
| | isolate JS1501, complete | | |
| | genome | | |
| 111 | 111. Chicken anemia virus | 2,298 bp circular DNA | KU645517.1 GI:1078570389 |
| | isolate SD1513, complete | | |
| | genome | | |
| 112 | 112. Chicken anemia virus | 2,298 bp circular DNA | KU645516.1 GI:1078570385 |
| | isolate HB1517, complete | | |
| | genome | | |
| 113 | 113. Chicken anemia virus | 2,298 bp circular DNA | KU645515.1 GI:1078570381 |
| | isolate SD1515, complete | | |
| | genome | | |
| 114 | 114. Chicken anemia virus | 2,298 bp circular DNA | KU645514.1 GI:1078570377 |
| | isolate HB1404, complete | | |
| | genome | | |
| 115 | 115. Chicken anemia virus | 2,298 bp circular DNA | KU645513.1 GI:1078570373 |
| | isolate BJ1406, complete | | |
| | genome | | |
| 116 | 116. Chicken anemia virus | 2,298 bp circular DNA | KU645512.1 GI:1078570369 |
| | isolate HN1504, complete | | |
| | genome | | |
| 117 | 117. Chicken anemia virus | 2,298 bp circular DNA | KU645511.1 GI:1078570365 |
| | isolate LN1402, complete | | |
| | genome | | |
| 118 | 118. Chicken anemia virus | 2,298 bp circular DNA | KU645510.1 GI:1078570361 |
| | isolate SD1509, complete | | |
| | genome | | |
| 119 | 119. Chicken anemia virus | 2,298 bp circular DNA | KU645509.1 GI:1078570357 |
| | isolate JS1503, complete | | |
| | genome | | |
| 120 | 120. Chicken anemia virus | 2,298 bp circular DNA | KU645508.1 GI:1078570353 |
| | isolate SD1502, complete | | |
| | genome | | |
| 121 | 121. Chicken anemia virus | 2,298 bp circular DNA | KU645507.1 GI:1078570349 |
| | isolate SD1507, complete | | |
| | genome | | |
| 122 | 122. Chicken anemia virus | 2,298 bp circular DNA | KU645506.1 GI:1078570345 |
| | isolate SD1512, complete | | |
| | genome | | |
| 123 | 123. Chicken anemia virus | 2,298 bp circular DNA | KU641015.1 GI:1073547898 |
| | isolate SD1511, complete | | |
| | genome | | |
| 124 | 124. Chicken anemia virus | 2,298 bp circular DNA | KU641014.1 GI:1073547894 |
| | isolate JN1503, complete | | |
| | genome | | |
| 125 | 125. Chicken anemia virus | 2,298 bp circular DNA | KU641013.1 GI:1073547890 |
| | isolate LN1401, complete | | |
| | genome | | |
| 126 | 126. Chicken anemia virus | 2,298 bp circular DNA | KU598851.1 GI:1073547886 |
| | isolate SD1510, complete | | |
| | genome | | |
| 127 | 127. Chicken anemia virus | 2,298 bp linear DNA | KU050680.1 GI:1021673541 |
| | strain GD-101, complete | | |
| | genome | | |
| 128 | 128. Chicken anemia virus | 2,298 bp linear DNA | KU050679.1 GI:1021673537 |
| | strain GD-104, complete | | |
| | genome | | |
| 129 | 129. Chicken anemia virus | 2,298 bp linear DNA | KU050678.1 GI:1021673533 |
| | strain GD-103, complete | | |
| | genome | | |
| 130 | 130. Chicken anemia virus | 2,298 bp linear DNA | KU050677.1 GI:1021673529 |
| | strain GD-102, complete | | |
| | genome | | |
| 131 | 131. Chicken anemia virus | 2,298 bp circular DNA | KU221054.1 GI:1004125710 |
| | isolate SD1403, complete | | |
| | genome | | |
| 132 | 132. Chicken anemia virus | 2,298 bp circular DNA | KU845735.1 GI:1002623535 |
| | isolate CIAV-F10, complete | | |
| | genome | | |
| 133 | 133. Chicken anemia virus | 2,298 bp circular DNA | KU845734.1 GI:1002623531 |
| | isolate FAdV-N22, | | |
| | complete genome | | |
| 134 | 134. Chicken anemia virus | 2,298 bp circular DNA | KJ872514.1 GI:701168412 |
| | isolate CAV-18, complete | | |
| | genome | | |
| 135 | 135. Chicken anemia virus | 2,298 bp circular DNA | KJ872513.1 GI:701168408 |
| | isolate CAV-10, complete | | |
| | genome | | |
| 136 | 136. Chicken anemia virus | 2,298 bp circular DNA | KJ728830.1 GI:660450622 |
| | isolate 22, complete | | |
| | sequence | | |
| 137 | 137. Chicken anemia virus | 2,298 bp circular DNA | KJ728829.1 GI:660450618 |
| | isolate 20, complete | | |
| | sequence | | |
| 138 | 138. Chicken anemia virus | 2,298 bp circular DNA | KJ728828.1 GI:660450614 |
| | isolate 19, complete | | |
| | sequence | | |
| 139 | 139. Chicken anemia virus | 2,298 bp circular DNA | KJ728827.1 GI:660450610 |
| | isolate 18, complete | | |
| | sequence | | |
| 140 | 140. Chicken anemia virus | 2,298 bp circular DNA | KJ728826.1 GI:660450606 |
| | isolate 17, complete | | |
| | sequence | | |
| 141 | 141. Chicken anemia virus | 2,298 bp circular DNA | KJ728825.1 GI:660450602 |
| | isolate 16, complete | | |
| | sequence | | |
| 142 | 142. Chicken anemia virus | 2,298 bp circular DNA | KJ728824.1 GI:660450598 |
| | isolate 14, complete | | |
| | sequence | | |
| 143 | 143. Chicken anemia virus | 2,298 bp circular DNA | KJ728823.1 GI:660450594 |
| | isolate 13, complete | | |
| | sequence | | |
| 144 | 144. Chicken anemia virus | 2,298 bp circular DNA | KJ728822.1 GI:660450590 |
| | isolate 12, complete | | |
| | sequence | | |
| 145 | 145. Chicken anemia virus | 2,298 bp circular DNA | KJ728821.1 GI:660450586 |
| | isolate 10, complete | | |
| | sequence | | |
| 146 | 146. Chicken anemia virus | 2,298 bp circular DNA | KJ728820.1 GI:660450582 |
| | isolate 9, complete sequence | | |
| 147 | 147. Chicken anemia virus | 2,298 bp circular DNA | KJ728819.1 GI:660450578 |
| | isolate 8, complete sequence | | |
| 148 | 148. Chicken anemia virus | 2,298 bp circular DNA | KJ728818.1 GI:660450574 |
| | isolate 7, complete sequence | | |
| 149 | 149. Chicken anemia virus | 2,298 bp circular DNA | KJ728817.1 GI:660450570 |
| | isolate 6, complete sequence | | |
| 150 | 150. Chicken anemia virus | 2,298 bp circular DNA | KJ728816.1 GI:660450566 |
| | isolate 4, complete sequence | | |
| 151 | 151. Chicken anemia virus | 2,298 bp circular DNA | KJ728815.1 GI:660450562 |
| | isolate 2, complete sequence | | |
| 152 | 152. Chicken anemia virus | 2,298 bp circular DNA | KJ728814.1 GI:660450558 |
| | isolate 1, complete sequence | | |
| 153 | 153. Chicken anemia virus | 1,812 bp linear DNA | KJ621019.1 GI:648091733 |
| | isolate | | |
| | VRDC/CAV/MH/C32/11 | | |
| | VP2 protein (VP2), apoptin | | |
| | (VP3), and capsid protein | | |
| | (VP1) genes, complete cds | | |
| 154 | 154. Chicken anemia virus | 1,814 bp linear DNA | KJ621018.1 GI:648091729 |
| | isolate | | |
| | VRDC/CAV/HR/C31/11 | | |
| | VP2 protein (VP2), apoptin | | |
| | (VP3), and capsid protein | | |
| | (VP1) genes, complete cds | | |
| 155 | 155. Chicken anemia virus | 1,812 bp linear DNA | KJ621017.1 GI:648091725 |
| | isolate | | |
| | VRDC/CAV/AP/C30/11 | | |
| | VP2 protein (VP2), apoptin | | |
| | (VP3), and capsid protein | | |
| | (VP1) genes, complete cds | | |
| 156 | 156. Chicken anemia virus | 1,826 bp linear DNA | KJ621016.1 GI:648091721 |
| | isolate | | |
| | VRDC/CAV/HR/C29/11 | | |
| | VP2 protein (VP2), apoptin | | |
| | (VP3), and capsid protein | | |
| | (VP1) genes, complete cds | | |
| 157 | 157. Chicken anemia virus | 1,822 bp linear DNA | KJ621015.1 GI:648091717 |
| | isolate | | |
| | VRDC/CAV/HR/C28/11 | | |
| | VP2 protein (VP2), apoptin | | |
| | (VP3), and capsid protein | | |
| | (VP1) genes, complete cds | | |
| 158 | 158. Chicken anemia virus | 1,817 bp linear DNA | KJ621014.1 GI:648091713 |
| | isolate | | |
| | VRDC/CAV/HR/C27/11 | | |
| | VP2 Protein (VP2), apoptin | | |
| | (VP3), and capsid protein | | |
| | (VP1) genes, complete cds | | |
| 159 | 159. Chicken anemia virus | 1,821 bp linear DNA | KJ621013.1 GI:648091709 |
| | isolate | | |
| | VRDC/CAV/HR/C26/11 | | |
| | VP2 protein (VP2), apoptin | | |
| | (VP3), and capsid protein | | |
| | (VP1) genes, complete cds | | |
| 160 | 160. Chicken anemia virus | 1,816 bp linear DNA | KJ621012.1 GI:648091705 |
| | isolate | | |
| | VRDC/CAV/HR/C25/11 | | |
| | VP2 protein (VP2), apoptin | | |
| | (VP3), and capsid protein | | |
| | (VP1) genes, complete cds | | |
| 161 | 161. Chicken anemia virus | 1,820 bp linear DNA | KJ621011.1 GI:648091701 |
| | isolate | | |
| | VRDC/CAV/HR/C24/11 | | |
| | VP2 protein (VP2), apoptin | | |
| | (VP3), and capsid protein | | |
| | (VP1) genes, complete cds | | |
| 162 | 162. Chicken anemia virus | 1,811 bp linear DNA | KJ621010.1 GI:648091697 |
| | isolate | | |
| | VRDC/CAV/HR/C23/11 | | |
| | VP2 protein (VP2), apoptin | | |
| | (VP3), and capsid protein | | |
| | (VP1) genes, complete cds | | |
| 163 | 163. Chicken anemia virus | 1,807 bp linear DNA | KJ621009.1 GI:648091693 |
| | isolate | | |
| | VRDC/CAV/HR/C22/11 | | |
| | VP2 protein (VP2), apoptin | | |
| | (VP3), and capsid protein | | |
| | (VP1) genes, complete cds | | |
| 164 | 164. Chicken anemia virus | 1,833 bp linear DNA | KJ621008.1 GI:648091689 |
| | isolate | | |
| | VRDC/CAV/HR/C21/11 | | |
| | VP2 protein (VP2), apoptin | | |
| | (VP3), and capsid protein | | |
| | (VP1) genes, complete cds | | |
| 165 | 165. Chicken anemia virus | 1,822 bp linear DNA | KJ621007.1 GI:648091685 |
| | isolate | | |
| | VRDC/CAV/HR/C20/11 | | |
| | VP2 protein (VP2), apoptin | | |
| | (VP3), and capsid protein | | |
| | (VP1) genes, complete cds | | |
| 166 | 166. Chicken anemia virus | 1,822 bp linear DNA | KJ621006.1 GI:648091681 |
| | isolate | | |
| | VRDC/CAV/HR/C19/11 | | |
| | VP2 protein (VP2), apoptin | | |
| | (VP3), and capsid protein | | |
| | (VP1) genes, complete cds | | |
| 167 | 167. Chicken anemia virus | 1,811 bp linear DNA | KJ621005.1 GI:648091677 |
| | isolate | | |
| | VRDC/CAV/HR/C18/11 | | |
| | VP2 protein (VP2), apoptin | | |
| | (VP3), and capsid protein | | |
| | (VP1) genes, complete cds | | |
| 168 | 168. Chicken anemia virus | 1,825 bp linear DNA | KJ621004.1 GI:648091673 |
| | isolate | | |
| | VRDC/CAV/HR/C17/11 | | |
| | VP2 protein (VP2), apoptin | | |
| | (VP3), and capsid protein | | |
| | (VP1) genes, complete cds | | |
| 169 | 169. Chicken anemia virus | 1,816 bp linear DNA | KJ621003.1 GI:648091669 |
| | isolate | | |
| | VRDC/CAV/AP/C16/11 | | |
| | VP2 protein (VP2), apoptin | | |
| | (VP3), and capsid protein | | |
| | (VP1) genes, complete cds | | |
| 170 | 170. Chicken anemia virus | 1,786 bp linear DNA | KJ621002.1 GI:648091665 |
| | isolate | | |
| | VRDC/CAV/CHD/C14/11 | | |
| | VP2 protein (VP2), apoptin | | |
| | (VP3), and capsid protein | | |
| | (VP1) genes, complete cds | | |
| 171 | 171. Chicken anemia virus | 1,806 bp linear DNA | KJ621001.1 GI:648091661 |
| | isolate | | |
| | VRDC/CAV/HR/C13/11 | | |
| | VP2 protein (VP2), apoptin | | |
| | (VP3), and capsid protein | | |
| | (VP1) genes, complete cds | | |
| 172 | 172. Chicken anemia virus | 1,838 bp linear DNA | KJ621000.1 GI:648091657 |
| | isolate | | |
| | VRDC/CAV/HR/C12/11 | | |
| | VP2 protein (VP2), apoptin | | |
| | (VP3), and capsid protein | | |
| | (VP1) genes, complete cds | | |
| 173 | 173. Chicken anemia virus | 1,822 bp linear DNA | KJ620999.1 GI:648091653 |
| | isolate | | |
| | VRDC/CAV/HR/C11/11 | | |
| | VP2 protein (VP2), apoptin | | |
| | (VP3), and capsid protein | | |
| | (VP1) genes, complete cds | | |
| 174 | 174. Chicken anemia virus | 1,807 bp linear DNA | KJ620998.1 GI:648091649 |
| | isolate | | |
| | VRDC/CAV/HR/C10/11 | | |
| | VP2 protein (VP2), apoptin | | |
| | (VP3), and capsid protein | | |
| | (VP1) genes, complete cds | | |
| 175 | 175. Chicken anemia virus | 1,803 bp linear DNA | KJ620997.1 GI:648091645 |
| | isolate | | |
| | VRDC/CAV/HR/C9/11 | | |
| | VP2 protein (VP2), apoptin | | |
| | (VP3), and capsid protein | | |
| | (VP1) genes, complete cds | | |
| 176 | 176. Chicken anemia virus | 1,874 bp linear DNA | KJ620989.1 GI:648091593 |
| | isolate | | |
| | VRDC/CAV/TN/C1/08 | | |
| | VP2 protein (VP2), apoptin | | |
| | (VP3), and capsid protein | | |
| | (VP1) genes, complete cds | | |
| 177 | 177. Chicken anemia virus | 2,295 bp circular DNA | KF224938.1 GI:545561491 |
| | strain GD-N-12, complete | | |
| | genome | | |
| 178 | 178. Chicken anemia virus | 2,298 bp circular DNA | KF224937.1 GI:545561487 |
| | strain GD-M-12, complete | | |
| | genome | | |
| 179 | 179. Chicken anemia virus | 2,298 bp circular DNA | KF224936.1 GI:545561483 |
| | strain GD-L-12, complete | | |
| | genome | | |
| 180 | 180. Chicken anemia virus | 2,298 bp circular DNA | KF224935.1 GI:545561479 |
| | strain GD-K-12, complete | | |
| | genome | | |
| 181 | 181. Chicken anemia virus | 2,298 bp circular DNA | KF224934.1 GI:545561475 |
| | strain GD-J-12, complete | | |
| | genome | | |
| 182 | 182. Chicken anemia virus | 2,298 bp circular DNA | KF224933.1 GI:545561471 |
| | strain GD-I-12, complete | | |
| | genome | | |
| 183 | 183. Chicken anemia virus | 2,298 bp circular DNA | KF224932.1 GI:545561467 |
| | strain GD-H-12, complete | | |
| | genome | | |
| 184 | 184. Chicken anemia virus | 2,298 bp circular DNA | KF224931.1 GI:545561463 |
| | strain GD-G-12, complete | | |
| | genome | | |
| 185 | 185. Chicken anemia virus | 2,298 bp circular DNA | KF224930.1 GI:545561459 |
| | strain GD-F-12, complete | | |
| | genome | | |
| 186 | 186. Chicken anemia virus | 2,298 bp circular DNA | KF224929.1 GI:545561455 |
| | strain GD-E-12, complete | | |
| | genome | | |
| 187 | 187. Chicken anemia virus | 2,298 bp circular DNA | KF224928.1 GI:545561451 |
| | strain GD-D-12, complete | | |
| | genome | | |
| 188 | 188. Chicken anemia virus | 2,298 bp circular DNA | KF224927.1 GI:545561447 |
| | strain GD-C-12, complete | | |
| | genome | | |
| 189 | 189. Chicken anemia virus | 2,298 bp circular DNA | KF224926.1 GI:545561443 |
| | strain GD-B-12, complete | | |
| | genome | | |
| 190 | 190. Chicken anemia virus | 2,295 bp linear DNA | KC414026.1 GI:472320341 |
| | strain CAT-CAV, complete | | |
| | genome | | |
| 191 | 191. Chicken anemia virus | 2,292 bp circular DNA | JX964755.1 GI:429510192 |
| | isolate GXC060821, | | |
| | complete genome | | |
| 192 | 192. Chicken anemia virus | 2,298 bp circular DNA | JX260426.1 GI:403495278 |
| | isolate GD-1-12, complete | | |
| | genome | | |
| 193 | 193. Chicken anemia virus, | 2,316 bp circular DNA | JQ690762.1 GI:387600189 |
| | complete genome | | |
| 194 | 194. Chicken anemia virus | 2,298 bp circular DNA | JF507715.1 GI:345104687 |
| | isolate CIAV89-69, | | |
| | complete genome | | |
| 195 | 195. Chicken anemia virus | 2,298 bp linear DNA | HM134240.1 GI:299474029 |
| | VP2, VP3, and VP1 genes, | | |
| | complete cds | | |
| 196 | 196. Chicken anemia virus | 2,298 bp circular DNA | FJ172347.1 GI:205364143 |
| | isolate SDLY08, complete | | |
| | genome | | |
| 197 | 197. Chicken anemia virus | 2,279 bp linear DNA | EF683159.1 GI:152207654 |
| | vaccine strain | | |
| | 3711/chicken/Australia VP2 | | |
| | gene,complete cds; VP3 | | |
| | gene, complete cds; and | | |
| | VP1 gene, complete cds | | |
| 198 | 198. Chicken anemia virus | 2,298 bp circular DNA | EF176599.1 GI:121592425 |
| | isolate C14, complete | | |
| | genome | | |
| 199 | 199. Chicken anemia virus | 2,298 bp circular DNA | DQ991394.1 GI:116248476 |
| | isolate 01-4201, complete | | |
| | genome | | |
| 200 | 200. Chicken anemia virus | 2,298 bp circular DNA | AF311900.3 GI:114229361 |
| | isolate 98D06073, complete | | |
| | genome | | |
| 201 | 201. Chicken anemia virus | 2,298 bp circular DNA | DQ217401.1 GI:78068049 |
| | isolate SMSC-1P123WT, | | |
| | complete genome | | |
| 202 | 202. Chicken anemia virus | 2,298 bp circular DNA | DQ217400.1 GI:78068045 |
| | isolate SMSC-1P9WT, | | |
| | complete genome | | |
| 203 | 203. Chicken anemia virus | 2,298 bp circular DNA | DQ141673.1 GI:72536139 |
| | isolate SD22, complete | | |
| | genome | | |
| 204 | 204. Chicken anemia virus | 2,298 bp circular DNA | DQ141672.1 GI:72536135 |
| | isolate HN9, complete | | |
| | genome | | |
| 205 | 205. Chicken anemia virus | 2,298 bp circular DNA | DQ141671.1 GI:72536131 |
| | isolate SH16, complete | | |
| | genome | | |
| 206 | 206. Chicken anemia virus | 2,298 bp circular DNA | DQ141670.1 GI:72536127 |
| | isolate SH11, complete | | |
| | genome | | |
| 207 | 207. Chicken anemia virus | 2,298 bp circular DNA | DQ124936.1 GI:71738206 |
| | isolate AH4 from China, | | |
| | complete genome | | |
| 208 | 208. Chicken anemia virus | 2,298 bp circular DNA | DQ124935.1 GI:71738202 |
| | isolate AH6 from China, | | |
| | complete genome | | |
| 209 | 209. Chicken anemia virus | 2,298 bp circular DNA | DQ124934.1 GI:71738198 |
| | isolate BJ0401 from China, | | |
| | complete genome | | |
| 210 | 210. Chicken anemia virus | 2,298 bp circular DNA | AY839944.2 GI:68580448 |
| | strain LF4, complete | | |
| | genome | | |
| 211 | 211. Chicken anemia virus | 2,298 bp circular DNA | AY843527.2 GI:68577214 |
| | isolate TJBD33, complete | | |
| | genome | | |
| 212 | 212. Chicken anemia virus | 2,298 bp circular DNA | AY999018.1 GI:62823175 |
| | isolate SD24, complete | | |
| | genome | | |
| 213 | 213. Chicken anemia virus | 2,298 bp circular DNA | AY846844.1 GI:56791774 |
| | strain TJBD40, complete | | |
| | genome | | |
| 214 | 214. Chicken anemia virus | 2,298 bp linear DNA | AB119448.1 GI:45126694 |
| | genes for VP2, VP3, VP1, | | |
| | complete cds | | |
| 215 | 215. Chicken anemia virus | 2,298 bp circular DNA | AF311892.2 GI:32527857 |
| | isolate 98D02152, complete | | |
| | genome | | |
| 216 | 216. Chicken anemia virus | 2,298 bp circular DNA | AY040632.1 GI:22073877 |
| | isolate 3-1P60, complete | | |
| | genome | | |
| 217 | 217. Chicken anemia virus | 2,298 bp circular DNA | AF390102.1 GI:21666303 |
| | attenuated isolate SMSC- | | |
| | 1P60, complete genome | | |
| 218 | 218. Chicken anemia virus | 2,298 bp circular DNA | AF395114.1 GI:20378212 |
| | strain BD-3, complete | | |
| | genome | | |
| 219 | 219. Chicken anemia virus | 2,298 bp circular DNA | AF390038.1 GI:19851825 |
| | isolate 3-1 from Malaysia, | | |
| | complete genome | | |
| 220 | 220. Chicken anemia virus | 2,298 bp circular DNA | AF475908.1 GI:18874693 |
| | from China, complete | | |
| | genome | | |
| 221 | 221. Chicken anaemia virus | 2,297 bp circular DNA | AJ297685.2 GI:18643934 |
| | complete genome, clone 34 | | |
| 222 | 222. Chicken anaemia virus | 2,298 bp circular DNA | AJ297684.2 GI:18643933 |
| | complete genome, clone 33 | | |
| 223 | 223. Chicken anemia virus | 2,298 bp circular DNA | AF285882.1 GI:15076979 |
| | isolate SMSC-1 from | | |
| | Malaysia, complete genome | | |
| 224 | 224. Chicken anemia virus | 2,294 bp linear DNA | AF313470.1 GI:11321455 |
| | VP2, VP3, and VP1 genes, | | |
| | complete cds | | |
| 225 | 225. Chicken anemia virus | 2,298 bp circular DNA | AB027470.1 GI:4903007 |
| | DNA, complete genome, | | |
| | strain: TR20 | | |
| 226 | 226. Chicken anemia virus | 2,298 bp circular DNA | L14767.1 GI:4186172 |
| | unknown genes | | |
| 227 | 227. Chicken anemia virus | 2,319 bp circular DNA | U66304.1 GI:2558685 |
| | cloned isolate 10, complete | | |
| | genome | | |
| 228 | 228. Sequence 1 from | 2,319 bp circular DNA | A48606.1 GI:2302375 |
| | Patent WO9603507 | | |
| 229 | 229. Chicken anemia virus | 2,298 bp circular DNA | U65414.1 GI:1490881 |
| | complete genome: VP2, | | |
| | VP3 and VP1 genes, | | |
| | complete cds | | |
| 230 | 230. Chicken anemia virus | 2,298 bp circular DNA | D10068.1 GI:221116 |
| | DNA, complete genome | | |

### VP1 Molecules

In some embodiments, the CAVector comprises a proteinaceous exterior comprising a CAV VP1 molecule. Generally, an VP1 molecule comprises a polypeptide having the structural features and/or activity of an CAV VP1 protein (e.g., an CAV VP1 protein as described herein). In some embodiments, the VP1 molecule comprises a truncation relative to an CAV VP1 protein (e.g., an CAV VP1 protein as described herein). An VP1 molecule may be capable of binding to other VP1 molecules, e.g., to form a proteinaceous exterior (e.g., as described herein), e.g., a capsid. In some embodiments, the proteinaceous exterior may enclose a nucleic acid molecule (e.g., a genetic element as described herein). In some embodiments, a plurality of VP1 molecules may form a multimer, e.g., to form a proteinaceous exterior. In some embodiments, the multimer may be a homomultimer. In other embodiments, the multimer may be a heteromultimer.

An VP1 molecule may, in some embodiments, comprise one or more of: an arginine rich region, e.g., a region having at least 60% basic residues (e.g., at least 60%, 65%, 70%, 75%, 80%, 85%, 90%, 95%, or 100% basic residues; e.g., between 60%-90%, 60%-80%, 70%-90%, or 70-80% basic residues), and a jelly-roll domain.

Arginine-Rich Region

An arginine rich region has at least 70% (e.g., at least about 70, 80, 90, 95, 96, 97, 98, 99, or 100%) sequence identity to an arginine-rich region sequence described herein or a sequence of at least about 40 amino acids comprising at least 60%, 70%, or 80% basic residues (e.g., arginine, lysine, or a combination thereof).

Jelly Roll Domain

A jelly-roll domain or region comprises (e.g., consists of) a polypeptide (e.g., a domain or region comprised in a larger polypeptide) comprising one or more (e.g., 1, 2, or 3) of the following characteristics:
(i) at least 30% (e.g., at least 30%, 35%, 40%, 45%, 50%, 55%, 60%, 65%, 70%, 75%, 80%, 90%, or more) of the amino acids of the jelly-roll domain are part of one or more β-sheets;
(ii) the secondary structure of the jelly-roll domain comprises at least four (e.g., at least 4, 5, 6, 7, 8, 9, 10, 11, or 12) β-strands; and/or
(iii) the tertiary structure of the jelly-roll domain comprises at least two (e.g., at least 2, 3, or 4) β-sheets; and/or
(iv) the jelly-roll domain comprises a ratio of β-sheets to α-helices of at least 2:1, 3:1, 4:1, 5:1, 6:1, 7:1, 8:1, 9:1, or 10:1.

In certain embodiments, a jelly-roll domain comprises two β-sheets.

In certain embodiments, one or more (e.g., 1, 2, 3, 4, 5, 6, 7, 8, 9, or 10) of the β-sheets comprises about eight (e.g., 4, 5, 6, 7, 8, 9, 10, 11, or 12) β-strands. In certain embodiments, one or more (e.g., 1, 2, 3, 4, 5, 6, 7, 8, 9, or 10) of the β-sheets comprises eight β-strands. In certain embodiments, one or more (e.g., 1, 2, 3, 4, 5, 6, 7, 8, 9, or 10) of the β-sheets comprises seven β-strands. In certain embodiments, one or more (e.g., 1, 2, 3, 4, 5, 6, 7, 8, 9, or 10) of the β-sheets comprises six β-strands. In certain embodiments, one or more (e.g., 1, 2, 3, 4, 5, 6, 7, 8, 9, or 10) of the β-sheets comprises five β-strands. In certain embodiments, one or more (e.g., 1, 2, 3, 4, 5, 6, 7, 8, 9, or 10) of the β-sheets comprises four β-strands.

In some embodiments, the jelly-roll domain comprises a first β-sheet in antiparallel orientation to a second β-sheet. In certain embodiments, the first β-sheet comprises about four (e.g., 3, 4, 5, or 6) f-strands. In certain embodiments, the second β-sheet comprises about four (e.g., 3, 4, 5, or 6) β-strands. In embodiments, the first and second β-sheet comprise, in total, about eight (e.g., 6, 7, 8, 9, 10, 11, or 12) β-strands.

In certain embodiments, a jelly-roll domain is a component of a capsid protein (e.g., an VP1 molecule as described herein). In certain embodiments, a jelly-roll domain has self-assembly activity. In some embodiments, a polypeptide comprising a jelly-roll domain binds to another copy of the polypeptide comprising the jelly-roll domain. In some embodiments, a jelly-roll domain of a first polypeptide binds to a jelly-roll domain of a second copy of the polypeptide.

Exemplary VP1 Sequences

Exemplary CAV VP1 amino acid sequences, and the sequences of exemplary VP1 domains, include, without limitation, those encoded by the VP1 nucleic acid sequences listed in Tables 1-17. In some embodiments, a polypeptide (e.g., an VP1 molecule) described herein comprises an amino acid sequence having at least about 70%, 75%, 80%, 85%, 90%, 95%, 96%, 97%, 98%, 99%, or 100% sequence identity to one or more CAV VP1 molecules, e.g., as described in any of Tables 1-17, or a functional fragment thereof. In some embodiments, a CAVector described herein comprises an VP1 molecule comprising an amino acid sequence having at least about 70%, 75%, 80%, 85%, 90%, 95%, 96%, 97%, 98%, 99%, or 100% sequence identity to the arginine-rich region of one or more CAV VP1 molecules, e.g., as described in any of Tables 1-17. In some embodiments, a CAVector described herein comprises an VP1 molecule comprising an amino acid sequence having at least about 70%, 75%, 80%, 85%, 90%, 95%, 96%, 97%, 98%, 99%, or 100% sequence identity to the jelly roll domain of one or more CAV VP1 molecules, e.g., as described in any of Tables 1-17. In some embodiments, a CAVector described herein comprises a nucleic acid molecule (e.g., a genetic element) encoding an VP1 molecule comprising an amino acid sequence having at least about 70%, 75%, 80%, 85%, 90%, 95%, 96%, 97%, 98%, 99%, or 100% sequence identity to one or more CAV VP1 molecules, e.g., as described in any of Tables 1-17, or a functional fragment thereof.

In some embodiments, the one or more CAV VP1 subsequences comprises one or more of an arginine (Arg)-rich domain and/or a jelly-roll domain (e.g., as listed in any of Tables 1-17), or sequences having at least about 70%, 75%, 80%, 85%, 90%, 95%, 96%, 97%, 98%, 99%, or 100% sequence identity thereto. In some embodiments, the VP1 molecule comprises a plurality of subsequences from different CAVs (e.g., any combination of VP1 subsequences, such as arginine-rich region sequences and/or jelly roll domain sequences, selected from the CAV subsequences listed in any table herein).

In some embodiments, a VP1 molecule comprises at least one difference (e.g., a mutation, chemical modification, or epigenetic alteration) relative to a wild-type VP1 protein, e.g., as described herein (e.g., as listed in Table 1A, 1B, or 17).

### Genetic Elements

In some embodiments, the CAVector comprises a genetic element (e.g., a genetic element enclosed in a proteinaceous exterior, e.g., comprising a CAV capsid protein, e.g., a VP1 molecule). In some embodiments, the genetic element has one or more of the following characteristics: is substantially non-integrating with a host cell's genome, is an episomal nucleic acid, is a single stranded DNA, is circular, is about 1 to 10 kb, exists within the nucleus of the cell, can be bound by endogenous proteins, produces and/or encodes an effector, such as a polypeptide or nucleic acid (e.g., an RNA, iRNA, microRNA) that targets a gene, activity, or function of a host or target cell. In one embodiment, the genetic element is a substantially non-integrating DNA. In some embodiments, the genetic element comprises a packaging signal, e.g., a protein binding sequence as described herein, e.g., sequence that binds a capsid protein. In some embodiments, the packaging signal comprises the nucleic acid sequence of SEQ ID NO: 17. In some embodiments, outside of the packaging or capsid-binding sequence, the genetic element has less than 70%, 60%, 50%, 40%, 30%, 20%, 10%, 5% sequence identity to a wild type CAV nucleic acid sequence, e.g., has less than 70%, 60%, 50%, 40%, 30%, 20%, 10%, 5% sequence identity to an CAV nucleic acid sequence, e.g., as described herein, e.g., in any of Tables 1-17. In some embodiments, outside of the packaging or capsid-binding sequence, the genetic element has less than 500 450, 400, 350, 300, 250, 200, 150, or 100 contiguous nucleotides that are at least 70%, 75%, 80%, 8%, 90%, 95%, 96%, 97%, 98%, 99%, or 100% identical to an CAV nucleic acid sequence. In certain embodiments, the genetic element is a circular, single stranded DNA that comprises a promoter sequence, a sequence encoding a therapeutic effector, and a capsid binding protein.

In some embodiments, the genetic element has a length less than 20 kb (e.g., less than about 19 kb, 18 kb, 17 kb, 16 kb, 15 kb, 14 kb, 13 kb, 12 kb, 11 kb, 10 kb, 9 kb, 8 kb, 7 kb, 6 kb, 5 kb, 4 kb, 3 kb, 2 kb, 1 kb, or less). In some embodiments, the genetic element has, independently or in addition to, a length greater than 1000b (e.g., at least about 1.1 kb, 1.2 kb, 1.3 kb, 1.4 kb, 1.5 kb, 1.6 kb, 1.7 kb, 1.8 kb, 1.9 kb, 2 kb, 2.1 kb, 2.2 kb, 2.3 kb, 2.4 kb, 2.5 kb, 2.6 kb, 2.7 kb, 2.8 kb, 2.9 kb, 3 kb, 3.1 kb, 3.2 kb, 3.3 kb, 3.4 kb, 3.5 kb, 3.6 kb, 3.7 kb, 3.8 kb, 3.9 kb, 4 kb, 4.1 kb, 4.2 kb, 4.3 kb, 4.4 kb, 4.5 kb, 4.6 kb, 4.7 kb, 4.8 kb, 4.9 kb, 5 kb, or greater). In some embodiments, the genetic element has a length of about 2.5-4.6, 2.8-4.0, 3.0-3.8, or 3.2-3.7 kb. In some embodiments, the genetic element has a length of about 1.5-2.0, 1.5-2.5, 1.5-3.0, 1.5-3.5, 1.5-3.8, 1.5-3.9, 1.5-4.0, 1.5-4.5, or 1.5-5.0 kb. In some embodiments, the genetic element has a length of about 2.0-2.5, 2.0-3.0, 2.0-3.5, 2.0-3.8, 2.0-3.9, 2.0-4.0, 2.0-4.5, or 2.0-5.0 kb. In some embodiments, the genetic element has a length of about 2.5-3.0, 2.5-3.5, 2.5-3.8, 2.5-3.9, 2.5-4.0, 2.5-4.5, or 2.5-5.0 kb. In some embodiments, the genetic element has a length of about 3.0-5.0, 3.5-5.0, 4.0-5.0, or 4.5-5.0 kb. In some embodiments, the genetic element has a length of about 1.5-2.0, 2.0-2.5, 2.5-3.0, 3.0-3.5, 3.1-3.6, 3.2-3.7, 3.3-3.8, 3.4-3.9, 3.5-4.0, 4.0-4.5, or 4.5-5.0 kb. In some embodiments, the genetic element has a length between about 3.6-3.9 kb. In some embodiments, the genetic element has a length between about 2.8-2.9 kb. In some embodiments, the genetic element has a length between about 2.0-3.2 kb.

In some embodiments, the genetic element comprises one or more of the features described herein, e.g., a sequence encoding a substantially non-pathogenic protein, a protein binding sequence, one or more sequences encoding a regulatory nucleic acid, one or more regulatory sequences, one or more sequences encoding a replication protein, and other sequences.

In embodiments, the genetic element was produced from a double-stranded circular DNA (e.g., produced by in vitro circularization). In some embodiments, the genetic element was produced by rolling circle replication from the double-stranded circular DNA. In embodiments, the rolling circle replication occurs in a cell (e.g., a host cell, e.g., an avian cell (e.g., an MDCC cell) or a mammalian cell, e.g., a human cell, e.g., a HEK293T cell, an A549 cell, or a Jurkat cell). In embodiments, the genetic element can be amplified exponentially by rolling circle replication in the cell. In embodiments, the genetic element can be amplified linearly by rolling circle replication in the cell. In embodiments, the double-stranded circular DNA or genetic element is capable of yielding at least 2, 4, 8, 16, 32, 64, 128, 256, 518, 1024 or more times the original quantity by rolling circle replication in the cell. In embodiments, the double-stranded circular DNA was introduced into the cell, e.g., as described herein.

In some embodiments, the double-stranded circular DNA and/or the genetic element does not comprise one or more bacterial plasmid elements (e.g., a bacterial origin of replication or a selectable marker, e.g., a bacterial resistance gene, e.g., an ampicillin resistance gene). In some embodiments, the double-stranded circular DNA and/or the genetic element does not comprise a bacterial plasmid backbone.

In one embodiment, the invention includes a genetic element comprising a nucleic acid sequence (e.g., a DNA sequence) encoding (i) a substantially non-pathogenic exterior protein, (ii) an exterior protein binding sequence that binds the genetic element to the substantially non-pathogenic exterior protein, and (iii) a regulatory nucleic acid. In such an embodiment, the genetic element may comprise one or more sequences with at least about 60%, 70% 80%, 85%, 90% 95%, 96%, 97%, 98% and 99% nucleotide sequence identity to any one of the nucleotide sequences to a native viral sequence (e.g., a native CAV sequence, e.g., as described herein).

### Protein Binding Sequence

A strategy employed by many viruses is that the viral capsid protein recognizes a specific protein binding sequence in its genome. For example, in viruses with unsegmented genomes, such as the L-A virus of yeast, there is a secondary structure (stem-loop) and a specific sequence at the 5′ end of the genome that are both used to bind the viral capsid protein. However, viruses with segmented genomes, such as Reoviridae, Orthomyxoviridae (influenza), Bunyaviruses and Arenaviruses, need to package each of the genomic segments. Some viruses utilize a complementarity region of the segments to aid the virus in including one of each of the genomic molecules. Other viruses have specific binding sites for each of the different segments. See for example, Curr Opin Struct Biol. 2010 February; 20(1): 114-120; and Journal of Virology (2003), 77(24), 13036-13041.

In some embodiments, the genetic element encodes a protein binding sequence that binds to a protein comprised in a proteinaceous exterior (e.g., a capsid protein, e.g., a CAV VP1 molecule). In some embodiments, the protein binding sequence facilitates packaging the genetic element into the proteinaceous exterior. In some embodiments, the protein binding sequence specifically binds an arginine-rich region of the protein comprised in the proteinaceous exterior. In some embodiments, the genetic element comprises a protein binding sequence having the nucleic acid sequence AGCCCTGAAAAGGGGGGGGGGCTAAAGCCCCCCCCCCTTAAACCCCCCCCTGGGGGGGATT CCCCCCCAGACCCCCCCTTTATATAGCACTCAATAAACGCAGAAAATAGATTTATCGCACTA TC (SEQ ID NO: 17), or a reverse complement thereof. In some embodiments, the genetic element comprises a protein binding sequence having at least 70%, 80%, 85%, 90%, 95%, 96%, 97%, 98%, 99%, or 100% sequence identity to a 5′ UTR, 3′ UTR, or GC-rich domain of a CAV sequence, e.g., as described herein, e.g., in any of Tables 1-17.

### 5′ UTR Regions

In some embodiments, a nucleic acid molecule as described herein (e.g., a genetic element, genetic element construct, or genetic element region) comprises a 5′ UTR sequence, e.g., as described herein (e.g., in any of Tables 1-16, or a 5′ UTR of a CAV genome listed in Table 17), or a sequence having at least 75%, 80%, 85%, 90%, 95%, 96%, 97%, 98%, or 99% sequence identity thereto.

In embodiments, the 5′ UTR sequence comprises the nucleic acid sequence of the 5′ UTR listed in Table 1A, or a sequence having at least 75%, 80%, 85%, 90%, 95%, 96%, 97%, 98%, or 99% sequence identity thereto. In some embodiments, the nucleic acid molecule comprises a nucleic acid sequence having at least 95% sequence identity to the 5′ UTR listed in Table 1A. In embodiments, the 5′ UTR sequence comprises the nucleic acid sequence of the 5′ UTR listed in Table 1B, or a sequence having at least 75%, 80%, 85%, 90%, 95%, 96%, 97%, 98%, or 99% sequence identity thereto. In some embodiments, the nucleic acid molecule comprises a nucleic acid sequence having at least 95% sequence identity to the 5′ UTR listed in Table 1B. In embodiments, the 5′ UTR sequence comprises the nucleic acid sequence of the 5′ UTR of a CAV genome listed in Table 17, or a sequence having at least 75%, 80%, 85%, 90%, 95%, 96%, 97%, 98%, or 99% sequence identity thereto. In some embodiments, the nucleic acid molecule comprises a nucleic acid sequence having at least 95% sequence identity to the 5′ UTR of a CAV genome listed in Table 17.

### GC-Rich Regions

In some embodiments, a genetic element or genetic element construct as described herein comprises a nucleic acid sequence (e.g., a contiguous sequence having a length of 10-20, 20-30, 30-40, 40-50, 50-60, 60-70, 70-80, 80-90, 90-100, 100-150, or 150-200 nucleotides) having a GC content of at least 50%, 60%, 70%, 75%, 80%, 85%, 90%, 95%, 96%, 97%, 98%, 99%, or 100%. In embodiments, the genetic element or genetic element construct comprises a nucleic acid sequence (e.g., a contiguous sequence having a length of 10-20, 20-30, 30-40, 40-50, 50-60, 60-70, 70-80, 80-90, 90-100, 100-150, or 150-200 nucleotides) having at least about 75% (e.g., at least 75%, 80%, 85%, 90%, 95%, 96%, 97%, 98%, 99%, or 100%) identity to a GC-rich sequence of a CAV genome described herein (e.g., in Table 1A or 1B, or as listed in Table 17). In some embodiments, the GC-rich region forms a hairpin.

### Effectors

In some embodiments, the genetic element may include one or more sequences that encode an effector, e.g., a functional effector, e.g., an endogenous effector or an exogenous effector, e.g., a therapeutic effector. In some embodiments, the effector is a polypeptide or nucleic acid. In some embodiments, the effector has one or more of the following properties: (i) codon optimized for expression in a human cell, (ii) is a human polypeptide or nucleic acid, (iii) binds to a human polypeptide or nucleic acid, or (iv) has an activity in a human cell, e.g., modulates (e.g., increases or decreases) the activity and/or level of a human gene in the human cell).

The effector may modulate a biological activity, for example increasing or decreasing enzymatic activity, gene expression, cell signaling, and cellular or organ function. Effector activities may also include binding regulatory proteins to modulate activity of the regulator, such as transcription or translation. Effector activities also may include activator or inhibitor functions. For example, the effector may induce enzymatic activity. In some embodiments, the effector comprises an enzyme. In some embodiments, the exogenous effector comprises an antigen from an infectious agent (e.g., a virus or bacteria). In some embodiments, the effector is a cytotoxic or cytolytic RNA or protein. In some embodiments, the functional nucleic acid is a non-coding RNA. In some embodiments, the functional nucleic acid is a coding RNA. In some embodiments, the effector triggers increased substrate affinity in an enzyme, e.g., fructose 2,6-bisphosphate activates phosphofructokinase 1 and increases the rate of glycolysis in response to the insulin. In another example, the effector may inhibit substrate binding to a receptor and inhibit its activation, e.g., naltrexone and naloxone bind opioid receptors without activating them and block the receptors' ability to bind opioids. Effector activities may also include modulating protein stability/degradation and/or transcript stability/degradation. For example, proteins may be targeted for degradation by the polypeptide co-factor, ubiquitin, onto proteins to mark them for degradation. In another example, the effector inhibits enzymatic activity by blocking the enzyme's active site, e.g., methotrexate is a structural analog of tetrahydrofolate, a coenzyme for the enzyme dihydrofolate reductase that binds to dihydrofolate reductase 1000-fold more tightly than the natural substrate and inhibits nucleotide base synthesis.

In some embodiments, the sequence encoding an effector is part of the genetic element, e.g., it can be inserted at an insert site as described herein. In embodiments, the sequence encoding an effector is inserted into the genetic element at a noncoding region, e.g., a noncoding region disposed 3′ of the open reading frames and 5′ of the GC-rich region of the genetic element, in the 5′ noncoding region upstream of the TATA box, in the 5′ UTR, in the 3′ noncoding region downstream of the poly-A signal, or upstream of the GC-rich region. In some embodiments, the sequence encoding an effector replaces part or all of an open reading frame (e.g., an ORF as described herein, e.g., a CAV VP1, VP2, and/or Apoptin).

In some embodiments, the sequence encoding an effector comprises 100-2000, 100-1000, 100-500, 100-200, 200-2000, 200-1000, 200-500, 500-1000, 500-2000, or 1000-2000 nucleotides. In some embodiments, the sequence encoding an effector comprises at least 10, 20, 30, 40, 50, 60, 70, 80, 90, 100, 200, 300, 400, 500, 600, 700, 800, 900, 1000, 1100, 1200, 1300, 1400, 1500, 1600, 1700, 1800, 1900, or 2000 nucleotides. In some embodiments, the effector is a nucleic acid or protein payload, e.g., as described herein.

Regulatory Nucleic Acids

In some embodiments, the effector is a regulatory nucleic acid. Regulatory nucleic acids modify expression of an endogenous gene and/or an exogenous gene. In one embodiment, the regulatory nucleic acid targets a host gene. The regulatory nucleic acids may include, but are not limited to, a nucleic acid that hybridizes to an endogenous gene (e.g., miRNA, siRNA, mRNA, lncRNA, RNA, DNA, an antisense RNA, gRNA as described herein elsewhere), nucleic acid that hybridizes to an exogenous nucleic acid such as a viral DNA or RNA, nucleic acid that hybridizes to an RNA, nucleic acid that interferes with gene transcription, nucleic acid that interferes with RNA translation, nucleic acid that stabilizes RNA or destabilizes RNA such as through targeting for degradation, and nucleic acid that modulates a DNA or RNA binding factor. In embodiments, the regulatory nucleic acid encodes an miRNA. In some embodiments, the regulatory nucleic acid is endogenous to a wild-type CAV. In some embodiments, the regulatory nucleic acid is exogenous to a wild-type CAV.

In some embodiments, the regulatory nucleic acid comprises RNA or RNA-like structures typically containing 5-500 base pairs (depending on the specific RNA structure, e.g., miRNA 5-30 bps, lncRNA 200-500 bps) and may have a nucleobase sequence identical (or complementary) or nearly identical (or substantially complementary) to a coding sequence in an expressed target gene within the cell, or a sequence encoding an expressed target gene within the cell.

In some embodiments, the regulatory nucleic acid comprises a nucleic acid sequence, e.g., a guide RNA (gRNA). In some embodiments, the DNA targeting moiety comprises a guide RNA or nucleic acid encoding the guide RNA. A gRNA short synthetic RNA can be composed of a “scaffold” sequence necessary for binding to the incomplete effector moiety and a user-defined ˜20 nucleotide targeting sequence for a genomic target. In practice, guide RNA sequences are generally designed to have a length of between 17-24 nucleotides (e.g., 19, 20, or 21 nucleotides) and complementary to the targeted nucleic acid sequence. Custom gRNA generators and algorithms are available commercially for use in the design of effective guide RNAs. Gene editing has also been achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgRNAs have also been demonstrated to be effective in genome editing; see, for example, Hendel et al. (2015) Nature Biotechnol., 985-991.

The regulatory nucleic acid comprises a gRNA that recognizes specific DNA sequences (e.g., sequences adjacent to or within a promoter, enhancer, silencer, or repressor of a gene).

Certain regulatory nucleic acids can inhibit gene expression through the biological process of RNA interference (RNAi). RNAi molecules comprise RNA or RNA-like structures typically containing 15-50 base pairs (such as about 18-25 base pairs) and having a nucleobase sequence identical (complementary) or nearly identical (substantially complementary) to a coding sequence in an expressed target gene within the cell. RNAi molecules include, but are not limited to: short interfering RNAs (siRNAs), double-strand RNAs (dsRNA), micro RNAs (miRNAs), short hairpin RNAs (shRNA), meroduplexes, and dicer substrates (U.S. Pat. Nos. 8,084,599 8,349,809 and 8,513,207).

Long non-coding RNAs (lncRNA) are defined as non-protein coding transcripts longer than 100 nucleotides. This somewhat arbitrary limit distinguishes lncRNAs from small regulatory RNAs such as microRNAs (miRNAs), short interfering RNAs (siRNAs), and other short RNAs. In general, the majority (˜78%) of lncRNAs are characterized as tissue-specific. Divergent lncRNAs that are transcribed in the opposite direction to nearby protein-coding genes (comprise a significant proportion ˜20% of total lncRNAs in mammalian genomes) may possibly regulate the transcription of the nearby gene.

The genetic element may encode regulatory nucleic acids with a sequence substantially complementary, or fully complementary, to all or a fragment of an endogenous gene or gene product (e.g., mRNA). The regulatory nucleic acids may complement sequences at the boundary between introns and exons to prevent the maturation of newly-generated nuclear RNA transcripts of specific genes into mRNA for transcription. The regulatory nucleic acids that are complementary to specific genes can hybridize with the mRNA for that gene and prevent its translation. The antisense regulatory nucleic acid can be DNA, RNA, or a derivative or hybrid thereof.

The length of the regulatory nucleic acid that hybridizes to the transcript of interest may be between 5 to 30 nucleotides, between about 10 to 30 nucleotides, or about 11, 12, 13, 14, 15, 16, 17, 18, 19, 20, 21, 22, 23, 24, 25, 26, 27, 28, 29, 30 or more nucleotides. The degree of identity of the regulatory nucleic acid to the targeted transcript should be at least 75%, at least 80%, at least 85%, at least 90%, or at least 95%.

The genetic element may encode a regulatory nucleic acid, e.g., a micro RNA (miRNA) molecule identical to about 5 to about 25 contiguous nucleotides of a target gene. In some embodiments, the miRNA sequence targets a mRNA and commences with the dinucleotide AA, comprises a GC-content of about 30-70% (about 30-60%, about 40-60%, or about 45%-55%), and does not have a high percentage identity to any nucleotide sequence other than the target in the genome of the mammal in which it is to be introduced, for example as determined by standard BLAST search.

siRNAs and shRNAs resemble intermediates in the processing pathway of the endogenous microRNA (miRNA) genes (Bartel, Cell 116:281-297, 2004). In some embodiments, siRNAs can function as miRNAs and vice versa (Zeng et al., Mol Cell 9:1327-1333, 2002; Doench et al., Genes Dev 17:438-442, 2003). MicroRNAs, like siRNAs, use RISC to downregulate target genes, but unlike siRNAs, most animal miRNAs do not cleave the mRNA. Instead, miRNAs reduce protein output through translational suppression or polyA removal and mRNA degradation (Wu et al., Proc Natl Acad Sci USA 103:4034-4039, 2006). Known miRNA binding sites are within mRNA 3′ UTRs; miRNAs seem to target sites with near-perfect complementarity to nucleotides 2-8 from the miRNA's 5′ end (Rajewsky, Nat Genet 38 Suppl:S8-13, 2006; Lim et al., Nature 433:769-773, 2005). This region is known as the seed region. Because siRNAs and miRNAs are interchangeable, exogenous siRNAs downregulate mRNAs with seed complementarity to the siRNA (Birmingham et al., Nat Methods 3:199-204, 2006. Multiple target sites within a 3′ UTR give stronger downregulation (Doench et al., Genes Dev 17:438-442, 2003).

Lists of known miRNA sequences can be found in databases maintained by research organizations, such as Wellcome Trust Sanger Institute, Penn Center for Bioinformatics, Memorial Sloan Kettering Cancer Center, and European Molecule Biology Laboratory, among others. Known effective siRNA sequences and cognate binding sites are also well represented in the relevant literature. RNAi molecules are readily designed and produced by technologies known in the art. In addition, there are computational tools that increase the chance of finding effective and specific sequence motifs (Lagana et al., Methods Mol. Bio., 2015, 1269:393-412).

The regulatory nucleic acid may modulate expression of RNA encoded by a gene. Because multiple genes can share some degree of sequence homology with each other, in some embodiments, the regulatory nucleic acid can be designed to target a class of genes with sufficient sequence homology. In some embodiments, the regulatory nucleic acid can contain a sequence that has complementarity to sequences that are shared amongst different gene targets or are unique for a specific gene target. In some embodiments, the regulatory nucleic acid can be designed to target conserved regions of an RNA sequence having homology between several genes thereby targeting several genes in a gene family (e.g., different gene isoforms, splice variants, mutant genes, etc.). In some embodiments, the regulatory nucleic acid can be designed to target a sequence that is unique to a specific RNA sequence of a single gene.

In some embodiments, the genetic element may include one or more sequences that encode regulatory nucleic acids that modulate expression of one or more genes.

In one embodiment, the gRNA described elsewhere herein are used as part of a CRISPR system for gene editing. For the purposes of gene editing, the CAVector may be designed to include one or multiple guide RNA sequences corresponding to a desired target DNA sequence; see, for example, Cong et al. (2013) Science, 339:819-823; Ran et al. (2013) Nature Protocols, 8:2281-2308. At least about 16 or 17 nucleotides of gRNA sequence generally allow for Cas9-mediated DNA cleavage to occur; for Cpf1 at least about 16 nucleotides of gRNA sequence is needed to achieve detectable DNA cleavage.

### Therapeutic Effectors (e.g., Peptides or Polypeptides)

In some embodiments, the genetic element comprises a therapeutic expression sequence, e.g., a sequence that encodes a therapeutic peptide or polypeptide. In some embodiments, the genetic element includes a sequence encoding a protein e.g., a therapeutic protein. Some examples of therapeutic proteins may include, but are not limited to, a hormone, a cytokine, an enzyme, an antibody (e.g., one or a plurality of polypeptides encoding at least a heavy chain or a light chain), a transcription factor, a receptor (e.g., a membrane receptor), a ligand, a membrane transporter, a secreted protein, a peptide, a carrier protein, a structural protein, a nuclease, or a component thereof.

In some embodiments, the genetic element includes a sequence encoding a peptide e.g., a therapeutic peptide. The peptides may be linear or branched. The peptide has a length from about 5 to about 500 amino acids, about 15 to about 400 amino acids, about 20 to about 325 amino acids, about 25 to about 250 amino acids, about 50 to about 200 amino acids, or any range therebetween. Some examples of peptides include, but are not limited to, fluorescent tag or marker, antigen, peptide therapeutic, synthetic or analog peptide from naturally-bioactive peptide, agonist or antagonist peptide, anti-microbial peptide, a targeting or cytotoxic peptide, a degradation or self-destruction peptide, and degradation or self-destruction peptides. Peptides useful in the invention described herein also include antigen-binding peptides, e.g., antigen binding antibody or antibody-like fragments, such as single chain antibodies, nanobodies (see, e.g., Steeland et al. 2016. Nanobodies as therapeutics: big opportunities for small antibodies. Drug Discov Today: 21(7):1076-113). Such antigen binding peptides may bind a cytosolic antigen, a nuclear antigen, or an intra-organellar antigen.

In some embodiments, the genetic element comprises a sequence that encodes small peptides, peptidomimetics (e.g., peptoids), amino acids, and amino acid analogs. Such therapeutics generally have a molecular weight less than about 5,000 grams per mole, a molecular weight less than about 2,000 grams per mole, a molecular weight less than about 1,000 grams per mole, a molecular weight less than about 500 grams per mole, and salts, esters, and other pharmaceutically acceptable forms of such compounds. Such therapeutics may include, but are not limited to, a neurotransmitter, a hormone, a drug, a toxin, a viral or microbial particle, a synthetic molecule, and agonists or antagonists thereof.

In some embodiments, the composition or CAVector described herein includes a polypeptide linked to a ligand that is capable of targeting a specific location, tissue, or cell.

In some embodiments, the polypeptide encoded by the therapeutic expression sequence may be a functional variant or fragment thereof of any of the above, e.g., a protein having at least 80%, 85%, 90%, 95%, 967%, 98%, 99% identity to a protein sequence which disclosed in a table herein by reference to its UniProt ID.

In some embodiments, the therapeutic expression sequence may encode an antibody or antibody fragment that binds any of the above, e.g., an antibody against a protein having at least 80%, 85%, 90%, 95%, 967%, 98%, 99% identity to a protein sequence which disclosed in a table herein by reference to its UniProt ID. The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An “antibody fragment” refers to a molecule that includes at least one heavy chain or light chain and binds an antigen. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

Exemplary Intracellular Polypeptide Effectors

In some embodiments, the effector comprises a cytosolic polypeptide or cytosolic peptide. In some embodiments, the effector comprises cytosolic peptide is a DPP-4 inhibitor, an activator of GLP-1 signaling, or an inhibitor of neutrophil elastase. In some embodiments, the effector increases the level or activity of a growth factor or receptor thereof (e.g., an FGF receptor, e.g., FGFR3). In some embodiments, the effector comprises an inhibitor of n-myc interacting protein activity (e.g., an n-myc interacting protein inhibitor); an inhibitor of EGFR activity (e.g., an EGFR inhibitor); an inhibitor of IDH1 and/or IDH2 activity (e.g., an IDH1 inhibitor and/or an IDH2 inhibitor); an inhibitor of LRP5 and/or DKK2 activity (e.g., an LRP5 and/or DKK2 inhibitor); an inhibitor of KRAS activity; an activator of HTT activity; or inhibitor of DPP-4 activity (e.g., a DPP-4 inhibitor).

In some embodiments, the effector comprises a regulatory intracellular polypeptide. In some embodiments, the regulatory intracellular polypeptide binds one or more molecule (e.g., protein or nucleic acid) endogenous to the target cell. In some embodiments, the regulatory intracellular polypeptide increases the level or activity of one or more molecule (e.g., protein or nucleic acid) endogenous to the target cell. In some embodiments, the regulatory intracellular polypeptide decreases the level or activity of one or more molecule (e.g., protein or nucleic acid) endogenous to the target cell.

Exemplary Secreted Polypeptide Effectors

Exemplary secreted therapeutics are described herein, e.g., in the tables below.

TABLE 18A
Exemplary cytokines and cytokine receptors

| Cytokine | Cytokine receptor(s) | Entrez Gene ID | UniProt ID |
|---|---|---|---|
| IL-1α, IL-1β, or a | IL-1 type 1 receptor, IL-1 type | 3552, 3553 | P01583, P01584 |
| heterodimer thereof | 2 receptor | | |
| IL-1Ra | IL-1 type 1 receptor, IL-1 type | 3454, 3455 | P17181, P48551 |
| | 2 receptor | | |
| IL-2 | IL-2R | 3558 | P60568 |
| IL-3 | IL-3 receptor α + β c (CD131) | 3562 | P08700 |
| IL-4 | IL-4R type I, IL-4R type II | 3565 | P05112 |
| IL-5 | IL-5R | 3567 | P05113 |
| IL-6 | IL-6R (sIL-6R) gp130 | 3569 | P05231 |
| IL-7 | IL-7R and sIL-7R | 3574 | P13232 |
| IL-8 | CXCR1 and CXCR2 | 3576 | P10145 |
| IL-9 | IL-9R | 3578 | P15248 |
| IL-10 | IL-10R1/IL-10R2 complex | 3586 | P22301 |
| IL-11 | IL-11Rα 1 gp130 | 3589 | P20809 |
| IL-12 (e.g., p35, p40, or a | IL-12Rβ1 and IL-12Rβ2 | 3593, 3592 | P29459, P29460 |
| heterodimer thereof) | | | |
| IL-13 | IL-13R1α1 and IL-13R1α2 | 3596 | P35225 |
| IL-14 | IL-14R | 30685 | P40222 |
| IL-15 | IL-15R | 3600 | P40933 |
| IL-16 | CD4 | 3603 | Q14005 |
| IL-17A | IL-17RA | 3605 | Q16552 |
| IL-17B | IL-17RB | 27190 | Q9UHF5 |
| IL-17C | IL-17RA to IL-17RE | 27189 | Q9P0M4 |
| e | SEF | 53342 | Q8TAD2 |
| IL-17F | IL-17RA, IL-17RC | 112744 | Q96PD4 |
| IL-18 | IL-18 receptor | 3606 | Q14116 |
| IL-19 | IL-20R1/IL-20R2 | 29949 | Q9UHD0 |
| IL-20 | L-20R1/IL-20R2 and | 50604 | Q9NYY1 |
| | IL-22R1/IL-20R2 | | |
| IL-21 | IL-21R | 59067 | Q9HBE4 |
| IL-22 | IL-22R | 50616 | Q9GZX6 |
| IL-23 (e.g., p19, p40, or a | IL-23R | 51561 | Q9NPF7 |
| heterodimer thereof) | | | |
| IL-24 | IL-20R1/IL-20R2 and | 11009 | Q13007 |
| | IL-22R1/IL-20R2 | | |
| IL-25 | IL-17RA and IL-17RB | 64806 | Q9H293 |
| IL-26 | IL-10R2 chain and IL-20R1 | 55801 | Q9NPH9 |
| | chain | | |
| IL-27 (e.g., p28, EBI3, or | WSX-1 and gp130 | 246778 | Q8NEV9 |
| a heterodimer thereof) | | | |
| IL-28A, IL-28B, and IL29 | IL-28R1/IL-10R2 | 282617, 282618 | Q8IZI9, Q8IU54 |
| IL-30 | IL6R/gp130 | 246778 | Q8NEV9 |
| IL-31 | IL-31RA/OSMRβ | 386653 | Q6EBC2 |
| IL-32 | | 9235 | P24001 |
| IL-33 | ST2 | 90865 | O95760 |
| IL-34 | Colony-stimulating factor 1 | 146433 | Q6ZMJ4 |
| | receptor | | |
| IL-35 (e.g., p35, EBI3, or | IL-12Rβ2/gp130; | 10148 | Q14213 |
| a heterodimer thereof) | IL-12Rβ2/IL-12Rβ2; | | |
| | gp130/gp130 | | |
| IL-36 | IL-36Ra | 27179 | Q9UHA7 |
| IL-37 | IL-18Rα and IL-18BP | 27178 | Q9NZH6 |
| IL-38 | IL-1R1, IL-36R | 84639 | Q8WWZ1 |
| IFN-α | IFNAR | 3454 | P17181 |
| IFN-β | IFNAR | 3454 | P17181 |
| IFN-γ | IFNGR1/IFNGR2 | 3459 | P15260 |
| TGF-β | TβR-I and TβR-II | 7046, 7048 | P36897, P37173 |
| TNF-α | TNFR1, TNFR2 | 7132, 7133 | P19438, P20333 |

In some embodiments, an effector described herein comprises a cytokine of Table 18A, or a functional variant thereof, e.g., a homolog (e.g., ortholog or paralog) or fragment thereof. In some embodiments, an effector described herein comprises a protein having at least 80%, 85%, 90%, 95%, 967%, 98%, 99% sequence identity to an amino acid sequence listed in Table 50 by reference to its UniProt ID. In some embodiments, the functional variant binds to the corresponding cytokine receptor with a Kd of no more than 10%, 20%, 30%, 40%, or 50% higher or lower than the Kd of the corresponding wild-type cytokine for the same receptor under the same conditions. In some embodiments, the effector comprises a fusion protein comprising a first region (e.g., a cytokine polypeptide of Table 18A or a functional variant or fragment) and a second, heterologous region. In some embodiments, the first region is a first cytokine polypeptide of Table 18A. In some embodiments, the second region is a second cytokine polypeptide of Table 18A, wherein the first and second cytokine polypeptides form a cytokine heterodimer with each other in a wild-type cell. In some embodiments, the polypeptide of Table 18A or functional variant thereof comprises a signal sequence, e.g., a signal sequence that is endogenous to the effector, or a heterologous signal sequence. In some embodiments, an CAVector encoding a cytokine of Table 18A, or a functional variant thereof, is used for the treatment of a disease or disorder described herein.

In some embodiments, an effector described herein comprises an antibody molecule (e.g., an scFv) that binds a cytokine of Table 18A. In some embodiments, an effector described herein comprises an antibody molecule (e.g., an scFv) that binds a cytokine receptor of Table 18A. In some embodiments, the antibody molecule comprises a signal sequence.

Exemplary cytokines and cytokine receptors are described, e.g., in Akdis et al., “Interleukins (from IL-1 to IL-38), interferons, transforming growth factor β, and TNF-α: Receptors, functions, and roles in diseases” October 2016 Volume 138, Issue 4, Pages 984-1010, which is herein incorporated by reference in its entirety, including Table I therein.

TABLE 18B
Exemplary polypeptide hormones and receptors

| Hormone | Receptor | Entrez Gene ID | UniProt ID |
|---|---|---|---|
| Natriuretic Peptide, e.g., Atrial | NPRA, NPRB, NPRC | 4878 | P01160 |
| Natriuretic Peptide (ANP) | | | |
| Brain Natriuretic Peptide (BNP) | NPRA, NPRB | 4879 | P16860 |
| C-type natriuretic peptide | NPRB | 4880 | P23582 |
| (CNP) | | | |
| Growth hormone (GH) | GHR | 2690 | P10912 |
| Human growth hormone (hGH) | hGH receptor (human | 2690 | P10912 |
| | GHR) | | |
| Prolactin (PRL) | PRLR | 5617 | P01236 |
| Thyroid-stimulating hormone | TSH receptor | 7253 | P16473 |
| (TSH) | | | |
| Adrenocorticotropic hormone | ACTH receptor | 5443 | P01189 |
| (ACTH) | | | |
| Follicle-stimulating hormone | FSHR | 2492 | P23945 |
| (FSH) | | | |
| Luteinizing hormone (LH) | LHR | 3973 | P22888 |
| Antidiuretic hormone (ADH) | Vasopressin receptors, e.g., | 554 | P30518 |
| | V2; AVPR1A; AVPR1B; | | |
| | AVPR3; AVPR2 | | |
| Oxytocin | OXTR | 5020 | P01178 |
| Calcitonin | Calcitonin receptor (CT) | 796 | P01258 |
| Parathyroid hormone (PTH) | PTH1R and PTH2R | 5741 | P01270 |
| Insulin | Insulin receptor (IR) | 3630 | P01308 |
| Glucagon | Glucagon receptor | 2641 | P01275 |

In some embodiments, an effector described herein comprises a hormone of Table 18B, or a functional variant thereof, e.g., a homolog (e.g., ortholog or paralog) or fragment thereof. In some embodiments, an effector described herein comprises a protein having at least 80%, 85%, 90%, 95%, 967%, 98%, 99% sequence identity to an amino acid sequence listed in Table 51 by reference to its UniProt ID. In some embodiments, the functional variant binds to the corresponding receptor with a Kd of no more than 10%, 20%, 30%, 40%, or 50% higher than the Kd of the corresponding wild-type hormone for the same receptor under the same conditions. In some embodiments, the polypeptide of Table 18B or functional variant thereof comprises a signal sequence, e.g., a signal sequence that is endogenous to the effector, or a heterologous signal sequence. In some embodiments, an CAVector encoding a hormone of Table 18B, or a functional variant thereof, is used for the treatment of a disease or disorder described herein.

In some embodiments, an effector described herein comprises an antibody molecule (e.g., an scFv) that binds a hormone of Table 18B. In some embodiments, an effector described herein comprises an antibody molecule (e.g., an scFv) that binds a hormone receptor of Table 18B. In some embodiments, the antibody molecule comprises a signal sequence.

TABLE 18C
Exemplary growth factors

| | | |
|---|---|---|
| Growth Factor | Entrez Gene ID | UniProt ID |

PDGF family

| | | | |
|---|---|---|---|
| PDGF (e.g., PDGF-1, | PDGF receptor, e.g., | 5156 | P16234 |
| PDGF-2, or a | PDGFRα, PDGFRβ | | |
| heterodimer thereof) | | | |
| CSF-1 | CSF1R | 1435 | P09603 |
| SCF | CD117 | 3815 | P10721 |

VEGF family

| | | | |
|---|---|---|---|
| VEGF (e.g., isoforms | VEGFR-1, VEGFR-2 | 2321 | P17948 |
| VEGF 121, VEGF 165, | | | |
| VEGF 189, and VEGF | | | |
| 206) | | | |
| VEGF-B | VEGFR-1 | 2321 | P17949 |
| VEGF-C | VEGFR-2 and | 2324 | P35916 |
| | VEGFR -3 | | |
| PIGF | VEGFR-1 | 5281 | Q07326 |

EGF family

| | | | |
|---|---|---|---|
| EGF | EGFR | 1950 | P01133 |
| TGF-α | EGFR | 7039 | P01135 |
| amphiregulin | EGFR | 374 | P15514 |
| HB-EGF | EGFR | 1839 | Q99075 |
| betacellulin | EGFR, ErbB-4 | 685 | P35070 |
| epiregulin | EGFR, ErbB-4 | 2069 | O14944 |
| Heregulin | EGFR, ErbB-4 | 3084 | Q02297 |

FGF family

| | | | |
|---|---|---|---|
| FGF-1, FGF-2, FGF-3, | FGFR1, FGFR2, | 2246, 2247, 2248, 2249, | P05230, P09038, |
| FGF-4, FGF-5, FGF-6, | FGFR3, and FGFR4 | 2250, 2251, 2252, 2253, 2254 | P11487, P08620, |
| FGF-7, FGF-8, FGF-9 | | | P12034, P10767, |
| | | | P21781, P55075, P31371 |

Insulin family

| | | | |
|---|---|---|---|
| Insulin | IR | 3630 | P01308 |
| IGF-I | IGF-I receptor, IGF- | 3479 | P05019 |
| | II receptor | | |
| IGF-II | IGF-II receptor | 3481 | P01344 |

HGF family

| | | | |
|---|---|---|---|
| HGF | MET receptor | 3082 | P14210 |
| MSP | RON | 4485 | P26927 |

Neurotrophin family

| | | | |
|---|---|---|---|
| NGF | LNGFR, trkA | 4803 | P01138 |
| BDNF | trkB | 627 | P23560 |
| NT-3 | trkA, trkB, trkC | 4908 | P20783 |
| NT-4 | trkA, trkB | 4909 | P34130 |
| NT-5 | trkA, trkB | 4909 | P34130 |

Angiopoietin family

| | | | |
|---|---|---|---|
| ANGPT1 | HPK-6/TEK | 284 | Q15389 |
| ANGPT2 | HPK-6/TEK | 285 | O15123 |
| ANGPT3 | HPK-6/TEK | 9068 | O95841 |
| ANGPT4 | HPK-6/TEK | 51378 | Q9Y264 |

In some embodiments, an effector described herein comprises a growth factor of Table 18C, or a functional variant thereof, e.g., a homolog (e.g., ortholog or paralog) or fragment thereof. In some embodiments, an effector described herein comprises a protein having at least 80%, 85%, 90%, 95%, 967%, 98%, 99% sequence identity to an amino acid sequence listed in Table 52 by reference to its UniProt ID. In some embodiments, the functional variant binds to the corresponding receptor with a Kd of no more than 10%, 20%, 30%, 40%, or 50% higher than the Kd of the corresponding wild-type growth factor for the same receptor under the same conditions. In some embodiments, the polypeptide of Table 18C or functional variant thereof comprises a signal sequence, e.g., a signal sequence that is endogenous to the effector, or a heterologous signal sequence. In some embodiments, a CAVector encoding a growth factor of Table 18C, or a functional variant thereof, is used for the treatment of a disease or disorder described herein.

In some embodiments, an effector described herein comprises an antibody molecule (e.g., an scFv) that binds a growth factor of Table 18C. In some embodiments, an effector described herein comprises an antibody molecule (e.g., an scFv) that binds a growth factor receptor of Table 18C. In some embodiments, the antibody molecule comprises a signal sequence.

Exemplary growth factors and growth factor receptors are described, e.g., in Bafico et al., “Classification of Growth Factors and Their Receptors” Holland-Frei Cancer Medicine. 6th edition, which is herein incorporated by reference in its entirety.

TABLE 18D
Clotting-associated factors

| Effector | Indication | Entrez Gene ID | UniProt ID |
|---|---|---|---|
| Factor I | Afibrinogenomia | 2243, 2266, 2244 | P02671, P02679, P02675 |
| (fibrinogen) | | | |
| Factor II | Factor II Deficiency | 2147 | P00734 |
| Factor IX | Hemophilia B | 2158 | P00740 |
| Factor V | Owren's disease | 2153 | P12259 |
| Factor VIII | Hemophilia A | 2157 | P00451 |
| Factor X | Stuart-Prower Factor | 2159 | P00742 |
| | Deficiency | | |
| Factor XI | Hemophilia C | 2160 | P03951 |
| Factor XIII | Fibrin Stabilizing factor | 2162, 2165 | P00488, P05160 |
| | deficiency | | |
| vWF | von Willebrand disease | 7450 | P04275 |

In some embodiments, an effector described herein comprises a polypeptide of Table 18D, or a functional variant thereof, e.g., a homolog (e.g., ortholog or paralog) or fragment thereof. In some embodiments, an effector described herein comprises a protein having at least 80%, 85%, 90%, 95%, 967%, 98%, 99% sequence identity to an amino acid sequence listed in Table 53 by reference to its UniProt ID. In some embodiments, the functional variant catalyzes the same reaction as the corresponding wild-type protein, e.g., at a rate no less than 10%, 20%, 30%, 40%, or 50% lower than the wild-type protein. In some embodiments, the polypeptide of Table 18D or functional variant thereof comprises a signal sequence, e.g., a signal sequence that is endogenous to the effector, or a heterologous signal sequence. In some embodiments, a CAVector encoding a polypeptide of Table 18D, or a functional variant thereof is used for the treatment of a disease or disorder of Table 18D.

Exemplary Protein Replacement Therapeutics

Exemplary protein replacement therapeutics are described herein, e.g., in the tables below.

TABLE 19A
Exemplary enzymatic effectors and corresponding indications

| Effector | deficiency | Entrez Gene ID | UniProt ID |
|---|---|---|---|
| 3-methylcrotonyl-CoA | 3-methylcrotonyl-CoA | 56922, 64087 | Q96RQ3, Q9HCC0 |
| carboxylase | carboxylase deficiency | | |
| Acetyl-CoA- | Mucopolysaccharidosis MPS | 138050 | Q68CP4 |
| glucosaminide N- | III (Sanfilippo's syndrome) | | |
| acetyltransferase | Type III-C | | |
| ADAMTS13 | Thrombotic | 11093 | Q76LX8 |
| | Thrombocytopenia Purpura | | |
| adenine | Adenine | 353 | P07741 |
| phosphoribosyltransferase | phosphoribosyltransferase | | |
| | deficiency | | |
| Adenosine deaminase | Adenosine deaminase | 100 | P00813 |
| | deficiency | | |
| ADP-ribose protein | Glutamyl ribose-5-phosphate | 26119, 54936 | Q5SW96, Q9NX46 |
| hydrolase | storage disease | | |
| alpha glucosidase | Glycogen storage disease | 2548 | P10253 |
| | type 2 (Pompe's disease) | | |
| Arginase | Familial hyperarginemia | 383, 384 | P05089, P78540 |
| Arylsulfatase A | Metachromatic | 410 | P15289 |
| | leukodystrophy | | |
| Cathepsin K | Pycnodysostosis | 1513 | P43235 |
| Ceramidase | Farber's disease | 125981, 340485, 55331 | Q8TDN7, |
| | (lipogranulomatosis) | | Q5QJU3, Q9NUN7 |
| Cystathionine B | Homocystinuria | 875 | P35520 |
| synthase | | | |
| Dolichol-P-mannose | Congenital disorders of N- | 8813, 54344 | O60762, Q9P2X0 |
| synthase | glycosylation CDG Ie | | |
| Dolicho-P- | Congenital disorders of N- | 84920 | Q5BKT4 |
| Glc: Man9GlcNAc2-PP- | glycosylation CDG Ic | | |
| dolichol | | | |
| glucosyltransferase | | | |
| Dolicho-P- | Congenital disorders of N- | 10195 | Q92685 |
| Man: Man5GlcNAc2- | glycosylation CDG Id | | |
| PP-dolichol | | | |
| mannosyltransferase | | | |
| Dolichyl-P-glucose: Glc- | Congenital disorders of N- | 79053 | Q9BVK2 |
| 1-Man-9-GlcNAc-2-PP- | glycosylation CDG Ih | | |
| dolichyl-α-3- | | | |
| glucosyltransferase | | | |
| Dolichyl-P- | Congenital disorders of N- | 79087 | Q9BV10 |
| mannose: Man-7- | glycosylation CDG Ig | | |
| GlcNAc-2-PP-dolichyl- | | | |
| α-6-mannosyltransferase | | | |
| Factor II | Factor II Deficiency | 2147 | P00734 |
| Factor IX | Hemophilia B | 2158 | P00740 |
| Factor V | Owren's disease | 2153 | P12259 |
| Factor VIII | Hemophilia A | 2157 | P00451 |
| Factor X | Stuart-Prower Factor | 2159 | P00742 |
| | Deficiency | | |
| Factor XI | Hemophilia C | 2160 | P03951 |
| Factor XIII | Fibrin Stabilizing factor | 2162, 2165 | P00488, P05160 |
| | deficiency | | |
| Galactosamine-6-sulfate | Mucopolysaccharidosis MPS | 2588 | P34059 |
| sulfatase | IV (Morquio's syndrome) | | |
| | Type IV-A | | |
| Galactosylceramide β- | Krabbe's disease | 2581 | P54803 |
| galactosidase | | | |
| Ganglioside β- | GM1 gangliosidosis, | 2720 | P16278 |
| galactosidase | generalized | | |
| Ganglioside β- | GM2 gangliosidosis | 2720 | P16278 |
| galactosidase | | | |
| Ganglioside β- | Sphingolipidosis Type I | 2720 | P16278 |
| galactosidase | | | |
| Ganglioside β- | Sphingolipidosis Type II | 2720 | P16278 |
| galactosidase | (juvenile type) | | |
| Ganglioside β- | Sphingolipidosis Type III | 2720 | P16278 |
| galactosidase | (adult type) | | |
| Glucosidase I | Congenital disorders of N- | 2548 | P10253 |
| | glycosylation CDG IIb | | |
| Glucosylceramide β- | Gaucher's disease | 2629 | P04062 |
| glucosidase | | | |
| Heparan-S-sulfate | Mucopolysaccharidosis MPS | 6448 | P51688 |
| sulfamidase | III (Sanfilippo's syndrome) | | |
| | Type III-A | | |
| homogentisate oxidase | Alkaptonuria | 3081 | Q93099 |
| Hyaluronidase | Mucopolysaccharidosis MPS | 3373, 8692, 8372, 23553 | Q12794, Q12891, |
| | IX (hyaluronidase deficiency) | | O43820, Q2M3T9 |
| Iduronate sulfate | Mucopolysaccharidosis MPS | 3423 | P22304 |
| sulfatase | II (Hunter's syndrome) | | |
| Lecithin-cholesterol | Complete LCAT deficiency, | 3931 | 606967 |
| acyltransferase (LCAT) | Fish-eye disease, | | |
| | atherosclerosis, | | |
| | hypercholesterolemia | | |
| Lysine oxidase | Glutaric acidemia type I | 4015 | P28300 |
| Lysosomal acid lipase | Cholesteryl ester storage | 3988 | P38571 |
| | disease (CESD) | | |
| Lysosomal acid lipase | Lysosomal acid lipase | 3988 | P38571 |
| | deficiency | | |
| lysosomal acid lipase | Wolman's disease | 3988 | P38571 |
| Lysosomal pepstatin- | Ceroid lipofuscinosis Late | 1200 | O14773 |
| insensitive peptidase | infantile form (CLN2, | | |
| | Jansky-Bielschowsky | | |
| | disease) | | |
| Mannose (Man) | Congenital disorders of N- | 4351 | P34949 |
| phosphate (P) isomerase | glycosylation CDG Ib | | |
| Mannosyl-α-1,6- | Congenital disorders of N- | 4247 | Q10469 |
| glycoprotein-β-1,2-N- | glycosylation CDG IIa | | |
| acetylglucosminyltransferase | | | |
| Metalloproteinase-2 | Winchester syndrome | 4313 | P08253 |
| methylmalonyl-CoA | Methylmalonic acidemia | 4594 | P22033 |
| mutase | (vitamin b12 non-responsive) | | |
| N-Acetyl | Mucopolysaccharidosis MPS | 411 | P15848 |
| galactosamine α-4- | VI (Maroteaux-Lamy | | |
| sulfate sulfatase | syndrome) | | |
| (arylsulfatase B) | | | |
| N-acetyl-D- | Mucopolysaccharidosis MPS | 4669 | P54802 |
| glucosaminidase | III (Sanfilippo's syndrome) | | |
| | Type III-B | | |
| N-Acetyl- | Schindler's disease Type I | 4668 | P17050 |
| galactosaminidase | (infantile severe form) | | |
| N-Acetyl- | Schindler's disease Type II | 4668 | P17050 |
| galactosaminidase | (Kanzaki disease, adult-onset | | |
| | form) | | |
| N-Acetyl- | Schindler's disease Type III | 4668 | P17050 |
| galactosaminidase | (intermediate form) | | |
| N-acetyl-glucosaminine- | Mucopolysaccharidosis MPS | 2799 | P15586 |
| 6-sulfate sulfatase | III (Sanfilippo's syndrome) | | |
| | Type III-D | | |
| N-acetylglucosaminyl-1- | Mucolipidosis ML III | 79158 | Q3T906 |
| phosphotransferase | (pseudo-Hurler's | | |
| | polydystrophy) | | |
| N-Acetylglucosaminyl- | Mucolipidosis ML II (I-cell | 79158 | Q3T906 |
| 1-phosphotransferase | disease) | | |
| catalytic subunit | | | |
| N-acetylglucosaminyl-1- | Mucolipidosis ML III | 84572 | Q9UJJ9 |
| phosphotransferase, | (pseudo-Hurler's | | |
| substrate-recognition | polydystrophy) Type III-C | | |
| subunit | | | |
| N- | Aspartylglucosaminuria | 175 | P20933 |
| Aspartylglucosaminidase | | | |
| Neuraminidase 1 | Sialidosis | 4758 | Q99519 |
| (sialidase) | | | |
| Palmitoyl-protein | Ceroid lipofuscinosis Adult | 5538 | P50897 |
| thioesterase-1 | form (CLN4, Kufs' disease) | | |
| Palmitoyl-protein | Ceroid lipofuscinosis | 5538 | P50897 |
| thioesterase-1 | Infantile form (CLN1, | | |
| | Santavuori-Haltia disease) | | |
| Phenylalanine | Phenylketonuria | 5053 | P00439 |
| hydroxylase | | | |
| Phosphomannomutase-2 | Congenital disorders of N- | 5373 | O15305 |
| | glycosylation CDG Ia (solely | | |
| | neurologic and neurologic- | | |
| | multivisceral forms) | | |
| Porphobilinogen | Acute Intermittent Porphyria | 3145 | P08397 |
| deaminase | | | |
| Purine nucleoside | Purine nucleoside | 4860 | P00491 |
| phosphorylase | phosphorylase deficiency | | |
| pyrimidine 5′ | Hemolytic anemia and/or | 51251 | Q9H0P0 |
| nucleotidase | pyrimidine 5′ nucleotidase | | |
| | deficiency | | |
| Sphingomyelinase | Niemann-Pick disease type A | 6609 | P17405 |
| Sphingomyelinase | Niemann-Pick disease type B | 6609 | P17405 |
| Sterol 27-hydroxylase | Cerebrotendinous | 1593 | Q02318 |
| | xanthomatosis (cholestanol | | |
| | lipidosis) | | |
| Thymidine | Mitochondrial | 1890 | P19971 |
| phosphorylase | neurogastrointestinal | | |
| | encephalomyopathy | | |
| | (MNGIE) | | |
| Trihexosylceramide α- | Fabry's disease | 2717 | P06280 |
| galactosidase | | | |
| tyrosinase, e.g., OCA1 | albinism, e.g., ocular albinism | 7299 | P14679 |
| UDP-GlcNAc: dolichyl- | Congenital disorders of N- | 1798 | Q9H3H5 |
| P NAcGlc | glycosylation CDG Ij | | |
| phosphotransferase | | | |
| UDP-N- | Sialuria French type | 10020 | Q9Y223 |
| acetylglucosamine-2- | | | |
| epimerase/N- | | | |
| acetylmannosamine | | | |
| kinase, sialin | | | |
| Uricase | Lesch-Nyhan syndrome, gout | 391051 | No protein |
| uridine diphosphate | Crigler-Najjar syndrome | 54658 | P22309 |
| glucuronyl-transferase | | | |
| (e.g., UGT1A1) | | | |
| α-1,2- | Congenital disorders of N- | 79796 | Q9H6U8 |
| Mannosyltransferase | glycosylation CDG Il | | |
| | (608776) | | |
| α-1,2- | Congenital disorders of N- | 79796 | Q9H6U8 |
| Mannosyltransferase | glycosylation, type I (pre- | | |
| | Golgi glycosylation defects) | | |
| α-1,3- | Congenital disorders of N- | 440138 | Q2TAA5 |
| Mannosyltransferase | glycosylation CDG Ii | | |
| α-D-Mannosidase | α-Mannosidosis, type I | 10195 | Q92685 |
| | (severe) or II (mild) | | |
| α-L-Fucosidase | Fucosidosis | 4123 | Q9NTJ4 |
| α-l-Iduronidase | Mucopolysaccharidosis MPS | 2517 | P04066 |
| | I H/S (Hurler-Scheie | | |
| | syndrome) | | |
| α-l-Iduronidase | Mucopolysaccharidosis MPS | 3425 | P35475 |
| | I-H (Hurler's syndrome) | | |
| α-l-Iduronidase | Mucopolysaccharidosis MPS | 3425 | P35475 |
| | I-S (Scheie's syndrome) | | |
| β-1,4- | Congenital disorders of N- | 3425 | P35475 |
| Galactosyltransferase | glycosylation CDG IId | | |
| β-1,4- | Congenital disorders of N- | 2683 | P15291 |
| Mannosyltransferase | glycosylation CDG Ik | | |
| β-D-Mannosidase | β-Mannosidosis | 56052 | Q9BT22 |
| β-Galactosidase | Mucopolysaccharidosis MPS | 4126 | O00462 |
| | IV (Morquio's syndrome) | | |
| | Type IV-B | | |
| β-Glucuronidase | Mucopolysaccharidosis MPS | 2720 | P16278 |
| | VII (Sly's syndrome) | | |
| β-Hexosaminidase A | Tay-Sachs disease | 2990 | P08236 |
| β-Hexosaminidase B | Sandhoff's disease | 3073 | P06865 |

In some embodiments, an effector described herein comprises an enzyme of Table 19A, or a functional variant thereof, e.g., a homolog (e.g., ortholog or paralog) or fragment thereof. In some embodiments, an effector described herein comprises a protein having at least 80%, 85%, 90%, 95%, 967%, 98%, 99% sequence identity to an amino acid sequence listed in Table 54 by reference to its UniProt ID. In some embodiments, the functional variant catalyzes the same reaction as the corresponding wild-type protein, e.g., at a rate no less than 10%, 20%, 30%, 40%, or 50% lower than the wild-type protein. In some embodiments, a CAVector encoding an enzyme of Table 19A, or a functional variant thereof is used for the treatment of a disease or disorder of Table 19A. In some embodiments, a CAVector is used to deliver uridine diphosphate glucuronyl-transferase or a functional variant thereof to a target cell, e.g., a liver cell. In some embodiments, a CAVector is used to deliver OCA1 or a functional variant thereof to a target cell, e.g., a retinal cell.

TABLE 19B
Exemplary non-enzymatic effectors and corresponding indications

| Effector | Indication | Entrez Gene ID | UniProt ID |
|---|---|---|---|
| Survival motor neuron | spinal muscular atrophy | 6606 | Q16637 |
| protein (SMN) | | | |
| Dystrophin or micro- | muscular dystrophy | 1756 | P11532 |
| dystrophin | (e.g., Duchenne | | |
| | muscular dystrophy or | | |
| | Becker muscular | | |
| | dystrophy) | | |
| Complement protein, | Complement Factor I | 3426 | P05156 |
| e.g., Complement | deficiency | | |
| factor C1 | | | |
| Complement factor H | Atypical hemolytic | 3075 | P08603 |
| | uremic syndrome | | |
| Cystinosin (lysosomal | Cystinosis | 1497 | O60931 |
| cystine transporter) | | | |
| Epididymal secretory | Niemann-Pick disease | 10577 | P61916 |
| protein 1 (HE1; NPC2 | Type C2 | | |
| protein) | | | |
| GDP-fucose | Congenital disorders of | 55343 | Q96A29 |
| transporter-1 | N-glycosylation CDG | | |
| | IIc (Rambam-Hasharon | | |
| | syndrome) | | |
| GM2 activator protein | GM2 activator protein | 2760 | Q17900 |
| | deficiency (Tay-Sachs | | |
| | disease AB variant, | | |
| | GM2A) | | |
| Lysosomal | Ceroid lipofuscinosis | 1207 | Q13286 |
| transmembrane CLN3 | Juvenile form (CLN3, | | |
| protein | Batten disease, Vogt- | | |
| | Spielmeyer disease) | | |
| Lysosomal | Ceroid lipofuscinosis | 1203 | O75503 |
| transmembrane CLN5 | Variant late infantile | | |
| protein | form, Finnish type | | |
| | (CLN5) | | |
| Na phosphate | Infantile sialic acid | 26503 | Q9NRA2 |
| cotransporter, sialin | storage disorder | | |
| Na phosphate | Sialuria Finnish type | 26503 | Q9NRA2 |
| cotransporter, sialin | (Salla disease) | | |
| NPC1 protein | Niemann-Pick disease | 4864 | O15118 |
| | Type C1/Type D | | |
| Oligomeric Golgi | Congenital disorders of | 91949 | P83436 |
| complex-7 | N-glycosylation CDG | | |
| | IIe | | |
| Prosaposin | Prosaposin deficiency | 5660 | P07602 |
| Protective | Galactosialidosis | 5476 | P10619 |
| protein/cathepsin A | (Goldberg's syndrome, | | |
| (PPCA) | combined | | |
| | neuraminidase and β- | | |
| | galactosidase | | |
| | deficiency) | | |
| Protein involved in | Congenital disorders of | 9526 | O75352 |
| mannose-P-dolichol | N-glycosylation CDG If | | |
| utilization | | | |
| Saposin B | Saposin B deficiency | 5660 | P07602 |
| | (sulfatide activator | | |
| | deficiency) | | |
| Saposin C | Saposin C deficiency | 5660 | P07602 |
| | (Gaucher's activator | | |
| | deficiency) | | |
| Sulfatase-modifying | Mucosulfatidosis | 285362 | Q8NBK3 |
| factor-1 | (multiple sulfatase | | |
| | deficiency) | | |
| Transmembrane | Ceroid lipofuscinosis | 54982 | Q9NWW5 |
| CLN6 protein | Variant late infantile | | |
| | form (CLN6) | | |
| Transmembrane | Ceroid lipofuscinosis | 2055 | Q9UBY8 |
| CLN8 protein | Progressive epilepsy | | |
| | with intellectual | | |
| | disability | | |
| vWF | von Willebrand disease | 7450 | P04275 |
| Factor I (fibrinogen) | Afibrinogenomia | 2243, 2244, 2266 | P02671, P02675, |
| | | | P02679 |
| erythropoietin (hEPO) | | | |

In some embodiments, an effector described herein comprises an erythropoietin (EPO), e.g., a human erythropoietin (hEPO), or a functional variant thereof. In some embodiments, a CAVector encoding an erythropoietin, or a functional variant thereof is used for stimulating erythropoiesis. In some embodiments, a CAVector encoding an erythropoietin, or a functional variant thereof is used for the treatment of a disease or disorder, e.g., anemia. In some embodiments, a CAVector is used to deliver EPO or a functional variant thereof to a target cell, e.g., a red blood cell.

In some embodiments, an effector described herein comprises a polypeptide of Table 19B, or a functional variant thereof, e.g., a homolog (e.g., ortholog or paralog) or fragment thereof. In some embodiments, an effector described herein comprises a protein having at least 80%, 85%, 90%, 95%, 967%, 98%, 99% sequence identity to an amino acid sequence listed in Table 55 by reference to its UniProt ID. In some embodiments, a CAVector encoding a polypeptide of Table 19B, or a functional variant thereof is used for the treatment of a disease or disorder of Table 19B. In some embodiments, a CAVector is used to deliver SMN or a functional variant thereof to a target cell, e.g., a cell of the spinal cord and/or a motor neuron. In some embodiments, a CAVector is used to deliver a micro-dystrophin to a target cell, e.g., a myocyte.

Exemplary micro-dystrophins are described in Duan, “Systemic AAV Micro-dystrophin Gene Therapy for Duchenne Muscular Dystrophy.” Mol Ther. 2018 Oct. 3; 26(10):2337-2356. doi: 10.1016/j.ymthe.2018.07.011. Epub 2018 Jul. 17.

In some embodiments, an effector described herein comprises a clotting factor, e.g., a clotting factor listed in any table herein (e.g., Table 19A or 19B). In some embodiments, an effector described herein comprises a protein that, when mutated, causes a lysosomal storage disorder, e.g., a protein listed in any table herein (e.g., Table 19A or 19B). In some embodiments, an effector described herein comprises a transporter protein, e.g., a transporter protein listed in any table herein (e.g., Table 19A or 19B).

In some embodiments, a functional variant of a wild-type protein comprises a protein that has one or more activities of the wild-type protein, e.g., the functional variant catalyzes the same reaction as the corresponding wild-type protein, e.g., at a rate no less than 10%, 20%, 30%, 40%, or 50% lower than the wild-type protein. In some embodiments, the functional variant binds to the same binding partner that is bound by the wild-type protein, e.g., with a Kd of no more than 10%, 20%, 30%, 40%, or 50% higher than the Kd of the corresponding wild-type protein for the same binding partner under the same conditions. In some embodiments, the functional variant has at a polypeptide sequence at least 70%, 75%, 80%, 85%, 90%, 95%, 96%, 97%, 98%, or 99% identical to that of the wild-type polypeptide. In some embodiments, the functional variant comprises a homolog (e.g., ortholog or paralog) of the corresponding wild-type protein. In some embodiments, the functional variant is a fusion protein. In some embodiments, the fusion comprises a first region with at least 70%, 75%, 80%, 85%, 90%, 95%, 96%, 97%, 98%, or 99% identity to the corresponding wild-type protein, and a second, heterologous region. In some embodiments, the functional variant comprises or consists of a fragment of the corresponding wild-type protein.

Regeneration, Repair, and Fibrosis Factors

Therapeutic polypeptides described herein also include growth factors, e.g., as disclosed in Table 56, or functional variants thereof, e.g., a protein having at least 80%, 85%, 90%, 95%, 967%, 98%, 99% identity to a protein sequence disclosed in Table 56 by reference to its UniProt ID. Also included are antibodies or fragments thereof against such growth factors, or miRNAs that promote regeneration and repair.

TABLE 56
Exemplary regeneration, repair, and fibrosis factors

| Target | Gene accession # | Protein accession # |
|---|---|---|
| VEGF-A | NG_008732 | NP_001165094 |
| NRG-1 | NG_012005 | NP_001153471 |
| FGF2 | NG_029067 | NP_001348594 |
| FGF1 | Gene ID: 2246 | NP_001341882 |
| miR-199-3p | MIMAT0000232 | |
| miR-590-3p | MIMAT0004801 | |
| mi-17-92 | MI0000071 | https://www.ncbi.nlm.nih.gov/pmc/ |
| | | articles/PMC2732113/figure/F1/ |
| miR-222 | MI0000299 | |
| miR-302-367 | MIR302A And | https://www.ncbi.nlm.nih.gov/pmc/ |
| | MIR367 | articles/PMC4400607/ |

Transformation Factors

Therapeutic polypeptides described herein also include transformation factors, e.g., protein factors that transform fibroblasts into differentiated cell e.g., factors disclosed in Table 57 or functional variants thereof, .g., a protein having at least 80%, 85%, 90%, 95%, 967%, 98%, 99% identity to a protein sequence disclosed in Table 57 by reference to its UniProt ID.

TABLE 57
Exemplary transformation factors

| Target | Indication | Gene accession # | Protein accession # |
|---|---|---|---|
| MESP1 | Organ Repair by | Gene ID: 55897 | EAX02066 |
| | transforming fibroblasts | | |
| ETS2 | Organ Repair by | GeneID: 2114 | NP_005230 |
| | transforming fibroblasts | | |
| HAND2 | Organ Repair by | GeneID: 9464 | NP_068808 |
| | transforming fibroblasts | | |
| MYOCARDIN | Organ Repair by | GeneID: 93649 | NP_001139784 |
| | transforming fibroblasts | | |
| ESRRA | Organ Repair by | Gene ID: 2101 | AAH92470 |
| | transforming fibroblasts | | |
| miR-1 | Organ Repair by | MI0000651 | n/a |
| | transforming fibroblasts | | |
| miR-133 | Organ Repair by | MI0000450 | n/a |
| | transforming fibroblasts | | |
| TGFb | Organ Repair by | GeneID: 7040 | NP_000651.3 |
| | transforming fibroblasts | | |
| WNT | Organ Repair by | Gene ID: 7471 | NP_005421 |
| | transforming fibroblasts | | |
| JAK | Organ Repair by | Gene ID: 3716 | NP_001308784 |
| | transforming fibroblasts | | |
| NOTCH | Organ Repair by | GeneID: 4851 | XP_011517019 |
| | transforming fibroblasts | | |

Proteins that Stimulate Cellular Regeneration

Therapeutic polypeptides described herein also include proteins that stimulate cellular regeneration e.g., proteins disclosed in Table 58 or functional variants thereof, e.g., a protein having at least 80%, 85%, 90%, 95%, 967%, 98%, 99% identity to a protein sequence disclosed in Table 58 by reference to its UniProt ID.

TABLE 58
Exemplary proteins that stimulate cellular regeneration

| Target | Gene accession # | Protein accession # |
|---|---|---|
| MST1 | NG_016454 | NP_066278 |
| STK30 | Gene ID: 26448 | NP_036103 |
| MST2 | Gene ID: 6788 | NP_006272 |
| SAV1 | Gene ID: 60485 | NP_068590 |
| LATS1 | Gene ID: 9113 | NP_004681 |
| LATS2 | Gene ID: 26524 | NP_055387 |
| YAP1 | NG_029530 | NP_001123617 |
| CDKN2b | NG_023297 | NP_004927 |
| CDKN2a | NG_007485 | NP_478102 |

STING Modulator Effectors

In some embodiments, a secreted effector described herein modulates STING/cGAS signaling. In some embodiments, the STING modulator is a polypeptide, e.g., a viral polypeptide or a functional variant thereof. For instance, the effector may comprise a STING modulator (e.g., inhibitor) described in Maringer et al. “Message in a bottle: lessons learned from antagonism of STING signalling during RNA virus infection” Cytokine & Growth Factor Reviews Volume 25, Issue 6, December 2014, Pages 669-679, which is incorporated herein by reference in its entirety. Additional STING modulators (e.g., activators) are described, e.g., in Wang et al. “STING activator c-di-GMP enhances the anti-tumor effects of peptide vaccines in melanoma-bearing mice.” Cancer Immunol Immunother. 2015 August; 64(8):1057-66. doi: 10.1007/s00262-015-1713-5. Epub 2015 May 19; Bose “cGAS/STING Pathway in Cancer: Jekyll and Hyde Story of Cancer Immune Response” Int J Mol Sci. 2017 November; 18(11): 2456; and Fu et al. “STING agonist formulated cancer vaccines can cure established tumors resistant to PD-1 blockade” Sci Transl Med. 2015 Apr. 15; 7(283): 283ra52, each of which is incorporated herein by reference in its entirety.

Some examples of peptides include, but are not limited to, fluorescent tag or marker, antigen, peptide therapeutic, synthetic or analog peptide from naturally-bioactive peptide, agonist or antagonist peptide, anti-microbial peptide, a targeting or cytotoxic peptide, a degradation or self-destruction peptide, and degradation or self-destruction peptides. Peptides useful in the invention described herein also include antigen-binding peptides, e.g., antigen binding antibody or antibody-like fragments, such as single chain antibodies, nanobodies (see, e.g., Steeland et al. 2016. Nanobodies as therapeutics: big opportunities for small antibodies. Drug Discov Today: 21(7):1076-113). Such antigen binding peptides may bind a cytosolic antigen, a nuclear antigen, or an intra-organellar antigen.

In some embodiments, the genetic element comprises a sequence that encodes small peptides, peptidomimetics (e.g., peptoids), amino acids, and amino acid analogs. Such therapeutics generally have a molecular weight less than about 5,000 grams per mole, a molecular weight less than about 2,000 grams per mole, a molecular weight less than about 1,000 grams per mole, a molecular weight less than about 500 grams per mole, and salts, esters, and other pharmaceutically acceptable forms of such compounds. Such therapeutics may include, but are not limited to, a neurotransmitter, a hormone, a drug, a toxin, a viral or microbial particle, a synthetic molecule, and agonists or antagonists thereof.

In some embodiments, the composition or CAVector described herein includes a polypeptide linked to a ligand that is capable of targeting a specific location, tissue, or cell.

Gene Editing Components

The genetic element of the CAVector may include one or more genes that encode a component of a gene editing system. Exemplary gene editing systems include the clustered regulatory interspaced short palindromic repeat (CRISPR) system, zinc finger nucleases (ZFNs), Gene Writers, reverse transcriptases, epigenetic modifiers, recombinases, and Transcription Activator-Like Effector-based Nucleases (TALEN). ZFNs, TALENs, and CRISPR-based methods are described, e.g., in Gaj et al. Trends Biotechnol. 31.7(2013):397-405; CRISPR methods of gene editing are described, e.g., in Guan et al., Application of CRISPR-Cas system in gene therapy: Pre-clinical progress in animal model. DNA Repair 2016 October; 46:1-8. doi: 10.1016/j.dnarep.2016.07.004; Zheng et al., Precise gene deletion and replacement using the CRISPR/Cas9 system in human cells. BioTechniques, Vol. 57, No. 3, September 2014, pp. 115-124. Non-limiting examples of gene editing and gene writing systems are described, for example, in PCT Publication No. WO 2020/047124 (e.g., any sequence disclosed therein) and Anzalone et al. 2019, Nature 576: 149-157 (each of which is incorporated herein by reference in their entirety).

In some embodiments, the genetic element comprises a sequence encoding a Gene Writer, e.g., comprising one or more elements from a non-long terminal repeat (LTR) retrotransposon (e.g., an apurinic/apyrimidinic endonuclease (APE)-type or a restriction enzyme-like endonuclease (RLE)-type). In some embodiments, the Gene Writer comprises a retrotransposase. In some embodiments, the the Gene Writer comprises one or more of a DNA binding domain, a reverse transcription domain, and/or an endonuclease domain. Examples of Gene Writers, reverse transcriptases, and recombinases that may be encoded by a genetic element as described herein are described, for example, in PCT Publication No. WO 2020/047124 (incorporated herein by reference in its entirety).

CRISPR systems are adaptive defense systems originally discovered in bacteria and archaea. CRISPR systems use RNA-guided nucleases termed CRISPR-associated or “Cas” endonucleases (e.g., Cas9 or Cpf1) to cleave foreign DNA. In a typical CRISPR/Cas system, an endonuclease is directed to a target nucleotide sequence (e.g., a site in the genome that is to be sequence-edited) by sequence-specific, non-coding “guide RNAs” that target single- or double-stranded DNA sequences. Three classes (1-111) of CRISPR systems have been identified. The class II CRISPR systems use a single Cas endonuclease (rather than multiple Cas proteins). One class II CRISPR system includes a type II Cas endonuclease such as Cas9, a CRISPR RNA (“crRNA”), and a trans-activating crRNA (“tracrRNA”). The crRNA contains a “guide RNA”, typically about 20-nucleotide RNA sequence that corresponds to a target DNA sequence. The crRNA also contains a region that binds to the tracrRNA to form a partially double-stranded structure which is cleaved by RNase III, resulting in a crRNA/tracrRNA hybrid. The crRNA/tracrRNA hybrid then directs the Cas9 endonuclease to recognize and cleave the target DNA sequence. The target DNA sequence must generally be adjacent to a “protospacer adjacent motif” (“PAM”) that is specific for a given Cas endonuclease; however, PAM sequences appear throughout a given genome.

In some embodiments, the CAVector includes a gene for a CRISPR endonuclease. For example, some CRISPR endonucleases identified from various prokaryotic species have unique PAM sequence requirements; examples of PAM sequences include 5′-NGG (Streptococcus pyogenes), 5′-NNAGAA (Streptococcus thermophilus CRISPR1), 5′-NGGNG (Streptococcus thermophilus CRISPR3), and 5′-NNNGATT (Neisseria meningiditis). Some endonucleases, e.g., Cas9 endonucleases, are associated with G-rich PAM sites, e.g., 5′-NGG, and perform blunt-end cleaving of the target DNA at a location 3 nucleotides upstream from (5′ from) the PAM site. Another class II CRISPR system includes the type V endonuclease Cpf1, which is smaller than Cas9; examples include AsCpf1 (from Acidaminococcus sp.) and LbCpf1 (from Lachnospiraceae sp.). Cpf1 endonucleases, are associated with T-rich PAM sites, e.g., 5′-TTN. Cpf1 can also recognize a 5′-CTA PAM motif. Cpf1 cleaves the target DNA by introducing an offset or staggered double-strand break with a 4- or 5-nucleotide 5′ overhang, for example, cleaving a target DNA with a 5-nucleotide offset or staggered cut located 18 nucleotides downstream from (3′ from) from the PAM site on the coding strand and 23 nucleotides downstream from the PAM site on the complimentary strand; the 5-nucleotide overhang that results from such offset cleavage allows more precise genome editing by DNA insertion by homologous recombination than by insertion at blunt-end cleaved DNA. See, e.g., Zetsche et al. (2015) Cell, 163:759-771.

A variety of CRISPR associated (Cas) genes may be included in the CAVector. Specific examples of genes are those that encode Cas proteins from class II systems including Cas1, Cas2, Cas3, Cas4, Cas5, Cas6, Cas7, Cas8, Cas9, Cas10, Cpf1, C2C1, or C2C3. In some embodiments, the CAVector includes a gene encoding a Cas protein, e.g., a Cas9 protein, may be from any of a variety of prokaryotic species. In some embodiments, the CAVector includes a gene encoding a particular Cas protein, e.g., a particular Cas9 protein, is selected to recognize a particular protospacer-adjacent motif (PAM) sequence. In some embodiments, the CAVector includes nucleic acids encoding two or more different Cas proteins, or two or more Cas proteins, may be introduced into a cell, zygote, embryo, or animal, e.g., to allow for recognition and modification of sites comprising the same, similar or different PAM motifs. In some embodiments, the CAVector includes a gene encoding a modified Cas protein with a deactivated nuclease, e.g., nuclease-deficient Cas9.

Whereas wild-type Cas9 protein generates double-strand breaks (DSBs) at specific DNA sequences targeted by a gRNA, a number of CRISPR endonucleases having modified functionalities are known, for example: a “nickase” version of Cas endonuclease (e.g., Cas9) generates only a single-strand break; a catalytically inactive Cas endonuclease, e.g., Cas9 (“dCas9”) does not cut the target DNA. A gene encoding a dCas9 can be fused with a gene encoding an effector domain to repress (CRISPRi) or activate (CRISPRa) expression of a target gene. For example, the gene may encode a Cas9 fusion with a transcriptional silencer (e.g., a KRAB domain) or a transcriptional activator (e.g., a dCas9-VP64 fusion). A gene encoding a catalytically inactive Cas9 (dCas9) fused to FokI nuclease (“dCas9-FokI”) can be included to generate DSBs at target sequences homologous to two gRNAs. See, e.g., the numerous CRISPR/Cas9 plasmids disclosed in and publicly available from the Addgene repository (Addgene, 75 Sidney St., Suite 550A, Cambridge, MA 02139; addgene.org/crispr/). A “double nickase” Cas9 that introduces two separate double-strand breaks, each directed by a separate guide RNA, is described as achieving more accurate genome editing by Ran et al. (2013) Cell, 154:1380-1389.

CRISPR technology for editing the genes of eukaryotes is disclosed in US Patent Application Publications 2016/0138008A1 and US2015/0344912A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cpf1 endonuclease and corresponding guide RNAs and PAM sites are disclosed in US Patent Application Publication 2016/0208243 A1.

In some embodiments, the CAVector comprises a gene encoding a polypeptide described herein, e.g., a targeted nuclease, e.g., a Cas9, e.g., a wild type Cas9, a nickase Cas9 (e.g., Cas9 D10A), a dead Cas9 (dCas9), eSpCas9, Cpf1, C2C1, or C2C3, and a gRNA. The choice of genes encoding the nuclease and gRNA(s) is determined by whether the targeted mutation is a deletion, substitution, or addition of nucleotides, e.g., a deletion, substitution, or addition of nucleotides to a targeted sequence. Genes that encode a catalytically inactive endonuclease e.g., a dead Cas9 (dCas9, e.g., D10A; H840A) tethered with all or a portion of (e.g., biologically active portion of) an (one or more) effector domain (e.g., VP64) create chimeric proteins that can modulate activity and/or expression of one or more target nucleic acids sequences.

In some embodiments, the CAVector includes a gene encoding a fusion of a dCas9 with all or a portion of one or more effector domains (e.g., a full-length wild-type effector domain, or a fragment or variant thereof, e.g., a biologically active portion thereof) to create a chimeric protein useful in the methods described herein. Accordingly, in some embodiments, the CAVector includes a gene encoding a dCas9-methylase fusion. In other some embodiments, the CAVector includes a gene encoding a dCas9-enzyme fusion with a site-specific gRNA to target an endogenous gene.

In other aspects, the CAVector includes a gene encoding 1, 2, 3, 4, 5, 6, 7, 8, 9, 10, 11, 12, 13, 14, 15, 16, 17, 18, 19, 20, or more effector domains (all or a biologically active portion) fused with dCas9.

### Regulatory Sequences

In some embodiments, the genetic element comprises a regulatory sequence, e.g., a promoter or an enhancer, operably linked to the sequence encoding the effector.

In some embodiments, a promoter includes a DNA sequence that is located adjacent to a DNA sequence that encodes an expression product. A promoter may be linked operatively to the adjacent DNA sequence. A promoter typically increases an amount of product expressed from the DNA sequence as compared to an amount of the expressed product when no promoter exists. A promoter from one organism can be utilized to enhance product expression from the DNA sequence that originates from another organism. For example, a vertebrate promoter may be used for the expression of jellyfish GFP in vertebrates. In addition, one promoter element can increase an amount of products expressed for multiple DNA sequences attached in tandem. Hence, one promoter element can enhance the expression of one or more products. Multiple promoter elements are well-known to persons of ordinary skill in the art.

In one embodiment, high-level constitutive expression is desired. Examples of such promoters include, without limitation, the retroviral Rous sarcoma virus (RSV) long terminal repeat (LTR) promoter/enhancer, the cytomegalovirus (CMV) immediate early promoter/enhancer (see, e.g., Boshart et al, Cell, 41:521-530 (1985)), the SV40 promoter, the dihydrofolate reductase promoter, the cytoplasmic .beta.-actin promoter and the phosphoglycerol kinase (PGK) promoter.

In another embodiment, inducible promoters may be desired. Inducible promoters are those which are regulated by exogenously supplied compounds, e.g., provided either in cis or in trans, including without limitation, the zinc-inducible sheep metallothionine (MT) promoter; the dexamethasone (Dex)-inducible mouse mammary tumor virus (MMTV) promoter; the T7 polymerase promoter system (WO 98/10088); the tetracycline-repressible system (Gossen et al, Proc. Natl. Acad. Sci. USA, 89:5547-5551 (1992)); the tetracycline-inducible system (Gossen et al., Science, 268:1766-1769 (1995); see also Harvey et al., Curr. Opin. Chem. Biol., 2:512-518 (1998)); the RU486-inducible system (Wang et al., Nat. Biotech., 15:239-243 (1997) and Wang et al., Gene Ther., 4:432-441 (1997)]; and the rapamycin-inducible system (Magari et al., J. Clin. Invest., 100:2865-2872 (1997); Rivera et al., Nat. Medicine. 2:1028-1032 (1996)). Other types of inducible promoters which may be useful in this context are those which are regulated by a specific physiological state, e.g., temperature, acute phase, or in replicating cells only.

In some embodiments, a native promoter for a gene or nucleic acid sequence of interest is used. The native promoter may be used when it is desired that expression of the gene or the nucleic acid sequence should mimic the native expression. The native promoter may be used when expression of the gene or other nucleic acid sequence must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the genetic element comprises a gene operably linked to a tissue-specific promoter. For instance, if expression in skeletal muscle is desired, a promoter active in muscle may be used. These include the promoters from genes encoding skeletal α-actin, myosin light chain 2A, dystrophin, muscle creatine kinase, as well as synthetic muscle promoters with activities higher than naturally-occurring promoters. See Li et al., Nat. Biotech., 17:241-245 (1999). Examples of promoters that are tissue-specific are known for liver albumin, Miyatake et al. J. Virol., 71:5124-32 (1997); hepatitis B virus core promoter, Sandig et al., Gene Ther. 3:1002-9 (1996); alpha-fetoprotein (AFP), Arbuthnot et al., Hum. Gene Ther., 7:1503-14 (1996)], bone (osteocalcin, Stein et al., Mol. Biol. Rep., 24:185-96 (1997); bone sialoprotein, Chen et al., J. Bone Miner. Res. 11:654-64 (1996)), lymphocytes (CD2, Hansal et al., J. Immunol., 161:1063-8 (1998); immunoglobulin heavy chain; T cell receptor a chain), neuronal (neuron-specific enolase (NSE) promoter, Andersen et al. Cell. Mol. Neurobiol., 13:503-15 (1993); neurofilament light-chain gene, Piccioli et al., Proc. Natl. Acad. Sci. USA, 88:5611-5 (1991); the neuron-specific vgf gene, Piccioli et al., Neuron, 15:373-84 (1995)]; among others.

The genetic element may include an enhancer, e.g., a DNA sequence that is located adjacent to the DNA sequence that encodes a gene. Enhancer elements are typically located upstream of a promoter element or can be located downstream of or within a coding DNA sequence (e.g., a DNA sequence transcribed or translated into a product or products). Hence, an enhancer element can be located 100 base pairs, 200 base pairs, or 300 or more base pairs upstream or downstream of a DNA sequence that encodes the product. Enhancer elements can increase an amount of recombinant product expressed from a DNA sequence above increased expression afforded by a promoter element. Multiple enhancer elements are readily available to persons of ordinary skill in the art.

In some embodiments, the genetic element comprises one or more inverted terminal repeats (ITR) flanking the sequences encoding the expression products described herein. In some embodiments, the genetic element comprises one or more long terminal repeats (LTR) flanking the sequence encoding the expression products described herein. Examples of promoter sequences that may be used, include, but are not limited to, the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, and a Rous sarcoma virus promoter.

### Replication Proteins

In some embodiments, the genetic element of the CAVector, e.g., synthetic CAVector, may include sequences that encode one or more replication proteins. In some embodiments, the CAVector may replicate by a rolling-circle replication method, e.g., synthesis of the leading strand and the lagging strand is uncoupled. In such embodiments, the CAVector comprises three elements additional elements: i) a gene encoding an initiator protein, ii) a double strand origin, and iii) a single strand origin. A rolling circle replication (RCR) protein complex comprising replication proteins binds to the leading strand and destabilizes the replication origin. The RCR complex cleaves the genome to generate a free 3′OH extremity. Cellular DNA polymerase initiates viral DNA replication from the free 3′OH extremity. After the genome has been replicated, the RCR complex closes the loop covalently. This leads to the release of a positive circular single-stranded parental DNA molecule and a circular double-stranded DNA molecule composed of the negative parental strand and the newly synthesized positive strand. The single-stranded DNA molecule can be either encapsidated or involved in a second round of replication. See for example, Virology Journal 2009, 6:60 doi:10.1186/1743-422X-6-60.

The genetic element may comprise a sequence encoding a polymerase, e.g., RNA polymerase or a DNA polymerase.

### Other Sequences

In some embodiments, the genetic element further includes a nucleic acid encoding a product (e.g., a ribozyme, a therapeutic mRNA encoding a protein, an exogenous gene).

In some embodiments, the genetic element includes one or more sequences that affect species and/or tissue and/or cell tropism (e.g. capsid protein sequences), infectivity (e.g. capsid protein sequences), immunosuppression/activation (e.g. regulatory nucleic acids), viral genome binding and/or packaging, immune evasion (non-immunogenicity and/or tolerance), pharmacokinetics, endocytosis and/or cell attachment, nuclear entry, intracellular modulation and localization, exocytosis modulation, propagation, and nucleic acid protection of the CAVector in a host or host cell.

In some embodiments, the genetic element may comprise other sequences that include DNA, RNA, or artificial nucleic acids. The other sequences may include, but are not limited to, genomic DNA, cDNA, or sequences that encode tRNA, mRNA, rRNA, miRNA, gRNA, siRNA, or other RNAi molecules. In one embodiment, the genetic element includes a sequence encoding an siRNA to target a different loci of the same gene expression product as the regulatory nucleic acid. In one embodiment, the genetic element includes a sequence encoding an siRNA to target a different gene expression product as the regulatory nucleic acid.

In some embodiments, the genetic element further comprises one or more of the following sequences: a sequence that encodes one or more miRNAs, a sequence that encodes one or more replication proteins, a sequence that encodes an exogenous gene, a sequence that encodes a therapeutic, a regulatory sequence (e.g., a promoter, enhancer), a sequence that encodes one or more regulatory sequences that targets endogenous genes (siRNA, lncRNAs, shRNA), and a sequence that encodes a therapeutic mRNA or protein.

The other sequences may have a length from about 2 to about 5000 nts, about 10 to about 100 nts, about 50 to about 150 nts, about 100 to about 200 nts, about 150 to about 250 nts, about 200 to about 300 nts, about 250 to about 350 nts, about 300 to about 500 nts, about 10 to about 1000 nts, about 50 to about 1000 nts, about 100 to about 1000 nts, about 1000 to about 2000 nts, about 2000 to about 3000 nts, about 3000 to about 4000 nts, about 4000 to about 5000 nts, or any range therebetween.

Encoded Genes

For example, the genetic element may include a gene associated with a signaling biochemical pathway, e.g., a signaling biochemical pathway-associated gene or polynucleotide. Examples include a disease associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease.

Moreover, the genetic elements can encode targeting moieties, as described elsewhere herein. This can be achieved, e.g., by inserting a polynucleotide encoding a sugar, a glycolipid, or a protein, such as an antibody. Those skilled in the art know additional methods for generating targeting moieties.

Viral Sequence

In some embodiments, the genetic element comprises at least one viral sequence. In some embodiments, the sequence has homology or identity to one or more sequence from a single stranded DNA virus, e.g., CAV, Anellovirus, Bidnavirus, Circovirus, Geminivirus, Genomovirus, Inovirus, Microvirus, Nanovirus, Parvovirus, and Spiravirus. In some embodiments, the sequence has homology or identity to one or more sequence from a double stranded DNA virus, e.g., Adenovirus, Ampullavirus, Ascovirus, Asfarvirus, Baculovirus, Fusellovirus, Globulovirus, Guttavirus, Hytrosavirus, Herpesvirus, Iridovirus, Lipothrixvirus, Nimavirus, and Poxvirus. In some embodiments, the sequence has homology or identity to one or more sequence from an RNA virus, e.g., Alphavirus, Furovirus, Hepatitis virus, Hordeivirus, Tobamovirus, Tobravirus, Tricornavirus, Rubivirus, Birnavirus, Cystovirus, Partitivirus, and Reovirus.

In some embodiments, the genetic element may comprise one or more sequences from a non-pathogenic virus, e.g., a symbiotic virus, e.g., a commensal virus, e.g., a native virus, e.g., a CAV. In some embodiments, the genetic element may comprise a sequence with homology or identity to a the Cuxhaven 1 isolate of CAV (e.g, as listed in Table 1A or 1B). In some embodiments, the non-pathogenic virus is a non-enveloped, single-stranded DNA virus with a circular, negative-sense genome, e.g., CAV. In some embodiments, the genetic element may comprise one or more sequences or a fragment of a sequence from a non-pathogenic virus having at least about 60%, 70% 80%, 85%, 90% 95%, 96%, 97%, 98% and 99% nucleotide sequence identity to any one of the nucleotide sequences described herein.

Since, in some embodiments, recombinant retroviruses are defective, assistance may be provided order to produce infectious particles. Such assistance can be provided, e.g., by using cell lines that contain plasmids encoding all of the structural genes of the retrovirus under the control of regulatory sequences within the LTR. Suitable cell lines for replicating the CAVectors described herein include cell lines known in the art, e.g., A549 cells, which can be modified as described herein. Said genetic element can additionally contain a gene encoding a selectable marker so that the desired genetic elements can be identified.

In some embodiments, the genetic element includes non-silent mutations, e.g., base substitutions, deletions, or additions resulting in amino acid differences in the encoded polypeptide, so long as the sequence remains at least about 70%, 75%, 80%, 85%, 90%, 95%, 96%, 97%, 98%, or 99% identical to the polypeptide encoded by the first nucleotide sequence or otherwise is useful for practicing the present invention. In this regard, certain conservative amino acid substitutions may be made which are generally recognized not to inactivate overall protein function: such as in regard of positively charged amino acids (and vice versa), lysine, arginine and histidine; in regard of negatively charged amino acids (and vice versa), aspartic acid and glutamic acid; and in regard of certain groups of neutrally charged amino acids (and in all cases, also vice versa), (1) alanine and serine, (2) asparagine, glutamine, and histidine, (3) cysteine and serine, (4) glycine and proline, (5) isoleucine, leucine and valine, (6) methionine, leucine and isoleucine, (7) phenylalanine, methionine, leucine, and tyrosine, (8) serine and threonine, (9) tryptophan and tyrosine, (10) and for example tyrosine, tryptophan and phenylalanine. Amino acids can be classified according to physical properties and contribution to secondary and tertiary protein structure. A conservative substitution is recognized in the art as a substitution of one amino acid for another amino acid that has similar properties.

In some embodiments, identity of two or more nucleic acid or polypeptide sequences having the same or a specified percentage of nucleotides or amino acid residues that are the same (e.g., about 60%, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) may be measured using a BLAST or BLAST 2.0 sequence comparison algorithms with default parameters described below, or by manual alignment and visual inspection (see, e.g., NCBI web site www.ncbi.nlm.nih.gov/BLAST/ or the like). Identity may also refer to, or may be applied to, the compliment of a test sequence. Identity also includes sequences that have deletions and/or additions, as well as those that have substitutions. As described herein, the algorithms account for gaps and the like. Identity may exist over a region that is at least about 10 amino acids or nucleotides in length, about 15 amino acids or nucleotides in length, about 20 amino acids or nucleotides in length, about 25 amino acids or nucleotides in length, about 30 amino acids or nucleotides in length, about 35 amino acids or nucleotides in length, about 40 amino acids or nucleotides in length, about 45 amino acids or nucleotides in length, about 50 amino acids or nucleotides in length, or more. Since the genetic code is degenerate, a homologous nucleotide sequence can include any number of silent base changes, i.e., nucleotide substitutions that nonetheless encode the same amino acid.

### Proteinaceous Exterior

In some embodiments, the CAVector, e.g., synthetic CAVector, comprises a proteinaceous exterior that encloses a genetic element. The proteinaceous exterior can comprise capsid protein (e.g., a CAV VP1 molecule as described herein). In some embodiments, the protein is a capsid protein, e.g., has a sequence having at least about 60%, 65%, 70%, 75%, 80%, 85%, 90% 95%, 96%, 97%, 98%, 99%, or 100% sequence identity to a protein encoded by any one of the nucleotide sequences encoding a capsid protein described herein, e.g., a CAV VP1 molecule, e.g., as described herein. In some embodiments, the protein or a functional fragment of a capsid protein is encoded by a nucleotide sequence having at least about 60%, 70% 80%, 85%, 90% 95%, 96%, 97%, 98%, 99%, or 100% sequence identity to an CAV VP1 nucleic acid, e.g., as described herein. In some embodiments, the CAVector comprises a nucleotide sequence encoding a capsid protein or a functional fragment of a capsid protein or a sequence having at least about 60%, 70% 80%, 85%, 90% 95%, 96%, 97%, 98%, 99%, or 100% sequence identity to an CAV VP1 molecule as described herein. The proteinaceous exterior of the CAVectors may, in some embodiments, comprise a capsid protein that may self-assemble, e.g., to form the proteinaceous exterior. In some embodiments, the capsid protein may self-assemble into an icosahedral formation, e.g., that makes up the proteinaceous exterior.

In some embodiments, the proteinaceous exterior protein is encoded by a sequence of the genetic element of the CAVector (e.g., is in cis with the genetic element). In other embodiments, the proteinaceous exterior protein is encoded by a nucleic acid separate from the genetic element of the CAVector (e.g., is in trans with the genetic element).

In some embodiments, the ranges of amino acids with less sequence identity may provide one or more of the properties described herein and differences in cell/tissue/species specificity (e.g. tropism).

In some embodiments, the CAVector lacks lipids in the proteinaceous exterior. In some embodiments, the CAVector lacks a lipid bilayer, e.g., a viral envelope. In some embodiments, the interior of the CAVector is entirely covered (e.g., 100% coverage) by a proteinaceous exterior. In some embodiments, the interior of the CAVector is less than 100% covered by the proteinaceous exterior, e.g., 95%, 90%, 85%, 80%, 70%, 60%, 50% or less coverage. In some embodiments, the proteinaceous exterior comprises gaps or discontinuities, e.g., permitting permeability to water, ions, peptides, or small molecules, so long as the genetic element is retained in the CAVector.

In some embodiments, the proteinaceous exterior comprises one or more proteins or polypeptides that specifically recognize and/or bind a host cell, e.g., a complementary protein or polypeptide, to mediate entry of the genetic element into the host cell.

In some embodiments, the proteinaceous exterior comprises an arginine-rich region and/or a jelly-roll region, e.g., of an VP1 molecule, e.g., as described herein. In some embodiments, the proteinaceous exterior comprises one or more of the following: one or more glycosylated proteins, a hydrophilic DNA-binding region, an arginine-rich region, a threonine-rich region, a glutamine-rich region, a N-terminal polyarginine sequence, a variable region, a C-terminal polyglutamine/glutamate sequence, and one or more disulfide bridges. For example, the proteinaceous exterior comprises a protein encoded by a CAV VP1 nucleic acid, e.g., as described herein.

In some embodiments, the proteinaceous exterior comprises one or more of the following characteristics: an icosahedral symmetry, recognizes and/or binds a molecule that interacts with one or more host cell molecules to mediate entry into the host cell, lacks lipid molecules, lacks carbohydrates, is pH and temperature stable, is detergent resistant, and is substantially non-pathogenic in a host.

In some embodiments, the protein, e.g., substantially non-pathogenic protein and/or proteinaceous exterior protein, comprises one or more glycosylated amino acids, e.g., 2, 3, 4, 5, 6, 7, 8, 9, 10, or more. In some embodiments, the protein, e.g., substantially non-pathogenic protein and/or proteinaceous exterior protein comprises at least one hydrophilic DNA-binding region, an arginine-rich region, a threonine-rich region, a glutamine-rich region, a N-terminal polyarginine sequence, a variable region, a C-terminal polyglutamine/glutamate sequence, and one or more disulfide bridges.

### III. Genetic Element Constructs

The genetic element described herein may be included in a genetic element construct (e.g., a tandem construct, e.g., as described herein).

In one aspect, the invention includes a genetic element construct comprising a genetic element comprising (i) a sequence encoding a non-pathogenic exterior protein (e.g., an CAV VP1 molecule or a splice variant or functional fragment thereof), (ii) an exterior protein binding sequence that binds the genetic element to the non-pathogenic exterior protein, and (iii) a sequence encoding an effector. In some embodiments, the genetic element construct is a tandem construct further comprising a second copy of the genetic element, or a fragment thereof (e.g., comprising an uRFS or a dRFS, e.g., as described herein).

The genetic element or any of the sequences within the genetic element can be obtained using any suitable method. Various recombinant methods are known in the art, such as, for example screening libraries from cells harboring viral sequences, deriving the sequences from a genetic element construct known to include the same, or isolating directly from cells and tissues containing the same, using standard techniques. Alternatively or in combination, part or all of the genetic element can be produced synthetically, rather than cloned.

In some embodiments, the genetic element construct includes regulatory elements, nucleic acid sequences homologous to target genes, and various reporter constructs for causing the expression of reporter molecules within a viable cell and/or when an intracellular molecule is present within a target cell.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In some embodiments, the genetic element construct is substantially non-pathogenic and/or substantially non-integrating in a host cell.

In some embodiments, the genetic element construct is in an amount sufficient to modulate one or more of phenotype, virus levels, gene expression, compete with other viruses, disease state, etc. at least about 5%, 10%, 15%, 20%, 25%, 30%, 35%, 40%, 45%, 50%, or more.

### IV. Compositions

The CAVectors described herein may also be included in pharmaceutical compositions with a pharmaceutically acceptable carrier or excipient, e.g., as described herein. In some embodiments, the pharmaceutical composition comprises at least 10⁵, 10⁶, 10⁷, 10⁸, 10⁹, 10¹⁰, 10¹¹, 10¹², 10¹³, 10¹⁴, or 10¹⁵ CAVectors. In some embodiments, the pharmaceutical composition comprises about 10⁵-10¹⁵, 10⁵-10¹⁰, or 10¹⁰-10¹⁵ CAVectors. In some embodiments, the pharmaceutical composition comprises about 10⁸ (e.g., about 10⁵, 10⁶, 10⁷, 10⁸, 10⁹, or 10¹⁰) genomic equivalents/mL of the CAVector. In some embodiments, the pharmaceutical composition comprises 10⁵-10¹⁰, 10⁶-10¹⁰, 10⁷-10¹⁰, 10⁸-10¹⁰, 10⁹-10¹⁰, 10⁵-10⁶, 10⁵-10⁷, 10⁵-10⁸, 10⁵-10⁹, 10⁵-10¹¹, 10⁵-10¹², 10⁵-10¹³, 10⁵-10¹⁴, 10⁵-10¹⁵, or 10¹⁰-10¹⁵ genomic equivalents/mL of the CAVector, e.g., as determined according to the method of measuring viral titer described in Example 1. In some embodiments, the pharmaceutical composition comprises sufficient CAVectors to deliver at least 1, 2, 5, or 10, 100, 500, 1000, 2000, 5000, 8,000, 1×10⁴, 1×10⁵, 1×10⁶, 1×10⁷ or greater copies of a genetic element comprised in the CAVectors per cell to a population of the eukaryotic cells. In some embodiments, the pharmaceutical composition comprises sufficient CAVectors to deliver at least about 1×10⁴, 1×10⁵, 1×10⁶, 1× or 10⁷, or about 1×10⁴-1×10⁵, 1×10⁴-1×10⁶, 1×10⁴-1×10⁷, 1×10⁵-1×10⁶, 1×10⁵-1×10⁷, or 1×10⁶-1×10⁷ copies of a genetic element comprised in the CAVectors per cell to a population of the eukaryotic cells.

In some embodiments, the pharmaceutical composition has one or more of the following characteristics: the pharmaceutical composition meets a pharmaceutical or good manufacturing practices (GMP) standard; the pharmaceutical composition was made according to good manufacturing practices (GMP); the pharmaceutical composition has a pathogen level below a predetermined reference value, e.g., is substantially free of pathogens; the pharmaceutical composition has a contaminant level below a predetermined reference value, e.g., is substantially free of contaminants, e.g., as described herein.

In some embodiments, the pharmaceutical composition comprises below a threshold amount of one or more contaminants. Exemplary contaminants that are desirably excluded or minimized in the pharmaceutical composition include, without limitation, host cell nucleic acids (e.g., host cell DNA and/or host cell RNA), host cell protein, animal-derived components (e.g., serum albumin or trypsin), replication-competent viruses, non-infectious particles, free viral capsid protein, adventitious agents, and aggregates. In embodiments, the contaminant is host cell DNA. In embodiments, the composition comprises less than about 10 ng of host cell DNA per dose. In embodiments, the level of host cell DNA in the composition is reduced by filtration and/or enzymatic degradation of host cell DNA. In embodiments, the pharmaceutical composition consists of less than 10% (e.g., less than about 10%, 5%, 4%, 3%, 2%, 1%, 0.5%, or 0.1%) contaminant by weight. The disclosure thus includes methods of making CAVectors disclosed herein that include a step of evaluating a CAVector preparation for one or more (1, 2, 3, 4, 5, 6, 7, or all 8) of the following: adventitious agents, pyrogenic substances, endotoxin, mycoplasma, host cell DNA, host cell protein, non-infectious particles, or empty capsids.

The disclosure includes, in some instances, sterile pharmaceutical compositions that include a CAVector described herein and a pharmaceutical excipient, and wherein the compositions meet the requirements of 21 C.F.R. §§ 610.12 and 610.13. For example, the pharmaceutical compositions may, in some embodiments, have one, two, 3, 4, 5, 6, 7 or all 8 of the following characteristics:
(i) substantially lack adventitious agents,
(ii) substantially lack pyrogenic substances,
(iii) contains equal to or less endotoxin than a control reference or specification, e.g., a U.S. Pharmacopeia (USP) or FDA reference standard for endotoxin contamination,
(iv) contains equal to or less mycoplasma than a control reference or specification, e.g., a U.S. Pharmacopeia (USP) or FDA reference standard for mycoplasma contamination,
(v) contains less host cell DNA than a control reference standard, e.g., less than 10 ng of host cell DNA per dose, less than 5 ng of host cell DNA per dose,
(vi) contains less host cell protein (HCP) than a control reference standard, e.g., less than 100 ng/mL, less than 50 ng/mL, and/or less than 10 ng/dose, less than 5 ng/dose,
(vii) contain less than a threshold amount of non-infectious particles, e.g., meet a predetermined release specification for non-infectious particles relative to infectious particles, e.g., particles to infectious units <2000:1, <1000:1, <500:1, <300:1, <200:1, <100:1, or <50:1, and/or
(viii) contain less than a threshold amount of empty capsids (lacking a genome), e.g., meet a predetermined release specification for empty capsids.

In one aspect, the invention described herein includes a pharmaceutical composition comprising:
a) a CAVector comprising a genetic element comprising (i) a sequence encoding a non-pathogenic exterior protein, (ii) an exterior protein binding sequence that binds the genetic element to the non-pathogenic exterior protein, and (iii) a sequence encoding a regulatory nucleic acid; and a proteinaceous exterior that is associated with, e.g., envelops or encloses, the genetic element; and
b) a pharmaceutical excipient.

### Vesicles

In some embodiments, the composition further comprises a carrier component, e.g., a microparticle, liposome, vesicle, or exosome. In some embodiments, liposomes comprise spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are generally biocompatible, nontoxic, can deliver both hydrophilic and lipophilic drug molecules, protect their cargo from degradation by plasma enzymes, and transport their load across biological membranes (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Vesicles may comprise without limitation DOTMA, DOTAP, DOTIM, DDAB, alone or together with cholesterol to yield DOTMA and cholesterol, DOTAP and cholesterol, DOTIM and cholesterol, and DDAB and cholesterol. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

As described herein, additives may be added to vesicles to modify their structure and/or properties. For example, either cholesterol or sphingomyelin may be added to the mixture to help stabilize the structure and to prevent the leakage of the inner cargo. Further, vesicles can be prepared from hydrogenated egg phosphatidylcholine or egg phosphatidylcholine, cholesterol, and dicetyl phosphate. (see, e.g., Spuch and Navarro, Journal of Drug Delivery, vol. 2011, Article ID 469679, 12 pages, 2011. doi:10.1155/2011/469679 for review). Also, vesicles may be surface modified during or after synthesis to include reactive groups complementary to the reactive groups on the recipient cells. Such reactive groups include without limitation maleimide groups. As an example, vesicles may be synthesized to include maleimide conjugated phospholipids such as without limitation DSPE-MaL-PEG2000.

A vesicle formulation may be mainly comprised of natural phospholipids and lipids such as 1,2-distearoryl-sn-glycero-3-phosphatidyl choline (DSPC), sphingomyelin, egg phosphatidylcholines and monosialoganglioside. Formulations made up of phospholipids only are less stable in plasma. However, manipulation of the lipid membrane with cholesterol reduces rapid release of the encapsulated cargo or 1,2-dioleoyl-sn-glycero-3-phosphoethanolamine (DOPE) increases stability (see, e.g., Spuch and Navarro, Journal of Drug Delivery, vol. 2011, Article ID 469679, 12 pages, 2011. doi:10.1155/2011/469679 for review).

In embodiments, lipids may be used to form lipid microparticles. Lipids include, but are not limited to, DLin-KC2-DMA4, C12-200 and colipids disteroylphosphatidyl choline, cholesterol, and PEG-DMG may be formulated (see, e.g., Novobrantseva, Molecular Therapy-Nucleic Acids (2012) 1, e4; doi:10.1038/mtna.2011.3) using a spontaneous vesicle formation procedure. The component molar ratio may be about 50/10/38.5/1.5 (DLin-KC2-DMA or C12-200/disteroylphosphatidyl choline/cholesterol/PEG-DMG). Tekmira has a portfolio of approximately 95 patent families, in the U.S. and abroad, that are directed to various aspects of lipid microparticles and lipid microparticles formulations (see, e.g., U.S. Pat. Nos. 7,982,027; 7,799,565; 8,058,069; 8,283,333; 7,901,708; 7,745,651; 7,803,397; 8,101,741; 8,188,263; 7,915,399; 8,236,943 and 7,838,658 and European Pat. Nos. 1766035; 1519714; 1781593 and 1664316), all of which may be used and/or adapted to the present invention.

In some embodiments, microparticles comprise one or more solidified polymer(s) that is arranged in a random manner. The microparticles may be biodegradable. Biodegradable microparticles may be synthesized, e.g., using methods known in the art including without limitation solvent evaporation, hot melt microencapsulation, solvent removal, and spray drying. Exemplary methods for synthesizing microparticles are described by Bershteyn et al., Soft Matter 4:1787-1787, 2008 and in US 2008/0014144 A1, the specific teachings of which relating to microparticle synthesis are incorporated herein by reference.

Exemplary synthetic polymers which can be used to form biodegradable microparticles include without limitation aliphatic polyesters, poly (lactic acid) (PLA), poly (glycolic acid) (PGA), co-polymers of lactic acid and glycolic acid (PLGA), polycarprolactone (PCL), polyanhydrides, poly(ortho)esters, polyurethanes, poly(butyric acid), poly(valeric acid), and poly(lactide-co-caprolactone), and natural polymers such as albumin, alginate and other polysaccharides including dextran and cellulose, collagen, chemical derivatives thereof, including substitutions, additions of chemical groups such as for example alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water, by surface or bulk erosion.

The microparticles' diameter ranges from 0.1-1000 micrometers (μm). In some embodiments, their diameter ranges in size from 1-750 μm, or from 50-500 μm, or from 100-250 μm. In some embodiments, their diameter ranges in size from 50-1000 μm, from 50-750 μm, from 50-500 μm, or from 50-250 μm. In some embodiments, their diameter ranges in size from 0.05-1000 μm, from 10-1000 μm, from 100-1000 μm, or from 500-1000 μm. In some embodiments, their diameter is about 0.5 μm, about 10 μm, about 50 μm, about 100 μm, about 200 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, or about 1000 μm. As used in the context of microparticle diameters, the term “about” means+/−5% of the absolute value stated.

In some embodiments, a ligand is conjugated to the surface of the microparticle via a functional chemical group (carboxylic acids, aldehydes, amines, sulfhydryls and hydroxyls) present on the surface of the particle and present on the ligand to be attached. Functionality may be introduced into the microparticles by, for example, during the emulsion preparation of microparticles, incorporation of stabilizers with functional chemical groups.

Another example of introducing functional groups to the microparticle is during post-particle preparation, by direct crosslinking particles and ligands with homo- or heterobifunctional crosslinkers. This procedure may use a suitable chemistry and a class of crosslinkers (CDI, EDAC, glutaraldehydes, etc. as discussed in more detail below) or any other crosslinker that couples ligands to the particle surface via chemical modification of the particle surface after preparation. This also includes a process whereby amphiphilic molecules such as fatty acids, lipids or functional stabilizers may be passively adsorbed and adhered to the particle surface, thereby introducing functional end groups for tethering to ligands.

In some embodiments, the microparticles may be synthesized to comprise one or more targeting groups on their exterior surface to target a specific cell or tissue type (e.g., cardiomyocytes). These targeting groups include without limitation receptors, ligands, antibodies, and the like. These targeting groups bind their partner on the cells' surface. In some embodiments, the microparticles will integrate into a lipid bilayer that comprises the cell surface and the mitochondria are delivered to the cell.

The microparticles may also comprise a lipid bilayer on their outermost surface. This bilayer may be comprised of one or more lipids of the same or different type. Examples include without limitation phospholipids such as phosphocholines and phosphoinositols. Specific examples include without limitation DMPC, DOPC, DSPC, and various other lipids such as those described herein for liposomes.

In some embodiments, the carrier comprises nanoparticles, e.g., as described herein.

In some embodiments, the vesicles or microparticles described herein are functionalized with a diagnostic agent. Examples of diagnostic agents include, but are not limited to, commercially available imaging agents used in positron emissions tomography (PET), computer assisted tomography (CAT), single photon emission computerized tomography, x-ray, fluoroscopy, and magnetic resonance imaging (MRI); and contrast agents. Examples of suitable materials for use as contrast agents in MRI include gadolinium chelates, as well as iron, magnesium, manganese, copper, and chromium.

### Carriers

A composition (e.g., pharmaceutical composition) described herein may comprise, be formulated with, and/or be delivered in, a carrier. In one aspect, the invention includes a composition, e.g., a pharmaceutical composition, comprising a carrier (e.g., a vesicle, a liposome, a lipid nanoparticle, an exosome, a red blood cell, an exosome (e.g., a mammalian or plant exosome), a fusosome) comprising (e.g., encapsulating) a composition described herein (e.g., a CAVector, a CAV, or genetic element described herein).

In some embodiments, the compositions and systems described herein can be formulated in liposomes or other similar vesicles. Generally, liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes generally have one or more (e.g., all) of the following characteristics: biocompatibility, nontoxicity, can deliver both hydrophilic and lipophilic drug molecules, can protect their cargo from degradation by plasma enzymes, and can transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679; and Zylberberg & Matosevic. 2016. Drug Delivery, 23:9, 3319-3329, doi: 10.1080/10717544.2016.1177136).

Vesicles can be made from several different types of lipids; however, phospholipids are most commonly used to generate liposomes as drug carriers. Methods for preparation of multilamellar vesicle lipids are known (see, for example, U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by, e.g., extruding through filters of decreasing size, as described in Templeton et al., Nature Biotech, 15:647-652, 1997.

Lipid nanoparticles (LNPs) are another example of a carrier that provides a biocompatible and biodegradable delivery system for the pharmaceutical compositions described herein. See, e.g., Gordillo-Galeano et al. European Journal of Pharmaceutics and Biopharmaceutics. Volume 133, December 2018, Pages 285-308. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi:10.3390/nano7060122.

Exosomes can also be used as drug delivery vehicles for the compositions and systems described herein. For a review, see Ha et al. July 2016. Acta Pharmaceutica Sinica B. Volume 6, Issue 4, Pages 287-296; doi.org/10.1016/j.apsb.2016.02.001.

Ex vivo differentiated red blood cells can also be used as a carrier for a composition described herein. See, e.g., WO2015073587; WO2017123646; WO2017123644; WO2018102740; WO2016183482; WO2015153102; WO2018151829; WO2018009838; Shi et al. 2014. Proc Natl Acad Sci USA. 111(28): 10131-10136; U.S. Pat. No. 9,644,180; Huang et al. 2017. Nature Communications 8: 423; Shi et al. 2014. Proc Natl Acad Sci USA. 111(28): 10131-10136.

Fusosome compositions, e.g., as described in WO2018208728, can also be used as carriers to deliver a composition described herein.

### Combinations

In one aspect, the CAVector or composition comprising a CAVector described herein may also include one or more heterologous moiety. In one aspect, the CAVector or composition comprising a CAVector described herein may also include one or more heterologous moiety in a fusion. In some embodiments, a heterologous moiety may be linked with the genetic element. In some embodiments, a heterologous moiety may be enclosed in the proteinaceous exterior as part of the CAVector. In some embodiments, a heterologous moiety may be administered with the CAVector.

In one aspect, the invention includes a cell or tissue comprising any one of the CAVectors and heterologous moieties described herein.

In another aspect, the invention includes a pharmaceutical composition comprising a CAVector and the heterologous moiety described herein.

In some embodiments, the heterologous moiety may be a virus (e.g., an effector (e.g., a drug, small molecule), a targeting agent (e.g., a DNA targeting agent, antibody, receptor ligand), a tag (e.g., fluorophore, light sensitive agent such as KillerRed), or an editing or targeting moiety described herein. In some embodiments, a membrane translocating polypeptide described herein is linked to one or more heterologous moieties. In one embodiment, the heterologous moiety is a small molecule (e.g., a peptidomimetic or a small organic molecule with a molecular weight of less than 2000 daltons), a peptide or polypeptide (e.g., an antibody or antigen-binding fragment thereof), a nanoparticle, an aptamer, or pharmacoagent.

Targeting Moiety

In some embodiments, the composition or CAVector described herein may further comprise a targeting moiety, e.g., a targeting moiety that specifically binds to a molecule of interest present on a target cell. The targeting moiety may modulate a specific function of the molecule of interest or cell, modulate a specific molecule (e.g., enzyme, protein or nucleic acid), e.g., a specific molecule downstream of the molecule of interest in a pathway, or specifically bind to a target to localize the CAVector or genetic element. For example, a targeting moiety may include a therapeutic that interacts with a specific molecule of interest to increase, decrease or otherwise modulate its function.

Tagging or Monitoring Moiety

In some embodiments, the composition or CAVector described herein may further comprise a tag to label or monitor the CAVector or genetic element described herein. The tagging or monitoring moiety may be removable by chemical agents or enzymatic cleavage, such as proteolysis or intein splicing. An affinity tag may be useful to purify the tagged polypeptide using an affinity technique. Some examples include, chitin binding protein (CBP), maltose binding protein (MBP), glutathione-S-transferase (GST), and poly(His) tag. A solubilization tag may be useful to aid recombinant proteins expressed in chaperone-deficient species such as E. coli to assist in the proper folding in proteins and keep them from precipitating. Some examples include thioredoxin (TRX) and poly(NANP). The tagging or monitoring moiety may include a light sensitive tag, e.g., fluorescence. Fluorescent tags are useful for visualization. GFP and its variants are some examples commonly used as fluorescent tags. Protein tags may allow specific enzymatic modifications (such as biotinylation by biotin ligase) or chemical modifications (such as reaction with FlAsH-EDT2 for fluorescence imaging) to occur. Often tagging or monitoring moiety are combined, in order to connect proteins to multiple other components. The tagging or monitoring moiety may also be removed by specific proteolysis or enzymatic cleavage (e.g. by TEV protease, Thrombin, Factor Xa or Enteropeptidase).

Nanoparticles

In some embodiments, the composition or CAVector described herein may further comprise a nanoparticle. Nanoparticles include inorganic materials with a size between about 1 and about 1000 nanometers, between about 1 and about 500 nanometers in size, between about 1 and about 100 nm, between about 50 nm and about 300 nm, between about 75 nm and about 200 nm, between about 100 nm and about 200 nm, and any range therebetween. Nanoparticles generally have a composite structure of nanoscale dimensions. In some embodiments, nanoparticles are typically spherical although different morphologies are possible depending on the nanoparticle composition. The portion of the nanoparticle contacting an environment external to the nanoparticle is generally identified as the surface of the nanoparticle. In nanoparticles described herein, the size limitation can be restricted to two dimensions and so that nanoparticles include composite structure having a diameter from about 1 to about 1000 nm, where the specific diameter depends on the nanoparticle composition and on the intended use of the nanoparticle according to the experimental design. For example, nanoparticles used in therapeutic applications typically have a size of about 200 nm or below.

Additional desirable properties of the nanoparticle, such as surface charges and steric stabilization, can also vary in view of the specific application of interest. Exemplary properties that can be desirable in clinical applications such as cancer treatment are described in Davis et al, Nature 2008 vol. 7, pages 771-782; Duncan, Nature 2006 vol. 6, pages 688-701; and Allen, Nature 2002 vol. 2 pages 750-763, each incorporated herein by reference in its entirety. Additional properties are identifiable by a skilled person upon reading of the present disclosure. Nanoparticle dimensions and properties can be detected by techniques known in the art. Exemplary techniques to detect particles dimensions include but are not limited to dynamic light scattering (DLS) and a variety of microscopies such at transmission electron microscopy (TEM) and atomic force microscopy (AFM). Exemplary techniques to detect particle morphology include but are not limited to TEM and AFM. Exemplary techniques to detect surface charges of the nanoparticle include but are not limited to zeta potential method. Additional techniques suitable to detect other chemical properties comprise by ¹H, ¹¹B, and ¹³C and ¹⁹F NMR, UV/Vis and infrared/Raman spectroscopies and fluorescence spectroscopy (when nanoparticle is used in combination with fluorescent labels) and additional techniques identifiable by a skilled person.

### V. Host Cells

The invention is further directed to a host or host cell comprising a CAVector, CAV, genetic element, or genetic element construct, e.g., as described herein. In some embodiments, the host or host cell is a plant, insect, bacteria, fungus, vertebrate, avian (e.g., chicken), mammal (e.g., human), or other organism or cell. In certain embodiments, as confirmed herein, provided CAVectors infect a range of different host cells. Target host cells include cells of mesodermal, endodermal, or ectodermal origin. Target host cells include, e.g., epithelial cells, muscle cells, white blood cells (e.g., lymphocytes), kidney tissue cells, lung tissue cells.

In some embodiments, a host or a host cell is contacted with (e.g., infected with) an CAVector. In some embodiments, the host is a mammal, such as a human. The amount of the CAVector in the host can be measured at any time after administration. In certain embodiments, a time course of CAVector growth in a culture is determined.

In some embodiments, the CAVector, e.g., an CAVector as described herein, is heritable. In some embodiments, the CAVector is transmitted linearly in fluids and/or cells from mother to child. In some embodiments, daughter cells from an original host cell comprise the CAVector. In some embodiments, a mother transmits the CAVector to child with an efficiency of at least 25%, 50%, 60%, 70%, 80%, 85%, 90%, 95%, or 99%, or a transmission efficiency from host cell to daughter cell at least 25%, 50%, 60%, 70%, 80%, 85%, 90%, 95%, or 99%. In some embodiments, the CAVector in a host cell has a transmission efficiency during meiosis of at 25%, 50%, 60%, 70%, 80%, 85%, 90%, 95%, or 99%. In some embodiments, the CAVector in a host cell has a transmission efficiency during mitosis of at least 25%, 50%, 60%, 70%, 80%, 85%, 90%, 95%, or 99%. In some embodiments, the CAVector in a cell has a transmission efficiency between about 10%-20%, 20%-30%, 30%-40%, 40%-50%, 50%-60%, 60%-70%, 70%-75%, 75%-80%, 80%-85%, 85%-90%, 90%-95%, 95%-99%, or any percentage therebetween.

In some embodiments, the CAVector replicates within the host cell. In one embodiment, the CAVector is capable of replicating in a mammalian cell, e.g., human cell. In other embodiments, the CAVector is replication deficient or replication incompetent.

While in some embodiments the CAVector replicates in the host cell, the CAVector does not integrate into the genome of the host, e.g., with the host's chromosomes. In some embodiments, the CAVector has a negligible recombination frequency, e.g., with the host's chromosomes. In some embodiments, the CAVector has a recombination frequency, e.g., less than about 1.0 cM/Mb, 0.9 cM/Mb, 0.8 cM/Mb, 0.7 cM/Mb, 0.6 cM/Mb, 0.5 cM/Mb, 0.4 cM/Mb, 0.3 cM/Mb, 0.2 cM/Mb, 0.1 cM/Mb, or less, e.g., with the host's chromosomes.

### VI. Methods of Use

The CAVectors and compositions comprising CAVectors described herein may be used in methods of treating a disease, disorder, or condition, e.g., in a subject (e.g., a mammalian subject, e.g., a human subject) in need thereof. Administration of a pharmaceutical composition described herein may be, for example, by way of parenteral (including intravenous, intratumoral, intraperitoneal, intramuscular, intracavity, and subcutaneous) administration. The CAVectors may be administered alone or formulated as a pharmaceutical composition.

The CAVectors may be administered in the form of a unit-dose composition, such as a unit dose parenteral composition. Such compositions are generally prepared by admixture and can be suitably adapted for parenteral administration. Such compositions may be, for example, in the form of injectable and infusable solutions or suspensions or suppositories or aerosols.

In some embodiments, administration of a CAVector or composition comprising same, e.g., as described herein, may result in delivery of a genetic element comprised by the CAVector to a target cell, e.g., in a subject.

A CAVector or composition thereof described herein, e.g., comprising an effector (e.g., an endogenous or exogenous effector), may be used to deliver the effector to a cell, tissue, or subject. In some embodiments, the CAVector or composition thereof is used to deliver the effector to bone marrow, blood, heart, GI or skin. Delivery of an effector by administration of a CAVector composition described herein may modulate (e.g., increase or decrease) expression levels of a noncoding RNA or polypeptide in the cell, tissue, or subject. Modulation of expression level in this fashion may result in alteration of a functional activity in the cell to which the effector is delivered. In some embodiments, the modulated functional activity may be enzymatic, structural, or regulatory in nature.

In some embodiments, the CAVector, or copies thereof, are detectable in a cell 24 hours (e.g., 1 day, 2 days, 3 days, 4 days, 5 days, 6 days, 1 week, 2 weeks, 3 weeks, 4 weeks, 30 days, or 1 month) after delivery into a cell. In embodiments, a CAVector or composition thereof mediates an effect on a target cell, and the effect lasts for at least 1, 2, 3, 4, 5, 6, or 7 days, 2, 3, or 4 weeks, or 1, 2, 3, 6, or 12 months. In some embodiments (e.g., wherein the CAVector or composition thereof comprises a genetic element encoding an exogenous protein), the effect lasts for less than 1, 2, 3, 4, 5, 6, or 7 days, 2, 3, or 4 weeks, or 1, 2, 3, 6, or 12 months.

Examples of diseases, disorders, and conditions that can be treated with the CAVector described herein, or a composition comprising the CAVector, include, without limitation: immune disorders, interferonopathies (e.g., Type I interferonopathies), infectious diseases, inflammatory disorders, autoimmune conditions, cancer (e.g., a solid tumor, e.g., lung cancer, non-small cell lung cancer, e.g., a tumor that expresses a gene responsive to mIR-625, e.g., caspase-3), and gastrointestinal disorders. In some embodiments, the CAVector modulates (e.g., increases or decreases) an activity or function in a cell with which the CAVector is contacted. In some embodiments, the CAVector modulates (e.g., increases or decreases) the level or activity of a molecule (e.g., a nucleic acid or a protein) in a cell with which the CAVector is contacted. In some embodiments, the CAVector decreases viability of a cell, e.g., a cancer cell, with which the CAVector is contacted, e.g., by at least about 10%, 20%, 30%, 40%, 50%, 60%, 70%, 80%, 90%, 95%, 99%, or more. In some embodiments, the CAVector comprises an effector, e.g., an miRNA, e.g., miR-625, that decreases viability of a cell, e.g., a cancer cell, with which the CAVector is contacted, e.g., by at least about 10%, 20%, 30%, 40%, 50%, 60%, 70%, 80%, 90%, 95%, 99%, or more. In some embodiments, the CAVector increases apoptosis of a cell, e.g., a cancer cell, e.g., by increasing caspase-3 activity, with which the CAVector is contacted, e.g., by at least about 10%, 20%, 30%, 40%, 50%, 60%, 70%, 80%, 90%, 95%, 99%, or more. In some embodiments, the CAVector comprises an effector, e.g., an miRNA, e.g., miR-625, that increases apoptosis of a cell, e.g., a cancer cell, e.g., by increasing caspase-3 activity, with which the CAVector is contacted, e.g., by at least about 10%, 20%, 30%, 40%, 50%, 60%, 70%, 80%, 90%, 95%, 99%, or more.

### VII. Administration/Delivery

The composition (e.g., a pharmaceutical composition comprising a CAVector as described herein) may be formulated to include a pharmaceutically acceptable excipient, e.g., as described herein. Pharmaceutical compositions may optionally comprise one or more additional active substances, e.g. therapeutically and/or prophylactically active substances. Pharmaceutical compositions of the present invention may be sterile and/or pyrogen-free. General considerations in the formulation and/or manufacture of pharmaceutical agents may be found, for example, in Remington: The Science and Practice of Pharmacy 21st ed., Lippincott Williams & Wilkins, 2005 (incorporated herein by reference).

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product.

In one aspect, the invention features a method of delivering a CAVector to a subject. The method includes administering a pharmaceutical composition comprising a CAVector as described herein to the subject. In some embodiments, the administered CAVector replicates in the subject (e.g., becomes a part of the virome of the subject).

The pharmaceutical composition may include wild-type or native viral elements and/or modified viral elements. The CAVector may include one or more CAV sequences (e.g., nucleic acid sequences or nucleic acid sequences encoding amino acid sequences thereof) or a sequence with at least about 60%, 65%, 70%, 75%, 80%, 85%, 90% 95%, 96%, 97%, 98% and 99% nucleotide sequence identity thereto. The CAVector may comprise a nucleic acid molecule comprising a nucleic acid sequence with at least about 60%, 65%, 70%, 75%, 80%, 85%, 90% 95%, 96%, 97%, 98% and 99% sequence identity to one or more CAV sequences (e.g., a CAV VP1 nucleic acid sequence), e.g., as described herein. The CAVector may comprise a nucleic acid molecule encoding an amino acid sequence with at least about 60%, 65%, 70%, 75%, 80%, 85%, 90% 95%, 96%, 97%, 98% and 99% sequence identity to an CAV amino acid sequence (e.g., the amino acid sequence of an CAV VP1 molecule), e.g., as described herein. The CAVector may comprise a polypeptide comprising an amino acid sequence with at least about 60%, 65%, 70%, 75%, 80%, 85%, 90% 95%, 96%, 97%, 98% and 99% sequence identity to an CAV amino acid sequence (e.g., the amino acid sequence of an CAV VP1 molecule), e.g., as described herein.

In some embodiments, the CAVector is sufficient to increase (stimulate) endogenous gene and protein expression, e.g., at least about 5%, 10%, 15%, 20%, 25%, 30%, 35%, 40%, 45%, 50%, or more as compared to a reference, e.g., a healthy control. In certain embodiments, the CAVector is sufficient to decrease (inhibit) endogenous gene and protein expression, e.g., at least about 5%, 10%, 15%, 20%, 25%, 30%, 35%, 40%, 45%, 50%, or more as compared to a reference, e.g., a healthy control.

In some embodiments, the CAVector inhibits/enhances one or more viral properties, e.g., tropism, infectivity, immunosuppression/activation, in a host or host cell, e.g., at least about 5%, 10%, 15%, 20%, 25%, 30%, 35%, 40%, 45%, 50%, or more as compared to a reference, e.g., a healthy control.

In some embodiments, the subject is administered the pharmaceutical composition further comprising one or more viral strains that are not represented in the viral genetic information.

In some embodiments, the pharmaceutical composition comprising a CAVector described herein is administered in a dose and time sufficient to modulate a viral infection. Some non-limiting examples of viral infections include adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, Human enterovirus 70, Human herpesvirus 1, Human herpesvirus 2, Human herpesvirus 6, Human herpesvirus 7, Human herpesvirus 8, Human immunodeficiency virus, Human papillomavirus 1, Human papillomavirus 2, Human papillomavirus 16, Human papillomavirus 18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, and Zika Virus. In certain embodiments, the CAVector is sufficient to outcompete and/or displace a virus already present in the subject, e.g., at least about 5%, 10%, 15%, 20%, 25%, 30%, 35%, 40%, 45%, 50%, or more as compared to a reference. In certain embodiments, the CAVector is sufficient to compete with chronic or acute viral infection. In certain embodiments, the CAVector may be administered prophylactically to protect from viral infections (e.g. a provirotic). In some embodiments, the CAVector is in an amount sufficient to modulate (e.g., phenotype, virus levels, gene expression, compete with other viruses, disease state, etc. at least about 5%, 10%, 15%, 20%, 25%, 30%, 35%, 40%, 45%, 50%, or more). In some embodiments, treatment, treating, and cognates thereof comprise medical management of a subject (e.g., by administering a CAVector, e.g., a CAVector made as described herein), e.g., with the intent to improve, ameliorate, stabilize, prevent or cure a disease, pathological condition, or disorder. In some embodiments, treatment comprises active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to preventing, minimizing or partially or completely inhibiting the development of the associated disease, pathological condition, or disorder), and/or supportive treatment (treatment employed to supplement another therapy).

### Redosing

The CAVectors described herein can, in some instances, be used as a delivery vehicle that can be administered in multiple doses (e.g., doses administered separately). While not wishing to be bound by theory, in some embodiments, a CAVector (e.g., as described herein) induces a relatively low immune response (as measured, for example, as 50% GMT values, e.g., as observed in Example 12), e.g., allowing for repeated dosing of a subject with one or more CAVectors (e.g., multiple doses of the same CAVector or different CAVectors). In an aspect, the invention provides a method of delivering an effector, comprising administering to a subject a first plurality of CAVectors and then a second plurality of CAVectors. In some embodiments, the second plurality of CAVectors comprise the same proteinaceous exterior as the CAVectors of the first plurality. In another aspect, the invention provides a method of selecting a subject (e.g., a human subject) to receive an effector, wherein the subject previously received, or was identified as having received, a first plurality of CAVectors comprising a genetic element encoding an effector, in which the method involves selecting the subject to receive a second plurality of CAVectors comprising a genetic element encoding an effector (e.g., the same effector as that encoded by the genetic element of the first plurality of CAVectors, or a different effector as that encoded by the genetic element of the first plurality of CAVectors). In another aspect, the invention provides a method of identifying a subject (e.g., a human subject) as suitable to receive a second plurality of CAVectors, the method comprising identifying the subject has having previously received a first plurality of CAVectors comprising a genetic element encoding an effector, wherein the subject being identified as having received the first plurality of CAVectors is indicative that the subject is suitable to receive the second plurality of CAVectors.

In some embodiments, the second plurality of CAVectors comprises a proteinaceous exterior with at least one surface epitope in common with the CAVectors of the first plurality of CAVectors. In some embodiments, the CAVectors of the first plurality and the CAVectors of the second plurality carry genetic elements encoding the same effector. In some embodiments, the CAVectors of the first plurality and the CAVectors of the second plurality carry genetic elements encoding different effectors.

In some embodiments, the second plurality comprises about the same quantity and/or concentration of CAVectors as the first plurality (e.g., when normalized to the body mass of the subject at the time of administration), e.g., the second plurality comprises 90-110%, e.g., 95-105% of the number of CAVectors in the first plurality when normalized to body mass of the subject at the time of administration. In some embodiments, wherein the first plurality comprises a greater dosage of CAVectors than the second plurality, e.g., wherein the first plurality comprises a greater quantity and/or concentration of CAVectors relative to the second plurality. In some embodiments, wherein the first plurality comprises a lower dosage of CAVectors than the second plurality, e.g., wherein the first plurality comprises a lower quantity and/or concentration of CAVectors relative to the second plurality.

In some embodiments, the subject is evaluated between the administration of the first and second pluralities of CAVectors, e.g., for the presence (e.g., persistence) of CAVectors from the first plurality, or progeny thereof. In some embodiments, the subject is administered the second plurality of CAVectors if the presence of CAVectors from the first plurality, or the progeny thereof, are not detected.

In some embodiments, the second plurality is administered to the subject at least 1, 2, 3, or 4 weeks, or 1, 2, 3, 4, 5, 6, 7, 8, 9, 10, 11, or 12 months, or 1, 2, 3, 4, 5, 10, or 20 years after the administration of the first plurality to the subject. In some embodiments, the second plurality is administered to the subject between 1-2 weeks, 2-3 weeks, 3-4 weeks, 1-2 months, 3-4 months, 4-5 months, 5-6 months, 6-7 months, 7-8 months, 8-9 months, 9-10 months, 10-11 months, 11-12 months, 1-2 years, 2-3 years, 3-4 years, 4-5 years, 5-10 years, or 10-20 years after the administration of the first plurality to the subject. In some embodiments, the method comprises administering a repeated dose of anellovectors over the course of at least 1, 2, 3, 4, or 5 years.

In some embodiments, the method further comprises assessing, after administration of the first plurality and before administration of the second plurality, one or more of:
a) the level or activity of the effector in the subject (e.g., by detecting a protein effector, e.g., by ELISA; by detecting a nucleic acid effector, e.g., by RT-PCR, or by detecting a downstream effect of the effector, e.g., level of an endogenous gene affected by the effector);
b) the level or activity of the CAVector of the first plurality in the subject (e.g., by detecting the level of the VP1 of the CAVector);
c) the presence, severity, progression, or a sign or symptom of a disease in the subject that the anellovector was administered to treat; and/or
d) the presence or level of an immune response, e.g., neutralizing antibodies, against a CAV or CAVector.

In some embodiments, the method further comprises administering to the subject a third, fourth, fifth, and/or further plurality of CAVectors, e.g., as described herein.

In some embodiments, the first plurality and the second plurality are administered via the same route of administration, e.g., intravenous administration. In some embodiments, the first plurality and the second plurality are administered via different routes of administration. In some embodiments, the first and the second pluralities are administered by the same entity (e.g., the same health care provider). In some embodiments, the first and the second pluralities are administered by different entities (e.g., different health care providers).

All references and publications cited herein are hereby incorporated by reference.

The following examples are provided to further illustrate some embodiments of the present invention, but are not intended to limit the scope of the invention; it will be understood by their exemplary nature that other procedures, methodologies, or techniques known to those skilled in the art may alternatively be used.

### EXAMPLES

### Table of Contents

Example 1: Rescue of Recombinant Synthetic CAV and passage in avian cells
Example 2: Tandem CAV constructs
Example 3: CAV Binding to Avian and Human Cells
Example 4: Design and construction of exemplary CAVectors genetic elements
Example 5: Rescue of CAVectors using wild-type CAV
Example 6: Purification of CAVector from supernatant
Example 7: CAVectors transduce human cells
Example 8: Resistance of CAVectors to neutralizing antibodies
Example 9: Production of CAVectors without wild-type CAV
Example 10: Production of nLuc CAVector for injection into mice
Example 11: Administration of CAVector in vivo to mice, resulting in delivery of payload DNA in multiple organs
Example 12: Immunogenicity in mice receiving CAVectors versus AAV2
Example 13: CAV-like particles (VLPs) assemble in vitro from purified capsid proteins
Example 14: CAVector enter MDCC-MSB1 cells via the late endosome pathway
Example 15: CAVector viability after heat treatment and after storage at 4° C.
Example 16: Recovery of CAVector using tandem plasmid

### Example 1: Rescue of Recombinant Synthetic CAV and Passage in Avian Cells

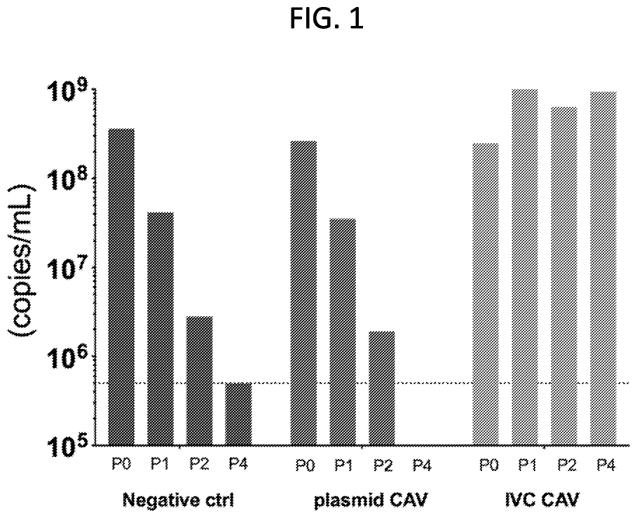

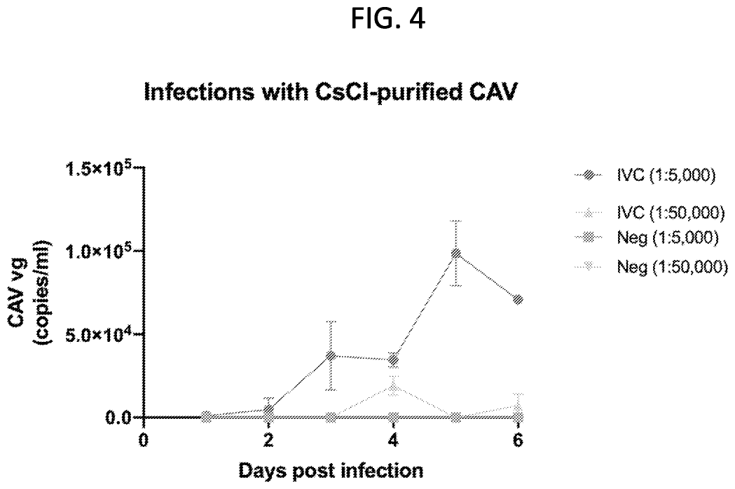

In this example, recombinant synthetic Chicken Anemia Virus (CAV) was produced from a synthetic genome. To rescue viable CAV from a synthetic genome, CAV plasmid DNA was in vitro circularized (IVC) to remove vector backbone, transfected into chicken cells, and the resulting infected cells passaged into fresh cells repeatedly. Briefly, MDCC-MSB1 cells were electroporated with p637(Neg), pCAV, or pCAV-IVC constructs. The cells were split into fresh medium with uninfected MDCC-MSB1 cells, with subsequent splitting of cells every 48 hours. The repeated splitting steps produced Passage 0 (P0), Passage 1 (P1), Passage 2 (P2), and beyond. Control DNA (described below) was similarly processed to show that virus recovery was only detected when circular double-stranded CAV genomic DNA was provided to the cells (FIGS. 1 and 4). Quantitative PCR (qPCR), microscopy of infected cells, electron microscopy of concentrated viral suspensions, and western blotting were used to show that CAV was replicating in the cells.

Prepare the Double-Stranded Genome

The genome of the wild-type CAV strain Cuxhaven 1 was synthesized in vitro as a 2,319 bp EcoRI fragment which was then cloned into the plasmid pUC57. The resulting plasmid (pRTx-708) was propagated in E. coli and purified according to standard methods. The plasmid (75 μg) was digested with EcoRI and PvuI and a 2.3 kb band excised from an agarose gel. A Gel Purification kit (Qiagen) was used to extract the CAV genomic dsDNA and a ligation reaction using T4 DNA ligase (NEB) was carried out in a volume of 5 mL to circularize the CAV double-stranded genome. Following a 24-hour incubation at 16° C., the DNA was ethanol precipitated according to standard methods and resuspended in 100 μL of water. The DNA solution was dialyzed against UltraPure water for 1 hour (Slide-A-Lyzer MINI Dialysis Units 10,000 MWCO, 10-100 μl Cat #69576).

Two negative control DNA samples were also prepared. One negative control was the CAV genome plasmid pRTx-708 which, after purification from E. coli, was not processed to remove the bacterial backbone as described above. Another negative control was plasmid pRTx-637, in which the gene coding for the capsid protein VP1 was deleted.

Transfection of CAV Genome

For each condition, 2.5 ug of DNA was added to 10⁶ live MDCC-MSB1 cells (ATCC), or about 2.5 pg/cell, and delivered by electroporation using a 4D-Nucleofector System (Lonza) with large cuvettes (10⁶ cells/cuvette) and program DS-137, following the manufacturer's instructions. For each condition, transfected cells were recovered as recommended, and transferred to a final culture volume of 5 mL RPMI containing 10% FBS in T25 flasks. Transfected cells were grown in a humidified 40° C./5% CO₂ incubator for 2 to 3 days. The product of this step is referred to herein as passage 0 (P0).

Harvest and Passage of Transfected/Infected Cells

The P0 cell suspension (˜5 mL) was harvested by taking 2 aliquots of the cell suspension for qPCR and western blot analysis, then transferring 1.5 mL of cell suspension into a new flask and bringing up the cell counts to 2×10⁵ cells/mL using fresh cells in a final volume of 7 mL and then incubated at 40° C., 5% CO2. This process was repeated for 4 passages.

Measuring Virus Titer

The cell suspension samples (200 μL) were purified using the PureLink viral DNA/RNA kit (Thermo Fisher) by adding 200 μL of lysis buffer and 25 μL of Proteinase K directly to the sample tube. Copies/ml were evaluated by TaqMan qPCR carried out using Thermo Fisher TaqMan reagents and a QuantStudio real time qPCR instrument according to manufacturer's recommendations.

Primers and probe used:
CAV Fwd:
5′-TTGGAAACCCCTCACTGCAGAG-3′
CAV Rev:
5′-CTGAATTGTCCGCAGTTGCAG-3′
CAV VP1 probe:
5′-6FAM-CTGGAATTACAATCACTCTAT-MGBNFQ-3′

Analysis was carried out for samples from passages 0, 1, 2, and 4 (P1-4; FIG. 1). Only samples from cells originally transfected with IVC CAV DNA showed an increase in CAV copies over passage number. This indicated that replication of CAV DNA and transmission to fresh cells occurred.

Detect Viral Proteins by Western Blot

To analyze CAV protein expression, 30 ul of each sample was reduced and denatured using SDS sample buffer and boiling for 10 minutes. The samples were then loaded on a BOLT 4-12% Bis-Tris gel (Thermo Fisher) and electrophoresed for 40 minutes as recommended by the supplier. The gel was washed in water for 5 minutes and transferred to a nitrocellulose membrane using the iBlot2 system. The membrane was washed with TBS-T for 5 minutes and blocked with Li-Cor TBS blocking buffer for 1 hour. Detection was carried out by incubating the blocked membrane with a mixture of rabbit anti-Apoptin (VP3; Abcam, Ab193612) and anti-VP2 sera (Cusabio Technology, CSB-PA302471LA01CID) diluted 1:100 in TBS blocking buffer 16 hours at 4° C. After washing, a goat anti-rabbit IRDye 680 secondary antibody (LI-COR Biotechnology) was applied to the membrane, washed, and then imaged using a Li-Cor Odyssey instrument. FIG. 1 shows that P1 and P2 cells derived from IVC CAV-transfected cells (P0) were positive for both CAV proteins, but not samples derived from other transfections.

Characterization of Infected Cells

Cell samples from P4 were observed by phase-contrast microscopy to examine cellular morphology. Cells in the IVC CAV sample were frequently found to be enlarged and to have multiple cytoplasmic inclusions, in contrast with the cells from the control samples which had a normal appearance.

Isolation of Rescued CAV

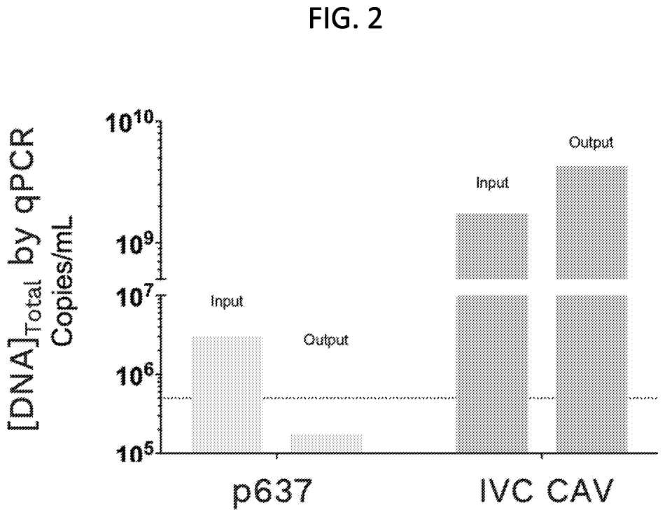

In a separate experiment, MDCC-MSB1 cells were transfected and passaged twice to P2 as described above. Following transfection of CAV IVC or p637 into MDCC-MSB1 cells by electroporation, transfected cells were passaged twice (P0 to P2; splitting into fresh medium with uninfected MDCC-MSB1 cells performed every 48 hours) and the resulting passaged cells at P2 were lysed by freeze-thaw to generate a lysate which was used to infect fresh MDCC-MSB1 cells. The virus harvest from these cells corresponded to P3. Cell suspension from CAV IVC or negative control cells was lysed by subjecting it to freeze-thaw and applied to fresh cells which were then incubated for 3 days at 40° C. and 5% CO₂. After incubation, the cells were inspected by phase contrast microscopy and showed clear cytopathic effects only in those which were infected with CAV IVC-derived lysate. In particular, cells exposed to P2 derived from p637 showed normal morphology while cells exposed to P2 derived from IVC CAV DNA showed a morphology typical of infected cells. Based on qPCR analysis of P2 and P3 aliquots, the number of CAV genome copies increased from P2 (labeled as “input” in FIG. 2) to P3 (“output”) only in the cells which received CAV IVC-derived lysate. These observations were consistent with CAV IVC DNA giving rise to infectious CAV.

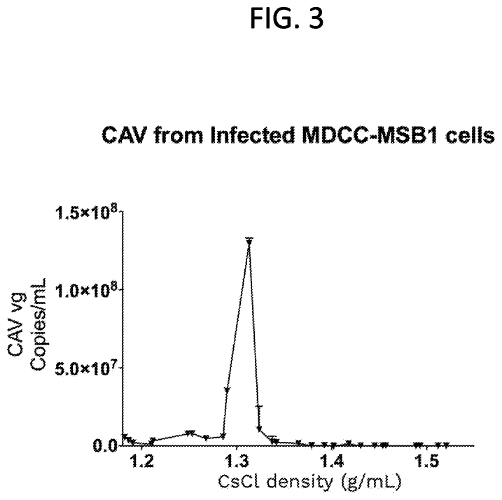

The CAV-infected P3 cell suspension was lysed with 0.5% Triton X-100, treated with Benzonase, and pelleted by sedimentation ultracentrifugation through a 20% sucrose cushion. The resuspended pellet was then applied to a linear CsCl gradient for isopycnic ultracentrifugation and fractions from the gradient harvested for characterization by qPCR (FIG. 3) and infectivity (FIG. 4). CAV genome copies were detected to peak in the density gradient at ˜1.32 g/mL (FIG. 3).

The peak fraction was pooled with the neighboring two fractions, dialyzed to remove CsCl, and the resulting purified viral suspension was added to fresh cells after either 5000-fold or 50,000-fold dilution. A mock purification and infection were carried out in parallel using negative control lysate diluted in the same way. Neither dilution of the negative control showed signs of infection, as measured by qPCR of cell culture samples taken daily over 6 days (FIG. 4). However, culture samples infected with the 5000-fold CAV dilution were clearly positive beginning after 2 days. This confirmed that infectious CAV was derived from synthetic IVC DNA-transfected MDCC-MSB1 cells.

Electron Microscopy

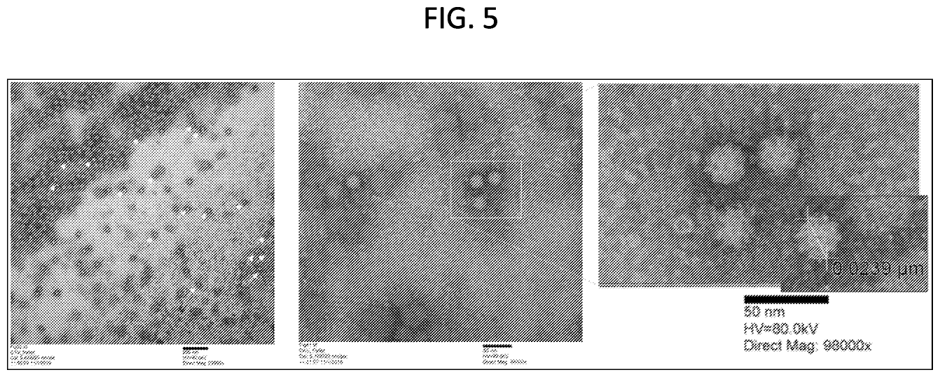

MDCC-MSB1 cells were infected with CAV, allowed to incubate and processed essentially as described by Todd et al. 1990. The viral suspension was imaged by negative stain electron microscopy, revealing a large number of viral particles (FIG. 5).

### Example 2: Tandem CAV Constructs

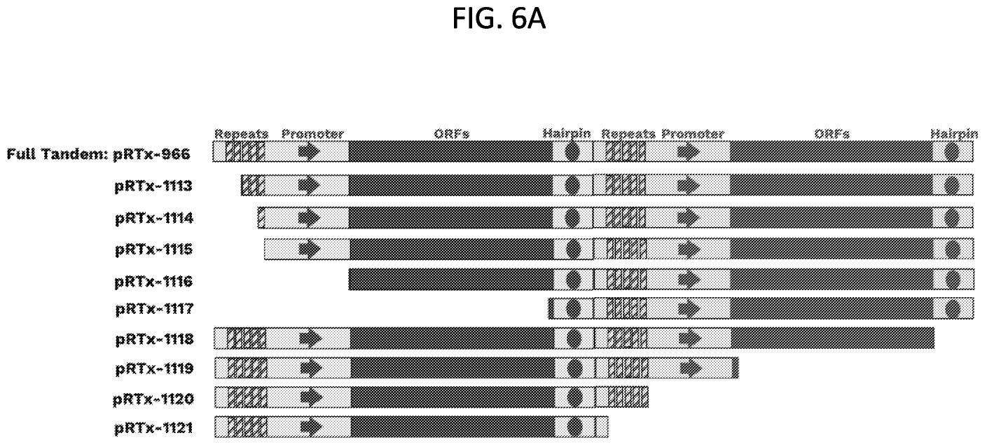

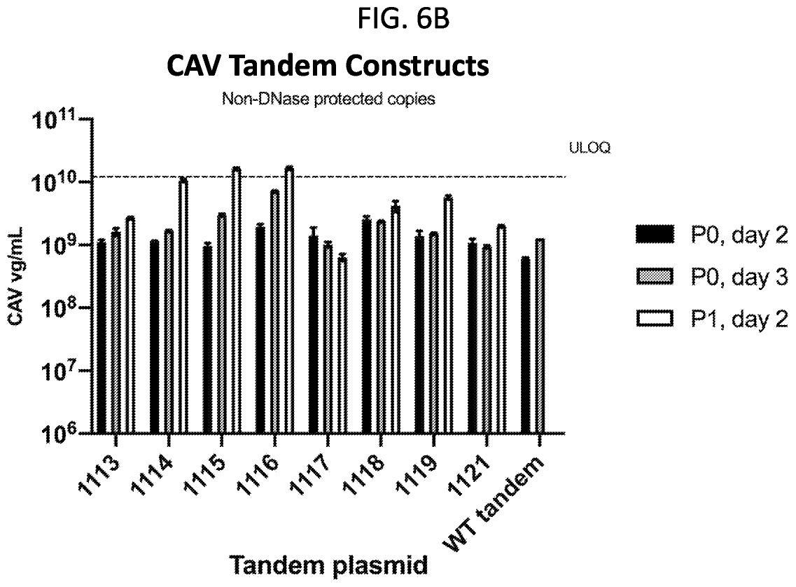

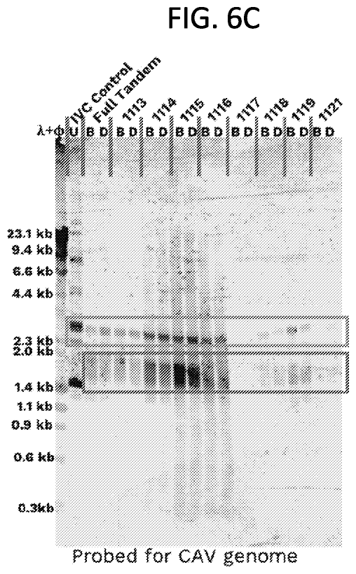

A series of exemplary CAV tandem constructs was generated (FIG. 6A). Briefly, the tandem constructs comprised a plasmid backbone, and either two full copies of the CAV genome, or one full copy and one partial copy. Each of the CAV tandem constructs was introduced into MDCC-MSB1 cells by nucleofection. This was the P0 culture. 200 μl of cell suspension were collected on days 2 and 3 post-nucleofection for downstream qPCR. On day 3, cell suspension was passaged in a ratio of 1:10 into fresh MDCC-MSB1 cells to start the P1 culture. On day 2 of the P1 culture, 200 μl of suspension were collected for qPCR, and the remaining cells were pelleted for DNA extraction and Southern blotting. qPCR was performed for the P0 and P1 culture samples using CAV probes. An increase in replication was observed upon passaging for pRTX-1114-1116. A smaller increase was observed for pRTX1113, 1118, and 1119 (FIG. 6B). Cell pellets from the P1 samples were analyzed by Southern blotting. Backbone cut or DpnI digested DNA was run on a gel, transferred to a membrane, and probed with biotin-labeled probes made by random hexamers against CAV and ladders. Streptavidin-IRDye-800 was added, and results were imaged on a LiCor Odyssey. The results of this experiment were consistent with the qPCR data. dsDNA circles were observed in all lanes except those that contain pRTX-1117 and pRTX-1121 (FIG. 6C). Bands were DpnI resistant, which indicated replication of the genome. Wild-type CAV virus was also recovered.

These results indicate that adding a full or partial copy of a CAV genome to a plasmid comprising backbone and a full CAV genome increases viral titer.

### Example 3: CAV Binding to Avian and Human Cells

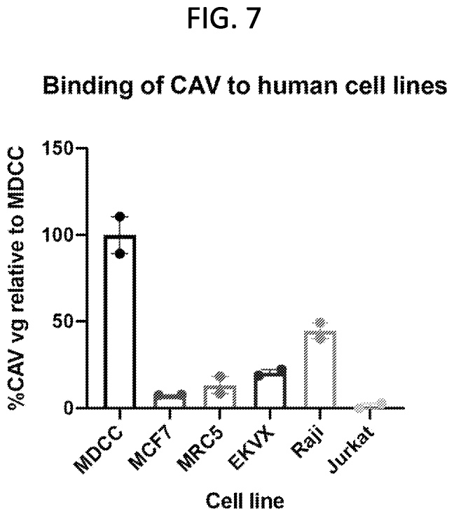

The ability of CAV to bind a panel of human cell lines was screened. MCF-7 (human breast cancer), MRC5 (lung fibroblast), EKVX (lung adenocarcinoma), Raji (B lymphoblast), Jurkat (T lymphoblast), and MDCC (chicken lymphoblast, positive control) cells were tested in duplicate. For each cell type, 2×10⁵ cells were incubated with CAV (MOI=1.5 CAV particles per cell) for one hour at 4° C. Cells were washed twice, and a trypsin control was performed where each condition was treated with trypsin to remove bound virions and establish a background. DNA was extracted, and qPCR was performed to determine the number of viral genomes bound to each cell type. The trypsin background was subtracted, and the values were plotted as percentage binding normalized to the MDCC control (FIG. 7). The results indicated that CAV bound to Raji cells and to EKVX cells. These data show that CAV can bind specifically to human cells, and are consistent with CAV being able to enter human cells.

### Example 4: Design and Construction of Exemplary CAVectors Genetic Elements

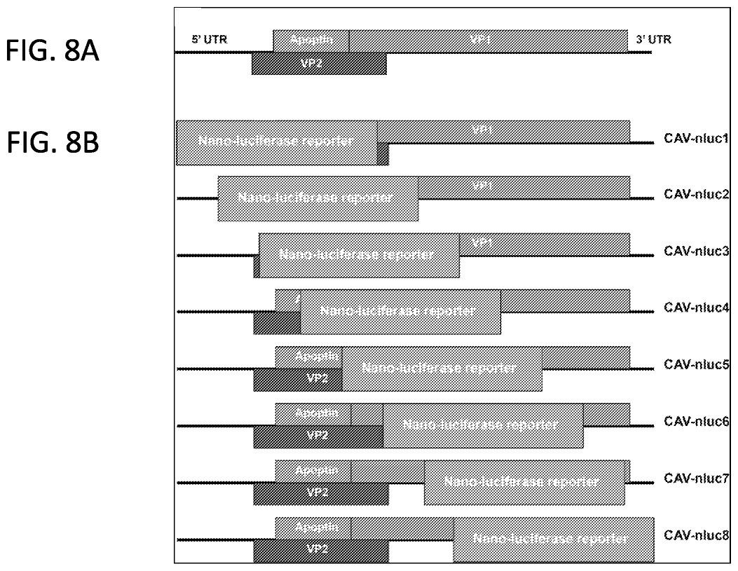

To generate putative vectors, a scanning approach was used to analyze the CAV genome to discover regions necessary for viral replication and packaging. Briefly, a 988 bp reporter cassette was inserted into the CAV genome at staggered 200 bp spacings (FIGS. 8A-8B). The reporter, which contained the SV40 promoter, the nano-luciferase (nLuc) ORF, and the SV40 terminator, replaced the corresponding region of the CAV genome, thus maintaining the wild-type CAV genome length. A total of eight nLuc insertion constructs were designed, as shown in FIG. 8A. The sequences of each nLuc construct are shown in Tables 2-9 above. The constructs were chemically synthesized, cloned into the pUC57-mini plasmid, restriction digested from the plasmid backbone, and in-vitro circularized (IVC). These plasmids could then be used to produce engineered CAV-based genetic elements to be tested for their ability to be packaged into a proteinaceous exterior, thereby forming an engineered CAV vector (referred to herein as a CAVector).

### Example 5: Rescue of CAVectors Using Wild-Type CAV

This example demonstrates the provision in trans of CAV proteins to a CAVector comprising an exogenous gene. The resulting CAVector was able to deliver the exogenous gene to a target cell.

CAVector genomes designed as described in Example 4 were produced by in vitro circularization (IVC) and then co-transfected into MDCC-MSB1 cells at a 1:1 ratio with plasmid encoding a tandem CAV genome (pRTX-966) or plasmid encoding a partial CAV genome in which the VP1 coding region is deleted but VP2 and VP3 are retained (pRTX-637). As a control, WT CAV IVC was also co-transfected with pRTX-966 or pRTX-637. 5×10⁶ total cells were transfected via nucleofection per condition and incubated in 25 mL RPMI with 10% FBS. After 3 days, the production of nLuc was confirmed, cell suspension was harvested, and supernatants were clarified by centrifugation. Clarified supernatants were then filtered through a 0.2 μm filter.

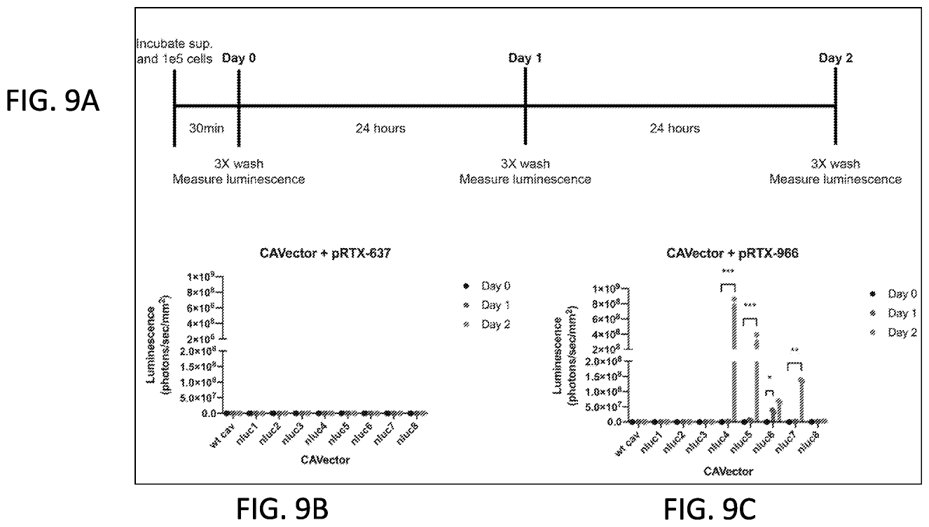

To test if vector rescue had occurred, filtered supernatants were incubated with 1×10⁵ MDCC-MSB1 cells for 30 minutes in a 48-well plate, followed by three washes. Luminescence was then measured to determine if transduction occurred (day 0), as well as at 24 hour increments thereafter (day 1 and day 2) (FIG. 9A). The day 0 reading provided a measurement of the nLuc background while day 1 and 2 measures the nLuc produced from transduction. Before measuring luminescence, the transduced cells were washed three times with PBS to reduce background nLuc in the samples. The luminescence measurements were performed on lysed cells to enrich for nLuc signal. In the control samples (pRTX-637), there was no increase in luminescence from day 0 to day 2, indicating that no vectors were formed in the absence of VP1 (FIG. 9B). In the samples that were co-transfected with tandem CAV, an increase in luminescence was observed from day 0 to 2 for CAV-nLuc4, 5, and 7, and an increase from day 0 to 1 was observed for CAV-nLuc6 (FIG. 9C). This indicated that these IVC CAVector genomes are capable of undergoing replication and packaging in the presence of WT CAV from a tandem genome. Further, that there was no increase in luminescence for CAV-nLuc1, 2, 3, or 8 suggests that the regions disrupted by the CAVector insert in these constructs play a necessary role in replication and/or packaging.

### Example 6: Purification of CAVector from Supernatant

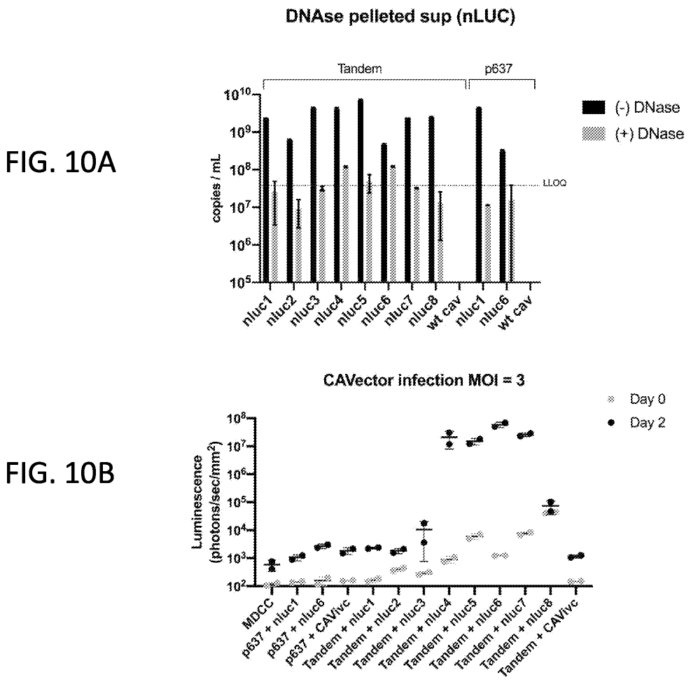

To concentrate supernatant CAVector particles and further reduce carryover nluc, 10 ml of CAVector supernatant was layered over a 20% sucrose cushion and centrifuged at 31,000 rpm for 3 hours to pellet virus and vector particles. Supernatant and sucrose were removed, and the pellet was resuspended overnight in PBS. A DNase protection assay was performed on the resuspended pellet using probes for CAV and CAVector. This confirmed successful purification of CAVector particles at a concentration of ˜1×10⁸ vectors/ml (FIG. 10A). Using the CAVector DNase protection assay as a guide, MDCC-MSB1 cells were transduced using a normalized amount of 3 CAVector genomes per cell. 3×10⁵ CAVector genomes were incubated with 1×10⁵ MDCC-MSB1 cells in a 48-well plate for 30 minutes or 48 hours and luminescence was measured after 3 PBS wash steps to determine if transduction occurred. The day 0 results indicated that the background nLuc signal was reduced (FIG. 10B). Further, the day 2 results confirmed that CAV-nLuc4-7 yielded vectors that transduced MDCC-MSB1 cells. Day 2 luminescence values were similar between CAV-nluc4-7, indicating these vectors transduced cells with similar efficiencies. This result also defines a window, spanning the 5′-most end of the nanoluciferase cassette of nluc4 and the 3′-most end of the cassette in construct nluc7, wherein a transgene can be inserted in the CAV genome to successfully recover a transduction-competent vector.

### Example 7: CAVectors Transduce Human Cells

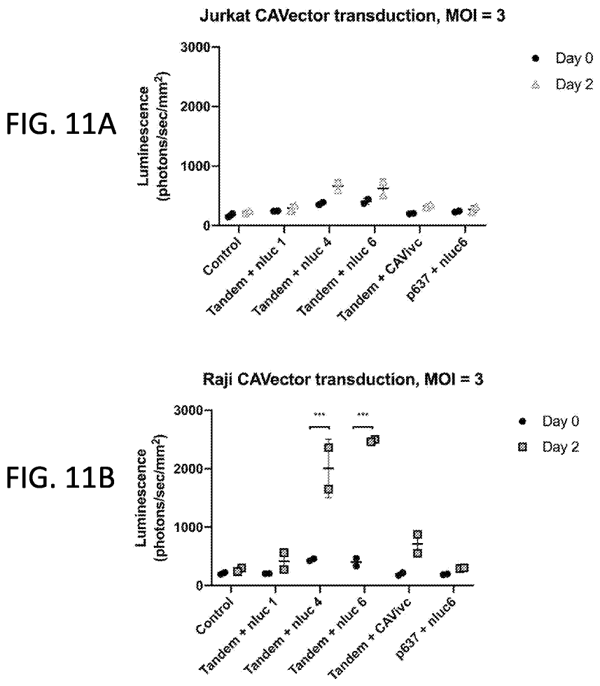

In this example, whether CAVectors could transduce Raji or Jurkat cells was assessed. CAV-nluc4 and CAV-nluc6 were used as test subjects, and CAV-nluc1, CAV WT, and p637+nluc 6 were used as expected negative controls. Mock-transduced cells were also used as a further negative control. 3×10⁵ CAVector genomes were incubated with 1×10⁵ Raji or Jurkat cells in a 48-well plate for 30 minutes or 48 hours and luminescence was measured after 3 PBS wash steps to determine if transduction occurred. In Raji cells, there was an increase in luminescence from day 0 to day 2 in cells transduced with CAV-nluc4 and CAV-nluc6 but not the negative controls (FIG. 11B). Jurkat cells transduced with CAV n-Luc4 and CAV-nLuc6 also showed an increase in luminescence from day 0 to day 2 (FIG. 11A). These data indicated that CAVectors are capable of transducing human cells.

In a second example, a transduction screen was performed in a large number of cell lines, using CAVector-nLuc at low (<10) multiplicity of infection (MOI) and using a sub-optimal promoter. The screens showed that, under these conditions, with an MOI of 3, transduction was detected in lymphoid cells, including MOLT-4 cells (luminescence increase of about 2.5× from day 0 to day 2), Jurkat cells (luminescence increase of about 0-2.5× from day 0 to day 2), and Raji cells (luminescence increase of about 4-13× from day 0 to day 2).

### Example 8: Resistance of CAVectors to Neutralizing Antibodies

### Resistance of CAVectors to Human IVIG and Neutralization of CAVectors by VP1-Specific Antibodies in Chicken Sera

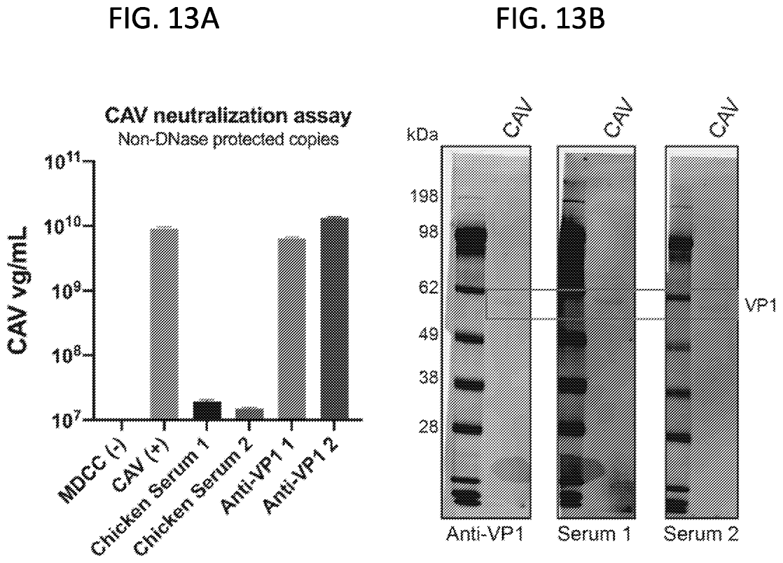

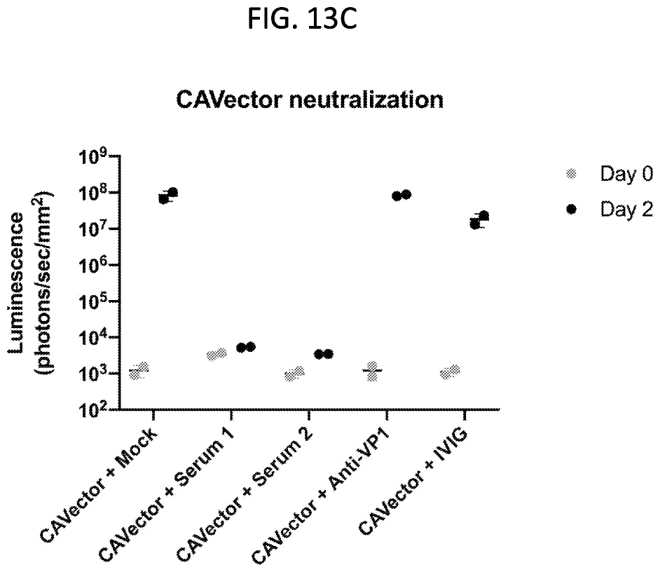

We assessed whether CAVectors were neutralized by anti-VP1 antibodies. Chicken sera from two independent sources were found to contain neutralizing antibodies to the VP1 capsid protein of CAV. CAV was incubated with either chicken serum or anti-VP1 antibodies raised against a VP1 peptide. Since many chickens are vaccinated against CAV, it was hypothesized that chicken sera would neutralize CAV. In addition, it was expected that the anti-VP1 peptide antibodies would not neutralize CAV since they are based on a peptide that is not conformationally relevant. Following incubation, cells were inoculated and total viral genomes were measured after 7 days. It was found that chicken sera neutralized CAV while anti-VP1 peptide antibodies did not (FIG. 13A). To prove that the neutralization was due to neutralizing VP1 antibodies in the chicken serum, western blots were performed on purified CAV particles using chicken serum as the source of antibody. Bands were observed that coincided with those detected by the anti-VP1 antibody, indicating that the neutralization was due to VP1-specific antibodies (FIG. 13B). To show that CAVectors were encapsidated by VP1, we tested if the neutralizing antibodies in chicken serum could block CAVector transduction. CAVector was incubated with chicken sera, the non-neutralizing VP1 antibody, or human intravenous immunoglobulin (IVIG). The transduction assay was then performed. Chicken serum neutralized CAVector transduction while anti-VP1 antibody and IVIG did not (FIG. 13C). This shows that CAVector transduction was mediated through VP1 and that CAVectors were encapsidated viral vectors.

### Resistance of CAVectors to Neutralizing Human Antibodies

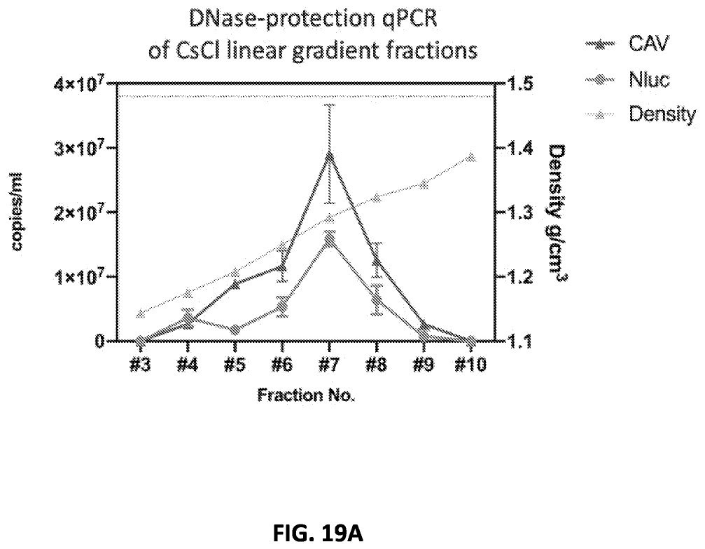

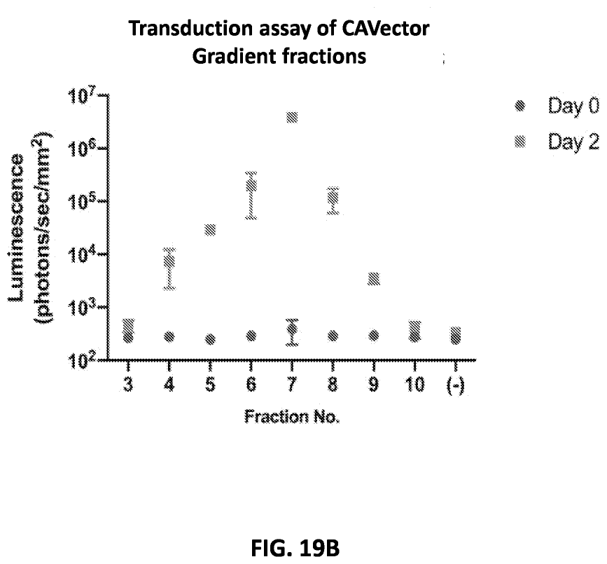

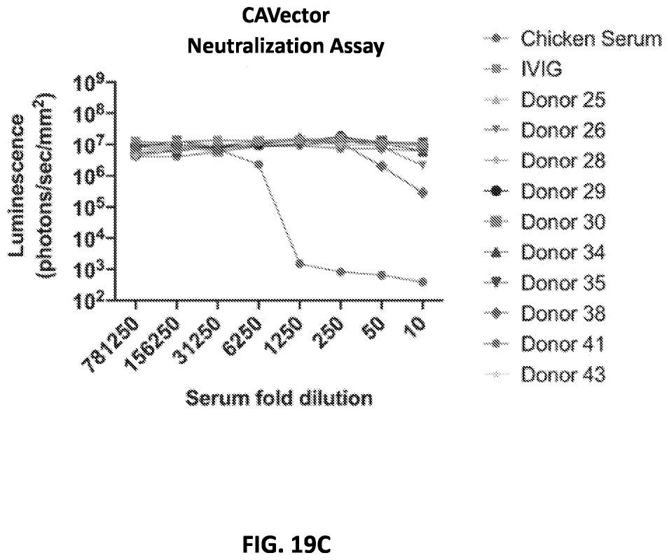

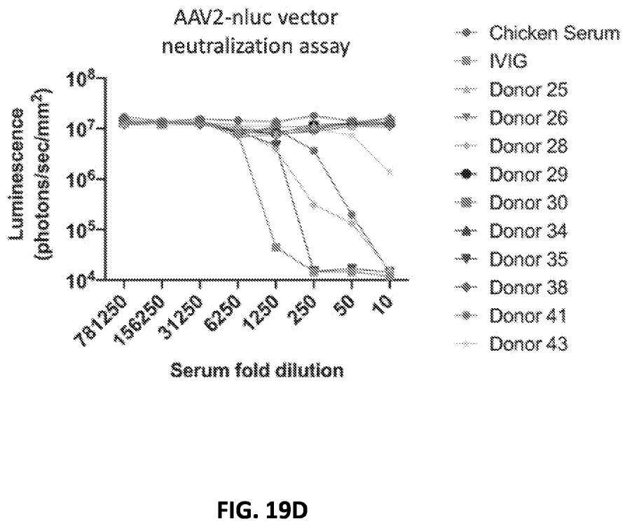

Further characterization was carried out by purifying the particles by isopycnic centrifugation in cesium chloride (CsCl) (FIG. 19A). A peak in DNase-protected vector copies (nanoluciferase amplicon) coincided with a peak in wildtype CAV particles as determined by qPCR, corresponding to a density of ˜1.29 g/mL. Fractions were dialyzed and found to transduce cells, yielding nanoluciferase luminescence signals proportional to the observed qPCR titers (FIG. 19B).

To further probe the susceptibility of CAVector to neutralization by human antibodies, 10 human serum samples were assayed along with positive and negative controls. Consistent with the initial IVIG observation, only one of the 10 samples showed any signs of neutralization at a dilution greater than 1:10 (FIG. 19C). By comparison, an adeno-associated virus 2 (AAV2) vector encoding the same nanoluciferase cassette was assayed with the same serum and control samples and shown to be neutralized in accordance with expectations based on previously published data (FIG. 19D). Three of 10 donor sera neutralized the AAV2 vector at dilutions greater than 1:10, and a 1:1250 dilution of IVIG potently neutralized the vector as well.

### Example 9: Production of CAVectors without Wild-Type CAV

This example demonstrates the provision in trans of CAV proteins to a CAVector comprising an exogenous gene, resulting in a CAVector preparation that did not detectably comprise wild-type CAV. The resulting CAVector was able to deliver the exogenous gene to a target cell.

As described in the Examples above, viral vectors have been produced based on CAV (CAVectors) by co-transfecting in vitro circularized (IVC) CAVector DNA expressing a nanoluciferase reporter with plasmid encoding a tandem CAV genome. From cell supernatants and lysates, bonafide CAVectors, were recovered, but the resultant preparations could include WT CAV particles due to the CAV tandem genome. Following this, efforts were undertaken to produce pure CAVectors in the absence of WT CAV. In place of the tandem CAV plasmid, cells were instead transfected with a plasmid encoding the full CAV genome (pCAV) that expressed the CAV proteins but could not make packaged particles. It was hypothesized that expression of the CAV proteins from their native promoter would be sufficient to replicate and package the co-transfected CAVector DNA. As a negative control, CAVector IVC was co-transfected with plasmid CAV in which the VP1 coding region has been deleted (pΔVP1). Thus, the negative control would not result in packaged CAVectors.

At 3 days post-transfection of pCAV and CAVector nLuc6 IVC into MDCC-MSB1 cells, the cell pellets were harvested and lysed and a partial purification was performed by ultracentrifugation over a 20% sucrose cushion. The post-centrifugation pellet was resuspended in PBS, and a transduction assay was performed on MDCC-MSB1 cells. To confirm that any transduction signal that might have been observed was due to VP1-encapsidated vectors, both conditions were treated with VP1-neutralizing antibodies (NAb) from chicken sera. Samples were pre-incubated with or without the VP1-neutralizing antibodies before transductions were performed. Samples were collected at 30 minutes or 2 days post-transduction to measure the change in luminescence as a readout for transduction.

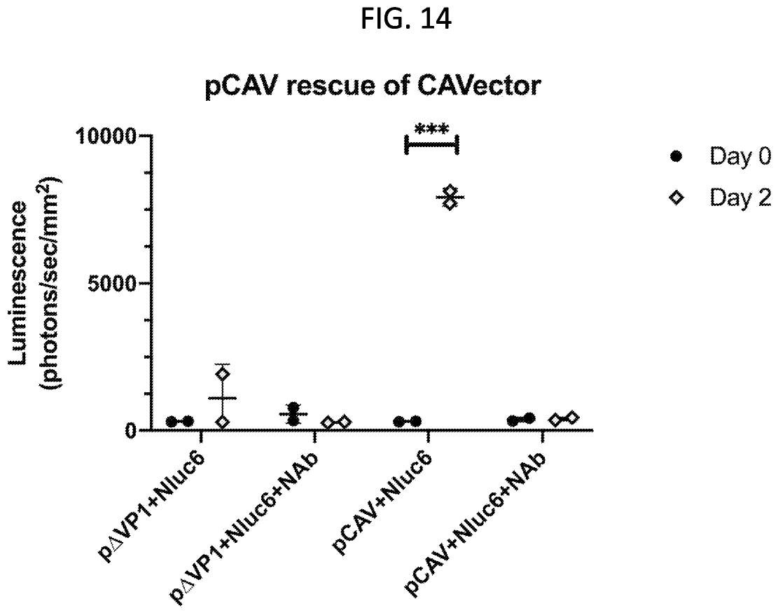

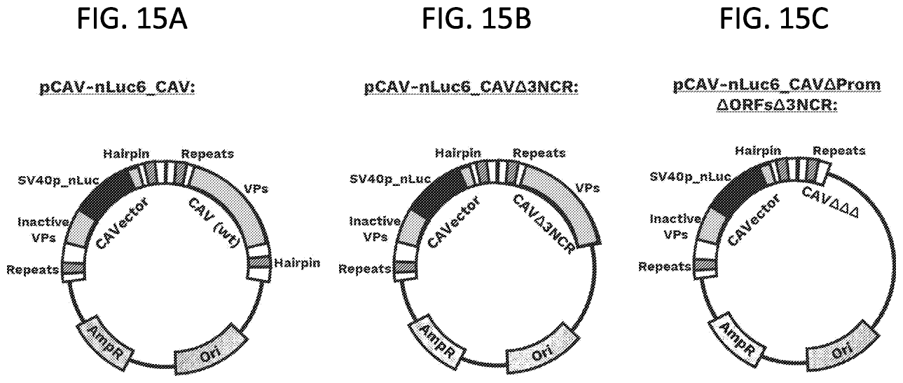

The negative control, pΔVP1, did not rescue CAVectors (FIG. 14). A 25-fold increase in luminescence was observed from day 0 to 2 in the pCAV+nLuc6 sample, and this signal was blocked by neutralizing antibodies, indicating that pCAV rescued CAVector successfully. There should not have been any replicating CAV in this sample, based at least on three lines of evidence. First, pCAV transfections had consistently showed a lack of wild-type CAV recovery. Second, excess DNase-protected CAV genomes were not detected in qPCR assays. Third, the observed luminescence signal was increased when wild-type CAV was added to the CAVector samples.

### Example 10: Production of nLuc CAVector for Injection into Mice

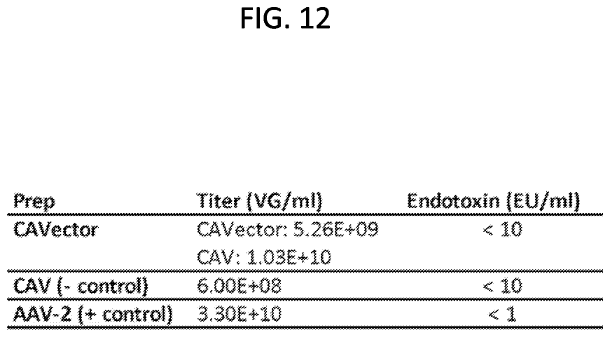

In this example, a CAVector carrying an nLuc transgene was produced and injected into mice. Briefly, 3e8 MDCC-MSB1 cells were transfected with nLuc6 CAVector constructs (as described herein, e.g., in Table 7) or CAV (negative control). Expi293 cells were transfected with AAV2 constructs as a positive control. The transfected cells were grown in 3 liters of media for 3 days. Supernatant was removed and the cell pellet was lysed in 0.5% SDS and treated with benzonase to digest unprotected nucleic acids. The lysed pellet was then run through a 0.45 μm filter. The filtered lysate is ultracentrifuged through a 20% sucrose cushion at 31,000 rpm for three hours, resuspended overnight, and then run through a CsCl linear gradient. The sample was then fractionated and the fractions were dialyzed, followed by quantification of the resultant concentrate. Quality checks were performed. The titer and endotoxin levels of the resultant preparations are shown in FIG. 12.

### Example 11: Administration of CAVector In Vivo to Mice, Resulting in Delivery of Payload DNA in Multiple Organs

In this example, a CAVector carrying a nano-luciferase (nLuc) payload was delivered into mouse tissues in vivo. Briefly, mice received comparable doses of either (i) a mixture of wild-type CAV (WT) and a CAVector carrying a genetic element encoding a nano-luciferase (Nluc) payload, or (ii) wild-type CAV alone, or (iii) AAV2 also encoding the nLuc payload. Various delivery routes were tested, including sub-retinal (SR), IV, IP, and IM (as shown in Table 23 below).

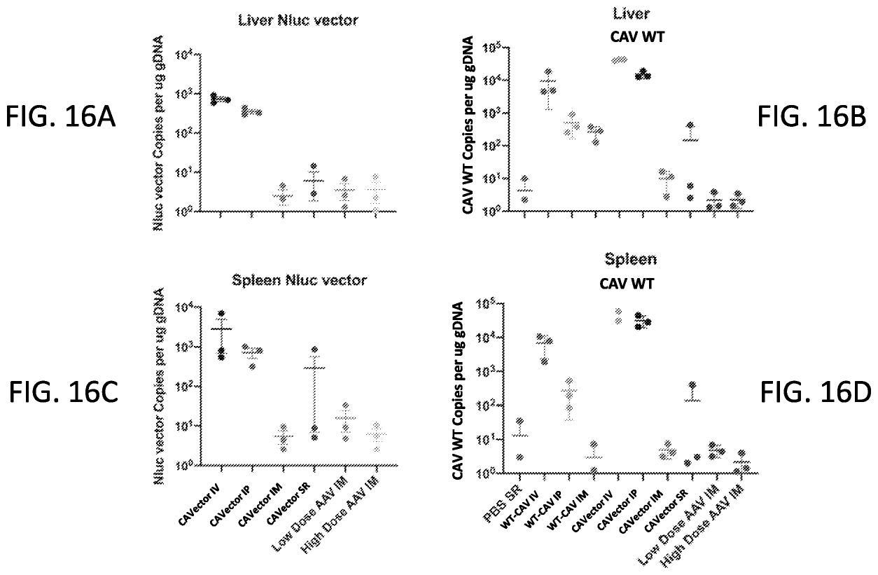

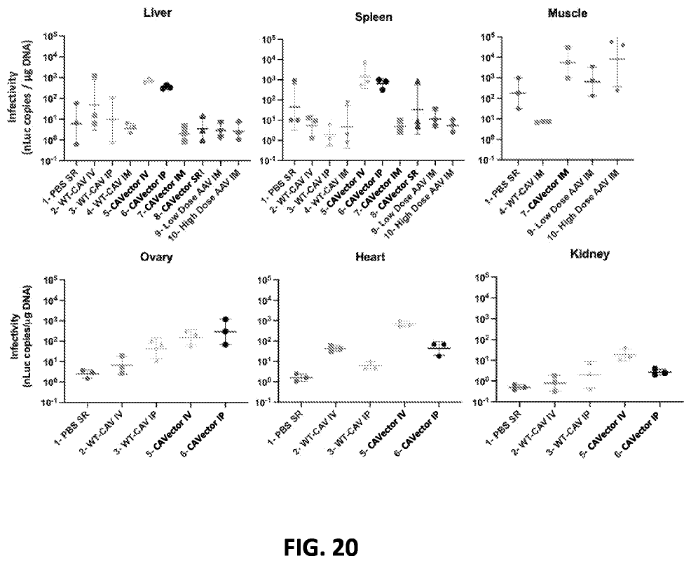

To assess tissue distribution of CAV and/or CAVector, animals were sacrificed 3 weeks after injection, and various tissues (blood, liver, spleen, lung, heart, ovary, muscle, brain, kidney, and retina) were collected. Total DNA was extracted from tissues, and assessed for CAVector genomes (using nLuc probes) or CAV WT virus genomes by qPCR. We detected elevated levels of CAVector (nLuc copies/μg DNA) in the spleens and livers of mice which received the CAVector via the IV and IP routes, relative to the AAV2 IM groups (FIGS. 16A and 16C). Furthermore, we detected wild-type CAV genomes in livers and spleens in mice administered either CAV WT or CAVector, including via IV and IP (FIGS. 16B and 16D). We also observed similar signals in the muscle, retinas, hearts, and ovaries, but not the brain. Vector was detected in tissues in a pattern consistent with the route of administration (FIG. 20). These data indicate successful in vivo delivery of CAVector DNA in mice.

TABLE 23
Dose and route of administration of CAVector and controls in mice

| | | | R46 Dose | R46 or AAV |
|---|---|---|---|---|
| Group # | Test Article | Route | (vg) | vector dose (vg) |
| 1 | PBS | Subretinal | 0 | 0 |
| 2 | CAV | IV | 6.00E+07 | 0 |
| 3 | CAV | IP | 6.00E+07 | 0 |
| 4 | CAV | IM | 1.50E+07 | 0 |
| 5 | CAVector-nLuc | IV | 9.51E+08 | 2.47E+07 |
| 6 | CAVector-nLuc | IP | 9.51E+08 | 2.47E+07 |
| 7 | CAVector-nLuc | IM | 2.38E+08 | 6.18E+06 |
| 8 | CAVector-nLuc | SR | 9.51E+06 | 2.47E+05 |
| 9 | AAV2-nLuc | IM | 0 | 1.30E+08 |
| 10 | AAV2-nLuc | IM | 0 | 8.30E+08 |

### Example 12: Immunogenicity in Mice Receiving CAVectors Versus AAV2

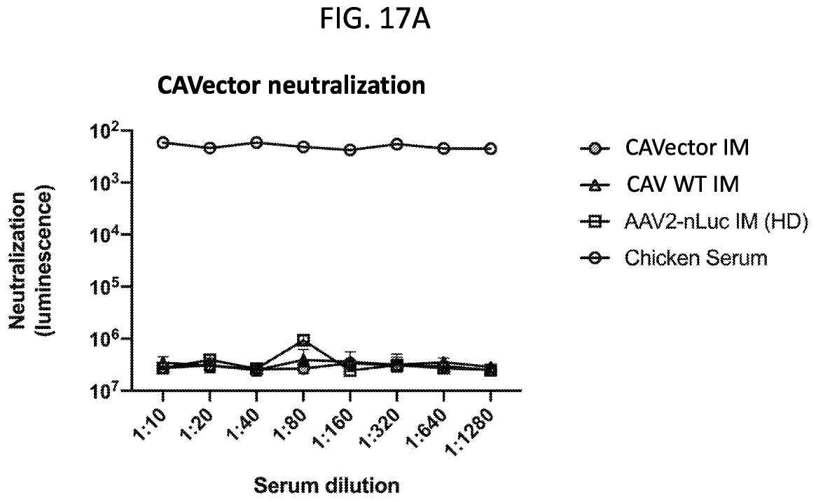

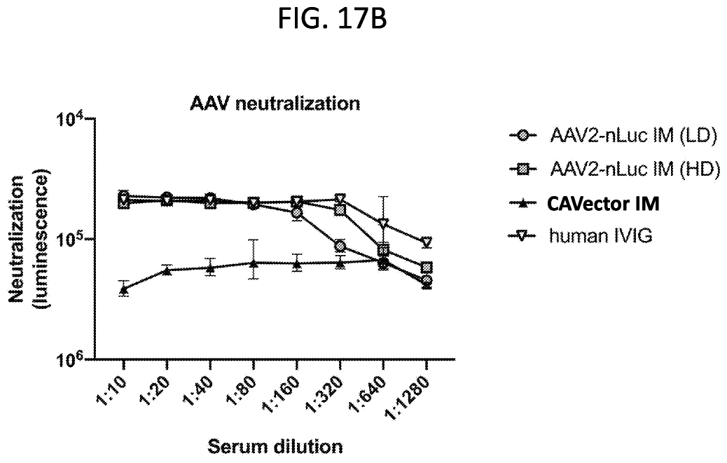

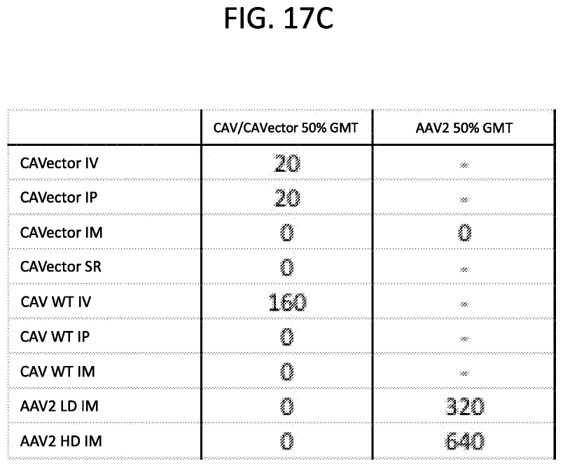

In this example, mice receiving wild-type CAV, CAVector, or AAV2 via intramuscular (IM) administration, as described in Example 11, were assessed for antibody responses against CAV or AAV2. Antibody responses against CAV or AAV2 were measured using an in vitro neutralization assay that relies on the detection of luminescence signal from the encoded nLuc transgene. When neutralizing antibodies against the administered vector (i.e., CAV or AAV2) is present in a sample, luminescence produced by the vector is reduced. Briefly, serum samples taken from the mice upon sacrifice were heat-inactivated and serially diluted. To compare the relative immunogenicity of CAV and AAV2, neutralization measurements were plotted (with luminescence on an inverted y-axis) for mice that received CAV or AAV2 by the IM route (FIG. 17A).

Chicken serum induced complete neutralization of CAVector at all dilutions tested, while the AAV2-nluc IM negative control, which would not be expected to elicit antibodies to CAV, did not (FIG. 19A). Remarkably, sera from mice which received CAVector and CAV WT IM did not neutralize CAVector. In contrast, animals given AAV2 IM produced high levels of neutralizing antibodies against AAV2, on par with titers present in human IVIG (FIGS. 17B and 17C). The 50% geometric mean neutralizing reciprocal titer (50% GMT) from AAV2-nLuc low dose and high dose animals was 320 and 640 respectively, compared with 1,280 for human IVIG. Given by other routes, CAVector and CAV WT elicited lower levels of neutralizing antibodies. The highest titer observed was 160 for CAV administered IV. These data suggest that CAVectors are less immunogenic than AAV2 vectors when administered intramuscularly.

### Example 13: CAV-Like Particles (VLPs) Assemble In Vitro from Purified Capsid Proteins

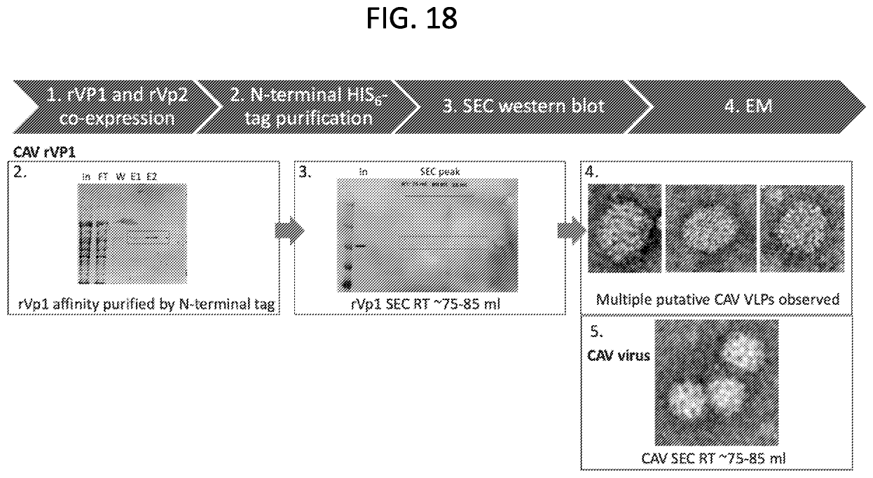

In this example, in vitro particle formation by the CAV capsid protein, VP1, was assessed as shown in the workflow at the top of FIG. 18. Briefly, recombinant VP1 (rVp1) was co-expressed in mammalian cells with CAV VP2 and was purified by virtue of an N-terminal affinity tag (FIG. 18, panel 2) followed by size exclusion chromatography (SEC). VP1 was eluted from SEC at a retention volume corresponding to the retention volume of wild type CAV virus (FIG. 18, panel 3). Particles similar to CAV were observed by electron microscopy in VP1 SEC fractions (FIG. 18, panel 4). The putative VP1 VLPs were approximately the same size as wild-type CAV particles and exhibited the same trumpet-shaped spike features present on wild type CAV virus (FIG. 18, panel 5). These results demonstrate that CAV VP1 protein can assemble into virus-like particles in vitro.

### Example 14: CAVector Enters MDCC-MSB1 Cells Via the Late Endosome Pathway

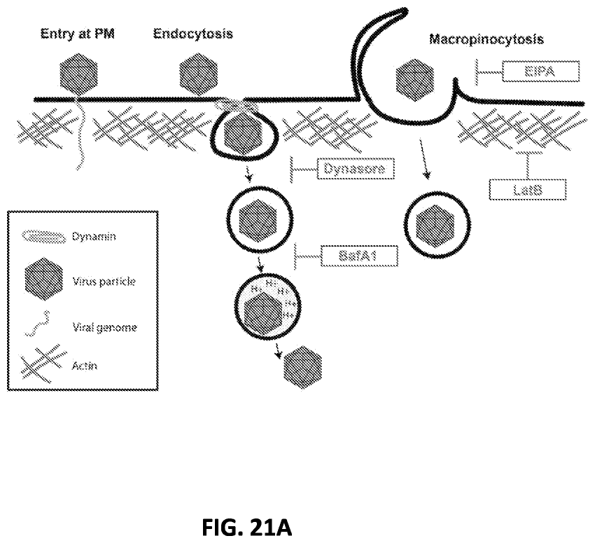

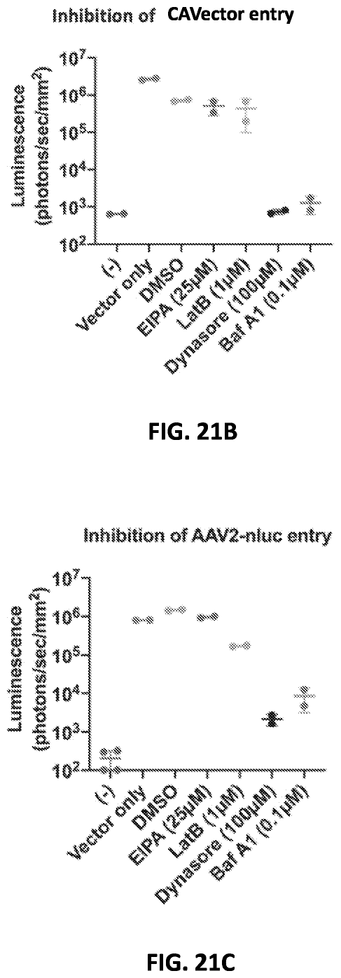

In this example, the mechanism of CAV viral entry into cells was assessed. Detection of CAVector transduction by luminescence assay enabled evaluation of the effect of four compounds known to inhibit different pathways for viral entry. The compounds tested included the macropinocytosis inhibitors amiloride hydrochloride (EIPA) and latrunculin B (LatB), dynasore, which inhibits an early event of endocytosis, and bafilomycin A1 (BafA1), an inhibitor of endosome acidification (FIG. 21A), were evaluated for their ability to inhibit transduction. The compounds, or a dimethylsulfoxide (DMSO) diluent control, were added to MDCC-MSB1 cells 15 minutes prior to and during a 20-hour transduction incubation with CAVector. A control experiment using AAV2-nLuc vector on Expi293 cells was also carried out.

Luminescence was measured to quantify CAVector transduction, showing that dynasore and BafA1 caused more than a thousandfold drop in signal relative to the DMSO diluent control while the other two compounds had minimal impact (FIG. 21B). In comparison, AAV2-nluc was inhibited to a similar extent by dynasore and BafA1, and about 3-fold by LatB (FIG. 21C). Cells were treated with each entry inhibitor in the absence of any viral vector as well as DMSO and no significant cell death was observed.

### Example 15: CAVector Viability after Heat Treatment and after Storage at 4° C.

To evaluate the stability of CAVectors, thermostability and storage stability were ascertained. The vector was purified by sedimentation of cell lysate through a 20% sucrose cushion, isopycnic CsCl centrifugation, and dialysis into phosphate-buffered saline (PBS) containing divalent cations and 0.001% poloxamer 188.

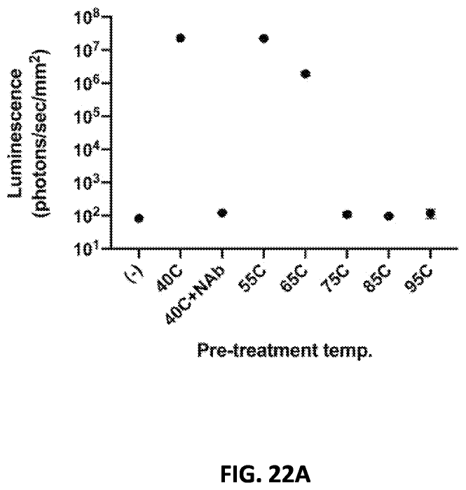

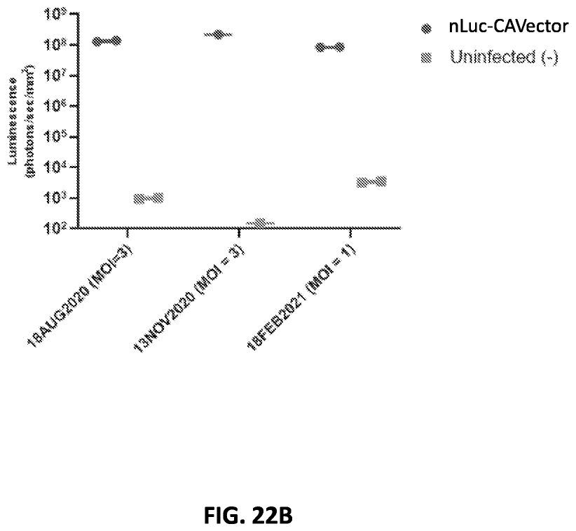

In a first example, a fifteen-minute heat treatment of the vector was carried out at temperatures ranging from 40° C. to 95° C. (FIG. 22A). Transduction signal was unchanged after a 55° C. treatment, was reduced 10-fold after 15 minutes at 65° C., and was eliminated after incubation at 75° C. A neutralization control (NAb) was included to rule out any spurious luminescence signal.

In a second example, the purified CAVector sample was stored at 4° C. over six months before the sample was depleted. During this time, 3 comparable tests of transduction potency were carried out on the material. The results are shown in FIG. 22B. The transduction signal remained unchanged during this time.

### Example 16: Recovery of CAVector Using Tandem Plasmid

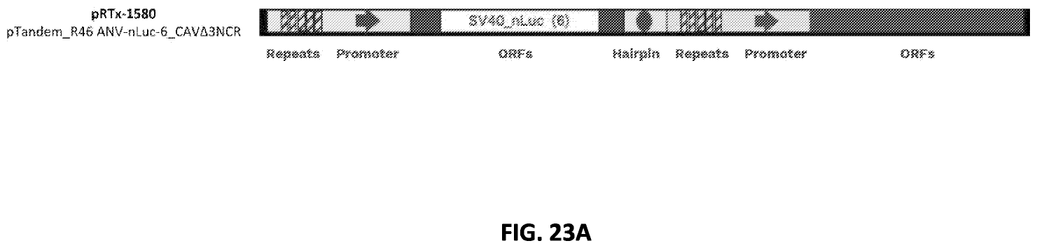

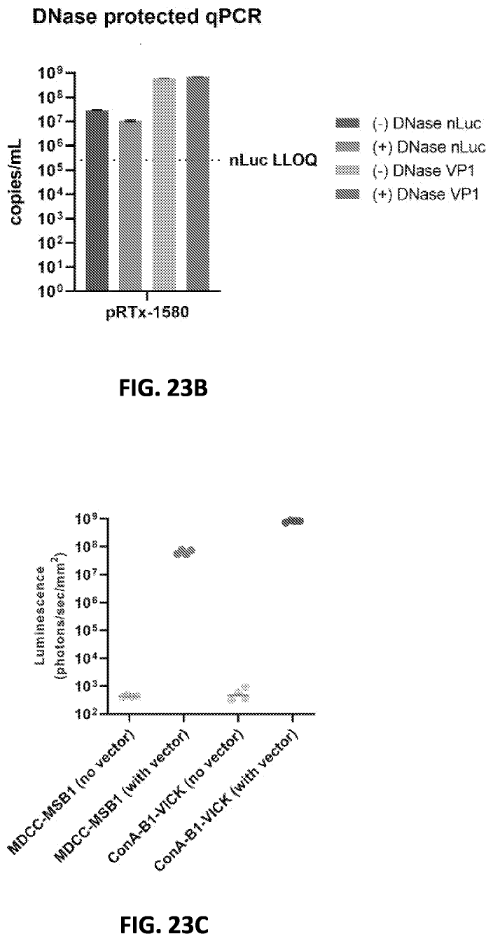

This example describes the recovery of an exemplary CAVector encoding the nano-luciferase (nLuc) gene using a tandem plasmid construct (pRTx-1580) comprising two CAVector genome copies. The schematic of the tandem construct is shown in FIG. 23A. The first genome copy encodes a nanoluciferase transgene in place of the partial ORFs but retaining the viral cis elements and rest of the ORFs. The second genome copy is the complete CAVector genome except for deletion of the 3′NCR.

To assess whether this construct can yield vectors, MDCC-MSB1 cells were transfected with the tandem vector construct. Transfected cells were harvested 3 days post transfection and lysed in using a 0.5% SDS lysis buffer containing 20 mM Tris (pH 8.0) for 30 min at 37° C. Lysates were then treated with 100 U/ml of benzonase for 20 minutes at 37 degrees, followed by clarification for 30 minutes at 10,000×g to pellet cell debris. The clarified lysate was subjected to sucrose cushion purification. Vector and wildtype genomes were quantified in the sucrose cushion purified material by qPCR following DNase treatment to remove residual non-encapsidated DNA.

As shown in FIG. 23B, DNAse-protected CAVector genome copies were recovered, indicating that that the pRTx-1580 tandem vector construct retains the ability for vector DNA replication and packaging in MDCC-MSB1 transfection. To test whether these DNAse protected vector genomes can transduce, sucrose cushion purified material was added to MDCC-MSB1 or ConA-B1-VICK cells. A clear increase of luminescence signal over background was observed in both MDCC-MSB1 and ConA-B1-VICK transductions (FIG. 23C), indicating that the pRTx-1580 construct generated vector particles that were capable of transduction.

What is claimed is:

1. A genetic element comprising:
a promoter element;
a nucleic acid sequence encoding an exogenous effector (e.g., a therapeutic exogenous effector), and
a protein binding sequence that specifically binds a CAV capsid polypeptide (e.g., a CAV VP1 molecule), e.g., with an affinity/specificity of less than about 10 μM (e.g., less than about 10, 9, 8, 7, 6, 5, 4, 3, 2, or 1 μM, e.g., less than about 900, 800, 700, 600, 500, 400, 300, 200, 100, 90, 80, 70, 60, 50, 40, 30, 20, or 10 nM).

2. A genetic element comprising:
a promoter element;
a nucleic acid sequence encoding an exogenous effector (e.g., a therapeutic exogenous effector), and
a protein binding sequence;
wherein the genetic element is capable of being packaged (e.g., specifically packaged) by a CAV VP1 molecule.

3. A genetic element comprising:
a promoter element;
a nucleic acid sequence encoding an exogenous effector (e.g., a therapeutic exogenous effector), and
a protein binding sequence that specifically binds to a CAV capsid polypeptide;
wherein the exogenous effector is:
(a) codon optimized for expression in a human cell,
(b) a human polypeptide or nucleic acid,
(c) binds a human polypeptide or nucleic acid, or
(d) has an activity in a human cell, e.g., modulates (e.g., increases or decreases) the activity and/or level of a human gene in the human cell).

4. A genetic element comprising:
a promoter element;
a nucleic acid sequence encoding an exogenous effector (e.g., a therapeutic exogenous effector), and
a nucleic acid sequence having at least 75%, 80%, 85%, 90%, 95%, 96%, 97%, 98%, 99%, or 100% sequence identity to nucleotides 1-374 of SEQ ID NO: 1, and/or a nucleic acid sequence having at least 75%, 80%, 85%, 90%, 95%, 96%, 97%, 98%, 99%, or 100% sequence identity to nucleotides 2195-2319 of SEQ ID NO: 100.

5. A genetic element comprising:
a promoter element;
a nucleic acid sequence encoding an exogenous effector (e.g., a therapeutic exogenous effector), and
at least 100, 200, 300, 400, 500, 600, 700, 800, 900, 1,000, 1,500, 2,000, 2,500, or 3,000 nucleotides having at least 70%, 75%, 80%, 85%, 90%, 95%, 96%, 97%, 98%, 99%, or 100% sequence identity to a contiguous portion of a e.

6. A genetic element comprising:
a protein binding sequence that specifically binds a CAV capsid polypeptide e.g., with an affinity/specificity of less than about 10 μM (e.g., less than about 10, 9, 8, 7, 6, 5, 4, 3, 2, or 1 μM, e.g., less than about 900, 800, 700, 600, 500, 400, 300, 200, 100, 90, 80, 70, 60, 50, 40, 30, 20, or 10 nM),
wherein the genetic element does not comprise one or more of:
(i) a full length CAV VP1 gene (e.g., wherein the genetic element comprises one or more fragments of the CAV VP1 gene, e.g., less than about 500, 400, 300, 200, or 100 nucleotides of CAV VP1 gene sequence);
(ii) a full length CAV VP2 gene (e.g., wherein the genetic element comprises one or more fragments of the CAV VP2 gene, e.g., less than about 500, 400, 300, 200, or 100 nucleotides of CAV VP2 gene sequence); or
(ii) a full length CAV Apoptin gene (e.g., wherein the genetic element comprises one or more fragments of the CAV Apoptin gene, e.g., less than about 500, 400, 300, 200, or 100 nucleotides of CAV Apoptin gene sequence).

7. A genetic element comprising:
a protein binding sequence that specifically binds a CAV capsid polypeptide e.g., with an affinity/specificity of less than about 10 μM (e.g., less than about 10, 9, 8, 7, 6, 5, 4, 3, 2, or 1 μM, e.g., less than about 900, 800, 700, 600, 500, 400, 300, 200, 100, 90, 80, 70, 60, 50, 40, 30, 20, or 10 nM),
wherein the genetic element comprises one or more of:
(i) a nonfunctional CAV VP1 gene or a fragment thereof (e.g., a contiguous fragment of at least 25, 50, 100, 200, 300, 400, 500 or more bp), e.g., comprising a stop codon within the sequence of the CAV VP1 coding sequence, e.g., at the 5′ end of the CAV VP1 coding sequence;
(ii) a nonfunctional CAV VP2 gene or a fragment thereof (e.g., a contiguous fragment of at least 25, 50, 100, 200, 300, 400, 500 or more bp), e.g., comprising a stop codon within the sequence of the CAV VP2 coding sequence, e.g., at the 5′ end of the CAV VP2 coding sequence; or
(ii) a nonfunctional CAV Apoptin gene or a fragment thereof (e.g., a contiguous fragment of at least 25, 50, 100, 200, 300, 400, 500 or more bp), e.g., comprising a stop codon within the sequence of the CAV Apoptin coding sequence, e.g., at the 5′ end of the CAV Apoptin coding sequence.

8. A nucleic acid construct comprising the nucleic acid sequence of a genetic element of any of claims 1-7.

9. A nucleic acid construct (e.g., a plasmid) comprising one, two, or all three of:
(a) a CAV VP1 gene, or a nucleic acid sequence having at least 75%, 80%, 85%, 90%, 95%, 96%, 97%, 98%, or 99% sequence identity thereto;
(b) a CAV VP2 gene, or a nucleic acid sequence having at least 75%, 80%, 85%, 90%, 95%, 96%, 97%, 98%, or 99% sequence identity thereto; and/or
(c) a CAV Apoptin gene, or a nucleic acid sequence having at least 75%, 80%, 85%, 90%, 95%, 96%, 97%, 98%, or 99% sequence identity thereto;
wherein the nucleic acid construct does not comprise a CAV packaging signal, and/or wherein the nucleic acid construct is incapable of being packaged by a CAV VP1 molecule.

10. A host cell (e.g., a vertebrate cell, e.g., (i) a mammalian cell, e.g., a human cell; or (ii) an avian cell, e.g., a chicken cell) comprising a genetic element of any of claims 1-7, or a nucleic acid construct of claim 8 or 9.

11. A CAVector comprising:
a) a proteinaceous exterior comprising a CAV VP1 molecule;
b) a genetic element comprising: (i) a promoter element, (ii) a nucleic acid sequence encoding an exogenous effector, and (iii) a protein binding sequence that specifically binds the CAV VP1 molecule.

12. A CAVector comprising:
a) a genetic element of any of claims 1-7, and
b) a proteinaceous exterior, e.g., a proteinaceous exterior comprising a CAV VP1 molecule.

13. A CAVector comprising:
a) a genetic element of any of claims 1-7, and
b) a capsid, e.g., a capsid comprising a CAV VP1 molecule.

14. A complex comprising:
a CAV VP1 molecule bound to a genetic element,
wherein the genetic element comprises: (i) a promoter element, (ii) a nucleic acid sequence encoding an exogenous effector, and (iii) a protein binding sequence.

15. A method of delivering an exogenous effector to a target cell (e.g., a vertebrate cell, e.g., a mammalian cell, e.g., a human cell), the method comprising introducing into the cell a CAVector of any of claims 11-13.

16. A method of delivering an exogenous effector to a target cell (e.g., a vertebrate cell, e.g., a mammalian cell, e.g., a human cell), the method comprising:
(a) assessing the target cell, or a subject comprising the target cell, for the presence of an unwanted immune response to CAV, e.g., an anti-CAV antibody, e.g., a CAV neutralizing antibody; and
(b) introducing into the cell a CAVector of any of claims 11-13.

17. A method of selecting a subject for receiving a CAVector, the method comprising assessing the subject for the presence of an unwanted immune response to CAV, e.g., an anti-CAV antibody, e.g., a CAV neutralizing antibody.

18. A method of modulating a biological activity in a subject in need thereof, the method comprising introducing into the subject a CAVector of any of claims 11-13, e.g., wherein the disease or disorder is cancer.

19. A method of treating a disease or disorder in a subject in need thereof, the method comprising introducing into the subject a CAVector of any of claims 11-13, e.g., wherein the disease or disorder is cancer.

20. A method of treating a disease or disorder in a subject in need thereof, the method comprising:
(a) assessing the subject for the presence of an unwanted immune response to CAV, e.g., an anti-CAV antibody, e.g., a CAV neutralizing antibody; and
(b) introducing into the subject a CAVector of any of claims 11-13.

21. A method of vaccinating a subject in need thereof, the method comprising introducing into the subject a CAVector of any of claims 11-13, wherein the exogenous effector comprises an antigen from an infectious agent (e.g., a virus or bacteria).

22. A method of making a CAVector, comprising:
a) providing a host cell comprising a genetic element of any of claims 1-7, and
b) incubating the host cell under conditions suitable for enclosure of the genetic element in a proteinaceous exterior (e.g., a proteinaceous exterior comprising a CAV VP1 molecule),
thereby making the CAVector.

23. A method of storing a composition comprising a CAVector, the method comprising maintaining a composition comprising a CAVector (e.g., a CAVector as described herein) at a temperature between 1-5° C., 5-10° C., 10-15° C., 15-20° C., or 20-25° C. for a period of at least about 1 week, 2 weeks, 3 weeks, 4 weeks, 2 months, 3 months, 4 months, 5 months, 6 months, 7 months, 8 months, 9 months, 10 months, 11 months, 1 year, or 2 years, or a period of about 1-2 weeks, 2-3 weeks, 3-4 weeks, 1-2 months, 2-3 months, 3-4 months, 4-5 months, 5-6 months, 6-7 months, 7-8 months, 8-9 months, 9-10 months, 10-11 months, 11-12 months, 12-18 months, 18-24 months, or 2-3 years.

24. A method of cooling a composition comprising a CAVector, the method comprising lowering the temperature of a composition comprising a CAVector (e.g., a CAVector as described herein) to about 1, 2, 3, 4, 5, 6, 7, 8, 9, or 10° C., or to between 1-5° C. (e.g., about 4° C.).

25. A method of heating a composition comprising a CAVector, the method comprising raising the temperature of a composition comprising a CAVector (e.g., a CAVector as described herein) to about 20, 21, 22, 23, 24, 25, 26, 27, 28, 29, or 30° C., or to between 20-25° C. or 25-30° C. (e.g., about 25° C.).

26. A method of heating a composition comprising a CAVector, the method comprising raising the temperature of a composition comprising a CAVector (e.g., a CAVector as described herein) to about 35, 36, 37, 38, 39, or 40° C., or to between 30-35° C. or 35-40° C. (e.g., about 37° C.).
